SEQUENCE LISTING (219 sequences)

SEQ ID NO: 1 - protein, 268 aa, Mirabilis jalapa
MKGTYYINHGDPLMYLKKHIKLRQFLEGWQENVVIEKPKSILIISAHWDTNVPTVNFVEHCDTIHDFDDYPDPLYQIQYRAPGAPNLAKKVEELLKESGMECEIDTKRGLDHAAWFPLMFMYPEANIPICELSVQPSKDGIHHYNVGKALSPLLQQGVLIIGSGGTVHPSDDTPHCPNGVAPWAIEFDNWLEDALLSGRYEDVNNFKKLAPNWEISHPGQEHLYPLHVALGAAGKNPKTQLIHRSWAANGVFGYSTYNFTPTTQKTDV

SEQ ID NO: 2 - DNA, 807 bp, Synthetic construct
  source 1..807: mol_type = other DNA
atgaagggaacctactacatcaaccacggtgacccattgatgtacttgaagaagcacatcaagttgagacaattcttggaaggttggcaagaaaacgttgttatcgaaaagccaaagtctatcttgatcatcagtgcacactgggacaccaacgttccaaccgttaacttcgttgaacactgtgacaccatccacgacttcgacgactacccagacccattgtaccaaatccaataccgcgctccaggtgctccaaacttggctaagaaggttgaagaattgcttaaggaatctggtatggaatgtgaaatcgacaccaagagaggtttggaccacgctgcttggttcccattgatgttcatgtacccagaagctaacatcccaatctgtgaattgtctgttcaaccatctaaggacggtatccaccactacaacgttggtaaagctttgtctccattgttgcaacaaggtgttttgatcatcggttctggtggaaccgttcacccatctgacgacaccccacactgtccaaacggtgttgctccatgggctatcgagttcgacaactggttggaagacgctttgctctcgggtagatacgaagacgttaacaacttcaagaagttggctccaaactgggaaatctctcacccaggtcaagaacacttgtacccattgcacgttgctttgggtgctgctggtaagaacccaaagacccaattgatccaccggtcttgggctgctaacggtgttttcggttactctacctacaacttcaccccaaccacccaaaagaccgacgtgtaa

SEQ ID NO: 3 - DNA, 807 bp, Synthetic construct
  source 1..807: mol_type = other DNA
atgaagggcacctactacatcaaccacggcgaccctctgatgtacctgaagaagcacatcaagctgcgacagtttctggaaggctggcaagagaacgtggtgatcgagaagcccaagtctatcctgatcatctctgcccactgggacaccaacgtgcccaccgtgaacttcgtcgagcactgcgacaccatccacgacttcgacgactaccccgatcctctgtaccagatccagtacagagcccctggcgctcccaacctggccaagaaggtcgaggaactgctgaaggaatctggcatggaatgcgagatcgacaccaagcgaggcctggaccacgccgcatggttccctctcatgttcatgtaccccgaggccaacattcccatctgcgagctgtctgtgcagccctctaaggacggcatccaccactacaacgtcggcaaggctctgtctcccctgctccagcagggcgtgctgatcatcggctctggcggcaccgtgcatccctctgacgacacccctcactgccccaacggcgtggctccctgggccatcgagttcgacaactggctcgaggacgctctgctgtctggccgatacgaggacgtgaacaacttcaagaagctggctcccaactgggagatctctcaccccggccaagagcatctgtaccctctgcacgtggccctgggcgctgccggcaagaaccccaagactcagctgatccaccgatcttgggccgctaacggcgtgttcggctactctacctacaacttcacccctaccactcaaaagaccgacgtgtaa

SEQ ID NO: 4 - protein, 497 aa, Beta vulgaris
MDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 5 - DNA, 1494 bp, Synthetic construct
  source 1..1494: mol_type = other DNA
atggatcacgcaacattagctatgatattagcaattttgtttatttcatttcattttattaaattattattctcacaacaaacaacaaaattgttacctccaggtcctaaacctttaccaattattggtaatatcttggaagtaggaaagaagcctcatagatcatttgcaaatttggctaagatccacggtcctttaatctctttgaggttaggatcagttaccaccattgttgtatcatctgctgatgtcgccaaagagatgttcttgaagaaggatcaccctttatcaaacaggacaattccaaattcagtaactgctggtgaccaccacaaattgactatgtcttggttgcctgtttcacctaaatggagaaacttcagaaagataactgctgttcatttattgtctccacaaagattagatgcatgccagacctttagacatgcaaaggttcagcaattatatgaatacgttcaagagtgcgctcagaagggtcaggcagttgacattggtaaagctgccttcacaacttctttgaatttattgtctaaattattcttttctgttgaattggcccatcataaatcacatacctctcaagagtttaaggaattaatttggaatattatggaagacatcggtaaaccaaactacgccgattacttcccaattttaggttgcgtcgatccatcaggaattagaagaaggttggcatgttcttttgataagttaattgcagtatttcaaggaataatatgtgagaggttagctccagattcatcaactacaactactacaactacagatgatgtcttggacgtattattgcaattgtttaagcagaatgaattaactatgggagagattaaccacttgttagtcgatattttcgatgccggtactgatactacatcatctacctttgagtgggttatgactgagttaataagaaaccctgaaatgatggaaaaggcccaagaggaaataaaacaggtattgggtaaggataagcaaatccaagagtcagacataataaatttgccatacttgcaagcaatcattaaggaaactttgaggttgcacccacctacagttttcttgttgcctagaaaagccgacacagacgtagaattgtacggatacattgttccaaaggatgctcaaattttggtaaacttgtgggccataggtagagatccaaatgcttggcaaaatgccgatattttctcacctgaaagatttataggttgtgagattgatgtaaagggaagagattttggtttattgccttttggtgctggtagaagaatttgccctggtatgaatttggccataaggatgttgacattgatgttggccacattattgcaattcttcaactggaaattagagggagacatttcacctaaagatttggacatggatgaaaagtttggtatagccttgcagaaaactaaacctttaaaattgatcccaattccaagatactaa

SEQ ID NO: 6 - protein, 494 aa, Ercilla volubilis
MDHTTLAMILSAIFLLYNLAKAIFSHSNTKLPPGPKPVPIFGNIFELGEKPHRSFANLAKIHGPLITLKLGSVTTIVVSSAEVAKEMFLKNDLPLANRNVPNSVTAGDHHKLTMSWLPVSPKWKTFRKITAVHLLSPQRLDSCQALRHTKVKQLHQYVQECAKRGEPVDIGKAAFTTSLNLLSNLFFSVELANHTSSSSQEFKELIWEIMEDIGKPNYADYFPILKCVDPWGIRRRLASNFDKLIEVFQGFIRKRLSTGSFSAITPNDVLDVLLNLLKEKELNMGEINHLLVDIFDAGTDTTSSTFEWAMAELVRNQEMMKKAQDEIEQVLGKDAIIQESDIPKMPYLQAIIKETLRLHPPTVFLLPRKATSNVELYGYVVPKNAQILVNLWAIGRDPKVWDNPNMFSPERFLNSEIDVKGRDFGLLPFGAGRRICPGMNLAYRMLTLMLATLLQSFDWKLGDGVNPKDLDMEEKFGIALQKTKPLQVIPVLKY

SEQ ID NO: 7 - DNA, 1485 bp, Synthetic construct
  source 1..1485: mol_type = other DNA
atggaccacaccactctggccatgatcctgtctgccatcttcctgctgtacaacctggccaaggccatcttctctcactctaacaccaagctgcctccaggacctaagcctgtgcctatcttcggcaacatcttcgagctgggcgagaagccccaccgatctttcgccaacctggctaagattcacggacccctgatcaccctgaagctgggctctgtgactaccatcgtggtgtcctctgccgaggtggccaaggaaatgttcctgaagaacgacctgcctctggccaaccgaaacgtgcccaactctgtgaccgctggcgaccaccacaagctgaccatgtcttggctgcccgtgtctcccaagtggaagaccttccgaaagatcaccgccgtgcatctgctgtctccccagcgactggactcttgtcaggccctgcgacacaccaaggtgaagcagctgcaccagtacgtgcaagagtgcgccaagcgaggcgagcccgtggacatcggcaaggccgccttcaccacctcgctgaacctgctgtctaacctgttcttctcggtcgagctggctaaccacacctcttcgtcctctcaagagttcaaggaactgatctgggagatcatggaagatatcggcaagcccaactacgccgactacttccccatcctgaagtgcgtggacccctggggcatccgacgacgactggcctctaacttcgacaagctgatcgaggtgttccagggcttcatccgaaagcgactgtctaccggctctttctctgccatcactcccaacgacgtgctggacgtgctcctgaacctcctgaaggaaaaggaactcaacatgggcgagatcaaccacctcctggtggacattttcgacgccggcaccgacaccacctcttctaccttcgagtgggccatggctgagctggtgcgaaaccaagagatgatgaagaaggcccaggacgagatcgagcaggtcctcggaaaggacgccatcatccaagagtctgacatccccaagatgccctacctgcaggccatcatcaaggaaaccctgcgactgcaccctcctaccgtgtttctgctgccccgaaaggccacctctaacgtcgagctgtacggctacgtggtgcccaagaacgcccagatcctggtgaacctgtgggccatcggtcgagatcccaaggtgtgggacaaccccaacatgttctcgcccgagcgattcctgaactccgagatcgacgtgaagggccgagacttcggactgctgcccttcggagccggccgacgaatctgccccggcatgaacctggcctaccgaatgctgaccctgatgctggctaccctgctgcagtctttcgactggaagctcggcgacggcgtgaaccccaaggacctggacatggaagagaagttcggcattgccctgcaaaagaccaagcctctgcaggtcatccccgtgctgaagtactaa

SEQ ID NO: 8 - protein, 476 aa, Beta vulgaris
MDDKSQQLHIVLFPFMAHGHMIPTLDIARLFAARGVKTTLITTPRNAPTFLTAIEKGNKSGAPTINVEVFNFQAQSFGLPEGCENLEQALGPGIRDRFFKAAAMLRDQLEHFLEKTRPNCLVADMFFPWATDSAAKFNIPRLVFHGHCLFALCALEIIRLHEPYNNASSDEEPFLLPHLPHEIELTRLQFSEELWKNGGDSDYKERSKAIKESELKCYGVLVNSFYELEPDYAEYFRKDLGRRAWNIGPVSLYNRSNEEKAQRGKQASIDEHECLKWLNSKKPNSVIYICFGSTMHMIPSQLNEIAMGLEASGKDFIWVVRNEDDLGEFEHRMEGKGLIIRGWAPQVLILEHEVIGAFVTHCGWNSTIEGIAAGVPMVTWPVFAEQFLNEKLITRVLRIGIPVGAKKWDCKPCEEYVVKKNDIEKALREVMEGNEAEERRTRAKEYKEMAWKALQEGGSSYSDLSALIDELRGLST

SEQ ID NO: 9 - DNA, 1431 bp, Synthetic construct
  source 1..1431: mol_type = other DNA
atggacgacaagtcccaacaattgcacatagttttgtttccattcatggcccatggtcatatgattccaactttggatattgctaggttgtttgctgctagaggtgttaagactactttgattactactccaagaaacgctccaactttcttgactgctattgaaaagggtaacaaatctggtgctccaaccattaacgttgaggtttttaacttccaagctcagtcttttggtttgcctgaaggttgtgaaaatttggaacaagctttaggtccaggtatcagagatagatttttcaaagctgctgccatgttgagagatcaattggaacatttcttggaaaagaccagaccaaattgcttggttgctgatatgttttttccatgggctactgattctgctgccaagttcaatattccaagattggtttttcatggccattgcttgttcgctttgtgtgctttggaaatcatcagattgcatgaaccttacaacaacgcctcttctgatgaagaaccatttttgttaccacacttgccacacgaaattgaattgaccagattgcagttctctgaagagttgtggaagaatggtggtgattccgattacaaagaaagatccaaggccatcaaagaatccgaattgaaatgttacggtgtcctggttaactccttctatgaattggaaccagattacgccgaatacttcagaaaagatttgggtagacgtgcatggaacattggtccagtttcattatacaacaggtccaacgaagaaaaggctcaaagaggtaaacaagcctccattgatgaacatgaatgtctgaagtggctgaactctaagaagccaaattccgttatctacatctgtttcggttctaccatgcatatgatcccatctcaattgaacgaaatcgctatgggtttagaagcttccggtaaagatttcatctgggttgttagaaacgaagatgacttgggtgaattcgaacatagaatggaaggtaagggtttgattattagaggttgggctccacaagtcttgatcttggaacatgaagttattggtgctttcgttactcattgtggttggaactctaccattgaaggtattgctgctggtgttccaatggttacttggccagtttttgctgaacaattcttgaacgagaagttgatcaccagagttttgagaattggtattccagttggtgctaaaaagtgggattgcaaaccatgtgaagaatacgttgttaagaagaacgatatcgaaaaggccttgagagaagttatggaaggcaatgaagctgaagaaagaagaactagagccaaagagtacaaagagatggcttggaaggctttacaagaaggtggttcatcttactctgatttgtccgctttgatcgatgaattgagaggtttgtctacctaa

SEQ ID NO: 10 - DNA, 1431 bp, Synthetic construct
  source 1..1431: mol_type = other DNA
atggacgacaagtctcagcagctgcacatcgtgctgttccctttcatggcccacggccacatgatccccactctggacattgcccgactgttcgccgctcgaggcgtcaagaccactctgatcaccactcctcgaaacgctcccaccttcctgaccgccatcgagaagggcaacaagtctggcgcccctaccatcaacgtcgaggtgttcaacttccaggctcagtctttcggactgcccgagggctgcgagaacctcgagcaggctctcggacccggcatccgagatcgattcttcaaggccgctgccatgctgcgagatcagctcgagcacttcctggaaaagacccgacctaactgcctggtggccgacatgttcttcccttgggccaccgactctgccgccaagttcaacatccctcgactggtgttccacggacactgcctgttcgccctgtgcgccctcgagatcatccgactgcacgagccctacaacaacgcctcttctgacgaggaacccttcctgctgcctcatctgccccacgagatcgagctgacccgactgcagttctctgaggaactgtggaagaacggcggcgactctgactacaaggaacgatctaaggccatcaaggaatctgagctgaagtgctacggcgtgctggtgaactctttctacgagctggaacccgactacgccgagtacttccgaaaggacctgggccgacgagcttggaacatcggccccgtgtctctgtacaaccgatctaacgaggaaaaggcccagcgaggcaagcaggcctctatcgacgagcacgagtgcctgaagtggctgaactctaagaagcccaactctgtcatctacatctgcttcggctctaccatgcacatgattccctcgcagctgaacgagatcgccatgggactcgaggcctctggcaaggacttcatctgggtcgtgcgaaacgaggacgacctgggcgagttcgagcaccgaatggaaggcaagggcctgatcattcgaggctgggctccccaggtgctgatcctggaacacgaggtgatcggcgccttcgtgacccactgcggctggaactctaccatcgagggcattgccgctggcgtgcccatggtgacctggcctgtgttcgccgagcagtttctcaacgagaagctgatcacccgagtcctgcgaatcggcatccccgtgggcgccaagaagtgggactgcaagccctgcgaggaatacgtggtgaagaagaacgacattgagaaggctctgcgagaggtgatggaaggtaacgaggccgaggaacgacgaacccgagccaaggaatacaaggaaatggcctggaaggccctgcaagaaggcggatcttcttactctgacctgtctgccctgattgacgaactgcgaggcctgtctacctaa

SEQ ID NO: 11 - protein, 479 aa, Bougainvillea glabra
MESESKTEREHILMVPFMAQGHLRPFLELGLLLHSRTHFSITLLTTPLNAQYLRQQHHPNTGVRIVELPFNSADHGLPPGIENTSKLPLPLVVSLFHSSSSLEPHLRNFISLNFTPTKPPLCIIHDVFFGWVDSVAKSVGSTAIAFTTGGAYGTAAYASIWNELPHRNLSDDHDFPLPGFPDTHRFRRTQLHRFVRYADGDDDWSRFFQPQIRLSMGSFGWLCNSVQEIEPLGFQILRNYINLPIWGIGPLIATPVHIDNECIQWLSTKQPDSVLYISFGSQNTVSPTQMMELAAGLESSEKPFLWVIRAPFGFDINGEMRPEWLPEGFEERIKEKKQGILVHKWGPQLEILNHKSTGGFLTHCGWNSVLESLREGVPMLGWPLAAEQAYNLKYMEEEMGVAVEIARGLEGEISKEKAKRIVEMVLEREQGSKGWEMKNRAVEMGNKLNEAMKECKGSSIKATDDFLDFIMSKRSKPIG

SEQ ID NO: 12 - DNA, 1440 bp, Synthetic construct
  source 1..1440: mol_type = other DNA
atggaatccgaatctaagaccgaaagggaacatattttgatggttccattcatggctcaaggtcatcttagaccatttttggaattgggtttgttgttgcattctaggacccatttctctattaccttgttgactactccattgaacgcccaatatttgagacaacaacatcatccaaacaccggcgttagaatagttgaattgccttttaactctgccgatcatggtttgccaccaggtattgaaaacacttctaaattgccattgcctctggtcgtttctttgttccattcttcttcatctttggaaccacacttgaggaacttcatctctttgaatttcactccaactaagccaccattgtgcattatccatgatgttttctttggttgggttgattccgttgctaaatctgttggttctactgctattgcttttactactggtggtgcttatggtactgctgcttatgcttctatttggaacgaattgccacacagaaacttgtccgatgatcatgattttccattgccaggttttccagatactcacagattcagaagaacccaattgcacagatttgttagatacgctgatggtgatgatgactggtctagatttttccaaccacagatcagattgtccatgggttcttttggttggttgtgtaactccgttcaagaaattgaaccattgggtttccagatcctgagaaactatattaacttgccaatttggggtatcggtccattgattgctactccagttcatattgataacgaatgcatccaatggttgtctaccaaacaaccagattccgtcttgtacatttcattcggttctcaaaacaccgtgtctccaactcaaatgatggaattggctgctggtttggaatcttctgaaaagccatttttgtgggttattagagccccatttggtttcgatattaacggtgaaatgaggccagaatggttgccagaaggttttgaagaaagaatcaaagaaaagaagcagggcatcttagttcataagtggggtccacaattggaaatcttgaaccataagtctacaggtggtttcttgactcattgcggttggaattctgttttggagtctttgagagaaggtgtcccaatgttaggttggccattagctgctgaacaagcttacaatttgaagtacatggaagaagaaatgggcgttgctgttgaaattgctagaggtttggaaggtgaaatctctaaagaaaaggccaagagaatcgtcgaaatggtcttggaaagagaacaaggttctaaaggttgggaaatgaagaacagagcagttgaaatgggtaacaagttgaacgaagctatgaaggaatgcaagggttcatctattaaggctaccgatgatttcttggacttcatcatgtctaagaggtctaagccaattggttaa

SEQ ID NO: 13 - protein, 357 aa, Yarrowia lipolytica
MSRSSSPNASSAEDVRILGYDPLLAPALLQTEVASTKNARETVSKGRKDSIDVITGKSDKLLCIVGPCSLHDPKAAMEYAQRLKELSDKLSGELVIVMRAYLEKPRTTVGWKGLINDPDMDESFNINKGLRLSRKVFCDLTDLGLPIASEMLDTISPQFLADLLSLGAIGARTTESQLHRELASGLSFPVGFKNGTDGTLGVAVDAVQAASHPHHFMGVTLQGVAAITTTKGNENCFIILRGGKKGTNYDAESVAECKKATESMLMVDCSHGNSNKDYRNQPKVSKAVAEQVAAGEKKIIGVMIESNIHEGNQKVPKEGPSALKYGVSITDACVSWETTVDMLTELANAVKERRNKN

SEQ ID NO: 14 - DNA, 1071 bp, Yarrowia lipolytica
  source 1..1071: mol_type = genomic DNA
atgtcccgttcctcctctcccaacgcctcctctgctgaggacgtgcgaattctgggctacgaccccctcctcgctcccgctcttctccagactgaggttgcctccaccaaaaacgcccgagagaccgtctccaagggccgaaaggactccattgatgtcatcaccggcaagtccgacaagttgctgtgcattgtcggtccctgctccctccacgaccccaaggccgccatggagtacgcccagcgactcaaggagctgtctgacaagctgtctggtgagctcgtcatcgttatgcgagcctacctcgagaagccccgaaccaccgttggctggaagggtctgatcaacgaccccgacatggacgagtctttcaacatcaacaagggtctgcgactctcccgaaaggtcttctgcgaccttaccgatctgggtctgcccattgcctccgagatgctcgataccatttctccccagttcctggccgacctgctctccctgggtgccattggtgctcgaaccaccgagtcccagctgcaccgagagctcgcctccggtctgtctttccccgttggtttcaagaacggaaccgacggtactctgggtgttgccgttgatgctgtccaggccgcctctcaccctcaccacttcatgggtgtcaccaagcagggtgttgccgccatcaccaccaccaagggtaacgagaactgcttcatcattctgcgaggaggtaagaagggcaccaactacgacgccgagtccgtcgccgagtgcaagaaggccaccgagtccatgctcatggttgactgctctcacggcaactccaacaaggactaccgaaaccagcccaaggtttccaaggccgttgctgagcaggttgctgctggcgagaagaagatcatcggtgtcatgatcgagagtaatatccacgagggcaaccagaaggtccccaaggagggcccctctgcccttaaatacggtgtctccatcaccgacgcctgtgtctcttgggagaccaccgtggacatgctcaccgagctggccaacgccgtcaaggagcgacgaaacaagaac

SEQ ID NO: 15 - protein, 256 aa, Yarrowia lipolytica
MDFTKADTVLDLANIRDSLVRMEDTIVFNLIERAQFCRSEFVYKAGNSDIPGFKGSYLDWFLQESEKVHAKLRRYAAPDEQAFFPDDLPEAILPPIDYAPILAPYSKEVSVNDEIKKIYTDDIVPLVCAGTGDQPENYSSVMVCDIETLQALSRRIHFGKFVAESKFLSETERFTELIKNKDIAGIEAAITNSKVEETILARLGEKALAYGTDPTLRWSQRTQGKVDSEVVKRIYKEWVIPLTKKVEVDYLLRRLE

SEQ ID NO: 16 - DNA, 771 bp, Yarrowia lipolytica
  source 1..771: mol_type = genomic DNA
atggacttcactaaagccgacaccgttctggatctcgccaacatccgagactcgctggtccgaatggaggacactattgtcttcaatctgattgagcgggctcagttctgccgttccgagtttgtgtacaaggccggcaactcggacattcccggcttcaagggctcttacctcgactggtttctgcaggagtcggaaaaggtgcacgccaaactgcgtcggtacgctgccccggacgagcaggccttcttccccgacgatctacccgaggccattctgccccccatcgattatgcgccaattctggcaccctacagcaaggaggtgagcgtcaacgacgagattaaaaagatttacaccgacgacattgtgcccctggtgtgtgctggcactggagatcagcccgagaactattcctcggtcatggtgtgcgacatcgagacgctgcaggcgctgtcgcgacgaatccactttggcaagtttgtggccgagtccaagtttctgagtgaaaccgagcgattcaccgagctcatcaagaacaaggacattgctggtattgaggcggccatcacaaactccaaggtggaagagacgattctggcccggctgggagaaaaggcactggcctacggcacagaccccactctccggtggtcgcagagaacccagggaaaggttgattccgaggttgtcaagcgaatctacaaggagtgggtgattccactcaccaagaaggtcgaggtggactacctgctccggcggttggagtag

SEQ ID NO: 17 - protein, 394 aa, Yarrowia lipolytica
MSPSVEVTPAHTPTSYEVTNSLDSYRGYDHVHWYVGNAKQAASFYITRMGFSPIAYKGLETGSRDVTTHVVGNGQVRFAFSSALRTGEPQADEIHAHLVKHGDAVKDVAFEVDNVEQLFSAAVKKGVRVISEPKVLKDAHGSVTYAVISTYGDTTHTLIERGSYEGAFLPGFVDTSANKDPIAAFLPNIELMHIDHCVGNQDWNEMDNACKYYEETLGFHRFWSVDDKDICTEFSALKSVVMASPNEKIKMPVNEPAVGKKKSQIEEYIDFYDGPGIQHIALRTDCILDTVRDLRARGVEFISVPGSYYENMKERLAKSSLKLEEKFEDIQALNILIDFDEGGYLLQLFTKPLMDRPTVFIEIIQRRNFEGFGAGNFKSLFEAIEREQAKRGNL

SEQ ID NO: 18 - DNA, 1185 bp, Yarrowia lipolytica
  source 1..1185: mol_type = genomic DNA
atgtcaccttccgtcgaagtcacccctgcacacacacccacctcgtacgaggtgaccaactcgctagacagctatcggggctatgaccacgtccactggtacgttggtaatgccaagcaggccgcctccttctacatcacccgaatgggattctctcccatcgcctacaagggtcttgagactggctctcgagacgtgaccacccatgtcgtgggcaacggccaggtcagatttgcattctcgtctgccctgagaaccggagagccccaagccgacgagatccacgcccatctggtcaagcacggtgacgccgtcaaggatgtggcctttgaggtcgacaatgtggagcagcttttctctgctgctgtcaagaagggcgtccgagtgatttccgagcccaaggtgctcaaggacgcacatggctccgtcacctacgccgtcatctctacctacggagataccactcacactctgattgagcgaggcagctacgagggcgcctttcttcccggatttgtcgacacctccgccaacaaggaccccatcgcggccttcctgcccaacattgagctcatgcacatcgaccactgcgttggaaaccaggattggaacgagatggataacgcatgcaagtactacgaggagactctgggcttccaccggttctggtcggtcgacgataaggacatttgcaccgagttctctgctctcaagtccgtggtcatggcctcacccaacgagaagatcaagatgcccgtcaacgagccggccgtgggcaagaaaaagtcccagattgaggagtacattgacttttacgacggccccggtatccagcacattgctctgcggaccgactgtatcctggacaccgtccgagatctgcgggctcgtggcgtggagttcatttcggtccctggatcttactacgagaacatgaaggagcggctggcaaagtcatctctgaagctcgaggagaagtttgaggatatccaggcgctcaacattctgattgatttcgacgagggcggatatctgctgcagctgttcaccaagcctttgatggaccggcccaccgtgttcattgagatcatccagcgacgaaactttgagggttttggcgccggcaacttcaagtctctgtttgaggccattgagcgggagcaggccaagcgaggcaacctttaa

SEQ ID NO: 19 - protein, 469 aa, Celosia cristata
MDNGIEKKLNGKMEKKDLDFSSIKVKITQKTHVQPNKKLGRKECQLITFDIPYLGFFYNQKLMIYKENGSDFEELVKKLKDGLSVVLEEFYQLAGKLGRDDDGVFKVMYDDDMDGVEVLEAIVDDNDDGMMVCVDDLASKEDITMMKDLVPYNGILNLEGLQRPLLAIQLTKLRDGLAIGCAFNHAVLDGSSTWHFMSSWAEICNGAHSISLLPFLERTKARSTRISIEIPDAPPQPDIAASGTAPELREKLFRFPASAVEKIKAAHNSMGPANGTITATDPPYSTFQSLSSHIWRAVTKARELKPEDPTVFTIFADCRKRVDPPMPESYFGNLIQAIFAGTATGLLLGHPPEFASGMVKAAIGAHDAAAIDKRNKEFEAKPFIFQYKDAGMNCVAVGSSPRFKVYDVDFGFGKPERVRSGGNNRFDGMVYLYPEKEGGKGIDVEISLEVKAMDNLEKDQEFLMDGEVV

SEQ ID NO: 20 - DNA, 1410 bp, Synthetic construct
  source 1..1410: mol_type = other DNA
atggataatggcatcgagaagaagttaaatggcaaaatggagaagaaagatctagacttctcgtccataaaagtaaagatcacacagaagactcacgtacagccaaacaagaagctaggaagaaaggagtgtcagctaataacttttgatatcccttacttagggttcttctacaaccagaaattaatgatttacaaggagaacgggtccgattttgaggagcttgtcaagaagctgaaggatgggctaagcgtcgtactagaggagttttaccagttggctgggaagcttggaagggatgatgacggtgttttcaaggtaatgtacgacgatgacatggacggtgttgaggtccttgaggcgattgttgatgacaacgatgatggcatgatggtatgtgttgacgacttagcttcgaaggaagacataacaatgatgaaggacttggttccttacaacggcatcttaaatctagaaggattgcagagacctctattagctatccaacttactaaattaagggacggactggccataggatgtgcctttaaccacgccgtactggacggatcatcgacgtggcatttcatgtcgtcatgggccgagatatgtaacggagcccattcaattagcctattaccattcttagagcgtacaaaagcgagaagcaccagaattagtatagagatcccggacgccccaccacagcctgacattgctgcatcaggtactgcgcccgagttacgtgagaagttgtttaggttccctgctagcgccgtcgagaagataaaagcggcccataattccatgggtcccgcgaacggcaccattacagccacagacccaccatactcgacttttcagagtttgtcctcacacatctggcgtgcagtgacaaaagcaagggagttgaagcctgaggacccaacagttttcaccattttcgctgactgtaggaagagggttgacccaccgatgccagagagttactttggcaatcttattcaagctattttcgctggaacggctactggacttctacttggtcatccacccgagttcgcctcgggtatggtaaaagcagccatcggggctcatgatgcagctgcaatagataaaaggaacaaagagttcgaggcaaaaccctttatatttcagtataaagatgcagggatgaattgcgtggccgtcggatcgtcaccccgtttcaaggtctatgacgttgatttcggatttggaaagccagaaagggtaaggtctggcggtaacaaccgtttcgacggtatggtttacctgtaccctgagaaggaaggtggtaaaggtatagacgttgagattagcctggaagtaaaggctatggacaatttagagaaggaccaggagtttcttatggatggtgaagtcgtgtaa

SEQ ID NO: 21 - protein, 491 aa, Celosia cristata
MTCSELLSSPSSSSVTIISKSTVFPSEPSAMEDLKLSVSDLPMLSCHYIQKGGLFTRPPFNSSNLISLLKDGLSTALSHFPALAGRFKTDSLGYVFITCNDAGVDFVHATAKTVFIRDILSPTDVPLVVKEFFTYDRTVSYKGHFQPLMAVQVTELADGVFISCSVNHSVTDGTSFWNFFNTFAEVCKGVKRITKSPDFSRNSVLISPAILKVPEGGPVVTFNEKEPLRERIFSFSREAILELKSKVNNRKFSASAIELLGKQSNDPWNNNKDGKMASKIENWIRNAVTVSKPQGLTETESMAVEISSFQSLCALLWRCVTRARKLNSTKTTTFRMAVNCRHRLEPKLAPLYFGNAIQSIPTFASVDDVLSKDLRWAAEQLNKNVKAHGNDTVRRYVAEWESNPRTFPLGNFDGAMITMGSSPRFPMYDNDFGWGRPVAVRSGWANKFDGKISAFPGREGGGSVDLEVVLSPETMAGLESDPEFMQFVSSC

SEQ ID NO: 22 - DNA, 1476 bp, Synthetic construct
  source 1..1476: mol_type = other DNA
atgacatgctctgagttgcttagtagtccttcatcttcctcagttactataatttccaaatcaactgttttcccatctgagccaagcgccatggaagatctaaagttaagtgtgagtgaccttccgatgttgagctgccattatattcagaaaggtggtttatttacaaggccgccattcaactcatccaaccttatatcgttactaaaggatggcttaagtacagccttgtcccactttccagcacttgcagggcgtttcaagactgattcactaggttatgtgttcattacttgcaatgacgccggagttgatttcgttcacgctacagctaaaaccgtgttcatacgtgatattttatcgccgactgacgtacctttagttgtaaaagaattcttcacttacgatagaaccgtaagttacaaaggccacttccagccacttatggctgtccaggtgactgaactagccgatggggtgtttatctcctgttcagtaaaccactctgttaccgacgggactagtttctggaatttcttcaacacttttgccgaggtttgcaaaggggtaaagagaataaccaagtctcctgatttctcaaggaacagcgtgcttatctccccagctatcctgaaggtcccagaaggcggtccagttgttacctttaacgagaaagagccactgagagagcgtattttcagcttttcaagagaggccattctggagttgaagagcaaggttaataaccgtaagttctccgcgtcagccatagagctattgggtaaacagagcaatgacccttggaataacaacaaggatggtaaaatggcgtccaaaatcgagaactggattcgtaacgcagttaccgttagtaagccgcaaggtctgacagaaactgagtcaatggcagttgaaatttcaagctttcaatctctgtgcgctttgctttggagatgtgttaccagggctcgtaagttaaacagcaccaagactacaacttttaggatggcagttaattgcaggcaccgtcttgagccaaaattggctcctctttatttcggcaacgccatacagtccatccctacgttcgcatctgtcgacgacgtcctttccaaggatttgagatgggctgcggagcagttaaacaagaatgtcaaagcacacggaaacgacactgtgcgtagatatgttgctgaatgggaatctaaccctaggacattcccccttggtaatttcgacggtgcgatgataacaatgggctcatctcctcgtttccctatgtacgacaacgacttcgggtggggcagaccggtcgcagtacgttctggctgggccaacaagtttgacggtaaaatctccgcttttccaggcagagagggcggtggttcggtggaccttgaggtggtactatctcctgagactatggccggcttagaaagtgacccggaattcatgcagttcgttagtagttgctag

SEQ ID NO: 23 - protein, 453 aa, Celosia cristata
MVVSSVKRNPNCTNNEENLVYGHRLSSVGCAKVTGNLSYEPNNMDLAMKLHYLRVVYFLESQAIEGLMPLKLKEGIFEWLNYYYESCGRFRRSEKDGRPYIKCNDCGVRFIEAKSSKTLKEWILMEDSSSQRLLVADNVLGPELTFSPLVFLQITSLKCGGLALGLSWAHVLGDAFSASNYMNTLAEILNGKKPNYPPNYDKSPPQNLKPSKPIISDPLSLKKVGPVEDHWFNPLPCEMDTFSFHVKPNTLAQLQEKLSKNDSTPSPIGPFEMISSIIWRATARIRNGPELNTVTIIEKDRTSPIEEATTLRNNQVVGSVKAGFSVAQAQPEKLAALIHDSIKEEKAQIGEVVGQDPGGSDFIVYGSNLTFVNWEEVGFYGFELKGLSPRFVNCFVDGIGEGGVVLVLPSPKDESSDKLNGKIKSEGRVVTLIMPKTYMVELKEELKEWCLIA

SEQ ID NO: 24 - DNA, 1362 bp, Synthetic construct
  source 1..1362: mol_type = other DNA
atggtagtttcgagtgtgaaaagaaaccccaactgtacgaacaacgaggaaaacttggtctacggacacagattatcctctgtagggtgtgctaaagtgacagggaacttaagctacgagccaaacaacatggacttagccatgaagttgcattacttaagggtcgtgtacttcctggagagccaggctatcgaaggcttgatgcccctaaaattaaaggaaggaatctttgagtggttgaattattactatgagagttgtggaagatttcgtaggagtgagaaagacggtcgtccatatattaagtgcaatgactgtggtgtacgttttatagaggctaagtctagtaaaactcttaaagagtggatattgatggaagattcttctagccagagattgctggtcgcagacaatgtattgggtccagaattgacgttctcacctctggtcttccttcaaattacctctttaaaatgtggtggattagcgttgggccttagctgggcacacgtgctgggtgacgcgttctccgcttctaattacatgaacacacttgccgaaatattaaatggaaagaagcccaactacccaccaaattacgataagtcaccgccacagaatttgaaaccgtcaaagccaataatttcggaccctttgtcgcttaagaaggtgggaccggttgaggaccactggtttaacccactaccctgcgaaatggacacattctctttccatgttaagccaaacaccttggctcagctgcaggagaagctatccaagaacgactcaacaccttcacctataggaccgttcgagatgatctcttctattatttggcgtgcaactgcaaggattaggaatggtcctgaattaaacacagttacaatcatcgagaaagataggactagtcccatcgaagaggctacgacattgcgtaacaaccaggtcgtagggtcagttaaggcagggttttcggtagcccaagctcagcctgaaaagttagctgcattaatacatgactcgattaaagaggagaaggcccagattggcgaggtcgtgggacaggaccctggtggttcagacttcatagtttacgggtcgaatctaactttcgtcaactgggaagaggtgggtttctatggtttcgagttaaagggattatcgcccaggttcgtgaactgcttcgtggacggaataggcgagggcggcgtcgtgcttgtcctaccatctccaaaagatgaatcttctgacaagctaaatggcaaaataaaatctgaaggtagggttgtcaccctaatcatgccaaagacgtacatggtggagcttaaagaggaattaaaagaatggtgcctaattgcctag

SEQ ID NO: 25 - protein, 490 aa, Hylocereus polyrhizus
MREKMPCSEPLPTSSSSVMIISKSTVYPSQASSMADLRLSVSDLPMLSCHYIQKGNLFTRPPHSFSTLISQIKDALSETLSHFPALAGRLKTDADGYVYITCNDAGVDFVVAAAKNVHIRDTLSPIDVPDVVRGFFTYDRTVSYKGHFLPIIAVQVTELADGVFIGCSVNHSATDGTSFWNFFNTLAEVCRGMRKITRAPDFSRNSVLISPAVLKVPEGGPPVTFNEDEPLRERIFSFTREAILELKAKVNNRKLGGDDWGISISTVELLGKKSNDHWKESKLPAKLENWIRTASKTETPMAFEISSFQSLSALLWRCVTRARKLNPSKTTTFRMAVNCRHRLEPKLNPLYFGNAIQSIPTYASAGDLLSTDLRWAAEQLNKNVRNHGNETVRKCVSEWESNPRCFPLGNFDGAMITMGSSPRFPMYDNDFGWGRPVAVRSGRANKFDGKISAFPGREGGGSVDLEVVLSPETMAGLESDPEFMQYVSRF

SEQ ID NO: 26 - DNA, 1473 bp, Synthetic construct
  source 1..1473: mol_type = other DNA
atgagggaaaagatgccatgcagcgagccgttacccacctcttcctcgagcgtcatgataatttctaagagtacggtttatccatctcaggcatcaagtatggctgatcttcgtttaagtgtgtcagacctgcctatgttgtcttgtcattacatacagaaggggaatttatttacaaggcccccgcattctttctctaccttaatctcgcaaattaaggacgctttgtcggaaacattatctcacttcccggcgctagccggcaggcttaagactgacgccgatgggtatgtgtacattacctgcaatgatgcgggagtagacttcgttgtagccgcggcgaagaatgtgcatataagggacactttgtcacctatagacgtgcctgatgtagtcaggggtttcttcacatacgataggacagtgtcttacaaaggccacttcttgcctattatcgccgtccaggtaaccgagttggcagacggtgttttcataggttgttcggtgaatcactccgcaactgacggtacatctttctggaacttcttcaacacgttagcggaagtatgccgtgggatgaggaagatcacaagagcacccgatttctcgaggaactcagtccttatttcaccagctgtcttaaaggtccctgaaggtggcccaccagtgaccttcaacgaagacgagcccttaagggaaagaattttctcattcaccagggaagcaatactagaactaaaagcaaaggtgaacaacaggaagctgggcggcgatgactggggcataagtatatccaccgttgaattgttgggaaagaagtccaatgaccactggaaggagtcaaaattacccgcaaaattggagaattggataagaaccgcgagcaagacggaaacacctatggctttcgaaatcagtagtttccagtctctgagtgcccttttatggcgttgtgtcactcgtgctaggaaattgaatccatcgaaaactacaacttttagaatggcggtaaactgtaggcatcgtttggaaccgaaactgaacccattgtattttggtaacgctattcagtcgatacctacttatgcgtctgcaggtgaccttctgagtacagacctgaggtgggcagcggagcaactaaacaagaacgtcaggaaccacgggaacgagacagtgaggaagtgtgtgtccgagtgggagtcaaacccgaggtgcttccctttgggcaacttcgacggcgctatgatcacaatgggttcgtctccgagatttcctatgtacgataacgacttcgggtggggacgtccagttgcggtaagaagtggtagagcaaacaagttcgacggtaagatatcagcattccccggacgtgagggaggagggtctgttgacctagaggttgttctgagcccggaaacaatggctggcttagagtctgacccggagttcatgcagtacgtaagcaggttctag

SEQ ID NO: 27 - protein, 480 aa, Hylocereus polyrhizus
MANEIEKKQVDPSINVKITKKSHVQPKKKIGKKECQLVTFDLPYLAFFYNQKLLIYRIGNGSDFEQVVEKLKDGLSQVLVDFYQLAGKLNQDDDGVFKVMYDDDMEGVELVEAIVDGNHDHHDDGNGNGNGNVDDGKMMMSVDDLASMEDITMMKHLIPYNFILNVEGRHRPLLAIQLTKLRDGLAMGCAFNHAVLDGTSTWHFMSSWAEICNGVHSVSVPPFLDRTKARSTRVKLDMSRAPPPDSNGHNAAAAAAAAPVLREKLFRFSDSAIEKLKSDLNARLQPSNGTPTKPFSTFQCLSTHIWRHVTAARGLKLEDATVFTLFADCRRRVDPPMPETYFGNLIQAVFTGTAAGILLGNPPEFAAGMIQSAIAAHDAAAIEKRNKEWEENPIIFQFKDAGVNCVAVGSSPRFKVYDVDFGFGRPERVRSGSNNRFDGMVYLYPGKCGGKSIDVEISLEEKAMENLERDMEFLMNLEVA

SEQ ID NO: 28 - DNA, 1443 bp, Synthetic construct
  source 1..1443: mol_type = other DNA
atggctaacgaaattgagaagaagcaagtcgatccttccattaacgttaaaattacaaagaaatctcatgtccagcccaagaagaagatcggtaagaaggagtgccagttagtcacttttgatctaccgtatttggccttcttctataatcagaagttattaatttaccgtattggcaacggttctgacttcgaacaggtggttgagaagctaaaagacgggttgagtcaggtgcttgtggacttttaccagttagcggggaagttaaatcaggacgacgacggggtattcaaagtcatgtatgacgatgacatggaaggtgttgaactggttgaggcaatagtagacgggaaccatgaccaccacgatgatggcaatggcaatggtaatggtaacgtagatgatggaaagatgatgatgagcgtcgatgatctagctagtatggaagacattaccatgatgaaacatctaattccttataatttcatactgaacgtcgaggggagacaccgtcctcttcttgccatccagttaactaagctaagggatgggttggctatgggatgcgcttttaaccacgcagtgttagacgggaccagcacttggcatttcatgtcgtcctgggctgagatatgcaatggcgtccattcggtgtcagttccgccattcttggacaggacgaaagcaagatcaactcgtgttaagctagacatgtcaagagcacctcctcctgactccaatggacacaatgctgctgctgctgccgcggctgcaccggtgctgcgtgaaaagttatttagatttagcgactccgcaatagaaaagctaaagtccgatttaaatgctcgtctacagccgtcgaacggtacacctaccaaacctttcagcaccttccagtgtttatcaacgcatatttggagacatgtcacagcggccagaggactgaaacttgaggacgcaactgtattcacgctattcgcagattgtcgtagaagggtagatccacccatgcctgaaacttacttcggcaacttaatccaagctgtgttcaccggaacagcagcaggaattctgttgggaaacccacctgagttcgcagcaggaatgatccagagcgcaatcgcggctcacgacgctgccgctatcgagaagaggaacaaagaatgggaagagaatccaataatatttcaattcaaagacgcaggggttaactgcgtcgcagttggcagcagccctagatttaaggtctatgacgttgattttggatttggacgtcctgagagggtcaggtctggttcaaacaacagattcgacggcatggtatatttatatcctggtaaatgcggtggcaagtctatcgatgttgagatttcactagaggagaaagctatggagaatctagagagggacatggagttcttgatgaacctggaagttgcttag

SEQ ID NO: 29 - protein, 459 aa, Hylocereus polyrhizus
MVNQEGIKVISECFVRPEHEVEAAKNPLSLGPVDLAFLSIDPIQKGLLFPFVTNDTRPEISSIVERLKRSLSLALVYFYPLAGRFETNKYEDEHACWIFLDCTKGPGARLIHASYEDLSVSDILSSTDVHPAVRSFFDLGERVVNYDGHTKALLSIQVTELLDGVFIGFSMNHSVVDGTSFIHFVSSLSEIFRSDPQGNEGPVKISRVPLYKMHAPEGCGPISKLPYLEHDEFIVRYDPGPLRERIFHFSPESMARLKAKANEECGSGTQEISSYMALSSLVWKSITRAYNPAGHDETCCFTALNARPRHNPPLSDDYFGNILVRAKGVSKVDELLSQSLGWGARILSQEVKKHTHETICETLKMFAEHPSVIRRGLNQPGFYSVDNGGSHRFDMYGPEFGLGRAVAVLMGYGNKAHGKVTANPGREGRGSVDLEICLRPQIMSTLEADEEFMNFVSMG

SEQ ID NO: 30 - DNA, 1380 bp, Synthetic construct
  source 1..1380: mol_type = other DNA
atggtgaatcaagaggggataaaagttataagtgagtgctttgtgcgtccagagcacgaagttgaagcggcaaagaatcctcttagccttgggcctgtggaccttgcgttcctatctatagacccaattcagaaaggtttactatttcccttcgtaactaacgacactcgtccagagatttctagtattgtcgaacgtctgaagagatctttgtctttagcattagtttatttctatcctctggctggaaggttcgagaccaataaatatgaggatgagcatgcatgttggattttcctggactgtacaaaaggtccaggcgccaggttaatacacgctagctacgaggacctatctgtcagcgacatcttgagctctacagacgtacaccctgctgtgagatcgttctttgacctgggagagagggttgtcaattatgacgggcacactaaggcattgttatccattcaggtcacagagttattggacggtgtattcatcgggttttctatgaaccattcagtggtagacggcacaagctttatacattttgtaagcagtctatccgagatatttcgtagtgaccctcaaggtaacgagggtccagtcaagatttctagagttcccttatacaaaatgcacgcgccagaggggtgcggccctattagtaaattgccatacctagaacacgacgagttcatagttagatacgaccctggcccattgagagagaggattttccatttcagtccagagtctatggcccgtctaaaggccaaggctaacgaagaatgtggctcaggtacacaggagatcagctcctacatggccttgtccagtctagtatggaagtctatcacaagagcctacaacccagcgggacatgacgagacgtgctgctttactgcattgaatgctcgtcctaggcacaatcccccgttgtcagacgactacttcggcaacatcttagtacgtgctaaaggggtttcaaaagtcgacgagttgctatcacagagcttggggtggggtgctagaatcttgagtcaagaggtaaagaagcatacgcacgagaccatctgtgagaccttgaagatgtttgcagaacacccttccgtaatacgtcgtggattaaaccaaccgggtttctactcagttgacaacggcgggtcccatcgtttcgatatgtacgggccggagtttggtcttggaagagctgttgctgttttgatgggctatggtaataaggctcacggaaaggtcacagcaaatcccggtcgtgaggggagaggttctgttgatctagagatttgcttaaggccccaaattatgtccactctagaggcagatgaagaatttatgaacttcgtctctatgggctaa

SEQ ID NO: 31 - DNA, 1380 bp, Synthetic construct
  source 1..1380: mol_type = other DNA
atggtgaaccaggaaggaatcaaggtcatttctgagtgttttgttcgacccgagcatgaggtcgaagctgctaagaaccctttgtcactgggtccggtggacctggcattcctttccatcgatcccatccagaaaggactcctgtttccttttgttactaatgacaccagacccgaaatctcatcgattgttgagcgactgaagcgatccctctcgctcgcgcttgtgtacttctatcccttggccggccgattcgagaccaataagtacgaggacgagcacgcgtgctggatctttctggactgcacgaaaggacccggtgcccgattgatccatgcttcctatgaggacttgtccgtgtccgacattctgtccagcacagacgtgcaccctgccgtccgatccttcttcgatctcggagagcgagtggtgaactatgacggtcataccaaggcactgctgtcaatccaggttaccgagcttcttgatggcgtctttatcggtttctctatgaaccactctgtggtggacggcacctctttcattcactttgtgtcttcgctgagcgaaatctttcgatccgatccccagggtaacgagggtcccgttaagatctctcgggtgcccctgtataagatgcacgcccctgagggctgcggtcctatcagcaagcttccctacctggagcacgatgaatttatcgtgcgatacgaccctggccctctgcgagagcggattttccacttctctcccgagagcatggcccgacttaaggctaaagctaacgaagagtgcggttctggcacacaagagattagctcgtacatggccctgtcatccctggtgtggaagtcaatcacccgtgcctacaaccccgcgggccacgacgagacctgttgctttaccgctctgaatgcccgaccgcgacataacccaccattgtccgatgactatttcggcaacatccttgttcgagctaagggagtctctaaagtcgatgagcttctgtcccagtcgctgggctggggtgctcgaattctctctcaggaagtgaagaagcacacgcacgagactatctgtgagaccctgaaaatgttcgcagagcacccctctgtgattcgacggggcctcaatcagcccggtttctatagcgttgacaacggtggctctcaccgatttgacatgtacggcccagaatttggcctgggacgagctgttgcggtcctcatgggctacggtaataaagcccacggtaaggtcaccgctaaccctggcagagaaggccgaggctctgttgacttggagatctgtcttcgacctcagatcatgtccacattggaagccgatgaggagttcatgaattttgtctccatgggctaa

SEQ ID NO: 32 - protein, 435 aa, Hylocereus polyrhizus
MEISSFNGKTELEIKLGEPTRVPPLEKREREFYFLDNLDQNIAVTMRTLYLYKCPTKGNEDAPKVVKESLARVLVHYYPLAGRLTISPEGKLVVDCTGEGAIFVEAEANCSLAEMVAMATGDPIILKNLVYELPGAKNILETPLFTVQVTKFNCGAFSVAIAMNHCMADGLSAVEFVKSWGEVARGLPLTTRPSLDRTILKARDPPKIEFSHDEFLDIEDISNTHELLYDEEMVYGSFSFDPERLIRLKNKAIEDGTLSKCTTFEGLTALVWRARCKALGMHPDQQAKLLFAVDGRSKFNPPLPKGFFGNGIVLACALCKVNDLLKKPLSYAVELVQKAIKSATDSYMRSAIDYFEVTRARPSLSATLVITAWSRLSFHTTNFGWGETIYTGPVGLPDKEVALFLPHGVDKKSINIHLGMPASAMEVFEEEIMQV

SEQ ID NO: 33 - DNA, 1308 bp, Synthetic construct
  source 1..1308: mol_type = other DNA
atggagataagttctttcaacggaaagacagagttggaaataaagcttggagaaccaactcgtgtaccacctttagagaaacgtgaacgtgagttctactttctagacaacttagatcaaaacatcgctgtgacaatgcgtaccttatacttatacaaatgccctaccaagggaaacgaggacgcaccaaaggtcgtcaaggagagtcttgcccgtgtcttagtccactattacccattagcgggaagactaacaatatcgcctgagggtaaattagtggtagactgtacaggtgagggtgcaatattcgtagaggccgaggcaaattgtagccttgccgaaatggtggcaatggccacgggtgatcctataattttaaagaacttagtctatgagcttcctggggcaaagaatattttagagacaccactatttacggtccaggtaacaaaattcaactgtggcgctttcagtgtcgctatcgcgatgaatcactgcatggctgacggattaagtgccgttgagtttgtgaaatcctggggtgaagtcgcaaggggcttacccctaaccaccaggcccagcctggaccgtacgatcttaaaggcaagagacccaccaaagattgagttcagtcacgacgagttcttagacatcgaggacatctcaaatacccatgaattactttacgatgaggaaatggtgtacggctccttctctttcgacccggagcgtttgataaggttaaagaacaaggcgatagaggacgggacccttagcaagtgtactacattcgagggcttaactgctttggtttggcgtgcgaggtgtaaagctctgggcatgcatccagatcagcaggctaagttattgtttgccgtagatggaaggtccaaatttaacccaccattacccaaaggtttcttcggtaacggcattgtacttgcctgcgctctatgcaaggtaaacgaccttcttaagaagccactttcttatgctgttgagctagtacagaaagcaattaaatccgcgaccgactcgtacatgcgttccgccattgactactttgaggtgactagggcaaggccatccttaagtgctacacttgtgataaccgcttggtctaggttgtcatttcataccaccaatttcggttggggcgagaccatatacactggtccggtgggattacccgacaaggaagtcgcactattcttaccgcacggtgtagataagaagtctatcaacattcatttagggatgcccgcaagcgcgatggaagtgtttgaggaagagatcatgcaggtataa

SEQ ID NO: 34 - protein, 463 aa, Hylocereus polyrhizus
MVNQEGTSKVISECFVRPEHEVEAAKRPYRLGPVDLSMLSIDPIQKGLLFPFETDSTRPEIELLVERLKRSLSIALVHFYPLAGRFETIKYEEEHACFIFLDCTKGPGAGFIHASVDLTVSDILSSTDVHSAVRSFFDLGEKMVNYDGHTKPLLSIQVTELLDGVFIGFTMNHSVVDGTSFIHFVSTLSEIFRSEGEGGANPTQISRVPLYKMYAPEGYGPIFKLPYLEPEEFIIRYDPGPLRERIFHFSPDSMAKLKAKANGECGTETQVISSFMALSGLVWKSITRARNSPAHENTACSLLLNARPRLEPPLSENYFGNFLGQVKGVCKVEELLGQSLGWAAGVLGQELKRSTHEVIMDLIKTVSERPIVVPPGLERADLYGAANSVVIGGSHRFDMYGPEFGLGRAVAARMGYPNKDNGKVTANPGREGGGSVDLEICLRPQIMKALEEDEEFMGFVTVV

SEQ ID NO: 35 - DNA, 1392 bp, Synthetic construct
  source 1..1392: mol_type = other DNA
atggtgaaccaagaagggacctcgaaagttattagtgaatgctttgtccgtccagagcatgaggtcgaggctgctaagaggccctataggcttggccccgtagacctatctatgttgtcaatcgatcccattcaaaagggcctgctattccccttcgagacagactccacccgtccagagatagagttacttgttgaaaggctgaaaagatccttgtctatagctctagtacacttttacccgttagcaggcaggtttgaaaccatcaagtacgaggaagagcacgcatgcttcatattcttagactgcactaagggtccgggcgcaggattcattcatgcatctgtagatttaacggtgagtgacatattatcatctaccgacgtacattctgcggtgagatccttctttgatttaggggagaaaatggttaattacgatggtcataccaaacctctgttgtctatacaagttacagaattacttgacggcgtcttcattgggttcaccatgaatcactctgttgttgacggtacttcgtttatccatttcgtttcaacattatcagagatattccgttccgaaggtgaaggtggtgcaaatccgactcaaatatcccgtgtcccattatataaaatgtacgcaccggaaggctacggaccaattttcaagttgccgtaccttgagccggaagagttcattattagatacgaccctggtcctctaagggagcgtattttccactttagtcctgacagcatggcaaaattgaaggccaaggcaaatggtgaatgcggcactgaaactcaggttattagctcgttcatggcactgtcaggccttgtttggaagagcatcacgagagcccgtaactctccggcacacgaaaacactgcttgcagtttattgctaaacgcgagacctaggttggagccacccctatcggagaactacttcggtaattttcttggacaagttaaaggggtttgtaaagtagaggaactactgggccagtctctaggctgggccgcaggggtattggggcaggagcttaaaagatctactcatgaggttataatggacttgattaaaactgtctcggagagacccattgtcgtgccgcccggtctagagagggccgacttgtatggcgcagctaattcagtagttattggtggcagccacagattcgacatgtacggtcctgagttcggcttgggtagagccgttgctgcccgtatgggttatcccaacaaagataacggaaaagtcactgccaacccaggcagagagggcggcgggagtgttgacttagagatttgtctaaggccccagataatgaaggccctggaagaggacgaagagttcatgggcttcgtaactgttgtctga

SEQ ID NO: 36 - protein, 466 aa, Hylocereus polyrhizus
MQNYEKSSQTQSPLVFKVRRAAPELIPPIAPTPKEFKELSDIDNQEGLRLQFPFLQFYRGNESRGWNQDPAKVIREAVGKTLVPYYPFAGRLREKAGRKLVVDCNGQGVLFIEANADVTLDDFGEEIHPPFPCLEELIFDVPGNGGTLDSPLIIIQVTRLKCGGIILAWSFNHAMADGAGFFQFLSAVAELGHGGQLSVQPIWERHLLCAPDPPRVTCVHHEYDTMAETRGNVVPLDDNMVHRSFFFGPRDVSALRCLIPPDLKSSTAFEVLTACLWRCRTIALQPDPNATVRFHCIVNARSILNPPVPNGYYGNSITFSTVLSTAGDLCQKPVGYVVELVKMAKANVTDEYMRSFADFQVITGRPCLSTSGCCFAVSNLTRIGVDELDYGWGLPEYAGIAGQVFGICTFYLPYKNKKREKGILCPICLPAPAMERFVMELNSMLDGSSARPNTGSVSSAFSSSLF

SEQ ID NO: 37 - DNA, 1401 bp, Synthetic construct
  source 1..1401: mol_type = other DNA
atgcagaattacgagaaaagttcacagactcaatccccattggtcttcaaagtgcgtcgtgctgcccctgagctgatacccccaatcgcccctacgcctaaagagtttaaagagctatcagacatagacaaccaggaaggattaaggttgcaatttccatttcttcagttttataggggcaacgagagtcgtgggtggaaccaagatcctgcaaaagttataagagaggcggtagggaagaccttagtcccttactatccttttgccggcagactgcgtgaaaaggcaggcaggaaactggtcgtcgactgtaatggacagggagttttgtttatagaggccaatgctgacgtcaccctagacgatttcggcgaagaaattcacccaccttttccctgtctagaagagttgatatttgacgtgcccggaaatggtggtactttggactctcctctaattataatccaggttactaggttaaagtgcggcggtatcatacttgcttggtcattcaatcacgccatggcagacggtgcaggtttcttccagtttctaagtgcagtcgcggagttgggccatggtggccagttatcagtacagccgatctgggaaaggcaccttctatgcgccccagatccacctcgtgtgacttgtgtacaccacgagtacgacacaatggctgagacgagagggaatgttgtgcctttagacgataacatggttcaccgtagtttcttctttggccctagagacgtcagtgccttaaggtgcttgattccgcccgacttaaagtcttccacagcttttgaggtgctgactgcttgcttgtggagatgccgtacaatagcacttcagccggacccaaacgctacggttaggtttcactgcatcgttaatgctagatcaatcctgaacccacccgtcccgaacgggtattacggtaattcaattacattctccacggtgttgtcgacagcaggcgacttgtgtcagaagccagttggttacgtggtcgagttggtgaagatggccaaggccaacgtgacggacgagtatatgagatcgttcgcagatttccaggtcattacgggcaggccttgcctgtccacttcaggttgttgcttcgcggtaagcaacctgacgaggataggtgtggacgagctagactacggttggggtttaccagaatacgcaggcatagcgggtcaggttttcgggatatgtacattttatttgccatataagaataagaaaagagagaagggtatattgtgccctatttgtttacctgcccctgctatggagcgtttcgtcatggagcttaattcaatgctggatggttcatcagcaagacccaatactggatctgtttccagtgctttctcttcatcattgttctag

SEQ ID NO: 38 - protein, 461 aa, Amaranthus hypochondriacus
MSHNKESNPNLHVAFYPWFALGHLTSFLRLANKLAERGLQVSYFIPSKTQPKLSPHNHHPNHLTFIPITVPHVDGLPPGAETTNDVPGSAVPLIMTAMDLTQDIIEAHLAQLKPNFVFYDFTYWIPKLGQKLGFKSIHYFTAFISRYGYLAPYKKEGYLPTAADLLRPPPGYPSPIRMKPYEAKIMAGAGKTAFGLGGFTLAERLAVSFIECDAFGVKTCKEMEGEHCKFFEDVFGKPVLLAGPVVPKLPSSKLDEHFDEWLNGFDESSVIFCALGSECSLEINQFHELLLGLELTGRPFLAALKPPKNYKTIESALPEGFASRTRERGIVHEGWVQQQLILQHRSVGCFITHCGVGSLSEAMISKCQVVMIPQAIDQFINARMMSLEWKIGVEIETREDDGWFTREDVHKAITMVMDGESDVGREVRANHAKWRDFILTQGVEDSYISSFIESLQQLLIV

SEQ ID NO: 39 - DNA, 1386 bp, Synthetic construct
  source 1..1386: mol_type = other DNA
atgagccacaataaagagagtaacccgaaccttcacgtcgccttttatccatggtttgcacttgggcacctgacttcattcctaaggttggcaaacaagctagcagaaaggggtctgcaggtttcatattttattccgtctaagacacagcctaagttatccccacataaccatcatcccaatcatttaacctttataccaattacggtgcctcacgttgatggtttgccgcctggtgccgaaacgactaacgatgtacccggttcggctgttcccctgataatgactgcaatggacttaacccaggatattattgaggcccacttggcgcagttgaagcccaacttcgttttctatgatttcacctactggattccgaaattgggccagaagttgggattcaagagtattcattacttcacggcctttatcagcaggtacggttatttagcaccgtacaagaaagagggatatttacccactgctgctgacttgttgaggccacccccaggatatcccagtccgattcgtatgaagccatacgaggctaaaataatggcgggtgctggtaagaccgcttttggattaggtggtttcaccctggctgagagactagccgtatcatttatcgagtgcgacgcttttggcgtaaagacatgtaaagagatggaaggagagcattgtaagttcttcgaagatgtgtttgggaagccagtacttctggccgggcctgttgttccaaaactgccatcaagcaagctagacgaacacttcgatgagtggctaaacggcttcgacgagtcatctgtgatattctgtgctcttggatcagagtgttctcttgagattaaccagttccatgagcttcttttgggtttggagttgaccggtaggccattcctagcagccctaaagccaccaaagaactataaaaccatagagagcgccctgccagaaggatttgcttcaagaacccgtgagagagggatcgtacacgaaggttgggtccaacagcaattaatactgcagcacaggagtgtgggctgcttcataacccactgcggcgtaggcagtttgagcgaggctatgatctcgaaatgtcaggtcgttatgattcctcaagctatagaccagttcattaacgctagaatgatgtcgttagagtggaaaataggagtcgaaatagaaactcgtgaggacgacggttggtttactagagaggacgttcacaaagccataacaatggttatggatggcgagtccgacgttggcagggaagttagagcaaaccacgcaaaatggagagatttcatactgactcagggtgtcgaggactcctacatcagttcctttatcgaatccttacagcaattattgatcgtgtga

SEQ ID NO: 40 - DNA, 1386 bp, Synthetic construct
  source 1..1386: mol_type = other DNA
atgtcccacaacaaggagtcgaatcctaacctgcacgtggctttctatccctggtttgcccttggacatctgacttcctttctccgactcgccaacaagctggccgagcgaggccttcaggtttcctacttcattccctctaaaacccaacccaagctctcgcctcacaaccaccaccccaaccacctgacctttatccctatcacagttccccatgtcgatggactgccacccggcgcggaaaccactaacgatgtccccggatcagcggtccccctgatcatgaccgccatggacctgacccaagacattatcgaggcccatctggcacaactgaagcctaatttcgtcttttatgatttcacttactggattccgaaacttggtcagaagctgggattcaaatccattcattatttcaccgccttcatttcacgatacggttacctcgccccatacaagaaagagggctaccttcccactgccgccgacctgctccgacccccacccggttacccctcgcccattcgaatgaagccttatgaggctaagattatggctggagccggaaagactgcttttggtctgggcggcttcacactcgctgagcgattggctgtgtcttttattgaatgtgatgctttcggcgtgaagacctgcaaggagatggaaggcgaacattgcaagttcttcgaggacgtgttcggcaaacccgttctgctggccggccctgttgtgcctaagcttccatcctcaaagctggacgagcatttcgatgagtggttgaatggctttgacgagtcttctgtcatcttctgcgcactgggtagcgagtgctcgcttgagattaatcagttccacgaactgctcctgggcctcgaactcacaggccgaccgttcctggctgcccttaaaccacccaagaactacaagacaatcgagagcgcccttccagagggattcgcttcgcgaacgcgagagcgtggaatcgtccacgagggatgggtgcaacaacagctcattctgcaacaccggtctgtgggatgctttattactcactgcggcgtcggctctctgtcagaggctatgatctcgaagtgtcaggtcgttatgatcccacaggccatcgaccagtttattaacgctcgaatgatgtccctcgagtggaagattggcgtcgagattgaaacccgagaggatgacggatggttcactagagaggacgttcacaaagcgatcaccatggtcatggatggagaaagcgatgtgggaagagaagtgcgagccaatcacgccaaatggcgggacttcattctcacccagggcgtggaagactcctacatctcttcttttattgagtccctgcagcaactcctcatcgtctga

SEQ ID NO: 41 - protein, 462 aa, Chenopodium quinoa
MSQNKDNQILNVTFYPWFALGHLTSFLRLANKLAERGHNVSYFLPPKTQSKLASHNHYPTHLTFIPIPVPPVEGLPPGAETTNDVPASLGPLIMTAMDMTRDTIESHLVRLKPDIVFYDFTCWMPELGRKHGFKAMHYITAYIARYAYLAPYKKIPGYHPNADDLLTPPPEFPSQSIRMLPQEAEIIAGALKTPFGLGGLTLAERLGVSFRECDAFGVKTCAEMEGEYCKFFEKIFGKPVLLAGPMVPKRPSSELDNYFDDWLNSFRTSSVIYCALGSECALNLNQFQELVLGLELTGRPFLAALKPPMNYQTIESALPEGLAERIKGRGLIHGGWVQQQLILQHPSVGCFITHCGAGSLSEAMVSECQVVLMPQAIDQFISARMMSLEWKVGVEVEKRKNYGLFTKEAVHKAVSLVMEEDSEVGRDVRANHAKWREFILTEGLEDSYISSFIQSLQQLIGS

SEQ ID NO: 42 - DNA, 1389 bp, Synthetic construct
  source 1..1389: mol_type = other DNA
atgtcacagaacaaggacaaccagatcctaaacgttacgttctatccttggttcgcattgggacatcttacatctttcttgaggttggctaacaagttggcggagagaggccacaatgtgagctatttcttgccacccaaaacacagtccaaacttgcatctcataaccactatccaactcacctgacatttatacctattccagtcccaccggtagagggcctgccacctggggcggagaccaccaatgacgtgcccgccagtttaggcccgttgattatgacggccatggatatgacccgtgacacaatagagtctcatttggtcaggcttaagccagatattgttttctacgatttcacatgctggatgccagagctaggccgtaagcacggcttcaaagcgatgcattacataaccgcatacattgctcgttatgcttatctagccccctataagaagattccaggctatcatccaaatgccgatgatcttcttacaccgccccctgagttcccctctcagtcgattcgtatgcttccacaagaggccgagattatagctggagccctaaagacgccattcggacttggtggattgacgctggcggagagattaggcgtttcattcagagagtgtgacgctttcggagtgaagacgtgcgctgagatggaaggtgagtactgcaaattcttcgagaagatcttcggaaagcctgtgttattggcaggcccaatggttcctaagagaccttcttctgagttggacaactactttgacgattggttaaattccttcagaacctcgagcgtcatatattgcgcattgggttctgagtgtgcattgaacctgaaccagttccaggagttggtcttaggtctagagttgactggaagaccattcttggcagctcttaagcccccgatgaattaccaaacgattgagagcgccctgcctgaaggtctggcagagcgtattaaggggcgtggacttatacatggcggctgggtccaacaacagttaatactacagcaccctagcgtaggatgcttcattacacactgcggtgccggttctttgtctgaggcaatggtgtcggaatgccaagttgtactaatgcctcaagccatcgaccagtttatctcggcgcgtatgatgtccttagagtggaaggtaggagtggaagtagaaaagagaaagaactacggtctattcactaaagaagcagttcacaaggctgtatcactagtaatggaagaagacagcgaagttggtagagacgttagggccaatcatgccaagtggcgtgagttcattctaacagaggggttagaggattcatacatatcttcgtttatccaatcattacaacagctgatcggatcttag

SEQ ID NO: 43 - DNA, 1389 bp, Synthetic construct
  source 1..1389: mol_type = other DNA
atgtcccaaaataaagacaaccagattctgaatgtcaccttttatccctggttcgctttgggccatctcacatcctttcttagacttgcgaataaactggccgaaagaggtcataatgttagctacttccttccccctaaaacccaatcaaagctggcgtcccataaccactaccccacccatttgactttcatccctattccggtgccacccgttgagggcctgccgcctggtgccgaaactacgaatgacgtgccagcttcccttggtcccctgattatgacagctatggatatgactcgagacaccatcgagagccacctcgtccggctgaaaccagatattgtgttttacgacttcacctgctggatgcccgaactcggtcgaaagcatggattcaaggccatgcactacattacggcatatattgcccggtatgcttacctcgcaccctacaagaaaatcccgggatatcacccaaatgcagacgaccttcttaccccaccccctgaatttccaagccaatctattagaatgcttcctcaggaagctgaaattattgctggtgctctgaagacacccttcggcctgggtggtttgactcttgctgaaagattgggagtctcgttccgtgagtgtgacgcctttggagttaagacttgcgccgaaatggaaggtgaatactgtaagttctttgagaaaatctttggtaaacccgttctcctcgcaggtcccatggtccccaaacgaccttcctcggaacttgataactattttgatgactggttgaactctttccgaacctcgtcggtcatctattgtgccctcggcagcgaatgcgcattgaatctcaatcaatttcaggaactcgtcctcggactcgagctgactggacgtccttttctggccgcccttaaaccgcccatgaattatcaaacgatcgaatccgcccttcccgagggcctcgccgaacgaatcaagggtagaggccttattcacggcggatgggtgcaacaacaactcattcttcagcacccctcagtgggctgctttattacacactgtggagcaggatcgcttagcgaagctatggtgtccgaatgtcaagttgttctgatgccacaagctatcgaccaattcatctctgctcggatgatgtctttggaatggaaggttggagttgaagtggaaaagcggaagaactatggccttttcactaaagaggccgtccacaaagccgtgtcgcttgttatggaagaagactcggaagttggccgtgatgtccgtgcaaatcacgcaaaatggcgtgaatttattctcactgaaggacttgaggactcttacatttcttcatttattcaatcactgcagcagctgattggaagctaa

SEQ ID NO: 44 - protein, 459 aa, Celosia argentea var. cristata
MSSNQDSSVLHVAFYPWFALGHLTSFLRLANKLAERGHKVSYFLPAKTQPKLAPHNHYPNHLTFIPITVPPVDGLPPGAETTNDVPGPSVPLIMTAMDLTRDTIEAHLTHLKPNCVFYDFTHWMPELGRKLGFKSIHYFTAFISRYGYLAPYKKAGYLPTAADLLGPPVGYPSPIRMKAHEAEIMAAAGKNAFGLGGLTLAERLGVSFLECDAFGVKTCEEMEGSHCKYFEEIFGKPVLLAGPVVPKLPSSKLDEYFDGWLNGFGNATVIFCALGSECSLEVNQFQQLVLGLELTGRPFLAALKSPKKCKTIDEALPEGFAKRTRGRGIIYEGWVQQQLILHHPSVGCFITHCGVGSLSEAMISKCQVVMMPQAIDQFINARMMSLEWKIGVEVETREIDGLFTKEAVYKAVSTVMDEESEVGREVRANHAKWREFISMEGVEDSYISSFIESLRQLLG

SEQ ID NO: 45 - DNA, 1380 bp, Synthetic construct
  source 1..1380: mol_type = other DNA
atgagttctaatcaggattcttcggtactacacgtagccttttatccctggttcgcgttaggccacttgaccagtttcctaagattagctaataaactggccgagagaggccacaaagtttcctatttcttacccgccaagacacagcccaaactagcaccacacaatcactaccccaaccatttaacattcattcctatcacagttcccccagtagacggtcttccaccaggtgccgaaacgacgaacgacgtcccaggacctagtgtacccttgattatgactgcgatggatctaacgcgtgatacaatcgaagcccacttgactcatttgaagcctaactgcgttttctatgactttacgcactggatgccagagctgggtaggaaactgggctttaagtcaattcactacttcaccgcattcatatctcgttacggatatttggcaccttacaagaaagcgggttacctgccaacggcagccgacttgcttggcccacccgtgggttacccttcacctatcagaatgaaagctcacgaggcagagataatggctgccgcaggtaagaatgcgttcggtttgggcggactaactttagcagagagattgggtgtctcattcttagaatgtgacgcgttcggtgtcaaaacttgcgaagagatggaaggatctcactgtaagtatttcgaagagattttcggtaagcccgttctgttagcaggccccgttgttccgaaactaccttcctctaagctggacgagtactttgatggctggctaaacggatttggtaacgccaccgttatcttctgcgcactggggagtgagtgtagcctagaggtcaaccaatttcagcaacttgtcttgggtctagagttaacaggtaggccgtttctagcagccttaaaatcccctaagaaatgcaagactatcgacgaggcccttcccgaggggtttgctaagcgtacaagaggtaggggaatcatctatgagggttgggtacaacaacaactgatacttcaccacccttctgtggggtgtttcattactcactgcggagtagggagcttgtcagaggcaatgatatctaaatgccaggttgtcatgatgcctcaggcgatagaccaattcataaacgccagaatgatgtccttagaatggaagattggagttgaggtagaaacacgtgagatcgatggtttgttcacaaaggaagctgtatataaagccgtttccacggtcatggacgaggaatccgaagttggaagagaggtacgtgctaaccacgcaaagtggcgtgagttcatctccatggaaggtgtagaggattcttacatctcttcatttattgagtccttgaggcaattacttggatga

SEQ ID NO: 46 - DNA, 1380 bp, Synthetic construct
  source 1..1380: mol_type = other DNA
atgtcgtccaaccaggactcgtctgttctccatgtggccttttatccctggtttgctctcggccacttgacgtcgttcctgcgattggctaataaactggccgagcgaggacacaaggtgtcgtactttctccccgcaaagactcagcccaagctggcaccccataaccactatcccaatcatctgaccttcatccctatcacagtgccgccggttgacggactgccacccggagctgaaactacgaacgatgtccctggcccttccgtcccgctgatcatgaccgccatggatctcactcgagacactattgaggcccatcttacacatctcaaacccaattgtgttttctatgactttacccactggatgcccgaactgggccgaaagctgggtttcaagtccattcactacttcactgctttcatctcccgatacggatatctcgcgccgtacaagaaggctggttatctgcccaccgctgcagacctgctgggaccgcccgtgggttacccttcccctatcagaatgaaggcccatgaggccgaaattatggccgctgccggaaagaatgcatttggcctcggcggtctcactctggccgaacgactgggagtctcctttctggaatgcgacgccttcggcgtgaagacctgtgaggagatggaaggttctcactgtaaatacttcgaagagatctttggaaagcctgttctgctggcgggtcccgtggtccctaaactcccctcatcgaagctcgacgaatacttcgatggttggctgaatggatttggtaacgcgacggtgatcttttgtgctctgggctcggagtgcagcctggaagtcaaccagtttcaacagctcgtgctgggcctcgaactcacgggccgtccctttctggccgccctcaagtcccctaagaaatgtaagacgattgatgaggcgctccctgagggattcgctaaacgaacccggggccgaggaattatttatgagggctgggttcaacaacaactgattctccaccacccctctgtgggctgcttcatcacgcactgcggcgttggatctctgtctgaggctatgatctccaaatgtcaggttgtgatgatgcctcaggcgatcgaccaatttatcaacgcccggatgatgtctctggagtggaagattggtgtcgaggtggaaacacgagagatcgatggtctgttcaccaaagaagctgtgtacaaggccgtttcgactgtcatggacgaagagtcagaagtgggccgagaggtccgggccaaccacgccaaatggcgagaattcatctccatggaaggtgtcgaggattcgtatatttcgtcgttcatcgaaagcctcagacagctgcttggctag

SEQ ID NO: 47 - protein, 481 aa, Arabidopsis thaliana
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKAADLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 48 - DNA, 1446 bp, Synthetic construct
  source 1..1446: mol_type = other DNA
atggtaaagatttgctgtataggagcaggatacgttggtggacctaccatggcggtaatggcattgaagtgcccagagatcgaagttgtagtcgtagacatatctgaaccaaggataaacgcttggaactcggatagactaccgatatacgaaccaggacttgaggatgttgttaaacaatgtagagggaagaatcttttcttttccacagacgtcgaaaagcacgttttcgagtctgatatagtctttgtatcggttaatacaccaacgaagacacaggggcttggtgcaggaaaagcggctgatctgacttactgggagtcagcagctagaatgattgccgatgtgtccaaatcatcgaagatcgtagttgagaaatctaccgtcccagtacgtacagcagaagcaatcgaaaagatattgacccataattcgaagggaatagagttccagattttgtctaacccggagttcttagccgagggaactgccataaaggacctatacaaccctgaccgtgtattgataggtgggagagacaccgctgctggacagaaagcaatcaaggccctgagggacgtttatgcacactgggtgccagttgagcaaataatatgcaccaatttgtggtcagctgaactgtcaaagttagccgcgaatgcattcttggcgcagagaatctcatctgtcaacgcaatgtccgcgttgtgcgaggcgacaggagccgatgtgacccaagttgcacatgctgtcggtacagatactcgtattggacccaagttcttgaatgcatcggtaggtttcggcggtagttgcttccagaaagatatcttgaatttgatttatatatgtgagtgcaacggattacctgaagcggctaattactggaagcaggtggttaaggttaacgactaccagaaaatacgttttgcaaaccgtgtcgtgtctagtatgtttaacaccgttagtggaaagaagatcgcaatccttggctttgcctttaagaaagacactggggacaccagggaaacaccagccattgacgtctgcaatagactagtagccgacaaggcaaaactatctatctacgatccccaggttttggaagaacagatcaggagagaccttagtatggcaaggttcgactgggaccatccagttccattgcagcaaattaaagccgagggaatcagtgaacaggttaatgtagtgagtgacgcttatgaggctaccaaggacgcacatggtttatgcgtcttgacagagtgggacgaattcaagagtttggatttcaagaagatcttcgacaacatgcagaagcctgcatttgttttcgacggacgtaatgtggtggacgcagttaagttaagagagattggtttcattgtctattctataggtaagcccttggacagctggcttaaagacatgcctgcagtggcttga

SEQ ID NO: 49 - protein, 495 aa, Abronia nealleyi
MDQTTLAMLLSALYLLYNLYKVIFTQSNSKLPPGPKPLPIFGNISELGAKPHRSFANLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYEYVYDCAKKGEAVDIGKAAFTTSLNLLSNLFFSVELAQHTSTSSQHFKQLIWDIMEDIGKPNYADYFPALKCVDPWGIRRRLAANFERLIDVFQDFIRPRLSMNPSSVTSASDVLDVLLNLYKEKELNMGEVNHLLVDIFDAGTDTTSSTFEWAMAELVRHPETMKKAQDEIEQVLGKDATIQEADIPKMPYLQAIIKETLRLHPPTVFLLPRKAATNVELYGYVVPKDAQILVNLWAIGRDPLVWDQPNVFSPERFLNSDVDVKGRDFGLLPFGAGRRICPGMNLAYRMLTLMLATLLQSFEWKVENGEKAEDLDMDEKFGIALQKTKPLQIIPVLKYC

SEQ ID NO: 50 - DNA, 1488 bp, Synthetic construct
  source 1..1488: mol_type = other DNA
atggatcaaactactttggctatgttgttgtctgccctctacttgttgtataacttgtataaagttattttcacccagagcaactccaaattgccaccaggtccaaaacctttaccaatttttggtaatatctctgaattaggtgctaagccccatagatcttttgctaatttagctaaaatccatggcccgttgattactttgaaattgggatctgtgaccacgatcgtggtttcttcagctaaagtggccgaagaaatgttcctgaagaatgatttaccattagctaataggaacgttccaaattctgttactgctggtgaccaccataaattaactatgtcttggttaccagtctctccaaagtggaaaacttttagaaaaattaccgccgtgcatttgttatcaccacaaagattagatgcttgtcaagctttgaggcacgcaaaagtaaaacaattgtatgaatatgtgtacgattgcgctaaaaagggtgaagctgttgatattggtaaagcagctttcactacatcgttgaatttattatctaatttgttcttctcagttgaattggctcaacatacttcgacttcttcccaacattttaaacaattgatttgggacattatggaagatattggtaagccaaactacgctgactactttccagctttgaaatgtgtcgatccatggggtattcgtagaagattggctgctaattttgaaaggttgattgacgtttttcaagattttattagaccgagattgtcaatgaatccatcctccgtcacttctgcttctgatgttctcgacgttttgttaaatttgtataaagaaaaagagcttaatatgggcgaagttaatcacttgttagtcgatatttttgatgctggtaccgatactacttcttctacatttgaatgggctatggctgagttggtccgtcatccagaaactatgaagaaagctcaggatgagatcgaacaagttttgggtaaagatgccactattcaagaagctgatattccaaaaatgccctatctgcaggctattattaaagaaacattgagattgcacccaccaactgttttcttgttgccaagaaaagcagctaccaacgtcgaactgtatggttatgttgttcctaaggatgctcagatcttggttaatttgtgggctattggtagagatccattggtttgggatcaaccaaacgtcttctctccagaaagatttttgaattcagatgtggatgttaaaggtagagattttggtttgttaccttttggcgctggtagaagaatttgtccaggtatgaacctagcttaccggatgttgactttgatgttagccactttattgcaatcttttgaatggaaagttgaaaatggtgaaaaagccgaagatttggatatggatgagaaattcggtatcgctttacagaaaacaaaaccattacaaattattccagttttgaaatattgttaa

SEQ ID NO: 51 - DNA, 1488 bp, Synthetic construct
  source 1..1488: mol_type = other DNA
atggaccagaccactctggccatgctgctgtctgccctgtacctgctgtacaacctgtacaaggtgatcttcacccagtctaactctaagctgcctcctggacctaagcctctgcctatcttcggcaacatctctgagctgggcgctaagccccaccgatctttcgccaacctggccaagattcacggacccctgatcaccctgaagctgggctctgtgactaccatcgtggtgtcctctgccaaggtggccgaggaaatgttcctgaagaacgacctgcctctggccaaccgaaacgtgcccaactctgtgaccgctggcgaccaccacaagctgaccatgtcttggctgcccgtgtctcccaagtggaagaccttccgaaagatcaccgccgtgcatctgctgtctccccagcgactggacgcctgtcaggccctgcgacacgccaaggtgaagcagctgtacgagtacgtgtacgactgcgccaagaagggcgaagccgtcgacatcggcaaggccgccttcaccacctcgctgaacctgctctcgaacctgttcttctcggtcgagctggcccagcacacctctacctcttcgcagcacttcaagcagctgatctgggacatcatggaagatatcggcaagcccaactacgccgactacttccccgctctgaagtgcgtggacccctggggcatccgacgacgactggccgccaacttcgagcgactgatcgacgtgttccaggacttcattcgaccccgactgtctatgaacccctcttctgtgacctctgcctctgacgtgctggacgtgctcctgaacctctacaaggaaaaggaactgaacatgggcgaagtgaaccacctcctggtggacatcttcgacgccggcaccgacaccacctcttctaccttcgagtgggccatggctgagctggtgcgacaccccgagactatgaagaaggcccaggacgagatcgagcaggtcctcggaaaggacgccaccatccaagaggccgacattcccaagatgccctacctgcaggccatcatcaaggaaaccctgcgactgcaccctcctaccgtgtttctgctgccccgaaaggccgctaccaacgtcgagctgtacggctacgtcgtgcccaaggacgcccagatcctggtgaacctgtgggctatcggtcgagatcccctggtgtgggaccagcctaacgtgttctcgcccgagcgattcctgaactccgacgtggacgtgaagggccgagacttcggactgctgcccttcggagccggccgacgaatctgccccggcatgaacctggcctaccgaatgctgaccctgatgctggctaccctgctgcagtctttcgagtggaaggtcgagaacggcgagaaggctgaggacctggacatggacgagaagttcggcattgccctgcaaaagactaagcccctgcagatcatccccgtgctgaagtactgctaa

SEQ ID NO: 52 - protein, 268 aa, Bougainvillea glabra
MGGEKKMKGTYYIAHGDPIMYINKSIKLRHFLEEWKENVVMEKPICILVISAHWDTDVPTVNLVEHCDTIHDFDDYPDPLYQIKYPAPGAPKLAMKVQELLKGGGFKCEVDTKRGLDHAAWFPLMLMYPEADIPICELSVQTNKDGTHHYNLGKALSPLLNDDVLIIGSGGAVHPSDDTPHCPNGVAPWALQFDNWLEDALLSGRYEDVKEFKKMAPNWEISHPGQEHLYPLHVALGAAGNNVKTELIHQTWAANGVFGYSSYKFTST

SEQ ID NO: 53 - DNA, 807 bp, Synthetic construct
  source 1..807: mol_type = other DNA
atgggtggtgaaaagaaaatgaaaggtacttattatattgctcatggtgacccaattatgtatattaataagtctattaaattgaggcattttctagaagaatggaaagaaaatgttgtcatggaaaaaccaatttgtattttagttatttctgctcattgggatactgatgtcccaacagtcaatttggttgaacattgcgacactattcacgactttgatgattatcccgatccattgtatcaaattaaatatccagctccaggtgctcccaaattggccatgaaagttcaagaattgttgaagggtggtggtttcaaatgtgaagttgatactaaaagaggcttagaccatgctgcttggtttccattaatgctgatgtatccagaagctgatattccaatttgtgaattgtctgttcaaaccaacaaagatggtactcatcattataacttgggtaaagctttgtctccattactgaatgatgacgttctgattattggttctggtggtgccgttcatccttctgatgatactccacattgtccaaatggtgtcgctccctgggctttgcaatttgataattggttggaagacgccttgctatctggtaggtatgaagacgttaaagaatttaaaaagatggcccctaattgggaaatctctcatccaggtcaagaacatctctatcccttgcacgttgctttgggtgctgctggtaacaacgttaaaactgaattaattcatcaaacatgggctgctaacggtgtttttggttattcttcttataaatttacttctacttaa

SEQ ID NO: 54 - DNA, 807 bp, Synthetic construct
  source 1..807: mol_type = other DNA
atgggcggcgagaagaagatgaagggcacctactacattgcccacggcgaccccatcatgtacatcaacaagtctatcaagctgcgacacttcctggaagagtggaaggaaaacgtggtgatggaaaagcccatctgcatcctggtgatctctgcccactgggacaccgacgtgcccaccgtgaacctggtcgagcactgcgacaccatccacgacttcgacgactaccccgatcctctgtaccagatcaagtaccccgctcctggcgctcccaagctggccatgaaggtgcaagagctgctgaagggcggaggcttcaagtgcgaggtggacaccaagcgaggcctggaccacgccgcatggttccctctgatgctgatgtaccccgaggccgacattcccatctgcgagctgtctgtgcagaccaacaaggacggcacccaccactacaacctcggcaaggctctgtctcccctgctgaacgacgacgtgctgatcatcggctctggcggcgctgtgcacccctctgacgacacccctcactgccccaacggcgtggctccctgggctctgcagttcgacaactggctcgaggacgccctgctgtctggccgatacgaggacgtgaaggaattcaagaagatggctcccaactgggagatctctcaccccggccaagagcatctgtaccctctgcacgtggccctgggagccgccggaaacaacgtcaagaccgagctgatccaccagacctgggccgctaacggcgtgttcggctactcttcttacaagttcacctctacctaa

SEQ ID NO: 55 - protein, 271 aa, Portulaca grandiflora
MGVGKEVSFKESFFLSHGNPAMLADESFIARNFLLGWKKNVFPVKPKSILVVSAHWETDVPCVSAGQYPNVIYDFTEVPASMFQMKYPAPGCPKLAKRVQELLIAGGFKSAKLDEERGFDHSSWVPLSMMCPEADIPVCQLSVQPGLDATHHFNVGRALAPLKGEGVLFIGSGGAVHPSDDTPHWFDGVAPWAAEFDQWLEDALLEGRYEDVNNYQTKAPEGWKLAHPIPEHFLPLHVAMGAGGEKSKAELIYRTWDHGTLGYASYKFTSI

SEQ ID NO: 56 - DNA, 816 bp, Synthetic construct
  source 1..816: mol_type = other DNA
atgggtgtaggtaaagaagtttcttttaaagaatccttcttcttgtctcacggtaatccagctatgttggccgacgaatcctttattgctagaaatttcttactgggctggaaaaagaatgtttttccagttaaacccaaatctattctggttgtttctgctcactgggaaactgatgttccatgtgtttctgctggtcaataccccaacgtgatttatgattttactgaagttccagcttccatgttccaaatgaaatatccagctccaggttgtccaaaattggctaaaagagttcaagaattgttgattgcaggtggctttaaatctgctaagctggatgaagaaaggggttttgatcattcttcttgggttcccttatctatgatgtgtccagaagctgatattccagtttgtcagttatctgttcaaccaggtttggatgctactcatcattttaatgttggtagagctttagccccattgaaaggtgaaggtgttttatttattggctccggtggcgctgttcatccatctgatgatactccacattggtttgatggcgttgctccatgggctgctgaattcgaccaatggttggaagatgctttgttagaaggtagatatgaagacgtcaataattatcaaactaaagctccagaaggttggaaattggctcatccaattccagagcacttcttgccattgcatgttgctatgggtgctggcggcgagaaatctaaagctgaattgatttatagaacttgggatcatggtaccttaggttatgcttcttataaatttacttctatttaa

SEQ ID NO: 57 - protein, 476 aa, Chenopodium quinoa
MGAEPQQLHAVFFPFLAHGHMIPTLDIARLFAARGVKTTIITTPLNAHTFTTAVEKGNKSGAPLIDIEVFRFPAQEAGLPEGCENLEQALGPGEMEKFFKAAAMLREQLEQFLEKIRPNCLVADMFFTWATDSAAKFNIPRLIFHGTSYFSLCALEVVRLYEPYTKVTSDDEPFLLPYLPHEIHMTRLQLSEDLWKNGGKTEYKKRNNAVKESELKSFGVIVNSFYELEPDYADYFRKDLGRRAWHIGPVSLYNRSIEQKSNRGKQASIGEHECLKWLDSRKPDSVIYICFGSTIHLIPPQLHEVAMALEASGQEFIWVVRNEDDLGGFEQKMSGKGLIIRGWAPQVLILEHEAIGAFVTHCGWNSTLEGISAGLPMVTWPMFAEQFYNEKLITRILKIGVPVGAKKWTTTPSIEDVIPQNAIAKALREIMVGDAAEERRCRAKEYKKMAWKALQEGGSSYSDLSALIDELRGLST

SEQ ID NO: 58 - DNA, 1431 bp, Synthetic construct
  source 1..1431: mol_type = other DNA
atgggcgctgaaccccaacagttacacgccgtgttcttccctttcttagctcacggacacatgatacctacgttagatatcgctaggttgtttgctgccaggggtgtgaaaaccacaattattaccacgccacttaacgctcacactttcactacagccgtagagaagggtaataaatctggtgctccattgattgacattgaggtgttccgttttccagcgcaagaggcaggactacctgagggctgtgagaacctagagcaggctttaggccccggagagatggaaaagttcttcaaagcagcggccatgctgagggaacagctagagcaatttctagaaaagattagacccaactgtctagtcgcagacatgttcttcacatgggcaaccgattcagccgccaaattcaacatccctcgtctgatatttcatgggacctcatacttctccctatgcgctctagaggtcgttcgtttatacgagccatatactaaggttacaagcgatgacgaacctttcttacttccgtatttgccgcatgagattcacatgacgcgtttgcagctaagtgaggatttgtggaagaacggtggtaagacggaatacaagaagagaaataacgctgtgaaggagtcggagcttaaatcattcggtgtaatcgtgaacagtttctacgaacttgagccggactacgccgattacttcaggaaggatttgggtagaagggcgtggcatatcgggcccgtgtccttatacaacagaagcatcgagcaaaagtcaaacaggggcaagcaagcgtctattggtgagcatgaatgcctgaagtggctggacagtagaaagccggactccgtgatctatatctgtttcggctcaactattcacctgattcccccgcaattacacgaggtcgcaatggccctggaagcatcgggccaggagtttatttgggttgtgaggaatgaagatgaccttggcggattcgagcagaagatgtctggcaaaggtttaattataaggggatgggcgcctcaggtcttgattttggaacacgaagctatcggtgcatttgtcacgcattgtggctggaactccaccctggaaggaatttcagctggattacctatggtaacgtggcctatgttcgctgaacagttttacaacgaaaagttgataaccaggattttaaagatcggtgtgcctgtgggagcaaagaagtggacgacaaccccatccattgaagacgtgattccgcagaacgcaatagcaaaggctttacgtgagattatggtaggggatgcagcagaggagagaagatgccgtgcaaaggagtacaagaagatggcgtggaaggctctgcaggagggaggaagttcttatagtgacttgtctgcacttattgatgaattgcgtgggcttagtacctaa

SEQ ID NO: 59 - protein, 485 aa, Beta vulgaris
MGAEEFHVVLFPFMAHGHMIPILDIARLFAVRDNVKATIITTPLNVTNFTKDNESNMESSAPIVNIEVFRFPAQEVGLPEGFENLEKVMKPELLNKFFKAAGMLNEQLEQYLEKIRPNCLVADMFFPWATESAAKFNIPRLVFHGTSNFALCAQEIVRLYKPFKNVTSDEEPFVLPSLPHDIQMRRLQIPEDLWKYDGTEFEKRMDSLKDSEIKSYGVLVNSFYELEPDYAEFFKKELGRKTWNIGPVSLSNRSVKDKSQREKEPLIDQHECLKWLNSKKPDSVVYICFGSTASFIAPQLHEMALALEVSGQEFIWIVKNDENGRSKEWLPPGFEQRTEGKGLIIRGWVPQVLILEHEAIGAFVTHCGWNSTLEGISAGVPMVTWPIFAEQFYNEKLVNQILNTGVPVGAKKWSRTPSIEDIIKMEAIEEALTDIMVGTEAEERRTRAKKLKILAYKAVEEDGSSYSDLTALINELSANRLQEQE

SEQ ID NO: 60 - DNA, 1458 bp, Synthetic construct
  source 1..1458: mol_type = other DNA
atgggtgccgaagaattccacgttgttttgtttccattcatggctcatggtcatatgatcccaattttggatattgctaggttgttcgccgttagagataatgttaaggctaccattattacgaccccattgaatgttaccaacttcaccaaggataacgagtccaatatggaatcttctgctccaatcgttaacatcgaagtttttagattcccagctcaagaagttggtttgccagaaggttttgaaaacttggaaaaggttatgaagcccgagttgttgaacaagttttttaaggctgctggtatgttgaacgaacagttggaacaatacttggagaagattagaccaaactgcttggttgctgatatgttttttccatgggctactgaatctgctgccaagtttaacattccaagattggtttttcatggcacctctaactttgctttgtgtgctcaagaaatcgtcaggttgtacaagccttttaagaacgttacctctgacgaagaaccattcgttttgccatctttgccacatgatatccagatgagaagattgcaaatccctgaagatttgtggaagtacgatggtactgaattcgaaaagagaatggactccttgaaggactctgaaatcaaatcttacggtgtcttggtcaactccttctatgaattggaaccagattacgccgagttcttcaaaaaagaattgggtagaaagacctggaacatcggtccagtttctttgtctaatagatccgttaaggacaagtcccagagagaaaaagaacctttgattgatcaacacgagtgcttgaagtggttgaattctaaaaagccagattccgttgtctacatctgttttggttctaccgcttcttttattgctccacaattgcacgaaatggctttggctttggaagtttctggtcaagaattcatttggatcgtcaagaatgacgagaacggtagatctaaagaatggttgccacctggttttgaacaaagaactgaaggtaagggtttgatcattagaggttgggttccacaagtcttgatcttggaacatgaagctattggtgctttcgttactcattgtggttggaactctactttggaaggtatttctgctggtgttccaatggttacttggccaatttttgctgaacagttctacaacgagaaactggtcaatcagattttgaataccggtgttccagttggtgctaaaaaatggtctagaaccccatccattgaggacattattaagatggaagccattgaagaagccttgaccgatattatggttggtacagaagctgaagaaagaagaactagagccaagaagctaaagattttggcttacaaggctgtcgaagaagatggttcatcttactctgatttgaccgccttgattaacgaattgtctgctaacagattgcaagagcaagagtga

SEQ ID NO: 61 - DNA, 2805 bp, Yarrowia lipolytica
  source 1..2805: mol_type = genomic DNA
atgttgcagttcctcttgctgctcaacctcgccctgtcggcatctgctcgagtcatccacaaaaaggtcactgtggtcaacgatgtggttgtcactccttctacaaccagatacgtgaccgttactggcggccagtccgagcacgtgactcttgatcagactatcgagtccgtcgagactgtgtttgtggcccacaccgttcccacaaccgtcattaccggcactctcatctccaccgtccagtctattatcactgccaacccggtgacccagactgattcgtaccccaccaccgcagatgctccccagatcaacatcaacactctgaccgagcccaagaccctggatccctttgagggagttgtaactgatgctgagcccattcccacagaggccaccgtcgcttcttccgcctctgcaacccccaccgctactcctactcagtcttccttctcggtgtctatccggtcttcttctactcctggtcctgttgagtccactcccggtgttgtttccatccctgatggtccctccaccacttctgctctgatctctagctccgtcgttgcatcttccgttgcatcttccgttgcctcttctgcttccgccgctgcttcgtcttccgccgctgctcccaagcccaagcccaacccccctcctgcttacaatggcgacctgttcgcagccattgacacctctgcccctcccggagttttcaagcaggagcctctgaacatccagatccccaacggcgccgtcaaagagcttggtcagtcccccgtccacaccaacaagttctacttcaacatgttcctcggtgaccgaaccatgcccgtctacacccagccctactcggtgtggtggtccaagaccgacaagttccccggtatcggtgtctcttactcccaggctaaggacaaggtctacggcccccagaagaccaaccccatggagtacatgctgaaccccgttggaatcatgtcctttgtcttctccgccaaggagttcaccaccgacaacatggagatgcagctgtctgaccccgacactttctctgccaccaccactgtttctaccggtgacggctccatggaacttcctcttgttcagggaatgggttttgtcactgccaagtacaacggtctgactcctcttctgctgacccaggtcggctttgactccgtcgtcaagggcaactcccccgtgcagggcacccttaagtacgtggctaagctcttcaacggagtcacttggaccatctacgtgaccacctctgatcctgacttcaagttcctcttccccgaccctcacaccatcaagactcactccaccaagccctgtgtgattcagatggccaccactggtgacgctgactctgatgctggctctgctgtgtctggcttcgacaaggcagctggttcctaccccgtttctgcctctctcaacggccaggctaacggaggttctgctcaatactccatcgactacaagacggagggatcttctgcttctggctcaactctgctgtttgctctgtctcaccacaagagctccatggtttctcagtccggtgctgtttccggccctaagatccagtccaccaacaagggtcctatgtacgcctacgtggccaactctctgaccctcgctgagaccctcgagactgatctgcagttccttccctggtcccaggtgtctggatttgctggtaaggccaccctgactgctgcgcagcagaagctgattgccgaggtggccaactcagagctcaagcaggatattggaagccagaccaacctggacacgaactacttctccggcaaggctctggacaagttcgcctacattctgctggttctgtctgatatgattgaggatgagaccaccaccaagtctgttctggaccagctcaagaaggccttcgccgtcttcactaagaacactcaggtatctcccttcatctacgacactctgttcaagggagtcacctccggtgctgcccaagcctctggcgactctggtgctgactttggttctccttactacaatgaccaccacttccactacggctatttccttcatgccgcagctgtgattggccatgttgatgccaagtacggagatggtcagtgggtcaacgagaacaaggactgggtcaactctcttatccgagacaccgccaacccttccaaggacgactcttacttccctgtctaccgatcttttgactggttctccggccactcttgggccaagggtctcttccctgctgccgatggtaaggacgaggagtcaacctctgaggactacaaccacgcctacggaatgaagctgtggggtaacgttgtcggcgataaggctatggaggcccgaggagacctgatgctggctgtcatgaagagatctatgaatgactatttctacatgaaggacgacaacaagatccagcccgagcagctcattggcaacaagatccccggtatcacctttgagaacaagctggactacaccacctactttggcaccaaccccgagtacatccacggtatccacatgatccccgtcactcctgtctcttctctgctccgagaccctacattcgtccaggaggagtgggagcagaacgtgtccaagttcattggagacgtcaactctggatggctcggaatcctacactctaaccaggctctttacgaccccaagagtgcctacgagtttttctctcaggacaatttccagactctttggcttgatggaggtgcttctcgaacctggtatctcgcctactctgccgcagtgtccgcc

SEQ ID NO: 62 - DNA, 2532 bp, Yarrowia lipolytica
  source 1..2532: mol_type = genomic DNA
atgatcttctctctgcaactactactgacgacggcactagcggcctcttcgcctgactggtaccccacacccgaaatcggttcaattaccagtgattgggccgatgctcttggtgagtccatggacatcctggcccaattgactcttcccgaaaaggtcaacatcaccaccggtaccggatggagaggtgggcagtgtgttggaaacacaggggctgttcctcgtctcggaatcaagggcctgtgtctccaagacggtcctctgggcgtccgttttgccgactttgtcaatgtcttcccttgccagaacgcaatggccgccacctttgatcgtatcctagttcaccaacgaggaaccgctattggacgtcagtctagactcaagggagtcgatgttcatctcggaccagtggtgggacctcttggacgacacgctaccggcggaagaaactgggaaggtttctcccccgacccatatctgtctggaaagctcgccttcgaagcaattcttggagtccaggaagagggagttcttgcaaccatcaagcacttcattggaaacgaacaagaacattaccggcgagccgaagagtggagagacggctttggatttaaagacctgaaggacgccgtctcttccaacattgaagacagagctctacatgagttgtacatgtggccgtttgccgatgctgttagggctaatgtcggctcagtcatgtgctcctacaactacgtgaacggaacccaggcttgccagaacagtgacttgctcaacggaaagctcaagtccgagctcggtttccagggctttgtcatgtccgactggtttgctcagggaagcggagtgtctaacgctctggctggaatggacatgagtatgcctggaaatgacgttgatgagttagaaactgtcttctggggagaacagctgacccgaatggttgccaacggtacccttccagaagcccgtctcgatgatatggttctgcgaattctgacccctctcatctactttgggatcgacgatcgaacacccaacttctcctcctttgtcgacactacagtgggaagtccccaccccgctgctaagcactccaagaaagtcaaggatgttatcaccaactaccatctcgatgtgcgagaccagtttgcagccaatgttgctcttgatagtgctcgaggagctgttgttctgcttttcaatgacggtattcttcccctgaagaacatttccgctattggagtatttggagtcggctctagacttggccccaatggagctgtttgtggggaaaacatgcagtgctcagacggagctcttatcgaaggatggggaagtggaactgcttaccctacggagtacgaaagcccttacgaagctctccacaaaaaggcttctctacttgaagtgtcagtgacaggaaccaccgagtcatgggatatgagacttcctcttgagctggctggagacactgacgtgaatattgtttatgtattggcaaattcaggagagtctactgccaacgttgataagaaccttggggatcgccgaaatgtcagtctatggcacaatggcgatgaacttattaatacagttgccagtcagggacagactgtcgttgttgtcactacggttggacaagttgacatgaccgcttggctcaaccaccccaacatcagtgccgttcttctgactgctcctgctggtgattacggaggaaaagccatcgctgatgtgttatttggagaggttaatccctcaggaaagctgccttatactatcgcagcaaatacttctgattatattcctattgtcaccaagatccctcgagatggagctccccagtccgactttgtggagggaatctatcttgactacaagtggtacgacaagtttgaaaggactcccctctacgaatttggttacggtctgtcatacaccacctactccttcagcaatctgcatcttgatgttaaggagattagtgagttccttcctccccggcctgttcctgtacaggttactaaacccaagatgaccaatatcgacattgaggacctttatgtccccaatgactttaaaatgattgacggtcttgtttacccttggattctcaacgccagtgcgcccctcgcggactctcagactcagttcccctttgcaaatggagctggacatgtcagtgacgcttctggaggagtgggtggtcacccctggctttggtctaacgctgttactgtcactcacaacaccaccaactgcggtgatgttgctggacgagtagtttctcagctgtacgttgccttccctgaaacccttatcgactctcctccggtgcagcttcgaggattcgacaagtccaagctcctgaaccctggtgagtctcagatgaccgagtacaacctcaactggcgagatctggccatttgggacgtggaactgcagagctggagagtgcagcgaggcgagtattccgtgtacattggtcattccagccgagaattcgagctatgtgagactttcactttg

SEQ ID NO: 63 - DNA, 939 bp, Yarrowia lipolytica
  source 1..939: mol_type = genomic DNA
atgaagttcacatttgctgccgttaccgccgcgctggcctcgtccgccatggccctcggaggcctcggagtcgacctcggagtcaagcgagagtccgacggagagtgcaagaacgccggcgactacaaggccgatcttgaggctctcaagggtctcaccgacaccatccgaatctacgccgccggcgattgcgacgccctgcgagagctcggccctgttgctgaggccgctaacttcaagctcatgattggtgtgtggcccaacgacgacaaccactttgcctccgagcagtttgctctcaagagctacctgccctggctctccaagtccaccgtgccctacatcaccgtgggctccgaggctctgtaccgaaaggacatgactccccagcagctggccgacaagatcaacgacatcaagaaccagctcaagggcatcaaggacaagaacggccagacctttgacgtccccgtcggtaccgtcgactcctggaacgtgattgtcgatggctactccagccccgccgtcaaggccgccgacgttgtcttcgccaacgccttctcctactggcagggacagaccatggctaacgcctcttactccttcttcgatgacatcatgcaggctctccagaccatccagaccaccaagggtaccaccgacattgacttctgggttggtgagaccggatggcccaccgatggcggtgctttcggcgactctcagcctggcgtgaagcaggctgctcagttctggcaggagggtatctgtgccatccgagcctggggtatcaacactctggttttcgaggctttcgacgagacctggaagcccgacactaagggtgacaacggtgaggaggtctccggcgttgagaagtactggggtgtttacgactccaacctcaagcccaagttcgacaccacttgcaagtttgac

SEQ ID NO: 64 - DNA, 2607 bp, Yarrowia lipolytica
  source 1..2607: mol_type = genomic DNA
atgctcgcattcgtcctactgctgacgatgctgctcgcagcagcgcttgctgacccgttctcagataaagacgcttacaaacacagccctccatactaccctgctccggagattggcagagttcccaccgacctgcgatggagagctgccttgaaggtggcccagggtatggtcgctaacatgacactgcttgagaaggtgaacatcaccaccggtacgggctgggagatgggtccttgtgttggaaacactggtaccgtcgaacgactgggtataaaatcgctgtgccttcaagacggccctcttgggattcgatttgctgacctcattaccacattccctgctggtatcactattgcctctaccttctctcgacagctggttagagagcgaggtgctgctatgggacgggagaatagacgcaagggagtggatatcactctcagccctgtggttggaccactgggaagacatgctaacggaggtcgaatctgggagggcttctctgctgacccctaccttgctggaaagctcgccgccgaggccgtgacaggtatacagggccagaacgtcatggctgtggtaaagcatatggttggaaacgagcaggaacattttcgacaacttggcgagtggcagggattcggattcaaggatctgaagcagcccctctcttcaaacatcgacgaccgaactcttaacgaagcgtacctttggccctttgctgatgctgttcgagccaatgttggatctgtcatgtgttcctatcagcagatcaatggctctcagggttgtcaaaacgcccacattctgaacggtaagctcaaggaggaaatgggtttccagggctttgtcatgtccgactggcttgcccagcgaagtggcgtggcgtctgttcttgctggtctcgatatgagcatgcctggagacggtcttgtctgggcggacggtgttccgctcatgggatacgagttgaccaggagtgtgctgaatggaaccattgatgaaagccgagtggacgacatggttacccgaatccttacccccatactgtatctctcaattactccgaccgaccccaacttcagctcttggaccaacgacactactagctacaagtactacggagccaaggctggtggaaacgtcactgttaaccgacatattgatgtgagagaccagtacactaccaaggctgctcttgatggagcaaacgctgcgcttgttcttctcaagaatgagaagaagactcttcctctgaaccctaccaatattggaaacctcaacattttcggtattggttctaaaaccggcccacttggagctgtctgtggagaaaatatgcagtgtagtgatggcgcccttattgagggatgggggtccggttccgtctaccctaccgattatcaatctccttacgacgccattaaggagagagcctccaaggacaacatcaccatcggaggcactacgcaatcttggggtaacctgtcgaacgttgagatcctttcagctgctgccgacgccagtgtcgtctttgttctttccgactccggtgagagtactggtattgttgacggcaacattggggatcgaaacaacttgacgctgtggcacaatggagacgaggttgtcaaggctgtggcatctaagaaccccaacactatcgttgttgttaccactgtcggccctgtgaacctcgaaaagtggatcgacaacccaaacgtcactgccgtgcttctcactggacccgctggtgactttggaggaagagctgcagcctctattctcttcggcgacatcgccccttcaggaaaactccctttcactattgccaagaatgacaccgactacattcctcttactactaagatccctgaagacggccttcctcaagactatttcactgagggtactcttttggactacaaacggttcgacgagaaccaggtgactcctaggtttgaatttggctacggtctgtcttactctaacattacggtggagaatctcgaagcccggtatgctttccctagcattcctgagttcttgcccactccctttgccccttcgaaccccaacaagcctaagaacgcatttactcctcacgccaatgagtccgtcttccccagtgacattgatcctttgaacaagtacgtctatccatacctgaacgatacctcggagatcttctctaacgagacccattatccctatcctgaggggtactccagtgagcagtccaacagtaccaacattaacggcggggctgtcggaggcaaccctgctctgtggctctctgcagtctacattgtccacagcgtgtctaactatggtccctatgatactggagtggtcacccagatgtacattgccttccctcaggataacgacgatcttaaaaccgctcctagacagcttcgaggattcgaacggtccgagctcaaggtgggagaacggcagggaattctatacgatgttcaatggcgagatctcgcggtctgggatgtcaaacttcagagctggcgggtccaacgaggagagtacaaggtttacgtaggccacagttcgcgagactttgttctgaccaccagcttcactctcaag

SEQ ID NO: 65 - DNA, 2817 bp, Yarrowia lipolytica
  source 1..2817: mol_type = genomic DNA
atgagatacacaaatacccaagcgctggtgggtgtctgtctactcctgctgctggcttgcgtggccaaagcacagagagaaccccctcaccctcacccccgatacccaatggctccacccccgcctcctcactacccttactactaccccaaccgtcatggagcgtacagacgtactcgtcaagcagcaggcagatcccccgtgggtctccgaaatgaccgacgtcccaaagcgcgactcgataaccacaagaagaagaccgcccagcaccacgacggagagaagaaggccaagcccaagtccgataccattatcatccgaattgcaagcaatgtcggtggcgatattcccaagctctcagacccggagcccttcgattccaacccctttgtgaatctcacaaactacatgcattcccccacctattaccccacccccatggctggtcggatcaatgatcactgctggcgatcggcgtttctcaaggccaaggcgtttgtcacccaactcacgaacgaagagaaggctaacctgaccactgacttcagcactgcagattccccttggtatggcgagactggcgagattccccgactcaacttgtcctctctgcgtctccaatggggtctccatggtgtcggcggcgactctcacttcaccaccttccttcccgccggtataaccacagcatctaccttcaacaaaggactgatgtactctcgtggtgccatcattgggaaggaggctcggaagaagggcatggacatcgttctcggtcctctcattgatcccactggtcgaagtgccgctggcggacgaaactgggagggattcggtcctgatccttacatggctggtgtcgttgcaactgagtccgtcactggtatccaagatcagggagttgttgctactgtgatgcactacgttgggtacagccaggagcacttccgaaacttggaagagtggcaggcccacggttacaacaacctcacatcctctggctcctcattcatcgatgacagaacaatgaacgaggtgtacatctggcccttcgccaacgctgtcaaggctaatgccggtgctatcatgtgtgcccctcaaaagctcaacaacacccagggctgcaagaactcctacatgatgaattacaagctcaaaagagagttgggattccaagggttcgtcctctccgcgggtatgtctcagaacgaaccccctgctgcgctcgccggtatggatatgtcaatgcctcggctcaaggcgtctgaaaacaagctgcgtgaaatgatgctccgaaattatgacaaagacggattccctcaatcacgcctagacgacatggctacacgagttctcacctcctactactacgttacacagcaccgaaacggccaccagtcctttgtcggcgacgaggagaatctctccaacaagtcagcctacgacaacatcttagttgatgttcgtgatggttttcactaccgggtcgctctcgagatcgctctcgaaggtatcgttatgctaaagaatgatgatagtgcccttcctatcgagggcatgagaaccattggagtgctgggagccgctgccaaccttggacccagtggctccaagtgcgacgacaagtttgggtgccacgatggtgctatcttccaaggatggggagacggtggcgtcaaccctccgttcgttgtgaccccctacgaggctgtgaacgcccgtgctgctcgagaccgaattgttgttcggtccaacttcgattcttgggatctcaaccaggccgaggccgttgccagcagcactgatgccaatatcatcttcgttgccgccaactccggcgagggtaaccacgtggtggatgataacaagggcgacagaaagaacttcaccctctggcacaatggcgacgagctcatcaagaaggctgttgaggtcaacgaccacaacattgtcgttgtcactgctgttggtcctgttgacatggagaagtggatcgagcacccgcatgtcaaggcagttctgttcactggccctggcggcgaggaggctggggctgctcttgctcacgtgctctttggagatttcaaccccagtggtaagcttccctttaccattgcacggaacgtcaaccactacatccccattgagaccgaggtacctcgggatgggattcccaaggcttactttggtgagatgagcttgactgattacaagtggtttgatcagaacctcatctctcctcgattcgagtttggtttcggcatgtccttctctgagttctcatactctgacatgaagattaccacacagagaaaccccagtctcacccttggagctcccccggactactggggtaaagacggcaacgcgactactgctaaggcgttccagcaagagttcaaggccgcttcaggggacttccagtttccttctggctataatgatgaccacaacgacgatcctgtcctgaatggtggcgctgtgggtggtaatcccatgctctgggatgtcatgtaccaagttgctgtgagcgtgaccaaccacggcccctttgatggtgcagtcgtgtcacagctgtatgtcagcttccctcaggatgaccaggctctccggacagcccccaaacagctgcgaggcttctctaagacttctcttgttgttggcgagacagccaacgttctttttgatctcatgtggcgtgatctcgcagtttgggatgttgtcaagcagacctgggtggtacagcggggcgactatgatgtgttcatcggtggttcgagtcggcagttagaaacccttggtcgcatcacgattgct

SEQ ID NO: 66 - DNA, 2907 bp, Yarrowia lipolytica
  source 1..2907: mol_type = genomic DNA
atgattgcaaaaatacccctacttccgttcgtggtaggagctctggctgtgtccaactccaccgactccactctcaccccgactccatctcaccaactcaactctcaccaaactcaactctcaccaaactcaactctcaccaactcaactctcaccaacttcaccaactctacccatgttcccttcacttctcccgatttctatcctactcccgaaattggcactctggacgcggacaagcggtggagagctgctctgaacgagtccctggagattctgtctcagctgactcttgtcgaaaaggtcaacatcaccacaggtcttggttggggaggaggcacctgtgtcggcaacactggcggagttcccagactcggtcttaagggtctctgtctccaggacggacctctgggaattgctcagaccgactacgtgactgttttcccctgcggaattgccatggcggccacctttgacagaaacctggtccaccagagaggcaccgccattggacaggaggccaaggccaagggagtcgatgtccatctgggacccgtcgtgggacctctgggacgacacgcaacgggcggccgaaactgggagggcttttctccggacccctacctggccggaaagctcgtgtccgaagctatccgaggtatccagtccgagaacgtcatggccaccgtcaaacacttcattggcaatgagcaggagcactaccggctctactccgagtgggctcgtttcggattcgacaacctcaccacttcggtatcctccaacattgacgatcgaaccatgcacgaggcctacctgtggccctttgccgacgccgtcaaggccggagtggcttcagtcatgtgttcgtaccagcagatcaacggctcgtcgggctgccaaaacagcgccacgctcaacggcaagctcaagtccgagctcgggttccagggctttgtggtgtcggactggcaggcccagctcagcggagtgtccaatgctctagccggactggacatgagcatgcctggtaacgacgtggacggaaacatcttctggggaccggacttgaccaaaatggtggctaacggcactcttcccgagtccagactcgacgatatggtgctgcgaattctcacagccaccatctacacaggcatcgacgaaagagagcccaccttctccgccttcactactgaaacctttggcaaccccaaccccgtgctcatgttcaacatcaactacacctccaccttggtcaacctccatctggacacccgaaccgccttttcttcccgtgtggcgctggagggagccgaggctgcagcagtccttctcaagaacgacggaattctgcctctgcagggccccgagaatgtcggagtgtttggagtcggctcacaaatcgggccaaagggagcctactgcgggttctccatgcaatgctcggatggagctctcatcgagggatggggaagtggaactgccaacccaacagagtacacttctccgtacgaagcactccgacaacgagccaaccaagctggaggccacgtgattggaaccaccgagtcctggaacatgacgctgcctctgatcatggctgacaactcggacgttaacatcatctatgtgctgtccaactccggagagggaggttcgaccgttctggacaacatgggagacagaaacaacgtgtctctgtggcacaatggcgacgaactgatcaccactcttgccaacagctccagaaacaacattgtcgtcgttaccaccgttggtcaggttgatttggagccttggatctcgcatcccaacgtatcggccgtggttctcactggtcctgctggtgcctatggaggtaaagccatggccgaggttctctatggagacgtcaacccatcgggaaagctgccttataccattgccaaggacccccaagactacattccggtggttgtggacattcccgaagatggagctcctcaggcctactttgatgagggtgtctacatggactacaagatgtttgacaagctcaacaagacagttcggttcgagtttggttacgggctgtcttattcggatttcgagtttggagacctggatgtggttgtagacgagttcatttccgagcttcttccttctcctcctcggcctatcattgttgacaagcccagcagcaccagctccaactcgtcccacggcgatctcaaggctcccaagggtttcaagcccattcgaggagttgtctacccgtggattgacgttaattccctgactgccggcgaagctctttcgggagtgagtcctgctcttgccactttgctgggtctaggtaaacctgatacctgtgctgttcccgccaagcacgagactggcgatgatgacaaggactccaagtctgacgataagaccaagagagacaccgagtcggatgctgccgatgatgaaattgaaacagtcagcacctccaacgaaaccgccgttgaaaccaccattggaatgaagaacacctccgagcccttccactttgccaacggaacagggacgtcaacaaacgccgccggaggagttggaggaaacccatctctctggaaaaccgtcgccaccgtgtctcacaccatccgaaacctcggtccctatccaggagccgcagtcacccagatgtacattgccttccctcaggacgacattgattcgcctctcattcaacttcggggatttgacaagactcgggttctggacgtcggagaactcgaaactaattcctacgacatcttgtggagagacctggctgtttgggacgtggtcatccagagctggagagtccagagaggagagtacaagatctacgtcggcaactcctccagagactttgttctcatcgagtcctttactctccag

SEQ ID NO: 67 - DNA, 2031 bp, Yarrowia lipolytica
  source 1..2031: mol_type = genomic DNA
atgaagttcacaactctcctttctatggcctctgtggccatgtctgccgccgtcactgtggagattcactctacggtgctagaaaccgtgttcgaaacagcctctgagacgataatttccgacgccgcaacccccgaaccctcccccacagtcagatatgttaccattctgcccaaaccctttcctgcattcaacggcgttgccctgagcggcactgtccacaacaagcgagacaccgtcgagacttctgtcgactgcaatctcggactggcctctgcctctgctgctgctctccgggagcaaagttcagctggtacctcgtccgcagcatataccgcaaccttaacttctggtagcaatcttagctccgagacccacaccattgaggtttcttcttctacttcttccattcccgaacctgccaccgcatcaattgagaccgagcctaaccccagtctcatccccctctttatccccgttcaccaggaagactccccccccgacactaccaccgacactcacaacatgggacttggaaccaaaatcagagacatggtcaacaacgttggccccgtggccgctgccgacctcagcaagcccaaccaggctcccaatgaccgaaccatcttccagtcgcgaaacaaccatggccccaacattggctccatgttccttctcgagaagtggctgacccctggcatgttccccgactccgccaagggagacgctgagctcgacgctgtgaccgctctcgttgccgaacagggtgccggcggcgcccagaagaagtttgaggaccactggaacacctggatcaccgacgacgacttctcgtacctgcagtctgtgggagccaacgccatccgagtgcccatgggctactggaccatcaacggaggcgccttcacccagggcacccccttccagcagtaccagtccgtctaccagaacgcctggtccattttcaagaccaacattctcgacaaggcccgagccgccaacattgctgttctgattgatctgcatgccgttcccggcggcgccaacggagacgctcactctggaacctcttccggtaaggtcgagttctgggactctcgaagcgaccagaagattgctattgacgctctccagtgggtcgccaaggacgtgctttcatacgacaacgttctcggtatcgaggtcgtcaacgaggccgtctacgacgcttccacttccaaggagggctcttactacctgcgagctctcgaggccatccgacaggtgaaccctgacgttcccgtctacatctccgatggctgggctcctaccgaatggaacgagtgggtccaggagcagaaccagaagctttctgccggtcagaacactggttttgttgtcgactcgcacgtctacaaggccttctccgagcaggacaagggcaactctccccagcagaacattgccaacgttcccgcctacctcaatgttggaaaggcccaggctgattctattgtcggcgagttctcctgtgtcttctccgaggagacctgggctaaggccggcggtgaggaccgagagcagcttgccatcaagtacggccaggttcaaattgagtccttcaaccagaatgcccgagccggatggttcttctggacttacaagttccagtacggcgatggaggcgattggggcttcaaacccatgacccagaagggcgccctcccgaccttctccaacggaggctccaacaaggatcccaaggccggcatcaaggaggcagccaccaaggctaccgaggagcactctgcctactgggaccagcatcagggtaactacgagcactggcgatacgccgatggatacgtttcgggatggcaggacgctgccgccttctacgagttccacggctccaccattggccgaatcaacgcctggcaggacgcccgacgagcccagcacattgctgctaagggtaactccggtgccatctgggagtgggaccagggcttcgacgctggtattcagggtctgctcaactacatgcgatct

SEQ ID NO: 68 - DNA, 2586 bp, Yarrowia lipolytica
  source 1..2586: mol_type = genomic DNA
atgtttggagggggtgaaaaaaaagacgatcaaatatataaagaagggcgggggctctttcacaaatctacacactctcaacaaacaacctcaaacatgccaatcgacgtgcaaaacaccctggaacagctcgagctgtcggagaaaatcgcactcaccgctggagtcgacatctggcataccgtccctatcgaacggcttggcgtaccttctgcccgaacctctgatggccccaatggaatcagaggtacccagttcttcaactccgagcccgcagcctgtcttccggcctccctaggcctcggtgctacgtgggatcaggatctcttgtaccaggtaggagagctgctggccgaggagtcgcgtgccaaaagcgcccatgtcgtgcttgcgcccacaattaacatccagcggtcccctctaggaggtcgctccttcgagtctttctctgaagatccgcttttgtctggaaagctggctactcagtacgtcaagggtctacaggacaacaaggtggcagcttgtattaagcactttgtgaccaacgaccaggagaatggccgcatgggatccaactcagtcgtaacagatcgagctttgcgagaaatctatctcaagccctttgagatcgcagtcagagaagccaaccctaaggccttcatgaccgcctacaacaagctcaatggcacccatgttagcgagcacgacatcttgggcagtgtgctgcgaggcgagtggaaatggcgagggttggtgatgagtgactggttcggcacttactccaccagtgacgctgttaatgccggtctggaccttgagatgccaggtcctcctcgatggagaggcgagcagctcacccatgctgttctcagcaacaaggtgaccactgagacgcttgatgagcgggtcacaaatgttcttgagctcgtgaagtacgcccaagagtctggaatcccgttcaatggtcccgaatccaccaacaacacgcctaagactcgggctctgctacggaagctggtggcagactccactgtcctgctcaaaaacgatgcaaacatcttgcctctcgacaagaccaagaaagtggccgtgattggccccaacgcaaaggccactgctttctgtggaggtggttcagcctctctcagaccctactatactgtttctccgtttgagggaatcacttccaagactggcagcgaacctgattacgccgtaggagcctacgctcataaggagctgcctgacttcgcatcatacttgaagtcagagtctggagacgagggaatttgggacgttcgattctacaacgacaagaggggtgctcaggatcgaaagtgctttgaccaattgactatcgagcagaccaaactgtttctgttcgattacagtcacaaggatatccccaagaacaagatcttttatgtggacgccactgccaccctcaaggttcctaaggacggagtgtacgactttggaatgacccttctgggtactgccaagttctttgtcgatgacaagtgcgtgctggactgcactaaggaccaggagaacggcaattcgtttttcggcgaaggcactaaggagaagattgggtctattgagctcaaggccaacaaggactatgccattcgcctcgagtttggatccgaggccacttcccctcgagagcgaggaggacttgtttccaacggtggaggcgctgttcaggccggcatggctcgacaggctgccgccgaagatacgatcgccgaggctgtcgagctggccaaacgttccgaacaggtaattctcttttcgggactcaacatggcgtgggagtctgagggattcgaccgaccgaacttgtcgcttcctcctcacaatgatgcactcatcgaggccgtgctcgacgcgaatcccaataccgttattgtcattcagagtggagctccagtggagatgccctgggcatccacggccaagaccatcgtgcatgccacctttggaggcaacgagactggaaacggtattgccgatgtgctatttggcgacgtgaaccctagcgctaagctgcccatcacctaccctctcaaggttcaacatactccctcttactacaacttcggccatcctaaacgaactttgtatggagaagatgtgtttgtgggctaccgacactacgagaaggttgatcgagaagtgttgtttccctttggccatggtctttcatacacttcatttgagctgtctaacctgagtgtttccaagaaggatgatactgtcactgtaactgtgaacgtcaagaacaccggatccaaaacgggagctgaaatcgtccaggtttacgtttctcaagagaaaccatccatcgtgcggcctgtaaaggagctccggggcttctccaaggtcgagcttgatgctggcaagtccgataccgtcactgtggaactggatgtcgatcaggccacctcattctggaacgagtatatcagcaagtggaccagtgaagctgacaagtaccatattcatgtcggtaccagcagtgcaggtaagcatcttgagggtgagtttgaagtcaagaagactaagaattggttaggtctt

SEQ ID NO: 69 - DNA, 1263 bp, Yarrowia lipolytica
  source 1..1263: mol_type = genomic DNA
atgaaactaaccaaacttgtagctctggcaggagccgctctggcttcgcctatccagctggtccctcgagaaggctctttcctgggattcaactacggcagcgaaaaggtgcacggcgtcaaccttggcggctggtttgtgctggagcccttcatcacgccctctctgttcgaagcgtttggcaacaacgacgccaacgtgcccgtcgacgagtaccactataccgcctggctgggcaaggaggaggccgaaaagcgactgacggaccactggaacacgtggatcaccgagtacgacatcaaggccattgccgaaaactacaagctcaacctggtgcgaatacccattggctactgggccttttcgcttctcccaaacgacccctacgtccagggccaggaggcatacctcgaccgagccctgggctggtgtagaaagtacggagtcaaggcgtgggtcgatgtccatggagtccccggctcccagaacggcttcgacaactccggactgcgagaccactgggactggcccaacgcagacaatgtgcagcattccatcaacgtgatcaactacattgctggcaagtacggcgctcccgagtacaacgacattgtggtgggtatcgagctggtcaacgagcctctgggccccgccattggcatggaggtgattgagaagtacttccaggagggcttctggaccgtgcgacacgcgggctccgacactgccgtggtcatccacgacgcctttcaggagaagaactacttcaacaactttatgaccaccgagcagggtttctggaacgtggttctggaccatcaccagtaccaggtcttttctcccggcgagctggcccggaacattgaccagcacattgccgaggtgtgcaacgtgggtcgacaggcttccacagaataccactggcgaatctttggcgagtggtccgctgctctgaccgattgtacccattggctcaacggtgtgggcaagggccctcgtctggacggctccttccccggctcgtactaccagcgatcgtgccagggtcgaggcgacattcagacgtggtccgagcaggacaagcaggagtctcgacggtacgtggaggcccagctcgatgcgtgggagcatggaggtgatggctggatctactggacttacaagacggagaacgcccttgagtgggacttccgacggctcgtggacaatggcattttccccttcccatactgggaccgacagttccccaaccagtgtggtttt

SEQ ID NO: 70 - DNA, 2145 bp, Yarrowia lipolytica
  source 1..2145: mol_type = genomic DNA
atgttccccataggaaggaagggcaatagccaggacaaggggcgcaagagacccctgctacggagccacctgttgcccataggaacttcgcgtgatctccccgatgctgccaacacggcctccaccggcatggccacggccacggcgccgtctgcggtcgcttccgacctctcaagctccatgtttgtgcgcaaggccgtgccgctacgctacggaaccgcccccaacacgggcgtttctagcgtacacagatccaacacggtgcccgcccatgccgtctccagcagagcgccatcggtgcacccagacaaggagcgctccaagagcgcagagatggcagccgcagtagctggaatcagacctaccccgccgcccgggcgtcggctgcttcccgtacctgcccacactcctgcccccgtccgttcccattctcgcgtccagccgcagacaccccgaatcgactttctgccacaaaagaacccgctgcgaagagcagcaactgatacctgggatacccgcacgtcgacacaactgtcgacaatgcgctcgtcgtcgttcgggtcggtgcaaaggactgaagacgacgaagacaacgacaacgacaacagcaacgacaacgacgacgacggcgaagacagcgacacgacgccccagcccacgcccatcgccaacaagtacctcaacactgctgttcctctgggcgacgtggcaactgcagtggcccccaataagaatgcaaactccaacccggggttcgacatcaaattcagctctcccacgttcgaccccaagaacagcagcaccgaaaaggacgtggaaaagggcgagggtggagaagacgatgcagatggcaacgaaagcactgacgacaagaacgaggtggaaatggcctctcgcactgccaacatccactccgcacagcccacctttagctccaaccccgagtacttgtgggagctcgaagactttcagaaacgacgagacgagcgcaacaacaacgccagacacctgatacgaaagacagaggacgagaagcgacatgccgaacgacgcaacaaactcctactagttatttgttgcgtgcttgcagcggtgctgattggcatcactgtaggtatgacttcgaccatccatcggcttgaaagatcaaatcaaaaacaaggactggtcaaccagaacaaacagcacaacgatacccaaagaaccgacggcggcatgttacccattggaatcgataccctgacagatgcagccaagctcgcaagcattctgtcggatacctctttgcacaacgttctctacggcattggctactcgccccagaacgtcatgacatcccaatgtggagtcaaccagacggaagtgaccttggatgttgctgcattgtctcgtgttacaaagcggatccgactttacggcaccagctgtgaccaagccctgtacgttttacgggcgattgattccctcaaggtagatatgaaagtgactctcggagtgtggatcaacggcaaccctgtagtttgcgctgcgcagattgctgaagcaatcactgctgctgagcagttcccccatttgattgaatccatcatggtgggtaatgaggtgtttttccgcggcgagctcgcgccagccaaacttgtgggctacatgcatgaagtgcggacggagctacagcttcgcaacctcacaatccccgtgggcacctccgagctgggctctcaatggtctccgtacatggcctcgaatgtggatattctaggagccaacatccatccattctttggaggccttgaagtgtctgaggcgaccacctggacactcaatttcctcgagtcatttgtcgtcaacgagattacagaagccgaaaaaataccccaaattgtgatatccgagattggatggcccagtggaggtggtcaacacggcgctgcaaagtcgggtgtagcgcaacagcagcgactcctacaagactgggtgtgcacggccaaggacttgccgcagattggatggtactggttcgaggcgtttgacgagccgtggaagcgtgtgtttgacaccccgactgaaaagtgggagtctcagtggggtattttgacccctgaccggcgtctaaaaaaggggttgtcgctgaaaatcagctgtaat

SEQ ID NO: 71 - protein, 357 aa, Yarrowia lipolytica
MSRSSSPNASSAEDVRILGYDPLLAPALLQTEVASTKNARETVSKGRKDSIDVITGKSDKLLCIVGPCSLHDPKAAMEYAQRLKELSDKLSGELVIVMRAYLEKPRTTVGWKGLINDPDMDESFNINKGLRLSRKVFCDLTDLGLPIASEMLDTISPQFLADLLSLGAIGARTTESQLHRELASGLSFPVGFKNGTDGTLGVAVDAVQAASHPHHFMGVTKQGVAAITTTKGNENCFIILRGGKKGTNYDAESVAECKKATESMLMVDCSHGNSNKDYRNQPKVSKAVAEQVAAGEKKIIGVMIESNIHEGNQKVPKEGPSALKYGVSITDACVSWETTVDMLTELANAVKERRNKN

SEQ ID NO: 72 - DNA, 1074 bp, Yarrowia lipolytica
  source 1..1074: mol_type = genomic DNA
atgtcccgttcctcctctcccaacgcctcctctgctgaggacgtgcgaattctgggctacgaccccctcctcgctcccgctcttctccagactgaggttgcctccaccaaaaacgcccgagagaccgtctccaagggccgaaaggactccattgatgtcatcaccggcaagtccgacaagttgctgtgcattgtcggtccctgctccctccacgaccccaaggccgccatggagtacgcccagcgactcaaggagctgtctgacaagctgtctggtgagctcgtcatcgttatgcgagcctacctcgagaagccccgaaccaccgttggctggaagggtctgatcaacgaccccgacatggacgagtctttcaacatcaacaagggtctgcgactctcccgaaaggtcttctgcgaccttaccgatctgggtctgcccattgcctccgagatgctcgataccatttctccccagttcctggccgacctgctctccctgggtgccattggtgctcgaaccaccgagtcccagctgcaccgagagctcgcctccggtctgtctttccccgttggtttcaagaacggaaccgacggtactctgggtgttgccgttgatgctgtccaggccgcctctcaccctcaccacttcatgggtgtcaccaagcagggtgttgccgccatcaccaccaccaagggtaacgagaactgcttcatcattctgcgaggaggtaagaagggcaccaactacgacgccgagtccgtcgccgagtgcaagaaggccaccgagtccatgctcatggttgactgctctcacggcaactccaacaaggactaccgaaaccagcccaaggtttccaaggccgttgctgagcaggttgctgctggcgagaagaagatcatcggtgtcatgatcgagagtaatatccacgagggcaaccagaaggtccccaaggagggcccctctgcccttaaatacggtgtctccatcaccgacgcctgtgtctcttgggagaccaccgtggacatgctcaccgagctggccaacgccgtcaaggagcgacgaaacaagaactaa

SEQ ID NO: 73 - protein, 256 aa, Yarrowia lipolytica
MDFTKADTVLDLANIRDSLVRMEDTIVFNLIERAQFCRSEFVYKAGNSDIPGFKGSYLDWFLQESEKVHAKLRRYAAPDEQAFFPDDLPEAILPPIDYAPILAPYSKEVSVNDEIKKIYTDDIVPLVCAGTGDQPENYGSVMVCDIETLQALSRRIHFGKFVAESKFLSETERFTELIKNKDIAGIEAAITNSKVEETILARLGEKALAYGTDPTLRWSQRTQGKVDSEVVKRIYKEWVIPLTKKVEVDYLLRRLE

SEQ ID NO: 74 - DNA, 771 bp, Yarrowia lipolytica
  source 1..771: mol_type = genomic DNA
atggacttcactaaagccgacaccgttctggatctcgccaacatccgagactcgctggtccgaatggaggacactattgtcttcaatctgattgagcgggctcagttctgccgttccgagtttgtgtacaaggccggcaactcggacattcccggcttcaagggctcttacctcgactggtttctgcaggagtcggaaaaggtgcacgccaaactgcgtcggtacgctgccccggacgagcaggccttcttccccgacgatctacccgaggccattctgccccccatcgattatgcgccaattctggcaccctacagcaaggaggtgagcgtcaacgacgagattaaaaagatttacaccgacgacattgtgcccctggtgtgtgctggcactggagatcagcccgagaactatgggtcggtcatggtgtgcgacatcgagacgctgcaggcgctgtcgcgacgaatccactttggcaagtttgtggccgagtccaagtttctgagtgaaaccgagcgattcaccgagctcatcaagaacaaggacattgctggtattgaggcggccatcacaaactccaaggtggaagagacgattctggcccggctgggagaaaaggcactggcctacggcacagaccccactctccggtggtcgcagagaacccagggaaaggttgattccgaggttgtcaagcgaatctacaaggagtgggtgattccactcaccaagaaggtcgaggtggactacctgctccggcggttggagtag

SEQ ID NO: 75 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 76 - DNA, 1113 bp, Saccharomyces cerevisiae
  source 1..1113: mol_type = genomic DNA
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 77 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 78 - DNA, 771 bp, Saccharomyces cerevisiae
  source 1..771: mol_type = genomic DNA
atggatttcacaaaaccagaaactgttttaaatctacaaaatattagagatgaattagttagaatggaggattcgatcatcttcaaatttattgagaggtcgcatttcgccacatgtccttcagtttatgaggcaaaccatccaggtttagaaattccgaattttaaaggatctttcttggattgggctctttcaaatcttgaaattgcgcattctcgcatcagaagattcgaatcacctgatgaaactcccttctttcctgacaagattcagaaatcattcttaccgagcattaactacccacaaattttggcgccttatgccccagaagttaattacaatgataaaataaaaaaagtttatattgaaaagattataccattaatttcgaaaagagatggtgatgataagaataacttcggttctgttgccactagagatatagaatgtttgcaaagcttgagtaggagaatccactttggcaagtttgttgctgaagccaagttccaatcggatatcccgctatacacaaagctgatcaaaagtaaagatgtcgaggggataatgaagaatatcaccaattctgccgttgaagaaaagattctagaaagattaactaagaaggctgaagtctatggtgtggaccctaccaacgagtcaggtgaaagaaggattactccagaatatttggtaaaaatttataaggaaattgttatacctatcactaaggaagttgaggtggaatacttgctaagaaggttggaagagtaa

SEQ ID NO: 79 - DNA, 2571 bp, Yarrowia lipolytica
  source 1..2571: mol_type = genomic DNA
atggaggaattatcggaggcggaacgaaaccaaatcggccagctattcatctgtggattacgtaatgcggagctcgacgaggacattgccgagctagtgcgcaccttcaaggttggctccatccaggtgagcatcaaaaacttaccttcggtggaccaggcacgcgagctcatccgcggcgtgcaacagctggcctatgaagccggtcacgaacagcccatggcgatttgcatcgaccaagagggcggcattctcaacaacctgaaaggcgtgacccaattcccatgccaaatggctctggcagcctccaaagacaccgatctagtggaggcggtggccgaggcggcggctaaggagctcctagcctgtggaatcaacttcatgtttgcaccgtgtatggacgtgctcaagtcgacgtcggccagtgcggacttcttgctggggacccgtgcctttggagacgatcacgaaattgtgtctcaattcggatgtgctttccttcgtggactgcagaaacaaaacatgatggcctgtggaaaacactttccgggctacggaacagcctcgctggactccatcttggggggagtgcctgtggtctcggataatgagctccaaatgcaggcacaggcgtttgtgccgtttaaggcgatcatcgatatggattgtgatgccatcatggtgggaggatgttctgctccaggtttggatcctacggaagctcatgcttgtttgtcctggaagatatgcactgatattcttagagaacagctgggattcaagaacgttattgtttcagagtgtctggagatggaggctctttaccaacaggttggagtgaaacaaggaaccgtctcagcaaagctggctggctgtgacatgattttgtgctgctcttcgttcaagttgcaaaagcaagccctggaaggtctatcaggcgcctatctcgacggacttgtggagcccgaaagcatcatggcatctgctgaacgcgttagacacatgaagtctagcaggaatctcacctggcagagtattctgggagagagaccgtttgtggcagagctgttgcaagaacacaaactgctgtctgcacgagcgtacgagtcgtgtgtggctattggcagagactatcttcacatgattcctttttccttgaaacgctcagacacgattctactgctgaccccgttatgtgaatccgacgacattcacagcggcagatgcactccagcgcaaacaaacggcaacggaaacccatccaacgtgcgcttgcttcctggcgaagcgacttttcaacatctgggaaagaccattgcctcatatcatactggaaagacactgcacacttcgtattcccaaaacggaattatcaagttacacgaacagctgatagccagagcaacggttgtgattctggtcaacacaacagcctactcgaacatgtatcaaacctcgataacaaagtacgtgcagcttctttgtgaccaaagacgtattccgtttgtactggtggctgcttcaagcccccatgacatggcgtcctccaaccatacatcggatacaaagacgtatgtctgtgcgtttgaatttaccccagaaatgcaggtcacatgtgcaaaggtcatgtttggtaagcttcctgcgctgggaactatcccattctctcacttctggggtagacaaaggtctggttccaagtcactgagtactacgtctaccagaaagacgtggatggtggaaacgtatgctggagagaacgtgcaacctctttgggatctatgtttcccgaaacgaaagctgccaaccgaggcgttctccgtgttaaatgatatcgactgctatgtggtgaaaaactcgtctactggaaagctgtatgggttctgtgcggctcacaagacccaggttgcactactcatggtggacccgtccagacggaaaatgggcatagctgtgagcctgtacaagcgagtacaaagagccaagggcagaaaggacccctctgcggctcagctgacctcgtttgggtcggtgcttcccgcgtactttgctggtgctacacccgagagtctggcgtggctacagaaggtggaccgggccatgatctttagagacaatgtggttgttctagtgaaccagaggctgcaggacttccgagtcgatgcggctgtgatccaggagctgcagcagaacaacctccggttctccatagccagtgttgcagaaacagacgcaaaagaccttccggaggagctgagagagtcggatgcctatgtggcgcagtgtctcaaggatggttcggtggtgggctcggtggtgctgttcaataagggttcttccatagcccggtggctgccttggatctatgagtttggtccgtcggtgggtggcatgaccagtttgcggtgcgactctgatttgataatgcaagggcttgtttgctgtgctatgagaagcttccgcattgagggctttcagacggtggtattggatcacatgagtgttgctcaacgcgacagtcttagtgggcttggcttttctgtctggcgggtggaagagaagttcagccgg

SEQ ID NO: 80 - DNA, 1242 bp, Yarrowia lipolytica
  source 1..1242: mol_type = genomic DNA
atgaagttttccactcttgctctggccgcaatggccgccattggctttgccgcccccgccactcccgacgctgactgcaacgaggagaaggaggtccgagacttccacgctcaccaccagcacaagcgagctgtgcaggtcgagtacgtctacgtcactgtccttgttgacggtaacggaaacaccattgagcagcagcagattgccaccaccgagctccaggaggctccctcccctgctcccgctacctccacccaggaggctgctcccgctccttccagcacccttgttgctgcttttgccgccaaggagcccgtcacccaggaggcttcttctgccgcttcttccgccgctccttcctccacctctgcccctaagtcttcttctgctccttccaagccttccaacggaggctccggaggcatcactggtgacctcgctgccttctctggtccttccgagaagttccaggacggtaccattgactgtggagatttcccctccggccagggtgtcattgccctgaaccagcttggcttcggcggctggtccggtatctaccaccccggttccactgccaccggtggcaactgtgccgagggtgcttactgctcttacgcttgccagtctggtatgtccaagacccagtggcctgatgagcagcccgccaacggtgtttccgttggtggtcttctctgcaagggcggtaagctttaccgatccaaccagcagtctgactacctctgcgagtggggcacccagtccgccattgtgacctcttctctcgataaggaggttgctatctgccgaaccgattaccccggtaccgagaacatggttatccccactgtcgttgaggccggttcctccggtgttcctcttgccgtcgtcaaccaggatgcttactacacctggatgggcaagcctacctccgctcagtactacgtcaacaacgctggtgtttcttaccaggatggctgtctctggggtacttcttccggtgatgtcggtaactgggctcctctgaacttcggtgctggcgctgccggcggtatctcttacctttccatcattcctaaccccaacaaccgaaactccgctaacttcaacgttgagatcatcgctactaacggtgctactgtcaacggtaagtgtgtctacgagaacggtaagtacaacggtggtgactccaacggttgcactgttgctgttaccggcggtgttgctgccttccacctttac

SEQ ID NO: 81 - DNA, 1350 bp, Yarrowia lipolytica
  source 1..1350: mol_type = genomic DNA
atgatgtcttaccaatcgaccaaggccccaccgccttttcgacggacccctacaaagtcgccggtacgatcctcgtttgagtcagtgtcttcgaataccccactggacttttctctgccgtcataccactcgccttcggagaagctccaaccaccacagaagggatccaagagcaaacgatatgtccttgttctggccacagcagcactatgttgcttcatgtacatactggccatgtctgcgcctgaaaagactggaagtgatgctctggtggccctcaagaacaaggtctcgcagtcaacagagactctcaagcagcttatccccgcccagccgttcgtatggtccgaacccacacccaacttccacagctacgccatcgcctacctgccactcaacaagaacagcaagtgccgccgacagagccagatcactgccgactttgcatctctggaggcttcaggccgcgtagaggccatccggctcttttctaccgactgcaaggtcattccggcctatctggactacctcaagaatagagactcgaagaagcgaagcgtggactccgctctcaagctagtacttggaatcgagcccagtgcactggaaaccacttcggatctggactccgaggccacgtctctcgagctgctgaccagatctgtcgacgcccagctcatggacatcatcgactctctggacgagtcttcttctgccaccgaggcctttcttcgacagctggatctgatcgtgttgggttccgagggcctggccaaccaaaattaccatcccaaggagctcgtcgaggtcctggctcacatccgacagcgtctcgacactctgtcttccgacattcgaatcccagtcaccacctccgagcctctgggcatctgggacgtgctgacacccaaggagcgacgagacaacgtccaggaaatgcttgcacgacaggacggagtgcactctgtcactcccgacgacattgaaggccccacgggtctgttttgcgactccgtcgatgtgattggtttgtctgtctctcccttcaacaaccctactgtcgacgctgcccatgccggtgccgaggtcgaggagtctgcctaccttgcctcgcttctgtgtaacaagcctgtgctcgccctggaggtgggatggccctcggctggttcggataacggcaacgccaagaccggcaaggacgaacagctacaagccgtgtctagtatcactgctgctgttgagcgaatctccggcaagcccctgagatccgttttgtatcagtactttgacgacgagtggctgtccgcagacgaggactttgcccgccatttcggcctgaagcggttgttc

SEQ ID NO: 82 - DNA, 957 bp, Yarrowia lipolytica
  source 1..957: mol_type = genomic DNA
atgcccctcctgcccccttcctgccctcctgcccctcagactacctctgacgctgccccctctccccagcctgcccctaccaccttttccacttctgttatcgagccctcttccacctcttcttctgctgcggaggccactcccactttcacgcccaacggaattctgccctactcgctcacctactcgccctacaacgacgactcgtcctgcaagactatggacgaggtcatgaaggacctcaaggagatcgtcgccaagggtatcaaggtgatccgtgtctacggcactgactgcggctctgtccagaccattgaacctgccgctaagcagcttggtctcaagatcaaccagggtttctggatcggacccgacggagttgactccattgactctggtgtccaggagttcattaactgggtccagcagaaccaggcctggggtatgattgactctatcactgttggaaatgaggctatcattgccggctacgtcagcccccagcaactccttggaaagatcggccaggtcaagagtcagctcaaggctgctggataccagggacaggtcaccaccgccgagcctgctgtctcctacaccacccaccccgagctgtgcactggccctgagctcgactacgttgggatcaactctcacgcctacttcaacccccaacagtctcctgagaccgccggccagtttgctctggacgagatggctcttactcagaagacttgtaacaacaaggttgtttttgtcaccgagacaggctacccttctgctggtaacaccaacggcaacaatgttcctactccccagaaccaggaaattgccatcaactctttgctcaaggccctcaatggctacggcacctttttcaccatgtacaacgacttctggaaggcccccggtccttacaacgttgagcagcattttggtatcattaacattcttcag

SEQ ID NO: 83 - protein, 495 aa, Acleisanthes obtusa
MDQTTLAMLLSALYLLYNLYKVIFTQSNSKLPPGPKPLPILGNIFEVGNKPHRSFANLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLHEYVQDCAKKGQAVDIGKAAFTTSLNLLSNLFFSVELAQHTSSSSQHFKELIWDIMEDIGKPNYADYFPALKCVDPWGIRRRLAANFERLIDVFQGFIRQRLSINSSTVTSASDVLDVLLNLYKEKELNMGEINHLLVDIFDAGTDTTSSTFEWAMAELVRHPEIMKKAQDEIEQVLGKDAIIQEADIPKMPYLQAIIKETLRLHPPTVFLLPRKATTNVELYGYVVPKNAQILVNLWAIGRDPLVWDNPNKFSPERFLNSDIDVKGRDFGLLPFGAGRRICPGMNLAYRILTLMLATLLQSFEWMVENGENPEDLDMDEKFGIALQKTKPLEIIPVIKHR

SEQ ID NO: 84 - protein, 506 aa, Basella alba
MDNTTLAILLSTSYFIIYLIITKLGFHYKVLPKNPKQRLRLPPGPKPLPIIGNVLELGSKPHRSFTNLAKVHGPLISLRLGSVTTIIVSSSHVAKEMFLKNDQSLSSNRTIPHSVTAGDHHKLTMSWLPVSPKWRSFRKITTFHLLSPQRLDACCSLRQAKVQQLFEYVLQCSRTGQPVDIGKAAFTTSLNLLSKLFFSLELAHHRSTKSQEFKDLIWNIMEDIGKPNIADHFPCLKYFDPSGIRRRLASSFERLIEVFQDIIRQRMSLSFGSSHNNDVLDVLLGLYNQKELTMDEINHLLVDIFDAGTDTTSSTFEWSMAELMKNRRIMEKAQAEILHVLGKNSYIQESDISKLPYLRAIIKETLRLHPPTVFLLPRKADADVELYGYVVPKDAQILVNLWALGRDPAVWENPDEFSPDRFMGSEIDVKGRDFGLLPFGAGRRICPGMNLAIRMLTLMLATLLRSFDWKLPEGEAPAQLDMDEKFGIALQKTTPLKIIPIFKNSI

SEQ ID NO: 85 - protein, 495 aa, Cleretum bellidiforme
MDYTTLVMILSIVFFCYNLFNLLFTRKNTKLPPGPKTIPIFGNIFELGKKPHQSFANLAKIHGPLMSLKLGSVTTIVVSSAEVAREMFLKNDQLLSNRTVPNSVTAGDHHKTTMSWLPVSQKWRNFRKITAVHLLSPQRLDSCQALRQAKVKQLFNYIHECAQKGEAVDIGKAAFTTSLNLLSNLFFSVELANHKSSSSQEFKQLIWNIMEDIGKPNYADYFPVLKYVDPSGIRRRLASNFNKLIDVFQGFIRLRMSTNSSCGATNPNDVLDVLLNLYKGDDLNMDEINHLLVDIFDAGTDTTSSTFEWAMAELVKNPKMMKKAQAEIQQVLGKDSIIRESDIPNMPYLQAIIKETLRLHPPTVFLLPRKADADVELYGYVVPKNAQILVNLWALGRDPLVWKSPNVFKPERFLGSEIDFKGRDFGLLPFGAGRRICPGMNLAYRMLTLMLATLLQSFDWKVADGTNPQDMDMDEKFGIALQKTTPLQIIPVYKY

SEQ ID NO: 86 - protein, 495 aa, Mirabilis multiflora
MDQTTLAMLLSALYLLYNLFKVIFTQSNSKLPPGPKPLPILGNIFELGDKPHRSFNNLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYQYVQDCAKKGEAVDIGKAAFTTSLNLLSNLFFSVELAQHTSSSSQHFKELIWDIMEDIGKPNYADYFPALKCVDPWGIRRRLAANFERLIQVFQNFIRQRLSTDPSSVTNASDVLDVLLNLYKEKELNMGEINHLLVDIFDAGTDTTSSTFEWAMAELVRHPEIMKKAQDEIEQVLGKDATIQEADIPKMPYLQAIIKETLRLHPPTVFLLPRKATTNVELYGYVVPKNAQILVNLWAIGRDPLVWDQPNKFSPERFLNSDIDVKGRDFGLLPFGAGRRICPGMNLAYRMLTLMLATLLQSFEWKVQNGEKPEDLDMDEKFGIALQKTKPLEIIPVLKYR

SEQ ID NO: 87 - protein, 496 aa, Mirabilis multiflora
MDFLTLVMILSMIFFFYNLLKMIFTTHSDAQLPPGPKPMPIFGNIFELGEKPHRSFANLAKTHGPLMSLRLGRVTTIVVSSAEVAKEMFLKNDQSLADRSVPNSVTAGDHHKLTMSWLPVSPKWKNFRKITAVHLLSPQRLDACNALRHAKVKQLYEYVQECALKGEAVDIGKAAFTTSLNLLSNLFFSVELANHTSNTSQEFKQLIWDIMEDIGKPNYADYFPLLKYVDPSGIRRRLAANFDKLIDVFQSFICKRLSSAYSSATSLDDVLDVLLKLYKEKELNMGEINHLLVDIFDAGTDTTSNTFEWAMSELIRNPTMMKRAQNEIALVLGKDNGTIQESDIANMPYLQAIIKETLRLHPPTVFLLPRKAITYVKLYGYIVPKNAQILVNLWAIGRDPKVWKNPNEFLPDRFLNSDIDVKGRDFGLLPFGAGRRICPGMNLAYRMLTLMLATLLQSFDWKLPHGNSPMDLDMDEKFGIALQKTKPLEIIPVIKY

SEQ ID NO: 88 - protein, 508 aa, Opuntia ficus-indica
MDTPTLSYFISAITFYYIAFQIVKLGFNVIMTSKKTKRRRLPLPPGPKPLPIIGNVFELGPKPHRSFASLAKVYGPLMSLRLGSVTTIIVSSSDVAKEMFLKNDQPLSSTRTIPNSVTAGDHHKLTMSWLPVSPKWRSFRKITTFHLLSPQRLDACSGLRQAKVQQLYEYVLECSRTGQAVDIGKAAFTTSLNLLSKLFFSLELANHTSDKSQEFKELIWNIMEDIGKPNYADYFPCLKYFDPSGIRRRLACSFEKLIEVFQVIIRQRLSLSSSGTNDHNNDVLDVLLDLYQQKELSMEEINHLLVDIFDAGTDTTSSTFEWAMAELIKNPRMMETAQAEIKLILGKDLHIQESDIPKLPYLRAIIKETLRLHPPTVFLLPRKADADVELYGYTVPKNAQILVNLWALGRDPKVWENPDVFSPERFLGCDIDVKGRNFGLLPFGAGRRICPGMNLAYRMLTLMLATLLQSFDWKLPNEMNPQNLDMDEKFGIALQKTKPLQIIPLSKD

SEQ ID NO: 89 - protein, 496 aa, Phytolacca americana
MDHTTLAMILSVIFLLYNLVKAIFSQSNTKLPPGPKPVPIFGNIFELGDKPHRSFANLAKIHGPLITLKLGSVTTIVVSSAEVAKEMFLTNDQLLANRNVPNSVTAGDHHKLTMSWLPVSPKWKTFRKITAVHLLSPQRLDACQALRHTKVKQLYEYVQECAKRGEAVDIGKAAFTTSLNLLSNLFFSVELANHTSSSSQEFKELIWDIMEDIGKPNYADYFPVLKCVDPWGIRRRLESNFDKLIEVFQSFIRKRLSTEPFSASAKTPNDVLDVLLNLLKEEELNMGEINHLLVDIFDAGTDTTSSTFEWAMAELVRNPEMMKKAQDEIEQVLGKDAIIQESDIPKMPYLQAIIKETLRLHPPTVFLLPRKASSNVELYGYVVPKNAQILVNLWAIGRDPTVWDNPNMFSPERFLNSDIDVKGRDFGLLPFGAGRRICPGMNLAYRMLTLMLATLLQSFDWKLGDGVNPKDLDMDEKFGIALQKTKPLQVIPVLKY

SEQ ID NO: 90 - protein, 497 aa, Phytolacca dioica
MDHTTLAMILSAIFLLYNLVKLAIFSQSNTKLPPGPKPLPIFGNIFELGDKPHRSFANLAKIHGPLITLKLGSITTIVVSSAEVAKEMFLKNDQPLANRNVPNSVTAGDHHKLTMSWLPVSPKWKTFRKITAVHLLSPQRLDACQALRHTNVKQLHEYVQECAQRGQPVDIGKAAFTTSLNLLSNLFFSVELANHTSSNSQEFKELIWDIMEDIGKPNYADYFPVLKCVDPWGIRRRLESNFDKLIEVFQGFIRKRLSTGSFSASETTPNDVLDVLLNLFKEKELNMGEINHLLVDIFDAGTDTTSSTFEWAMAELVRNPDMMKKAQDEIEQVLGRDAIIQESDIPKMPYLQAIIKETLRLHPPTVFLLPRKATTNVDLYGYVVPKNAQILVNLWAIGRDPTVWDNPNMFSPERFLNSDIDVKGRDFGLLPFGAGRRICPGMNLAYRMLTLMLATLLQSFNWKLGDGVNPKDLDMDEKFGIALQKTKPLQVIPVFKY

SEQ ID NO: 91 - protein, 268 aa, Amaranthus tricolour
MGSQEIIKETFFISHGTPRMTIEASKPARKFLESWRDKIYFKKPKAILVISAHWETDFPSVNAVDINDTIYDFYGFPAPMYQFKYPAPGSPDLAQRVQELLTASGFKSVNVDKKRGLDHGAWVPLMLMYPNADIPVCQLSVQSHLDGMYHYKLGRALAPLKEEGVLIIGSGSATHPSNNTPHYYDGVAPWAADFDHWLETALTNGSYEEVNKCESKAPNWKLAHPWPEHFYPLHVAMGAAGENWKAELIHNSWDHGTMSYGSYKFVSS

SEQ ID NO: 92 - protein, 268 aa, Amaranthus hypochondriacus
MGSQEIIKETFFISHGTPRMTIEESKPARKFLESWRDKIYCKKPKAILVISAHWETDFPSVNAVDINDTIYDFYGFPAPMYQFKYPAPGSPDLAQRVQELLTASGFKSVNVDKKRGLDHGAWVPLMLMYPNADIPVCQLSVQSHLDGMYHYKLGRALAPLKEEGVLIIGSGSATHPSNSTPHYYDGVAPWAADFDQWLETALTNGSYEEVNKCERKAPNWKLAHPWPEHFYPLHVAMGAAGENWKAELIHNSWDHGTMSYGSYKFVSS

SEQ ID NO: 93 - protein, 268 aa, Beta vulgaris
MGSEDNIKETFFISHGTPMMAIDDSKPSKKFLESWREKIFSKKPKAILVISAHWETDQPSVNVVDINDTIYDFRGFPARLYQFKYSAPGSPELANRIQDLLAGSGFKSVNTDKKRGLDHGAWVPLMLMYPEADIPVCQLSVQSHLDGTHHYKLGQALAPLKDEGVLIIGSGSATHPSNGTPPCSDGVAPWAAAFDSWLETALTNGSYEEVNKYETKAPNWKLAHPWPEHFYPLHVAMGAAGENSKAELIHNSWDGGIMSYGSYKFTST

SEQ ID NO: 94 - protein, 275 aa, Beta vulgaris
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 95 - protein, 275 aa, Beta vulgaris
MKMMNGEDATDQMIKESFFITHGNPILTVEDTHPLRPFFETWREKIFSKKPKAILIISGHWETVKPTVNAVHINDTIHDFDDYPAAMYLFKYPAPGAPELARKVEEILKKSGFETAETDEKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYETKAPNWKLAHPFPEHFYPLHVVLGAAGEKWKAELIHSSWDHGTLCHGSYKFTSA

SEQ ID NO: 96 - protein, 268 aa, Bougainvillea glabra
MGGEKKMKGTYYLAHGDPIMYINKSIKLRHFLEGWKENVLTEKPKCILVISAHWDTDVPTVNLVEQCDTIHDFDDYPDPLYQIKYPAPGAPKLAMKVQELLKGGGFKCEVDTKRGLDHAVWFPLMFMYPEADIPICELSIQTSKDGTHHYNVGKALSPLLNDDVLIIASGGAVHPSDDTPHFPNGVAPWALEFDNWLEGALLSGRYEDVKEFKKLAPNWEISHPGQEHLYPLHVALGAAGNNVKTELIHQTWAANGVFGYSSYKFTST

SEQ ID NO: 97 - protein, 266 aa, Phytolacca americana
MDVKDMIRETFYISHGTPMMAINKSVPARSFLKGWREEVYSKKPKSILVISAHWETDLPTISAVNHSDLIYDFYGFPAPMYQLKYPAPGAPDLATRVQELLTVSGFKCALDKKRGLDHGSWVPLMFMYPDANIPVCQLSLQSHLDGTHHYKLGRALAPLKEEGVLVIGSGSSVHPSNDTPHAVGVAPWAAEFDNWLEEALTSGRYEDVNNYQTKAPNWKIAHPWPEHFYPLHVAMGAAGENAKAELIHRSWEHGTLGYACYKFTSS

SEQ ID NO: 98 - protein, 310 aa, Spinacia oleracea
MILHNFQQPSTLLLQIPSFSATPEIKRTFLKSKFPKNSNLKIMAGQESIKETFFISHGTPMMAIDESKPSRKFLESWREKIYSKKPKAILVISAHWETDSPSVNSVDVNDTVYDFGGFPARLYQFKYPAPGFPDLAKRVQELLTASGFQSVHTDKKRGLDHGAWVPLMLMYPEADIPVCQLSVQSHLDGKYHFNLGRALAPLKDEGVLIIGSGSATHPSNGTPHCNDGVAPWAADFDLWLETALTSGRYEEVNKCERKAPNWKLAHPWPEHFYPLHVAMGAAGENSKAELIHNSWDHGTMSYGSYKFSPN

SEQ ID NO: 99 - protein, 271 aa, Suaeda salsa
MGSNNNNEKIKETFFLSHGTPMMIIEESQPARKFLESWTEKIYSKKPKAILVISAHWETDHPAVTSVEVNDTIHDFYGFPAPMYQFKYPAPGSPDLAKRVQELLTASGFKSVQTDKKRGLDHGAWVPLMFMYPEPEIPVCQLSVQSHLDGKFHYNLGRALAPLKDEGVLIIGSGSATHPSNATPHSYDGVAPWAADFDCWLETSLTNGRYEEVNKCETKAPNWKLAHPWPEHFYPLHVAMGAAGEDWKAELIHNSWDHGTMSYGSYKFTSS

SEQ ID NO: 100 - DNA, 25 bp, Synthetic construct
  source 1..25: mol_type = other DNA
cacgcgatgcacacaccatagcttc

SEQ ID NO: 101 - DNA, 26 bp, Synthetic construct
  source 1..26: mol_type = other DNA
atgacagatttgttttatatttgttg

SEQ ID NO: 102 - DNA, 35 bp, Synthetic construct
  source 1..35: mol_type = other DNA
acctgcactttgtaattaaaacttagattagattg

SEQ ID NO: 103 - DNA, 25 bp, Synthetic construct
  source 1..25: mol_type = other DNA
agtactgcaaaaagtgctggtcgga

SEQ ID NO: 104 - DNA, 27 bp, Synthetic construct
  source 1..27: mol_type = other DNA
acgtgcgatagagaccgggttggcggc

SEQ ID NO: 105 - DNA, 27 bp, Synthetic construct
  source 1..27: mol_type = other DNA
agcataagtagagaccgggttggcggc

SEQ ID NO: 106 - DNA, 42 bp, Synthetic construct
  source 1..42: mol_type = other DNA
acttatgctacgcaactaacatgaatgaatacgatatacatc

SEQ ID NO: 107 - DNA, 38 bp, Synthetic construct
  source 1..38: mol_type = other DNA
atctgtcataaaacaatggatcacgcaacattagctat

SEQ ID NO: 108 - DNA, 32 bp, Synthetic construct
  source 1..32: mol_type = other DNA
cacgcgatttagtatcttggaattgggatcaa

SEQ ID NO: 109 - DNA, 38 bp, Synthetic construct
  source 1..38: mol_type = other DNA
agtgcaggtaaaacaatgaagggaacctactacatcaa

SEQ ID NO: 110 - DNA, 29 bp, Synthetic construct
  source 1..29: mol_type = other DNA
cgtgcgatttacgtcggtcttttgggtgg

SEQ ID NO: 111 - DNA, 43 bp, Synthetic construct
  source 1..43: mol_type = other DNA
actttttgcagtactaaccgcaggaccacaccactctggccat

SEQ ID NO: 112 - DNA, 28 bp, Synthetic construct
  source 1..28: mol_type = other DNA
cacgcgatttagtacttcagcacgggga

SEQ ID NO: 113 - DNA, 35 bp, Synthetic construct
  source 1..35: mol_type = other DNA
agtgcaggtaaaacaatggacgacaagtcccaaca

SEQ ID NO: 114 - DNA, 28 bp, Synthetic construct
  source 1..28: mol_type = other DNA
cgtgcgatttaggtagacaaacctctca

SEQ ID NO: 115 - DNA, 35 bp, Synthetic construct
  source 1..35: mol_type = other DNA
atctgtcatgccacaatgaagggcacctactacat

SEQ ID NO: 116 - DNA, 28 bp, Synthetic construct
  source 1..28: mol_type = other DNA
cgtgcgatttacacgtcggtcttttgag

SEQ ID NO: 117 - DNA, 43 bp, Synthetic construct
  source 1..43: mol_type = other DNA
actttttgcagtactaaccgcaggacgacaagtctcagcagct

SEQ ID NO: 118 - DNA, 28 bp, Synthetic construct
  source 1..28: mol_type = other DNA
cacgcgatttaggtagacaggcctcgca

SEQ ID NO: 119 - DNA, 29 bp, Synthetic construct
  source 1..29: mol_type = other DNA
cgtgcgatttaggtagacaggcctcgcag

SEQ ID NO: 120 - DNA, 45 bp, Synthetic construct
  source 1..45: mol_type = other DNA
actttttgcagtactaaccgcaggacgacaagtctcagcagctgc

SEQ ID NO: 121 - DNA, 26 bp, Synthetic construct
  source 1..26: mol_type = other DNA
cgtgcgattcagctgaagcttcgtac

SEQ ID NO: 122 - DNA, 28 bp, Synthetic construct
  source 1..28: mol_type = other DNA
cctgcactcactagtggatctgatatca

SEQ ID NO: 123 - DNA, 22 bp, Synthetic construct
  source 1..22: mol_type = other DNA
tacttcaagacttgtgctgagg

SEQ ID NO: 124 - DNA, 32 bp, Synthetic construct
  source 1..32: mol_type = other DNA
atcgcacgtaataaataacgagtgtggtggtg

SEQ ID NO: 125 - DNA, 33 bp, Synthetic construct
  source 1..33: mol_type = other DNA
agtgcaggtgtgtcatttgaatagataccatac

SEQ ID NO: 126 - DNA, 19 bp, Synthetic construct
  source 1..19: mol_type = other DNA
cttgaaggagcaccttcag

SEQ ID NO: 127 - DNA, 20 bp, Synthetic construct
  source 1..20: mol_type = other DNA
catggcatggcgtacaactg

SEQ ID NO: 128 - DNA, 32 bp, Synthetic construct
  source 1..32: mol_type = other DNA
atcgcacgttttgaaagttattcagatgactg

SEQ ID NO: 129 - DNA, 32 bp, Synthetic construct
  source 1..32: mol_type = other DNA
agtgcaggtgattccacttgagagtgaatcac

SEQ ID NO: 130 - DNA, 22 bp, Synthetic construct
  source 1..22: mol_type = other DNA
cgtcgggactgtgttagtaatc

SEQ ID NO: 131 - DNA, 22 bp, Synthetic construct
  source 1..22: mol_type = other DNA
acattactggaaaccccatcac

SEQ ID NO: 132 - DNA, 33 bp, Synthetic construct
  source 1..33: mol_type = other DNA
atcgcacgtgtttttatttctggtgtgtaggag

SEQ ID NO: 133 - DNA, 30 bp, Synthetic construct
  source 1..30: mol_type = other DNA
agtgcaggtgtgagagagttcagtgctgag

SEQ ID NO: 134 - DNA, 20 bp, Synthetic construct
  source 1..20: mol_type = other DNA
ctggaacgtctctgagaacc

SEQ ID NO: 135 - DNA, 21 bp, Synthetic construct
  source 1..21: mol_type = other DNA
tgacacagcgacgatgtaatc

SEQ ID NO: 136 - DNA, 30 bp, Synthetic construct
  source 1..30: mol_type = other DNA
atcgcacgttgtgataggactgttgttgac

SEQ ID NO: 137 - DNA, 27 bp, Synthetic construct
  source 1..27: mol_type = other DNA
agtgcaggttcggacccgcagagcaaa

SEQ ID NO: 138 - DNA, 22 bp, Synthetic construct
  source 1..22: mol_type = other DNA
agcaactgtgtctaacaaacac

SEQ ID NO: 139 - DNA, 21 bp, Synthetic construct
  source 1..21: mol_type = other DNA
gctcatcgccttagcctaatc

SEQ ID NO: 140 - DNA, 28 bp, Synthetic construct
  source 1..28: mol_type = other DNA
atcgcacgtggagggtggttggatctag

SEQ ID NO: 141 - DNA, 35 bp, Synthetic construct
  source 1..35: mol_type = other DNA
agtgcaggtgaggatgaactaattactttagattc

SEQ ID NO: 142 - DNA, 21 bp, Synthetic construct
  source 1..21: mol_type = other DNA
cgaggaggttctccagatcac

SEQ ID NO: 143 - DNA, 22 bp, Synthetic construct
  source 1..22: mol_type = other DNA
gaggtttcgagagccttgttag

SEQ ID NO: 144 - DNA, 30 bp, Synthetic construct
  source 1..30: mol_type = other DNA
atcgcacgtgttgtgatggggtgggaatag

SEQ ID NO: 145 - DNA, 29 bp, Synthetic construct
  source 1..29: mol_type = other DNA
agtgcaggtcccgcatctattgtatgtat

SEQ ID NO: 146 - DNA, 22 bp, Synthetic construct
  source 1..22: mol_type = other DNA
agtggagattatcacccaatgg

SEQ ID NO: 147 - DNA, 20 bp, Synthetic construct
  source 1..20: mol_type = other DNA
tccagcccattgttcgtgtg

SEQ ID NO: 148 - DNA, 30 bp, Synthetic construct
  source 1..30: mol_type = other DNA
atcgcacgtcgcaaatgaaagtgaatagcg

SEQ ID NO: 149 - DNA, 34 bp, Synthetic construct
  source 1..34: mol_type = other DNA
agtgcaggtgcagatcgctagggccacttcaaaa

SEQ ID NO: 150 - DNA, 24 bp, Synthetic construct
  source 1..24: mol_type = other DNA
ggaggatctctgctgtcttctact

SEQ ID NO: 151 - DNA, 21 bp, Synthetic construct
  source 1..21: mol_type = other DNA
cgaaatctggctgtttccgta

SEQ ID NO: 152 - DNA, 38 bp, Synthetic construct
  source 1..38: mol_type = other DNA
atcgcacgtaaatactatcacccaaaagaagatctaac

SEQ ID NO: 153 - DNA, 29 bp, Synthetic construct
  source 1..29: mol_type = other DNA
agtgcaggttctcattatgtaacgtcata

SEQ ID NO: 154 - DNA, 20 bp, Synthetic construct
  source 1..20: mol_type = other DNA
tgtccaccttcgttgaagtc

SEQ ID NO: 155 - DNA, 22 bp, Synthetic construct
  source 1..22: mol_type = other DNA
tatgattgccaaggccaacagg

SEQ ID NO: 156 - DNA, 31 bp, Synthetic construct
  source 1..31: mol_type = other DNA
atcgcacgtgttggagagaccaagagaccgt

SEQ ID NO: 157 - DNA, 35 bp, Synthetic construct
  source 1..35: mol_type = other DNA
agtgcaggttaccccatagatatttattcagattc

SEQ ID NO: 158 - DNA, 23 bp, Synthetic construct
  source 1..23: mol_type = other DNA
agtctgtcaagatggaagtggac

SEQ ID NO: 159 - DNA, 23 bp, Synthetic construct
  source 1..23: mol_type = other DNA
gtacaagtagcttggagatggac

SEQ ID NO: 160 - DNA, 29 bp, Synthetic construct
  source 1..29: mol_type = other DNA
atcgcacgtgggtagcaatgggcagtgta

SEQ ID NO: 161 - DNA, 30 bp, Synthetic construct
  source 1..30: mol_type = other DNA
agtgcaggttgtcgtttccacgagcaaaat

SEQ ID NO: 162 - DNA, 23 bp, Synthetic construct
  source 1..23: mol_type = other DNA
caggtctggagctttattaacca

SEQ ID NO: 163 - DNA, 23 bp, Synthetic construct
  source 1..23: mol_type = other DNA
cgggttacttggtttgtaatctg

SEQ ID NO: 164 - DNA, 30 bp, Synthetic construct
  source 1..30: mol_type = other DNA
atcgcacgttcttgaggaacatcggatgag

SEQ ID NO: 165 - DNA, 30 bp, Synthetic construct
  source 1..30: mol_type = other DNA
agtgcaggttagtcagtcacccgtattaac

SEQ ID NO: 166 - DNA, 21 bp, Synthetic construct
  source 1..21: mol_type = other DNA
actttgtgtgttctgacggag

SEQ ID NO: 167 - DNA, 19 bp, Synthetic construct
  source 1..19: mol_type = other DNA
gaccctgctcgtattctgc

SEQ ID NO: 168 - DNA, 27 bp, Synthetic construct
  source 1..27: mol_type = other DNA
atcgcacgtgggaagtgggctggggag

SEQ ID NO: 169 - DNA, 27 bp, Synthetic construct
  source 1..27: mol_type = other DNA
agtgcaggtgtgcctttgaccacctac

SEQ ID NO: 170 - DNA, 21 bp, Synthetic construct
  source 1..21: mol_type = other DNA
acaggaccaaagtgtcgattg

SEQ ID NO: 171 - DNA, 20 bp, Synthetic construct
  source 1..20: mol_type = other DNA
cactgggttgagcgatcatc

SEQ ID NO: 172 - DNA, 27 bp, Synthetic construct
  source 1..27: mol_type = other DNA
atcgcacgtggctgaggtgttaggtgg

SEQ ID NO: 173 - DNA, 29 bp, Synthetic construct
  source 1..29: mol_type = other DNA
agtgcaggtaaggtctcagggtatttatg

SEQ ID NO: 174 - DNA, 22 bp, Synthetic construct
  source 1..22: mol_type = other DNA
cagtaaattgtccgcctcaact

SEQ ID NO: 175 - DNA, 24 bp, Synthetic construct
  source 1..24: mol_type = other DNA
cctagtttgacgtctaatacccaa

SEQ ID NO: 176 - DNA, 31 bp, Synthetic construct
  source 1..31: mol_type = other DNA
atcgcacgtgaagtgggatatatgaaaggtg

SEQ ID NO: 177 - DNA, 29 bp, Synthetic construct
  source 1..29: mol_type = other DNA
agtgcaggtacgacaaaacagtaactaac

SEQ ID NO: 178 - DNA, 19 bp, Synthetic construct
  source 1..19: mol_type = other DNA
tcagtcactgggccagatg

SEQ ID NO: 179 - DNA, 35 bp, Synthetic construct
  source 1..35: mol_type = other DNA
agtgcaggtaaaacaatggataatggcatcgagaa

SEQ ID NO: 180 - DNA, 28 bp, Synthetic construct
  source 1..28: mol_type = other DNA
cgtgcgatttacacgacttcaccatcca

SEQ ID NO: 181 - DNA, 35 bp, Synthetic construct
  source 1..35: mol_type = other DNA
agtgcaggtaaaacaatgacatgctctgagttgct

SEQ ID NO: 182 - DNA, 31 bp, Synthetic construct
  source 1..31: mol_type = other DNA
cgtgcgatttactagcaactactaacgaact

SEQ ID NO: 183 - DNA, 35 bp, Synthetic construct
  source 1..35: mol_type = other DNA
agtgcaggtaaaacaatggtagtttcgagtgtgaa

SEQ ID NO: 184 - DNA, 31 bp, Synthetic construct
  source 1..31: mol_type = other DNA
cgtgcgatttactaggcaattaggcaccatt

SEQ ID NO: 185 - DNA, 35 bp, Synthetic construct
  source 1..35: mol_type = other DNA
agtgcaggtaaaacaatgagggaaaagatgccatg

SEQ ID NO: 186 - DNA, 31 bp, Synthetic construct
  source 1..31: mol_type = other DNA
cgtgcgatttactagaacctgcttacgtact

SEQ ID NO: 187 - DNA, 35 bp, Synthetic construct
  source 1..35: mol_type = other DNA
agtgcaggtaaaacaatggctaacgaaattgagaa

SEQ ID NO: 188 - DNA, 31 bp, Synthetic construct
  source 1..31: mol_type = other DNA
cgtgcgatttactaagcaacttccaggttca

SEQ ID NO: 189 - DNA, 35 bp, Synthetic construct
  source 1..35: mol_type = other DNA
agtgcaggtaaaacaatggtgaatcaagaggggat

SEQ ID NO: 190 - DNA, 28 bp, Synthetic construct
  source 1..28: mol_type = other DNA
cgtgcgatttagcccatagagacgaagt

SEQ ID NO: 191 - DNA, 35 bp, Synthetic construct
  source 1..35: mol_type = other DNA
agtgcaggtaaaacaatggagataagttctttcaa

SEQ ID NO: 192 - DNA, 28 bp, Synthetic construct
  source 1..28: mol_type = other DNA
cgtgcgatttatacctgcatgatctctt

SEQ ID NO: 193 - DNA, 35 bp, Synthetic construct
  source 1..35: mol_type = other DNA
agtgcaggtaaaacaatggtgaaccaagaagggac

SEQ ID NO: 194 - DNA, 28 bp, Synthetic construct
  source 1..28: mol_type = other DNA
cgtgcgattcagacaacagttacgaagc

SEQ ID NO: 195 - DNA, 35 bp, Synthetic construct
  source 1..35: mol_type = other DNA
agtgcaggtaaaacaatgcagaattacgagaaaag

SEQ ID NO: 196 - DNA, 31 bp, Synthetic construct
  source 1..31: mol_type = other DNA
cgtgcgatttactagaacaatgatgaagaga

SEQ ID NO: 197 - DNA, 29 bp, Synthetic construct
  source 1..29: mol_type = other DNA
cgtgcgattcacacgatcaataattgctg

SEQ ID NO: 198 - DNA, 31 bp, Synthetic construct
  source 1..31: mol_type = other DNA
agtgcaggtaaaacaatgagccacaataaag

SEQ ID NO: 199 - DNA, 26 bp, Synthetic construct
  source 1..26: mol_type = other DNA
cgtgcgatctaagatccgatcagctg

SEQ ID NO: 200 - DNA, 31 bp, Synthetic construct
  source 1..31: mol_type = other DNA
agtgcaggtaaaacaatgtcacagaacaagg

SEQ ID NO: 201 - DNA, 24 bp, Synthetic construct
  source 1..24: mol_type = other DNA
cgtgcgattcatccaagtaattgc

SEQ ID NO: 202 - DNA, 31 bp, Synthetic construct
  source 1..31: mol_type = other DNA
agtgcaggtaaaacaatgagttctaatcagg

SEQ ID NO: 203 - DNA, 24 bp, Synthetic construct
  source 1..24: mol_type = other DNA
cgtgcgattcaagccactgcaggc

SEQ ID NO: 204 - DNA, 32 bp, Synthetic construct
  source 1..32: mol_type = other DNA
agtgcaggtaaaacaatggtaaagatttgctg

SEQ ID NO: 205 - DNA, 41 bp, Synthetic construct
  source 1..41: mol_type = other DNA
actttttgcagtactaaccgcaggtgaaccaggaaggaatc

SEQ ID NO: 206 - DNA, 31 bp, Synthetic construct
  source 1..31: mol_type = other DNA
acacgcgatttagcccatggagacaaaattc

SEQ ID NO: 207 - DNA, 28 bp, Synthetic construct
  source 1..28: mol_type = other DNA
acgtgcgattcagacgatgaggagttgc

SEQ ID NO: 208 - DNA, 36 bp, Synthetic construct
  source 1..36: mol_type = other DNA
agtgcaggtgccacaatgtcccacaacaaggagtcg

SEQ ID NO: 209 - DNA, 25 bp, Synthetic construct
  source 1..25: mol_type = other DNA
acgtgcgatttagcttccaatcagc

SEQ ID NO: 210 - DNA, 33 bp, Synthetic construct
  source 1..33: mol_type = other DNA
agtgcaggtgccacaatgtcccaaaataaagac

SEQ ID NO: 211 - DNA, 28 bp, Synthetic construct
  source 1..28: mol_type = other DNA
acgtgcgatctagccaagcagctgtctg

SEQ ID NO: 212 - DNA, 33 bp, Synthetic construct
  source 1..33: mol_type = other DNA
agtgcaggtgccacaatgtcgtccaaccaggac

SEQ ID NO: 213 - DNA, 38 bp, Synthetic construct
  source 1..38: mol_type = other DNA
actttttgcagtactaaccgcagtcgtccaaccaggac

SEQ ID NO: 214 - DNA, 29 bp, Synthetic construct
  source 1..29: mol_type = other DNA
acgtgcgatagagaccgggttggcggcgc

SEQ ID NO: 215 - DNA, 26 bp, Synthetic construct
  source 1..26: mol_type = other DNA
acacgcgatgacgcagtaggatgtcc

SEQ ID NO: 216 - DNA, 35 bp, Synthetic construct; identical to SEQ ID NO: 102
  source 1..35: mol_type = other DNA

SEQ ID NO: 217 - DNA, 2535 bp, Yarrowia lipolytica
  source 1..2535: mol_type = genomic DNA
atgatcttctctctgcaactactactgacgacggcactagcggcctcttcgcctgactggtaccccacacccgaaatcggttcaattaccagtgattgggccgatgctcttggtgagtccatggacatcctggcccaattgactcttcccgaaaaggtcaacatcaccaccggtaccggatggagaggtgggcagtgtgttggaaacacaggggctgttcctcgtctcggaatcaagggcctgtgtctccaagacggtcctctgggcgtccgttttgccgactttgtcaatgtcttcccttgccagaacgcaatggccgccacctttgatcgtatcctagttcaccaacgaggaaccgctattggacgtcagtctagactcaagggagtcgatgttcatctcggaccagtggtgggacctcttggacgacacgctaccggcggaagaaactgggaaggtttctcccccgacccatatctgtctggaaagctcgccttcgaagcaattcttggagtccaggaagagggagttcttgcaaccatcaagcacttcattggaaacgaacaagaacattaccggcgagccgaagagtggagagacggctttggatttaaagacctgaaggacgccgtctcttccaacattgaagacagagctctacatgagttgtacatgtggccgtttgccgatgctgttagggctaatgtcggctcagtcatgtgctcctacaactacgtgaacggaacccaggcttgccagaacagtgacttgctcaacggaaagctcaagtccgagctcggtttccagggctttgtcatgtccgactggtttgctcagggaagcggagtgtctaacgctctggctggaatggacatgagtatgcctggaaatgacgttgatgagttagaaactgtcttctggggagaacagctgacccgaatggttgccaacggtacccttccagaagcccgtctcgatgatatggttctgcgaattctgacccctctcatctactttgggatcgacgatcgaacacccaacttctcctcctttgtcgacactacagtgggaagtccccaccccgctgctaagcactccaagaaagtcaaggatgttatcaccaactaccatctcgatgtgcgagaccagtttgcagccaatgttgctcttgatagtgctcgaggagctgttgttctgcttttcaatgacggtattcttcccctgaagaacatttccgctattggagtatttggagtcggctctagacttggccccaatggagctgtttgtggggaaaacatgcagtgctcagacggagctcttatcgaaggatggggaagtggaactgcttaccctacggagtacgaaagcccttacgaagctctccacaaaaaggcttctctacttgaagtgtcagtgacaggaaccaccgagtcatgggatatgagacttcctcttgagctggctggagacactgacgtgaatattgtttatgtattggcaaattcaggagagtctactgccaacgttgataagaaccttggggatcgccgaaatgtcagtctatggcacaatggcgatgaacttattaatacagttgccagtcagggacagactgtcgttgttgtcactacggttggacaagttgacatgaccgcttggctcaaccaccccaacatcagtgccgttcttctgactgctcctgctggtgattacggaggaaaagccatcgctgatgtgttatttggagaggttaatccctcaggaaagctgccttatactatcgcagcaaatacttctgattatattcctattgtcaccaagatccctcgagatggagctccccagtccgactttgtggagggaatctatcttgactacaagtggtacgacaagtttgaaaggactcccctctacgaatttggttacggtctgtcatacaccacctactccttcagcaatctgcatcttgatgttaaggagattagtgagttccttcctccccggcctgttcctgtacaggttactaaacccaagatgaccaatatcgacattgaggacctttatgtccccaatgactttaaaatgattgacggtcttgtttacccttggattctcaacgccagtgcgcccctcgcggactctcagactcagttcccctttgcaaatggagctggacatgtcagtgacgcttctggaggagtgggtggtcacccctggctttggtctaacgctgttactgtcactcacaacaccaccaactgcggtgatgttgctggacgagtagtttctcagctgtacgttgccttccctgaaacccttatcgactctcctccggtgcagcttcgaggattcgacaagtccaagctcctgaaccctggtgagtctcagatgaccgagtacaacctcaactggcgagatctggccatttgggacgtggaactgcagagctggagagtgcagcgaggcgagtattccgtgtacattggtcattccagccgagaattcgagctatgtgagactttcactttgtag

SEQ ID NO: 218 - DNA, 2610 bp, Yarrowia lipolytica
  source 1..2610: mol_type = genomic DNA
atgctcgcattcgtcctactgctgacgatgctgctcgcagcagcgcttgctgacccgttctcagataaagacgcttacaaacacagccctccatactaccctgctccggagattggcagagttcccaccgacctgcgatggagagctgccttgaaggtggcccagggtatggtcgctaacatgacactgcttgagaaggtgaacatcaccaccggtacgggctgggagatgggtccttgtgttggaaacactggtaccgtcgaacgactgggtataaaatcgctgtgccttcaagacggccctcttgggattcgatttgctgacctcattaccacattccctgctggtatcactattgcctctaccttctctcgacagctggttagagagcgaggtgctgctatgggacgggagaatagacgcaagggagtggatatcactctcagccctgtggttggaccactgggaagacatgctaacggaggtcgaatctgggagggcttctctgctgacccctaccttgctggaaagctcgccgccgaggccgtgacaggtatacagggccagaacgtcatggctgtggtaaagcatatggttggaaacgagcaggaacattttcgacaacttggcgagtggcagggattcggattcaaggatctgaagcagcccctctcttcaaacatcgacgaccgaactcttaacgaagcgtacctttggccctttgctgatgctgttcgagccaatgttggatctgtcatgtgttcctatcagcagatcaatggctctcagggttgtcaaaacgcccacattctgaacggtaagctcaaggaggaaatgggtttccagggctttgtcatgtccgactggcttgcccagcgaagtggcgtggcgtctgttcttgctggtctcgatatgagcatgcctggagacggtcttgtctgggcggacggtgttccgctcatgggatacgagttgaccaggagtgtgctgaatggaaccattgatgaaagccgagtggacgacatggttacccgaatccttacccccatactgtatctctcaattactccgaccgaccccaacttcagctcttggaccaacgacactactagctacaagtactacggagccaaggctggtggaaacgtcactgttaaccgacatattgatgtgagagaccagtacactaccaaggctgctcttgatggagcaaacgctgcgcttgttcttctcaagaatgagaagaagactcttcctctgaaccctaccaatattggaaacctcaacattttcggtattggttctaaaaccggcccacttggagctgtctgtggagaaaatatgcagtgtagtgatggcgcccttattgagggatgggggtccggttccgtctaccctaccgattatcaatctccttacgacgccattaaggagagagcctccaaggacaacatcaccatcggaggcactacgcaatcttggggtaacctgtcgaacgttgagatcctttcagctgctgccgacgccagtgtcgtctttgttctttccgactccggtgagagtactggtattgttgacggcaacattggggatcgaaacaacttgacgctgtggcacaatggagacgaggttgtcaaggctgtggcatctaagaaccccaacactatcgttgttgttaccactgtcggccctgtgaacctcgaaaagtggatcgacaacccaaacgtcactgccgtgcttctcactggacccgctggtgactttggaggaagagctgcagcctctattctcttcggcgacatcgccccttcaggaaaactccctttcactattgccaagaatgacaccgactacattcctcttactactaagatccctgaagacggccttcctcaagactatttcactgagggtactcttttggactacaaacggttcgacgagaaccaggtgactcctaggtttgaatttggctacggtctgtcttactctaacattacggtggagaatctcgaagcccggtatgctttccctagcattcctgagttcttgcccactccctttgccccttcgaaccccaacaagcctaagaacgcatttactcctcacgccaatgagtccgtcttccccagtgacattgatcctttgaacaagtacgtctatccatacctgaacgatacctcggagatcttctctaacgagacccattatccctatcctgaggggtactccagtgagcagtccaacagtaccaacattaacggcggggctgtcggaggcaaccctgctctgtggctctctgcagtctacattgtccacagcgtgtctaactatggtccctatgatactggagtggtcacccagatgtacattgccttccctcaggataacgacgatcttaaaaccgctcctagacagcttcgaggattcgaacggtccgagctcaaggtgggagaacggcagggaattctatacgatgttcaatggcgagatctcgcggtctgggatgtcaaacttcagagctggcgggtccaacgaggagagtacaaggtttacgtaggccacagttcgcgagactttgttctgaccaccagcttcactctcaagtag

SEQ ID NO: 219 - DNA, 1266 bp, Yarrowia lipolytica
  source 1..1266: mol_type = genomic DNA
atgaaactaaccaaacttgtagctctggcaggagccgctctggcttcgcctatccagctggtccctcgagaaggctctttcctgggattcaactacggcagcgaaaaggtgcacggcgtcaaccttggcggctggtttgtgctggagcccttcatcacgccctctctgttcgaagcgtttggcaacaacgacgccaacgtgcccgtcgacgagtaccactataccgcctggctgggcaaggaggaggccgaaaagcgactgacggaccactggaacacgtggatcaccgagtacgacatcaaggccattgccgaaaactacaagctcaacctggtgcgaatacccattggctactgggccttttcgcttctcccaaacgacccctacgtccagggccaggaggcatacctcgaccgagccctgggctggtgtagaaagtacggagtcaaggcgtgggtcgatgtccatggagtccccggctcccagaacggcttcgacaactccggactgcgagaccactgggactggcccaacgcagacaatgtgcagcattccatcaacgtgatcaactacattgctggcaagtacggcgctcccgagtacaacgacattgtggtgggtatcgagctggtcaacgagcctctgggccccgccattggcatggaggtgattgagaagtacttccaggagggcttctggaccgtgcgacacgcgggctccgacactgccgtggtcatccacgacgcctttcaggagaagaactacttcaacaactttatgaccaccgagcagggtttctggaacgtggttctggaccatcaccagtaccaggtcttttctcccggcgagctggcccggaacattgaccagcacattgccgaggtgtgcaacgtgggtcgacaggcttccacagaataccactggcgaatctttggcgagtggtccgctgctctgaccgattgtacccattggctcaacggtgtgggcaagggccctcgtctggacggctccttccccggctcgtactaccagcgatcgtgccagggtcgaggcgacattcagacgtggtccgagcaggacaagcaggagtctcgacggtacgtggaggcccagctcgatgcgtgggagcatggaggtgatggctggatctactggacttacaagacggagaacgcccttgagtgggacttccgacggctcgtggacaatggcattttccccttcccatactgggaccgacagttccccaaccagtgtggtttttaa

(19) United States
(12) Patent Application Publication
(10) Pub. No.: US 2026/0258343 A1
(43) Pub. Date: Sep. 3, 2026

(54) METHODS FOR PRODUCING MODIFIED BETALAINS IN YEAST

(71) Applicant: Danmarks Tekniske Universitet, Kongens Lyngby (DK)

(72) Inventors: Irina Borodina, Kongens Lyngby (DK); Jane Dannow Dyekjær, Kongens Lyngby (DK); Christiane Ursula Glitz, Kongens Lyngby (DK); Mahsa Babaei, Kongens Lyngby (DK); Philip Tinggaard Thomsen, Kongens Lyngby (DK)

(21) Appl. No.: 19/489,992

(22) PCT Filed: Jun. 7, 2024
(86) PCT No.: PCT/EP2024/065691

§ 371 Date: Dec. 4, 2025

(30) Foreign Application Priority Data
Jun. 8, 2023 (EP) 23178170.9

(51) Int. Cl. C12N 1/16; C12N 9/02; C12N 9/10; C12N 9/42; C12N 9/90; C12P 19/18; C12P 19/44; C12R 1/645

(52) U.S. Cl. CPC C12N 1/16; C12N 9/0069; C12N 9/0071; C12N 9/1029; C12N 9/1048; C12N 9/1085; C12N 9/2445; C12N 9/90; C12P 19/18; C12P 19/44; C12Y 113/11027; C12Y 113/11029; C12Y 114/16002; C12Y 205/01054; C12Y 302/01021; C12Y 504/99005; C12R 2001/645

(57) ABSTRACT

The present invention relates to microbial cell factories, in particular yeast cell factories, for production of modified betalains, such as acylated and di-glycosylated betalains.

### TECHNICAL FIELD

The present invention relates to microbial cell factories, in particular yeast cell factories, for production of modified betalains, such as acylated and di-glycosylated betalains.

### BACKGROUND

Colour is an essential characteristic of food, associated with quality, freshness, and taste perception. Natural and synthetic food dyes are added to processed foods to enhance or correct variations and give an expected colour to colourless foods, such as soft drinks or candies. Many synthetic colours cause hyperactivity in children (McCann et al. 2007), trigger hypersensitivity reactions, and some are carcinogenic in animal studies (Kanner, Harel, and Granit 2001; Oplatowska-Stachowiak and Elliott 2017). Natural colours do not have such adverse effects and some, such as betalains, are even health-promoting (Pietrzkowski et al. 2014; Martin et al. 2016; Sadowska-Bartosz and Bartosz 2021).

While natural colours are in high demand, their natural sources are limited. There are over 75 known natural betalain colours, ranging from yellow to orange to red. However none of them, apart from betanin, is available on the market (Khan and Giridhar 2015). Extraction of these colours is commercially infeasible due to the low native content and difficulties with cultivating and harvesting flowers or other specific plant tissues at scale. Furthermore, alternative natural red compounds with high stability is one of the most challenging issues in food industry. According to studies on plant extracts, specific betalains such as acylated- and/or glycosylated-betalains are shown to have higher stability in terms of reduces racemization velocity compared to betanin (Schliemann and Strack 1998; Moyo and Mukanganyama 2015). This is described as a result of intramolecular stacking, as the U-shape folding of the molecule prevents the aldimine bond from hydrolytic attack. Therefore, the discovery of betanin-modifying enzymes and expressing these enzymes in recombinant hosts will provide an opportunity for developing the cell factories for production of different and probably new-to-nature betacyanins with modified characteristics.

### SUMMARY

The invention is as defined in the claims.

The invention presented herein relates to a yeast cell capable of producing betalains. Betalains are a class of yellow to violet pigments that can be used as natural food dyes. In particular, the invention relates to production of modified betalains, such as acylated and glycosylated betacyanins. Thus, the invention presented herein discloses a yeast cell platform for environment-friendly production of natural food dyes.

In one aspect, the invention provides a yeast cell capable of producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
a. a heterologous BAHD acyltransferase capable of acylating a betalain and/or glycosylated cyclo-DOPA, thereby producing an acylated betalain; and/or
b. a heterologous glycosyltransferase capable of glycosylating a betalain and/or glycosylated cyclo-DOPA, thereby producing a di-glycosylated betalain.

Thus, in one aspect, the present invention provides a yeast cell producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
a. a heterologous BAHD acyltransferase capable of acylating a betalain and/or glycosylated cyclo-DOPA, thereby producing an acylated betalain; and/or
b. a heterologous glycosyltransferase capable of glycosylating a betalain and/or glycosylated cyclo-DOPA, thereby producing a di-glycosylated betalain.

In another aspect, the invention provides a method of producing a modified betalain in the presence of betanin, isobetanin and/or glycosylated cyclo-DOPA in a yeast cell, wherein the modified betalain is selected from the group consisting of acylated betalains and di-glycosylated betalains, said method comprising the steps of:
a. incubating a yeast cell in a medium;
b. expressing in said yeast cell:
   i. a heterologous BAHD acyltransferase capable of acylating a betalain and/or glycosylated cyclo-DOPA, thereby obtaining an acylated betalain; and/or
   ii. a heterologous glycosyltransferase capable of glycosylating a betalain and/or glycosylated cyclo-DOPA, thereby obtaining a di-glycosylated betalain.

In another aspect, the invention provides a method of increasing the titer and/or purity of a modified betalain produced by a yeast cell, wherein the modified betalain is selected from the group consisting of acylated betalains and di-glycosylated betalains, said method comprising the steps of:
a. incubating a yeast cell in a medium in the presence of betanin, isobetanin and/or glycosylated cyclo-DOPA in a yeast cell, said yeast cell comprising a mutation resulting in reduced activity of one or more native β-glucosidases;
b. expressing in said yeast cell:
   i. a heterologous BAHD acyltransferase capable of acylating a betalain and/or glycosylated cyclo-DOPA, thereby obtaining an acylated betalain; and/or
   ii. a heterologous glycosyltransferase capable of glycosylating a betalain and/or glycosylated cyclo-DOPA, thereby producing a di-glycosylated betalain;
      thereby obtaining a modified betalain with an improved purity and/or titer as compared to the purity and/or titer of a modified betalain produced by a yeast cell expressing said heterologous BAHD acyltransferase and/or said heterologous glycosyltransferase, but not comprising a mutation resulting in reduced activity of one or more native β-glucosidases, wherein said yeast cells are cultivated under the same conditions.

In another aspect, the invention provides use of a BAHD acyltransferase as defined herein in a method of producing an acylated betalain, such as an acylated betanin and/or isobetanin.

In another aspect, the invention provides use of a glycosyltransferase as defined herein in a method of producing a di-glycosylated betalain, such as a glycosylated betanin and/or isobetanin.

In another aspect, the invention provides use of a BAHD acyltransferase to catalyse the acylation of glycosylated cyclo-DOPA and/or a betalain, such as betanin and/or isobetanin, thereby obtaining an acylated betalain.

In another aspect, the invention provides use of a glycosyltransferase to catalyse the glycosylation of glycosylated cyclo-DOPA and/or a betalain, such as betanin and/or isobetanin, thereby obtaining a di-glycosylated betalain.

In another aspect, the invention provides use of a BAHD acyltransferase as defined herein for acylation of position 4 of the glycosyl moiety of betanin and/or isobetanin.

In another aspect, the invention provides of a BAHD acyltransferase as defined herein for acylation of position 6 of the glycosyl moiety of betanin and/or isobetanin.

In another aspect, the invention provides use of a glycosyltransferase as defined herein for glucosylation of position 2 of the glycosyl moiety of betanin and/or isobetanin.

In another aspect, the invention provides use of a glycosyltransferase as defined herein for glucuronidation of position 2 of the glycosyl moiety of betanin and/or isobetanin.

In another aspect, the invention provides a system of nucleic acids encoding:
a. a BAHD acyltransferase capable of acylating glycosylated cyclo-DOPA and/or a betalain, such as a betanin and/or isobetanin; and/or
b. a glycosyltransferase capable of glycosylating glycosylated cyclo-DOPA and/or a betalain, such as a betanin and/or isobetanin.

In another aspect, the invention provides use of a polynucleotide as set forth in SEQ ID NO: 30 or SEQ ID NO: 31, or a polynucleotide having at least 70% sequence identity thereto, for obtaining a protein capable of acylating glycosylated cyclo-DOPA and/or a betalain, such as a betanin and/or isobetanin.

In another aspect, the invention provides use of a polynucleotide as set forth in SEQ ID NO: 39, SEQ ID NO: 40, SEQ ID NO: 42, SEQ ID NO: 43, SEQ ID NO: 45 or SEQ ID NO: 46, or a polynucleotide having at least 70% sequence identity thereto, for obtaining a protein capable of glycosylating glycosylated cyclo-DOPA and/or a betalain, such as a betanin and/or isobetanin.

In another aspect, the invention provides a yeast cell capable of producing a glycosylated betalain, said yeast cell expressing:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
   whereby said yeast cell is capable of producing a glycosylated betalain, wherein said yeast cell comprises a mutation resulting in reduced activity of one or more native β-glucosidases.

In another aspect, the invention provides a method for producing a glycosylated betalain, said method comprising the steps of:
a. providing a yeast cell as defined herein;
b. incubating said yeast cell in a medium;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity; thereby obtaining a glycosylated betalain.

In another aspect, the invention provides a method for improving the titer and/or purity of one or more glycosylated betalains produced by a yeast cell, said method comprising the steps of:
a. providing a yeast cell as defined herein, said yeast cell producing a glycosylated betalain;
b. incubating said yeast cell in a medium;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      thereby obtaining a glycosylated betalain with an improved purity and/or titer as compared to the purity and/or titer of a yeast cell expressing said TYH, DOD and enzyme having glycosyltransferase activity but not comprising a mutation resulting in reduced activity of one or more native β-glucosidases, wherein said yeast cells are cultivated under the same conditions.

In another aspect, the invention provides a method for producing a glycosylated betalain, said method comprising the steps of:
a. providing a yeast cell as defined herein;
b. incubating said yeast cell in a medium comprising a β-glucosidase inhibitor, such as cellobiose;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity; thereby obtaining a glycosylated betalain.

In another aspect, the invention provides a method for improving the titer and/or purity of one or more glycosylated betalains produced by a yeast cell, said method comprising the steps of:
a. providing a yeast cell as defined herein, said yeast cell producing a glycosylated betalain;
b. incubating said yeast cell in a medium comprising a β-glucosidase inhibitor, such as cellobiose;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      thereby obtaining a betalain with an improved purity and/or titer as compared to the purity and/or titer of the yeast cell incubated in the absence of the β-glucosidase inhibitor, such as cellobiose, but otherwise cultivated under the same conditions.

In another aspect, the present invention provides a method of increasing the titer and/or purity of a betalain produced by a yeast cell, wherein the betalain is selected from the group consisting of glycosylated betalains, acylated betalains and di-glycosylated betalains, said method comprising the steps of:
a. providing a yeast cell comprising a mutation resulting in reduced activity of one or more native β-glucosidases, said yeast cell producing a glycosylated betalain;
b. incubating said yeast cell in a medium;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
d. optionally, further expressing in said yeast cell:
   i. a heterologous BAHD acyltransferase capable of acylating a betalain and/or glycosylated cyclo-DOPA; and/or
   ii. a heterologous glycosyltransferase capable of glycosylating a betalain and/or glycosylated cyclo-DOPA;
e. optionally recovering the modified betalain;
   thereby obtaining a betalain with an improved purity and/or titer as compared to the purity and/or titer of a betalain produced by a yeast cell expressing said TYH, DOD, enzyme having glycosyltransferase activity and optionally said heterologous BAHD acyltransferase and/or heterologous glycosyltransferase, but not comprising a mutation resulting in reduced activity of one or more native β-glucosidases, and cultivated under the same conditions.

### DESCRIPTION OF THE DRAWINGS

FIG. 1: H PLC chromatogram of the plant extracts from Hylocereus polyrhizus, Bougainvillea glabra and Amaranthus cruentus. The different compounds were identified by LC-MS.

FIG. 2a: Biosynthetic pathway for acylated betalains with aliphatic compounds as acyl donors, HMG: 3-hydroxy-3-methyl glutaryl.

FIG. 2b: HPLC chromatogram of the red dragon fruit extract (Hylocereus polyrhizus extract) together with supernatant of yeast culture ST13955 (expressing betanin pathway together with HpBAHD3) compared with the parent strain (only expressing betanin pathway), the peak for the compound eluted at 9.78 min is similar in strain ST13955 and the red dragon fruit extract.

FIG. 2c: MS fragmentation of the betalain pigment in the red dragon fruit extract and culture supernatant of ST13955. The m/z ratio and the MS/MS fragmentation is identical in both samples and is according to phyllocactin/4′-O-malonyl betanin.

FIG. 3a: HPLC chromatogram of the supernatant of yeast culture ST13933 (ST12160+CcAmaSy1) and the parent strain ST12160 (expresses only the betanin pathway) compared to the Bougainvillea glabra extract. The peak at 7.75 min, present in the CcAmaSy1-expressing strain but not in the parent strain, corresponds to bougainvillein-r-I in the plant extract.

FIG. 3b: Quantification of betanin and bougainvillein-r-I produced upon expression of one of the glucuronosyltransferases AhAmaSy1, CqAmaSy1 or CcAmaSy1 in the betanin-producing S. cerevisiae strains ST12160 and ST12170. Betanin concentration was determined with a calibration standard, bougainvillein-r-I content is given as mAU*min, determined by HPLC.

FIG. 3c: MS and MS/MS data comparing the betalain compounds produced by the strains ST13934, ST13935 and ST13936 expressing AhAmaSy1, CqAmaSy1 or CcAmaSy1, respectively, in addition to the betanin pathway. A peak with RT of 6.98 min is present in the strains with AhAmaSy1 and CcAmaSy1 but not in the parental strain ST12170 or in the strain with CqAmaSy1. MS fragmentation data of the strain ST13936 (ST12170+CcAmaSy1) compared to the Bougainvillea glabra extract identifies this peak as bougainvillein-r-I.

FIG. 4a: Synthesis of UDP-glucuronic acid in S. cerevisiae from UDP-glucose by expression of the UDP-glucose dehydrogenase AtUGD1 leads to the formation of amaranthin instead of bougainvillein-r-I in S. cerevisiae strains expressing AhAmaSy1 or CcAmaSy1.

FIG. 4b: HPLC chromatogram of the supernatant of yeast cultures of ST14115, ST14116 and ST14117, which express AtUGD1 in addition to a glucuronosyltransferase and the betanin-pathway, compared to the strain ST14118, which expresses AtUGD1 and the betanin pathway but does not express a glucuronosyltransferase.

FIG. 4c: Comparison of the amount of betacyanins, bougainvillein-r-I and amaranthin produced upon expression of one of the glucuronosyltransferases AhAmaSy1, CqAmaSy1 or CcAmaSy1 in combination with the UDP-glucose dehydrogenase AtUGD1 in the betanin-producing S. cerevisiae strain ST12160. In all three strains that express AtUGD1 in combination with a glucuronosyltransferase, almost all the betanin is converted to amaranthin. Betacyanin concentration was determined with a calibration standard, bougainvillein-r-I and amranthin content are given as mAU*min, all determined by HPLC.

FIG. 4d: LC-MS confirmation of amaranthin production upon co-expression of AtUGD1 and one of the three glucuronosyltransferases (AhAmaSy1, CqAmaSy1, CcAmaSy1) in S. cerevisiae. Expression of AtUGD1 in the strain ST12160 does not lead to production of amaranthin or bougainvillein-r-I.

FIG. 5a: i) Hydrolysis of betanin to betanidin by wild-type strain of Y. lipolytica in MM (pABA⁻) medium supplemented with betanin from beetroot extract after 24 hours of incubation at 30° C. and 250 rpm. ii) Lack of spontaneous (non-enzymatic) hydrolysis of betanin to betanidin at acidic pH (condition that occurs in yeast cultivations in non-buffered medium). Betanidin formation was checked over 24 hours in MM (pABA⁻) medium supplemented with betanin with different pH values and no cells inoculated.

FIG. 5b: Proposed degradation pathway of betanin through deglycosylation in constructed yeast cell factories for betalain production.

FIG. 5c: Cultivation of β-glucosidase deleted strains in ST6512 background on esculin glycerol agar (EGA), 10 μl of biomass (OD₆₀₀=1) of transformants was radially streaked in 1 mm×4 mm area. Pictures were taken after 5 days incubation at 30° C. The deletion strains with smaller brown hue around the colony than the control strain may have decreased β-glucosidase activity.

FIG. 5d: Hydrolysis of betanin to betanidin by β-glucosidase-deleted strains compared to parent strain in MM (pABA⁻) medium supplemented with betanin from beetroot extract after 24 hours of incubation at 30° C. and 250 rpm.

FIG. 6a: HPLC chromatogram comparing the total betalain content of Y. lipolytica strains ST14105 (ST12603+CcAmaSy1), ST14100 (ST12603+AhAmaSy1) and ST14101 (ST12603+CqAmaSy1) with their parental strain ST12603 and the plant extract of Amaranthus cruentus (Example 2). While the parental strain ST12603 only made betanin and isobetanin, all three strains expressing a glucuronosyltransferase produced large amounts of amaranthin and isoamaranthin. In the strains ST1405 and ST14100, all betanin and isobetanin was converted to amaranthin while in the strain expressing CqAmaSy1, betanin and isobetanin could still be detected in the cultivation broth.

FIG. 6b: LC-MS confirmation of bougainvillein-r-I production by the strains ST14100, ST14101 and ST14105. MS fragmentation data of the strain ST14100 compared to the Bougainvillea glabra extract identified the peak as bougainvillein-r-I.

FIG. 6c: HPLC chromatogram comparing the total betalain content of Y. lipolytica strains ST14103 (ST12603+HpBAHD3) with its parental strain ST12603 and the plant extract of Hylocereus polyrhizus (Example 2). While the parental strain ST12603 only made betanin and isobetanin, ST14103 expressing the BAHD acyltransferase from H. polyrhizus (Example 3) mainly produced phyllocactin. The same was observed for the strain ST14099, expressing HpBAHD3 in the parental strain ST11193.

FIG. 7: Effect of β-glucosidase inhibitors (cellobiose (5 g/L and 10 g/L), ascorbic acid (ASC, 10 mM) and salicin (6.5 mM)) compared with control experiment (no supplementation) on betanin production and retainment at 72 hours. The cultivations were done in biological duplicates in shake flasks with mineral medium containing 40 g/L glucose and ST12603 inoculated.

FIG. 8a: UV-vis spectra of betanin producing rationally engineered platform strain ST12603, and the seven derived strains with knocked-out β-glucosidase genes. The cultivations were done in biological duplicates in shake flasks with mineral medium containing 40 g/L glucose for 96 hours.

FIG. 8b: UV-vis spectra and HPLC-measured titers of betanin-producing strain of Y. lipolytica ST14157 and the three derived strains with knocked-out β-glucosidase genes. The cultivations were done in biological duplicates in shake flasks with mineral medium containing 40 g/L glucose for 96 hours.

FIG. 9a: Fed-batch fermentation of the amaranthin-producing Y. lipolytica strain ST14102. Cultivation of ST14102 resulted in 3 g/L amaranthin after 66 h of fermentation, while almost no (iso-) betanin and bougainvillein-r-I were produced. The concentration of betalains in the total extract (intra-+extracellular) is shown. The products were quantified by HPLC. The average of two bioreactors is shown, shaded areas represent the corresponding standard deviations. F₀: feed initiation with D-glucose.

FIG. 9b: Fed-batch fermentation of the phyllocactin-producing Y. lipolytica strain ST14103. Cultivation of ST14103 resulted in the production of 1.9 g/L phyllocactin after 60 h of fermentation, while up to 77 mg/L (iso-)betanin were produced. The concentration of betalains in the total extract (intra-+extracellular) is shown. The products were quantified by HPLC. The average of two bioreactors is shown, shaded areas represent the corresponding standard deviations. F₀: feed initiation with D-glucose.

FIG. 10: Characterisation of pure betalain variants according to the CIELAB colour space. Biotechnologically produced amaranthin and phyllocactin were purified by preparative HPLC and compared to pure betanin (TCI) with a HunterLab spectrophotometer.

FIG. 11: Betanin production in glucosidase deletion strains. The strains were cultivated in triplicate on 24 deep well plates for 48 hours. Statistical differences were determined by the two-tailed student's t-test (*p<0.05, **p<0.01, ***p<0.001).

FIG. 12. Fed-batch fermentation of the parental strain ST14157 (a), double glucosidase mutant (b) and triple mutant (c). Bioreactor cultivation of each strain was performed in duplicate. Average values are provided.

### DETAILED DESCRIPTION

### Definitions

Acylated betalain: the term “acylated betalain” refers herein to a betalain that has been acylated, i.e. a betalain with an acyl group attached. In particular, an acylated betalain herein refers to a betalain with a malonyl group attached, such as a malonylated betalain, such as a malonylated betanin or a malonylated isobetanin, for example phyllocactin, isophyllocactin, phyllocactin II or isophyllocactin II.

Amaranthin: the term “amaranthin” as used herein refers to the compound with the IUPAC name (2S)-5-[(2S,3R,4S,5S,6R)-3-[(2R,3R,4S,5S,6S)-6-carboxy-3,4,5-trihydroxyoxan-2-yl]oxy-4,5-dihydroxy-6-(hydroxymethyl)oxan-2-yl]oxy-1-[(2E)-2-[(2S)-2,6-dicarboxy-2,3-dihydro-1H-pyridin-4-ylidene]ethylidene]-6-hydroxy-2,3-dihydroindol-1-ium-2-carboxylate and the structure below.

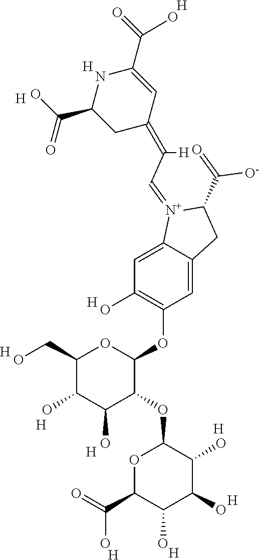

β-glucosidase: the term “β-glucosidase” refers herein to an enzyme that catalyses hydrolysis of terminal, non-reducing β-D-glucosyl residues with release of β-D-glucose (EC 3.2.1.21).

β-glucosidase inhibitor: the term “β-glucosidase inhibitor” refers herein to any type of compound that inhibits the activity of a β-glucosidase, such as for example cellobiose. It may be any compound that decreases the activity of said enzyme by at least 20%, such as at least 30%, such as at least 40%, such as at least 50%, such as at least 60%, such as at least 70%, such as at least 80%, such as at least 90%, for example by 100%, i.e. for example a compound that completely inhibits the activity of said enzyme.

Betacyanin: the term “betacyanin” refers herein to a category of betalains. Betacyanins include red to violet betalain pigments, such as for example betanin and isobetanin.

Betalain: the term “betalain” refers herein to a class of tyrosine-derived pigments found for example in plants of the Caryophyllales. There are two categories of betalains; betaxanthins and betacyanins.

Betaxanthin: the term “betaxanthin” refers herein to a category of betalains. Betaxanthins include yellow to orange betalain pigments.

Bougainvillein-r-I: the term “bougainvillein-r-I” as used herein refers to the compound with the IUPAC name (2S)-1-[(2E)-2-[(2S)-2,6-dicarboxy-2,3-dihydro-1H-pyridin-4-ylidene]ethylidene]-5-[(2S,3R,4S,5S,6R)-4,5-dihydroxy-6-(hydroxymethyl)-3-[(2S,3R,4S,5S,6R)-3,4,5-trihydroxy-6-(hydroxymethyl)oxan-2-yl]oxyoxan-2-yl]oxy-6-hydroxy-2,3-dihydroindol-1-ium-2-carboxylate and the structure below.

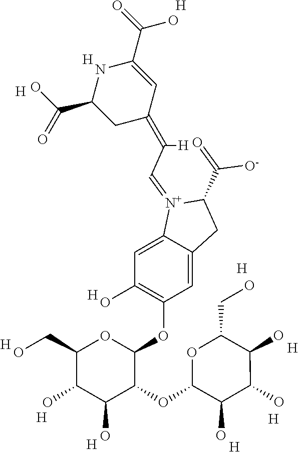

Di-glycosylated betalain: the term “di-glycosylated betalain” refers herein to a betalain that has been di-glycosylated, i.e. a betalain on which two carbohydrates, i.e. two glycosyl donors, have been attached. In particular, a di-glycosylated betalain herein refers to a di-glycosylated betacyanin, such as a glycosylated betanin or isobetanin, such as bougainvillein-r-I, isobougainvillein-r-I, amaranthin or isoamaranthin.

Functional variant: the term “functional variant” refers herein to functional variants of an enzyme, which retain at least some of the activity of the parent enzyme. Thus, a functional variant of a BAHD acyltransferase, a glycosyltransferase, a TYH, a DOD and/or an enzyme having glycosyltransferase activity, such as a glycosyltransferase, such as an SGT, can catalyse the same conversion as the BAHD acyltransferase, the glycosyltransferase, the TYH, the DOD, and/or the enzyme having glycosyltransferase activity, such as the glycosyltransferase, such as the SGT, respectively, from which they are derived, although the efficiency of the reaction may be different, e.g. the efficiency is decreased or increased compared to the parent enzyme or the substrate specificity is modified.

Glycosylated betalain: the term “glycosylated betalain” refers herein to a betalain that has been glycosylated, i.e. a betalain on which a carbohydrate, i.e. a glycosyl donor, has been attached. In particular, a glycosylated betalain herein refers to a glycosylated betacyanin, such as a betanin and/or isobetanin.

Glycosylated cyclo-DOPA: the term “glycosylated cyclo-DOPA” refers herein to cyclo-DOPA with a glycosyl group attached to it. The glycosylated cyclo-DOPA may for example be a cyclo-DOPA glycosylated at position 5 of cyclo-DOPA. The glycosyl group may be of any type, such as for example glucose. Thus, the glycosylated cyclo-DOPA may for example be cyclo-DOPA-5-O-glucoside.

Glycosyltransferase: the term “glycosyltransferase” refers herein to an enzyme that establishes glycosidic linkages. Glycosyltransferases catalyse the transfer of saccharide moieties from an activated nucleotide sugar (“glycosyl donor”) to a nucleophilic glycosyl acceptor molecule. Glycosyltransferases may for example use sugar nucleotide donors, such as UDP-glucose and/or UDP glucuronic acid, and may thus be e.g. glucosyltransferases and/or glucuronosyltransferases. In particular, the term as used herein refers to enzymes that are able to add a second glycosyl moiety to betalains and betalain-precursors, preferably enzymes that are able to add a glycosyl moiety to glycosylated betalains or betalain precursors, such as enzymes that catalyse the formation of di-glycosylated betalains from betanin, isobetanin and/or glycosylated cyclo-DOPA.

Enzyme having glycosyltransferase activity: the term “enzyme having glycosyltransferase activity” as used herein refers to an enzyme that establishes glycosidic linkages. An enzyme having glycosyl activity can catalyse the transfer of saccharide moieties from an activated nucleotide sugar (“glycosyl donor”) to a nucleophilic glycosyl acceptor molecule. Such enzymes may for example use sugar nucleotide donors, such as UDP-glucose and/or UDP glucuronic acid. In particular, the term as used herein refers to enzymes that are able to add a first glycosyl moiety to betalains and betalain-precursors, preferably enzymes that are able to add a glycosyl moiety to unglycosylated betalains or betalain precursors, such as enzymes that catalyse the formation of betanin or glycosylated cyclo-DOPA, preferably enzymes having betanidin-5-O-glucosyltransferase (B5OGT) activity and/or cyclo-DOPA-5-O-glucosyltransferase (cDOPA5OGT) activity.

Hence, both “glycosyltransferase” and “enzyme having glycosyltransferase activity” refers herein to glycosyltransferases, however, to glycosyltransferases that catalyse different types of glycosylation reactions.

Heterologous: the term “heterologous” when referring to a polypeptide, such as a protein or an enzyme, or to a polynucleotide, shall herein be construed to refer to a polypeptide or a polynucleotide that is not naturally present in a wild type cell. For example, the term “heterologous DOD” when applied to Saccharomyces cerevisiae refers to a DOD which is not naturally present in a wild type S. cerevisiae cell, e.g. a DOD derived from Portulaca grandiflora.

Identity/homology: the terms “identity and homology”, with respect to a polynucleotide (or polypeptide), are defined herein as the percentage of nucleic acids (or amino acids) in the candidate sequence that are identical or homologous, respectively, to the residues of a corresponding native nucleic acids (or amino acids), after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985)) as part of the sequence identity. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given homology between two sequences implies that the identity between these sequences is at least equal to the homology; for example, if two sequences are 70% homologous to one another, they cannot be less than 70% identical to one another—but could be sharing 80% identity.

Isomaranthin: the term “isoamaranthin” as used herein refers to the C15 stereoisomer of amaranthin.

Isobougainvillein-r-I: the term “isobougainvillein-r-I” as used herein refers to the C15 stereoisomer of bougainvillein-r-I.

Isophyllocactin: the term “isophyllocactin” as used herein refers to the C15 stereoisomer of phyllocactin.

Isophyllocactin II: the term “isophyllocactin II” as used herein refers to the C15 stereoisomer of phyllocactin II.

Modified betalain: the term “modified betalain” refers herein to a betalain that has one or more chemical group, such as one of more moiety, attached to it. In particular, the term refers herein to a betacyanin, such as a betanin and/or an isobetanin, which has one or more acyl groups and/or one or more glycosyl groups attached to it, i.e. acylated and/or di-glycosylated betalains, such as for example phyllocactin, phyllocactin II, amaranthin and/or bougainvillein-r-I and their respective isoforms isophyllocactin, isophyllocactin II, isoamaranthin and/or isobougainvillein-r-I.

Native to: the term “native to” as used herein when referring to a polypeptide or a polynucleotide native to an organism means that said polypeptide or polynucleotide is naturally found in said organism.

Phyllocactin: the term “phyllocactin” as used herein refers to the compound with the IUPAC name (2S)-4-[2-[(2S)-2-carboxy-5-[(2S,3R,4S,5S,6R)-6-[(2-carboxyacetyl)oxymethyl]-3,4,5-trihydroxyoxan-2-yl]oxy-6-hydroxy-2,3-dihydroindol-1-ium-1-ylidene]ethylidene]-2,3-dihydro-1H-pyridine-2,6-dicarboxylic acid and the structure below. The terms “phyllocactin” and “6′-O-malonyl-betanin” are used interchangeably herein.

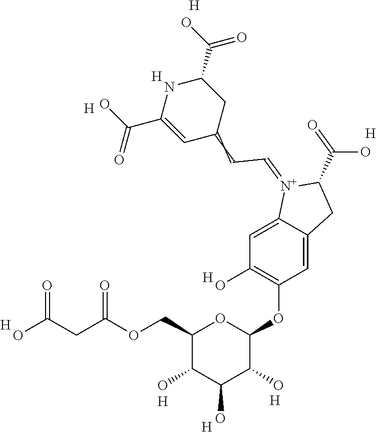

Phyllocactin II: the term “phyllocactin II” as used herein refers to the compound with the IUPAC name (2S)-4-[2-[(2S)-2-carboxy-5-[(2S,3R,4R,5S,6R)-5-(2-carboxyacetyl)oxy-3,4-dihydroxy-6-(hydroxymethyl)oxan-2-yl]oxy-6-hydroxy-2,3-dihydroindol-1-ium-1-ylidene]ethylidene]-2,3-dihydro-1H-pyridine-2,6-dicarboxylic acid and the structure below. The terms “phyllocactin II”, and “4′-O-malonyl-betanin” are used interchangeably herein.

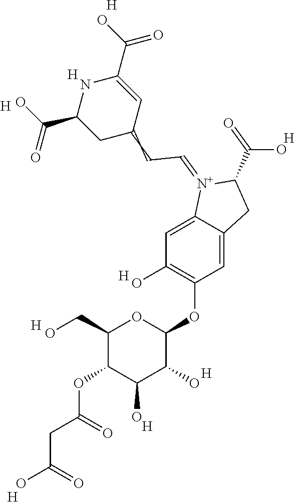

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound that may have evaporated from the culture medium, and it is thus determined by collecting the produced compound from the fermentation broth and from potential off-gas from the fermenter.

### Production of Modified Betalains

The inventors of the present invention have discovered that the expression of certain enzymes enables and/or improves production of modified betalains in yeast cells. Betalains are a class of red to violet (betacyanins) and yellow to orange (betaxanthins) pigments that can be used as natural colours. Modified betalains as used herein refers to a betalain that has one or more chemical group, such as one of more moiety, attached to it. In particular, the modified betalain described herein is an acylated or a di-glycosylated betacyanin, such as for example phyllocactin, phyllocactin II, amaranthin and/or bougainvillein-r-I, and/or their respective isoforms isophyllocactin, isophyllocactin II, isoamaranthin and/or isobougainvillein-r-I.

While natural colours are in high demand, their natural sources are limited. There are over 75 known natural betalain colours, ranging from yellow to orange to red. However, none of them, apart from betanin, is available on the market. Extraction of betalain colours is commercially infeasible due to the low native content and difficulties with cultivating and harvesting flowers or other specific plant tissues at scale. Moreover, the most commonly used extraction methods lead to the presence of pyrazine and geosmin in the red beetroot extract, giving it an undesirable earthy flavour.

Obtaining natural red compounds with high stability is one of the most challenging issues in food industry. According to studies on plant extracts, specific betalains such as acylated and/or di-glycosylated betalains are shown to have higher stability in terms of reduced racemization velocity compared to for example betanin.

Expression of enzymes capable of modifying betalains in recombinant yeast cells will provide an opportunity production of novel, modified betacyanins with new characteristics. Thus, the yeast cells disclosed herein provides a platform for improved and environment-friendly production of natural food dyes well suitable for food colouring.

In particular, the inventors have surprisingly discovered that certain BAHD acyltransferases can be used to acylate betalains or betalain precursors in order to generate acylated betalains, such as phyllocactin and phyllocactin II.

Furthermore, the inventors have surprisingly discovered that certain glycosyltransferases can be used to glycosylate glycosylated betalains or betalain precursors in order to generate di-glycosylated betalains, such as bougainvillein-r-I and amaranthin.

The inventors have also surprisingly discovered that the titer and/or the purity of glycosylated, acylated and/or di-glycosylated betalains produced in yeast cells can be improved by reducing the activity of one or more native β-glucosidases.

Thus, provided herein is a yeast cell capable of producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
a. a heterologous BAHD acyltransferase capable of acylating a betalain and/or glycosylated cyclo-DOPA, thereby producing an acylated betalain; and/or
b. a heterologous glycosyltransferase capable of glycosylating a betalain and/or glycosylated cyclo-DOPA, thereby producing a di-glycosylated betalain.

In other words, provided herein is a yeast cell producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
a. a heterologous BAHD acyltransferase capable of acylating a betalain and/or glycosylated cyclo-DOPA, thereby producing an acylated betalain; and/or
b. a heterologous glycosyltransferase capable of glycosylating a betalain and/or glycosylated cyclo-DOPA, thereby producing a di-glycosylated betalain.

Also provided herein is a kit-of-parts, said kit comprising:
a. the yeast cell as presented herein; and/or
b. the nucleic acid system as presented herein, wherein said construct is for modifying a yeast cell; and
c. instructions for use; and
d. optionally, the yeast cell to be modified.

Further provided herein is the use of modified betalains obtained by the methods disclosed herein as natural food dyes.

Also provided herein is a method for colouring foodstuff, comprising producing modified betalains according to the methods presented herein, and adding or mixing them with the foodstuff to be coloured.

### Betalains

Betalains are water-soluble, tyrosine-derived pigments, in which betalamic acid is the central chromophore. Betalains can be divided into two groups of compounds; betacyanins, which are red to violet pigments derived by condensation of betalamic acid with cyclo-dihydroxyphenylalanine (cyclo-DOPA); and betaxanthins, which are yellow to orange pigments derived from betalamic acid via conjugation with different amines and amino acids. Betacyanins have an absorbance spectrum with a maximal wavelength centred at 536 nm, while betaxanthins have a maximal wavelength centred at 480 nm.

The first committed step in the betalain biosynthesis pathway is a tyrosine hydroxylase reaction, where L-tyrosine is converted to L-3,4-dehydroxyphenylalanine (L-DOPA). L-DOPA may be converted to L-dopaquinone via oxidation, and further converted into cyclo-DOPA through spontaneous cyclization. All these reactions may be catalysed by the P450 cytochrome enzyme CYP76AD (cytochrome P450 76AD), such as by CYP76ADαβ. Throughout this document, CYP76ADs, hereunder CYP76Adαβs, are termed as TYHs. cyclo-DOPA may be glycosylated by an enzyme with cDOPA5OGT activity to form cyclo-DOPA-5-O-glucoside (cDOPA50G). cDOPA50G may spontaneously react with betalamic acid to form betanin. Alternatively, cyclo-DOPA may converted into betanidin via spontaneous reaction with betalamic acid.

L-DOPA may alternatively be converted into 4,5-seco-DOPA in a reaction catalysed by a 4,5-DOPA extradiol dioxygenase (DOD). 4,5-seco-DOPA may be further converted into betalamic acid through spontaneous cyclization. Betalamic acid may be further converted into a betaxanthin though spontaneous reaction with an amino or an amine group. Alternatively, betalamic acid may spontaneously be converted into betanidin via spontaneous reaction with cyclo-DOPA.

Betanidin may be converted into betanin and/or isobetanin in a reaction catalysed by an enzyme with B5OGT activity.

Modified betalains as used herein refers to betalains that have one or more chemical group, such as one of more moiety, attached to it. In particular, the modified betalains described herein are betacyanins with one or more acyl groups and/or one or more glycosyl groups attached to them, such as acylated and/or glycosylated betanin and/or isobetanin.

It should be understood that throughout the disclosure, yeast cells capable of producing modified betalains as disclosed herein are generally also capable of producing the isoforms of said modified betalains. For example, a yeast cell capable of producing phyllocactin, phyllocactin II, amaranthin and/or bougainvillein-r-I is generally also capable of producing the respective isoform of said compounds, i.e. isophyllocactin, isophyllocactin II, isoamaranthin and/or isobougainvillein-r-I.

In other words, yeast cells producing modified betalains as disclosed herein are generally also producing the isoforms of said modified betalains. For example, a yeast cell producing phyllocactin, phyllocactin II, amaranthin and/or bougainvillein-r-I is generally also producing the respective isoform of said compounds, i.e. isophyllocactin, isophyllocactin II, isoamaranthin and/or isobougainvillein-r-I.

It should further be understood that throughout the disclosure, methods for producing modified betalains as disclosed herein generally also comprise producing the respective isoforms of said modified betalains. For example, a method for producing phyllocactin, phyllocactin II, amaranthin and/or bougainvillein-r-I may further comprise producing the respective isoform of said compounds, i.e. isophyllocactin, isophyllocactin II, isoamaranthin and/or isobougainvillein-r-I. In some embodiments, a mixture of a modified betalain and of one or more of its isoforms can thus be produced.

It should further be understood that throughout the disclosure, uses of the BAHD acyltransferases and glycosyltransferases as disclosed herein for catalysing reactions with and/or modifying betalains as disclosed herein generally also comprise catalysing reactions with and/or modifying the respective isoforms of said betalains. For example, use of an enzyme for catalysing the formation of phyllocactin, phyllocactin II, amaranthin and/or bougainvillein-r-I may further comprise catalysing the formation of the respective isoform of said compounds, i.e. isophyllocactin, isophyllocactin II, isoamaranthin and/or isobougainvillein-r-I.

The skilled person will further now that in some embodiments, the respective modified betalains described above may spontaneously isomerize, i.e. spontaneously shift between its two C15 stereoisomers.

In some embodiments, a mixture of a modified betalain and of one or more of its isoforms can thus be produced.

Thus, in one embodiment, the modified betalain is an acylated betalain. Preferably, the modified betalain is an acylated betacyanin. Even more preferably, the modified betalain is an acylated betanin and/or isobetanin, such as a betanin and/or isobetanin acylated at position 4 of the glycosyl moiety of betanin and/or isobetanin, for example a betanin and/or isobetanin malonylated at position 4 of the glycosyl moiety of betanin and/or isobetanin. Most preferably, said modified betalain is phyllocactin II and/or isophyllocactin II.

In an equally preferred embodiment, the modified betalain is a betanin and/or isobetanin acylated at position 6 of the glycosyl moiety of betanin and/or isobetanin, for example a betanin and/or isobetanin malonylated at position 6 of the glycosyl moiety of betanin and/or isobetanin. Most preferably, said modified betalain is phyllocactin and/or isophyllocactin.

In yet another equally preferred embodiment, the modified betalain is a di-glycosylated betalain. Preferably, the modified betalain is a di-glycosylated betacyanin. Even more preferably, the modified betalain is a glycosylated betanin and/or isobetanin, such as a betanin and/or isobetanin glycosylated at position 2 of the glycosyl moiety of betanin and/or isobetanin, for example a betanin and/or isobetanin glucosylated at position 2 of the glycosyl moiety of betanin and/or isobetanin. Most preferably, said modified betalain is bougainvillein-r-I and/or isobougainvillein-r-I.

In an additionally equally preferred embodiment, the modified betalain is a betanin and/or isobetanin glycosylated at position 2 of the glycosyl moiety of betanin and/or isobetanin, for example a betanin and/or isobetanin glucuronidated at position 2 of the glycosyl moiety of betanin and/or isobetanin. Most preferably, said modified betalain is amaranthin and/or isoamaranthin.

### Yeast Cell

The yeast cell may be any type of yeast cell. In some embodiments, the genus of the yeast cell is selected from the group consisting of Saccharomyces, Pichia, Yarrowia, Kluyveromyces, Candida, Rhodotorula, Rhodosporidium, Cryptococcus, Trichosporon and Lipomyces, such as Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In a preferred embodiment, the genus is Saccharomyces or Yarrowia, most preferably the genus is Yarrowia. In some embodiments, the yeast is Saccharomyces cerevisiae. In some embodiments, the yeast is Yarrowia lipolytica.

The yeast cell to be modified, which will also be referred to as the host cell, may express native enzymes that are of the same or of a different class as the enzymes that are necessary for the production of betalains. In some cases, however, such native enzymes may have a negative impact on the titer of betalains that can be obtained; the native enzymes may thus be inactivated by methods known in the art, such as gene editing. For example, the genes encoding the native enzymes having a negative impact on the titer may be deleted or mutated, leading to total or partial loss of activity of the native enzyme.

The inventors have discovered that native β-glucosidases may degrade betalains, and/or precursors of said betalains. In particular, the inventors have discovered that deletion and/or mutation of such native β-glucosidases may have a positive impact on the betalain titer.

Thus, in one embodiment, the yeast cell has been modified by mutating and/or deleting one or more native β-glucosidases. Thus, in one embodiment, the yeast cell comprises a mutation resulting in reduced activity of one or more native β-glucosidases. Preferably, the activity of the mutated β-glucosidase is reduced as compared to the activity of the unmutated, native β-glucosidase. In other words, the yeast provided herein shows reduced activity of the mutated β-glucosidase compared to a yeast cell in which the one or more native β-glucosidases has not been mutated, but otherwise identical, when cultivated in the same conditions. A mutation resulting in reduced activity of β-glucosidase could be an insertion, for example an insertion resulting in a frameshift; a deletion, whether partial or total; a substitution, which could for instance disrupt the tertiary structure of the enzyme.

In one embodiment, the yeast cell is a Yarrowia lipolytica yeast cell, such as a Yarrowia lipolytica W29 yeast cell, and the native β-glucosidase is encoded by a gene selected from the group consisting of:
YALI1_B23300g (SEQ ID NO: 61);
YALI1_F21504g (SEQ ID NO: 62);
YALI1_B05024g (SEQ ID NO: 63);
YALI1_B18845g (SEQ ID NO: 64);
YALI1_B18887g (SEQ ID NO: 65);
YALI1_D22997g (SEQ ID NO: 66);
YALI1_E40502g (SEQ ID NO: 67);
YALI1_F02592g (SEQ ID NO: 68);
YALI1_F08075g (SEQ ID NO: 69);
YALI1_F17788g (SEQ ID NO: 70);
YALI1_E23994g (SEQ ID NO: 79);
YALI1_E39796g (SEQ ID NO: 80);
YALI1_F03075g (SEQ ID NO: 81); and
YALI1_E25163g (SEQ ID NO: 82).

In one embodiment, the yeast cell is a Yarrowia lipolytica yeast cell, such as a Yarrowia lipolytica W29 yeast cell or a Yarrowia lipolytica CLIB122 yeast cell, and the native β-glucosidase is encoded by a gene selected from the group consisting of:
YALI1_B23300g (SEQ ID NO: 61);
YALI1_F21504g (SEQ ID NO: 62);
YALI11B05024g (SEQ ID NO: 63);
YALI1_B18845g (SEQ ID NO: 64);
YALI1_B18887g (SEQ ID NO: 65);
YALI1_D22997g (SEQ ID NO: 66);
YALI1_E40502g (SEQ ID NO: 67);
YALI1_F02592g (SEQ ID NO: 68);
YALI1_F08075g (SEQ ID NO: 69);
YALI1_F17788g (SEQ ID NO: 70);
YALI1_E23994g (SEQ ID NO: 79);
YALI1_E39796g (SEQ ID NO: 80);
YALI1_F03075g (SEQ ID NO: 81);
YALI1_E25163g (SEQ ID NO: 82);
YALI0F16027g (SEQ ID NO: 217);
YALI0B14289g (SEQ ID NO: 218); and
YALI0F05390g (SEQ ID NO: 219).

In a preferred embodiment, the native β-glucosidase is encoded by a gene selected from the group consisting of YALI1_D22997g (SEQ ID NO: 66), YALI1_B23300g (SEQ ID NO: 61), YALI1_F21504g (SEQ ID NO: 62), and YALI1_E23994g (SEQ ID NO: 82) (SEQ ID NO: 219).

In an equally preferred embodiment, the native β-glucosidase is encoded by a gene selected from the group consisting of YALI1_D22997g (SEQ ID NO: 66), YALI1_B23300g (SEQ ID NO: 61), YALI1_F21504g (SEQ ID NO: 62), YALI1_E23994g (SEQ ID NO: 82), YALI0F16027g (SEQ ID NO: 217), YALI0B14289g (SEQ ID NO: 218) and YALI0F05390g (SEQ ID NO: 219).

In an equally preferred embodiment, the native β-glucosidase is encoded by a gene selected from the group consisting of YALI1_F08075g (SEQ ID NO: 69), YALI1_B18845g (SEQ ID NO: 64), and YALI1_F21504g (SEQ ID NO: 62).

In an equally preferred embodiment, the native β-glucosidase is encoded by a gene selected from the group consisting of YALI0F16027g (SEQ ID NO: 217, YALI0B14289g (SEQ ID NO: 218) and/or YALI0F05390g (SEQ ID NO: 219).

As mentioned above, preferably the activity is reduced as compared to the activity of the unmutated, native β-glucosidase. In other words, the mutated β-glucosidase with reduced activity is less efficient, or incapable, at degrading glycosylated betalains as compared to the corresponding unmutated, native β-glucosidase.

In some embodiments, the yeast cell has been modified for decreased production of byproducts i.e. decreased formation of side-products. In other words, the yeast cell has one or more mutations in genes involved in byproduct formation, such as in one or more genes encoding for one or more proteins that are involved in catalysing the formation of byproducts, wherein such mutations lead to partial or total loss of activity of said protein(s). In other words, said yeast cell having said mutation produces less or no byproducts. Thus, in said yeast cell, less or no products are produced in processes that are competitive with that of modified betalain production.

In a preferred embodiment, the yeast cell has a mutation resulting in reduced activity of 4-hydroxyphenylpyruvate dioxygenase (4-HPPD). Preferably, the activity of the mutated 4-HPPD is reduced as compared to the activity of the unmutated, native 4-HPPD. In other words, the activity of 4-HPPD is reduced as compared the activity in a yeast cell in which 4-HPPD has not been mutated, but otherwise identical, when cultivated in the same conditions. Preferably, the yeast cell has a mutation in the gene encoding for 4-HPPD, such as a mutation leading to partial or total loss of activity of 4-HPPD. In one embodiment, the yeast cell is a Yarrowia lipolytica yeast cell and the 4-HPPD is a Yarrowia lipolytica 4-HPPD (SEQ ID NO: 17). A mutation resulting in reduced activity of 4-HPPD could be an insertion, for example an insertion resulting in a frameshift; a deletion, whether partial or total; a substitution, which could for instance disrupt the tertiary structure of the enzyme; whereby 4-HPPD is no longer expressed or is no longer functional.

In some embodiments, the yeast cell has been modified for increased production of precursors, i.e. increased formation of metabolites upstream of betalains. The yeast cell may for example have additional copies of gene(s) encoding for enzymes involved in production of precursors, and/or one or more mutations in genes involved in production of precursors, such as in one or more genes encoding for one or more proteins that are involved in catalysing the formation of precursors, wherein such mutations lead to increased activity of said protein(s). In other words, said yeast cell having said additional copies of gene(s) and/or said mutation(s) produces an increased amount of betalain precursors.

In some embodiments, the yeast cell has been modified to produce high amounts of L-tyrosine. In other words, the yeast cell carries a modification enabling it to produce higher amounts of L-tyrosine as compared to a yeast cell not carrying said modification. Preferably, the production of L-tyrosine in the yeast cell that has been modified to produce high amounts of L-tyrosine is increased as compared the production in a yeast cell not carrying said modification, but otherwise identical, when cultivated in the same conditions. In some embodiments, the yeast cell has a mutation in at least one of the genes involved in L-tyrosine biosynthesis. For example, the yeast cell has one or more point mutation(s) in one or more enzyme(s) involved in L-tyrosine biosynthesis. In one embodiment, said one or more point mutation(s) results in said enzyme(s) being less sensitive to feedback inhibition by aromatic amino acids. In other words, said one or more enzyme(s) with said one or more point mutation(s) are not inhibited, or inhibited to a lesser degree than its native counterpart having no point mutation(s), by aromatic amino acids, such as by amino acids L-tyrosine, L-phenylalanine and/or L-tryptophan. Preferably, the yeast cell carries a mutation resulting in less sensitivity to feedback inhibition by aromatic amino acids, such as wherein the sensitivity to feedback inhibition by aromatic amino acids is reduced as compared the sensitivity in a yeast cell not carrying said mutation, but otherwise identical, when cultivated in the same conditions.

In one embodiment, the yeast cell has a point mutation in 3-deoxy-7-phosphoheptulonate synthase (Aro4). In one embodiment, the yeast cell has a point mutation in Yarrowia lipolytica Aro4 (SEQ ID NO: 71), such as a point mutation in the wild-type Aro4 from Yarrowia lipolytica, such as wherein amino acid no. 221 of Yarrowia lipolytica Aro4 is substituted with leucine. In another embodiment, the yeast cell has a point mutation in Saccharomyces cerevisiae Aro4 (SEQ ID NO: 75), such as a point mutation in the wild-type Aro4 from Saccharomyces cerevisiae, such wherein amino acid no. 229 of Saccharomyces cerevisiae Aro4 is substituted with leucine.

In yet another embodiment, the yeast cell has a point mutation in chorismate mutase (Aro7). In one embodiment, the yeast cell has a point mutation in Yarrowia lipolytica Aro7 (SEQ ID NO: 73), such as a point mutation in the wild-type Aro4 from Yarrowia lipolytica, such wherein amino acid no. 139 of Yarrowia lipolytica Aro4 is substituted with serine. In another embodiment, the yeast cell has a point mutation in Saccharomyces cerevisiae Aro7 (SEQ ID NO: 77), such as a point mutation in the wild-type Aro7 from Saccharomyces cerevisiae, such wherein amino acid no. 141 of Saccharomyces cerevisiae Aro7 is substituted with serine.

In some embodiments, the yeast cell is supplied with one or more precursors required for the production of modified betalains. Thus, in one embodiment, the yeast cell is supplied with a modified betalain precursor selected from the group consisting of glycosylated cyclo-DOPA, betanin, isobetanin and UDP-glucuronic acid. One or more of said compounds may for example be supplied to the growth medium of the yeast cell.

In other embodiments, the yeast cell is engineered to produce one or more precursors required for the production of modified betalains. Thus, in one embodiment, the yeast cell is capable of producing glycosylated cyclo-DOPA, UDP-glucuronic acid, betanin and/or isobetanin.

In one embodiment, the yeast cell expresses, in addition to the BAHD acyltransferase and/or the glycosyltransferase:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
   whereby the yeast cell is capable of producing betanin and/or isobetanin, such as wherein the yeast cell produces betanin and/or isobetanin.

In one embodiment, the TYH is as described in the section “TYH”. In particular, the TYH may be selected from the group consisting of the TYHs as set forth in SEQ ID NO: 4, SEQ ID NO: 6 and SEQ ID NO: 49 or functional variants thereof having at least 70% identity thereto.

In one embodiment, the DOD is as described in the section “DOD”. In particular, the DOD may be selected from the group consisting of the DODs as set forth in SEQ ID NO: 1, SEQ ID NO: 55 and SEQ ID NO: 52 or functional variants thereof having at least 70% identity thereto.

In one embodiment, the enzyme having glycosyltransferase activity, such as the glycosyltransferase is as described in the section “Enzyme having glycosyltransferase activity”. In particular, the enzyme having glycosyltransferase activity may be selected from the group consisting of the enzymes having glycosyltransferase activity as set forth in SEQ ID NO: 8, SEQ ID NO: 11, SEQ ID NO: 57 and SEQ ID NO: 59 or functional variants thereof having at least 70% identity thereto.

In one embodiment, the yeast cell is capable of producing UDP-glucuronic acid. The yeast cell may be naturally capable of producing UDP-glucuronic acid, such as wherein the yeast cell is a Yarrowia lipolytica yeast cell. Alternatively, the yeast cell may be engineered to produce UDP-glucuronic acid. In some embodiments, the yeast cell may be a natural UDP-glucuronic acid producer engineered for increased production of UDP-glucuronic acid.

Thus, in one embodiment, the yeast cell expresses a UDP-glucose dehydrogenase, whereby the yeast cell is capable of producing UDP-glucuronic acid. Thus, in some embodiments, the yeast cell as defined herein is capable of producing amaranthin.

In one embodiment, the UDP-glucose dehydrogenase is a heterologous UDP-glucose dehydrogenase. For example, the UDP-glucose dehydrogenase may be native to a plant, such as a plant of the genus Arabidopsis, for example Arabidopsis thaliana.

In one embodiment, the UDP-glucose dehydrogenase is AtUGD1 as set forth in SEQ ID NO: 47 or a functional variant thereof having at least 70%, such as at least 75% such as at least 80%, such as at least 85%, such as at least 90%, such as at least 95% sequence identity thereto.

In some embodiments, the yeast cell has been modified to express the BAHD acyltransferase and/or the glycosyltransferase, and optionally a TYH, a DOD and/or an enzyme having glycosyltransferase activity, at the genomic level, e.g. by gene editing in the genome. The yeast cell may also be modified by insertion of at least one nucleic acid construct such as at least one vector, for example a plasmid, or by introduction in the cell of a system comprising several nucleic acids as detailed herein below. The vector may be designed as is known to the skilled person either to enable integration of nucleic acid sequences in the genome, or to enable expression of a polypeptide encoded by a nucleic acid sequence comprised in the vector without genome integration.

In some embodiments, the genes encoding the BAHD acyltransferase and/or the glycosyltransferase, and optionally the TYH, the DOD and/or the enzyme having glycosyltransferase activity, have been codon optimized for said yeast cell. In other embodiments, the genes encoding the BAHD acyltransferase and/or the glycosyltransferase, and optionally the TYH, the DOD and/or the enzyme having glycosyltransferase activity are under control of an inducible promoter.

In some embodiments, the genes encoding the BAHD acyltransferase and/or the glycosyltransferase, and optionally the TYH, the DOD and/or the enzyme having glycosyltransferase activity are present in high copy number, and/or they are each independently comprised within the genome of the yeast cell or within a vector comprised in the yeast cell.

In some embodiments, at least one of the genes the BAHD acyltransferase and/or the glycosyltransferase, and optionally the TYH, the DOD and/or the enzyme having glycosyltransferase activity is present in at least two copies, such as at least three copies, such as at least four copies, such as at least five copies.

In one embodiment, the gene encoding the BAHD acyltransferase is present in at least two copies, such as at least three copies, such as at least four copies, such as at least five copies. In one embodiment, the gene encoding the glycosyltransferase is present in at least two copies, such as at least three copies, such as at least four copies, such as at least five copies. In one embodiment, the gene encoding the TYH is present in at least two copies, such as at least three copies, such as at least four copies, such as at least five copies. In one embodiment, the gene encoding the DOD is present in at least two copies, such as at least three copies, such as at least four copies, such as at least five copies. In one embodiment, the gene encoding the enzyme having glycosyltransferase activity is present in at least two copies, such as at least three copies, such as at least four copies, such as at least five copies.

In one embodiment, at least one of the nucleic acids encoding the BAHD acyltransferase and/or the glycosyltransferase, and optionally the TYH, the DOD and/or the enzyme having glycosyltransferase activity, is under the control of an inducible promoter.

In some embodiments, at least one of the nucleic acids encoding the BAHD acyltransferase and/or the glycosyltransferase, and optionally the TYH, the DOD and/or the enzyme having glycosyltransferase activity, is codon-optimized for said yeast cell.

The yeast cell according to any one of the preceding items, wherein the nucleic acids encoding the BAHD acyltransferase and/or the glycosyltransferase, and optionally the TYH, the DOD and/or the enzyme having glycosyltransferase activity, are each independently comprised within the genome of the yeast cell or within a vector comprised within the yeast cell.

In one embodiment, the yeast cell comprises a system of vectors, as described in the section “Nucleic acid”.

### BAHD Acyltransferase

In the present invention, BAHD acyltransferase refers to an enzyme with acyltransferase activity. The term “heterologous BAHD acyltransferase” refers to a BAHD acyltransferase that is not naturally expressed by the yeast cell.

BAHD acyltransferases are acyl CoA-utilizing enzymes that transfer acylated moieties (RC(O)R′) of an acyl-activated CoA thioester donor to an acceptor molecules. BAHD acyltransferases thus catalyse the following reaction:

CoA-activated acyl donor+acyl acceptor→acylated acceptor molecule

The EC number is EC 2.3.1

In one embodiment of the present invention, the BAHD acyltransferase is native to a a plant. In one embodiment, the BAHD acyltransferase is native to a plant of the genus Hylocereus, such as Hylocereus polyrhizus, or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to such BAHD acyltransferase.

In one embodiment, the BAHD acyltransferase is HpBAHD3 as set forth in SEQ ID NO: 29 or a functional variant thereof having at least 70% sequence identity thereto, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to said BAHD acyltransferase.

A functional variant of a BAHD acyltransferase refers to a variant of BAHD acyltransferase that retains at least some of the activity of the parent enzyme. Thus, a functional variant of a BAHD acyltransferase can catalyze the same conversion as the BAHD acyltransferase from which it is derived, although the efficiency of the reaction may be different, e.g. the efficiency may be decreased or increased compared to the parent enzyme. Testing whether or not an enzyme is a functional variant of a BAHD acyltransferase can be tested using methods known in the art. For example, the BAHD acyltransferase variant can be expressed in a cell, wherein the cell medium contains the substrate of BAHD acyltransferase, i.e. betanin, isobetanin and/or glycosylated cyclo-DOPA, or said substrate is produced in said cell. After incubating the cell for 24 hours, the amount of product, i.e. the amount of phyllocactin or phyllocactin II, generated by the cell, i.e. by the BAHD acyltransferase variant, can be measured. If the BAHD acyltransferase variant generates the same product as the BAHD acyltransferase does, i.e. phyllocactin or phyllocactin II, when said BAHD acyltransferase is tested under the same conditions, i.e. expressed in a cell and incubated for 24 h, the BAHD acyltransferase variant is a functional variant of said BAHD acyltransferase.

In one embodiment, the present invention further provides the use of a BAHD acyltransferase as defined in a method of producing an acylated betalain, such as an acylated betanin and/or isobetanin.

In one embodiment, the present invention also provides the use of a BAHD acyltransferase to catalyse the acylation of glycosylated cyclo-DOPA and/or a betalain, such as betanin and/or isobetanin, thereby obtaining an acylated betalain.

Preferably, the BAHD acyltransferase is as defined herein in the section “BAHD acyltransferase” and/or the acylated betalain is as defined herein in the section “Betalains”.

Thus, one embodiment of the present invention provides the use of a BAHD acyltransferase for acylation of position 4 of the glycosyl moiety of betanin and/or isobetanin.

One embodiment of the present invention provides the use of a BAHD acyltransferase for acylation of position 6 of the glycosyl moiety of betanin and/or isobetanin.

Thus, one embodiment of the present invention provides the use of a BAHD acyltransferase for acylation of position 4 of the glycosyl moiety of glycosylated cyclo-DOPA.

One embodiment of the present invention provides the use of a BAHD acyltransferase for acylation of position 6 of the glycosyl moiety of glycosylated cyclo-DOPA.

In one embodiment, the acylation comprises malonylation.

### Glycosyltransferase

In the present invention, glycosyltransferase refers to an enzyme with glycosyltransferase activity. The term “heterologous glycosyltransferase” refers to a glycosyltransferase that is not naturally expressed by the yeast cell.

Glycosyltransferases (EC 2.4) are enzymes that establish natural glycosidic linkages. They catalyze the transfer of saccharide moieties from an activated nucleotide sugar (“the glycosyl donor”) to a glycosyl acceptor molecule:

UDP-sugar substrate (glycosyl donor)+glycosyltransferase substrate (glycosyl acceptor)→UDP+glycosylated glycosyltransferase substrate (EC. 2.4)

Preferably, the glycosyltransferase presented herein is capable glucosylation and/or glucuronidation of position 2 of the glycosyl moiety of betanin, isobetanin and/or glycosylated cyclo-DOPA.

In one embodiment of the present invention, the glycosyltransferase is native to a plant. In one embodiment, the glycosyltransferase is native to a plant of the genus Amaranthus, Chenopodium or Celosia, such as Amaranthus hypochondriacus, Chenopodium quinoa or Celosia cristata, or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to such glycosyltransferase.

In one embodiment, the glycosyltransferase is AhAmaSy1 as set forth in SEQ ID NO: 38 or a functional variant thereof having at least 70% sequence identity thereto, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to said glycosyltransferase.

In one embodiment, the glycosyltransferase is CqAmaSy1 as set forth in SEQ ID NO: 41 or a functional variant thereof having at least 70% sequence identity thereto, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to said glycosyltransferase.

In one embodiment, the glycosyltransferase is CcAmaSy1 as set forth in SEQ ID NO: 44 or a functional variant thereof having at least 70% sequence identity thereto, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to said glycosyltransferase.

A functional variant of a glycosyltransferase refers to a variant of glycosyltransferase that retains at least some of the activity of the parent enzyme. Thus, a functional variant of a glycosyltransferase can catalyze the same conversion as the glycosyltransferase from which it is derived, although the efficiency of the reaction may be different, e.g. the efficiency may be decreased or increased compared to the parent enzyme. Testing whether or not an enzyme is a functional variant of glycosyltransferase can be tested using methods known in the art. For example, the glycosyltransferase variant can be expressed in a cell, wherein the cell medium contains the substrate of glycosyltransferase, i.e. betanin, isobetanin and/or glycosylated cyclo-DOPA, or said substrate is produced in said cell. After incubating the cell for 24 hours, the amount of product, i.e. the amount of bougainvillein-r-I, generated by the cell, i.e. by the glycosyltransferase variant, can be measured. If the glycosyltransferase variant generates the same product as the glycosyltransferase does, i.e. bougainvillein-r-I, when tested under the same conditions, i.e. expressed in a cell and incubated for 24 h, the glycosyltransferase variant is a functional variant of said glycosyltransferase.

In one embodiment, the present invention further provides the use of a glycosyltransferase as defined in a method of producing a di-glycosylated betalain, such as a glycosylated betanin and/or isobetanin.

In one embodiment, the present invention also provides the use of a glycosyltransferase to catalyse the glycosylation of glycosylated cyclo-DOPA and/or a betalain, such as betanin and/or isobetanin, thereby obtaining a di-glycosylated betalain.

Preferably, the glycosyltransferase is as defined herein in the section “Glycosyltransferase” and/or the di-glycosylated betalain is as defined herein in the section “Betalains”.

One embodiment of the present invention provides the use of a glycosyltransferase as defined in any one of the preceding items for glucosylation of position 2 of the glycosyl moiety of betanin and/or isobetanin.

One embodiment of the present invention provides the use of a glycosyltransferase as defined in any one of the preceding items for glucosylation of position 2 of the glycosyl moiety of glycosylated cyclo-DOPA.

One embodiment of the present invention provides the use of a glycosyltransferase as defined in any one of the preceding items for glucuronidation of position 2 of the glycosyl moiety of betanin and/or isobetanin.

One embodiment of the present invention provides the use of a glycosyltransferase as defined in any one of the preceding items for glucuronidation of position 2 of the glycosyl moiety of glycosylated cyclo-DOPA.

### Betanin Production

As described above in the section “Yeast cell”, the yeast cell capable of producing modified betalains, such as the yeast cell producing modified betalains, may be supplemented with betanin, isobetanin and/or betalain precursors, such as glycosylated cyclo-DOPA. Alternatively, or additionally, the yeast cell may be engineered for production of betanin, isobetanin and/or betalains precursors, such as glycosylated cyclo-DOPA.

Thus, in one embodiment of the invention, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
   whereby the yeast cell is capable of producing betanin and/or isobetanin.

In another embodiment, yeast cell expresses:
a. a heterologous enzyme which is a 4,5-DOPA extradiol dioxygenase (DOD); and
b. a heterologous enzyme having glycosyltransferase activity;
   whereby the yeast cell is capable of producing betanin and/or isobetanin in the presence of L-DOPA and/or L-dopaquinone.

In another embodiment, yeast cell expresses:
a. a heterologous enzyme having glycosyltransferase activity;
   whereby the yeast cell is capable of producing betanin and/or isobetanin in the presence of 4,5-seco-DOPA.

In one embodiment of the invention:
the TYH is capable of converting L-tyrosine to L-3,4-dihydroxyphenylalanine (L-DOPA) and/or converting L-DOPA to L-dopaquinone;
the DOD is capable of converting L-DOPA to 4,5-seco-DOPA;
the enzyme having glycosyltransferase activity is capable of converting cyclo-DOPA to cyclo-DOPA-5-O-glucoside and/or glycosylating betanidin, thereby converting betanidin to a glycosylated betalain such as betanin and/or isobetanin;
   wherein one or more of the following reactions are spontaneous reactions:
conversion of 4,5-seco-DOPA to betalamic acid;
conversion of betalamic acid to one or more of a betaxanthin, betanidin, betanin or isobetanin;
conversion of L-dopaquinone to cyclo-DOPA;
conversion of cyclo-DOPA to betanidin.

The yeast cell may express a TYH that is capable of converting L-tyrosine to L-DOPA. The L-DOPA may be converted into L-dopaquinone by the action of the TYH, and/or into 4,5-seco-DOPA by the action of the DOD if expressed by the yeast cell.

L-dopaquinone can be converted to cyclo-DOPA in a spontaneous reaction. Cyclo-DOPA can then be converted to cyclo-DOPA-5-O-glucoside by the action of an enzyme with glycosyltransferase activity, such as by the action of an enzyme with cyclo-DOPA-5-O-glucosyltransferase (cDOPA5OGT) activity or to betanidin by a spontaneous reaction with betalamic acid.

Cyclo-DOPA-5-O-glucoside can be converted to betanidin in a spontaneous reaction with betalamic acid. Betanidin can be converted to betanin and/or isobetanin by an enzyme with glycosyltransferase activity, such as by the action of an enzyme with betanidin-5-O-glucosyltransferase (B5OGT) activity.

4,5-seco-DOPA can be converted to betalamic acid in a spontaneous reaction. Betalamic acid can be converted to betaxanthins by spontaneous reaction with an amine or amino acid.

The TYH may be as defined herein in the section “TYH”.

The DOD may be as defined herein in the section “DOD”.

The enzyme having glycosyltransferase activity may be as defined here in the section “Enzyme having glycosyltransferase activity”.

### TYH

In the present invention, TYHs (tyrosine hydroxylases) refers to CYP76AD enzymes with tyrosine hydroxylase activity. The term ‘CYP76AD’ and ‘TYH’ will be used herein interchangeably. The term ‘heterologous TYH’ refers to a TYH that is not naturally expressed by the organism, such as by the yeast cell.

The TYHs presented herein catalyse the following reactions:

L-tyrosine→L-DOPA

L-DOPA→L-dopaquinone

The EC number for the overall reaction is EC 1.14.18.1.

L-dopaquinone subsequentially cyclizes to form cyclo-DOPA in a spontaneous reaction.

In some embodiments of the present invention the TYH is native to a plant, such as of the genus Abronia, Acleisanthes, Basella, Beta, Cleretum, Ercilla, Mirabilis, Optunia, or Phytolacca, such as Abronia nealleyi, Acleisanthes obtusa, Basella alba, Beta vulgaris, Cleretum bellidiforme, Ercilla volubilis, Mirabilis multiflora, Optunia ficus-indica, or Phytolacca dioica, or a functional variant thereof having at least 80% identity thereto.

In one embodiment, the TYH is a TYH selected from the group of TYH set forth in SEQ ID NO: 4, SEQ ID NO: 6, SEQ ID NO: 49 and SEQ ID NO: 83 to 90, or functional variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to a TYH selected from the group of TYHs as set forth in SEQ ID NO: 4, SEQ ID NO: 6, SEQ ID NO: 49 and SEQ ID NO: 83 to 90

In one embodiment, the heterologous TYH is an Abronia TYH. In one embodiment, the TYH is an Abronia nealleyi TYH, such as the TYH as set forth in SEQ ID NO: 49 (AnTYH). In some embodiments, the TYH is a functional variant of an Abronia TYH, a functional variant of an Abronia nealleyi TYH or a functional variant of the TYH as set forth in SEQ ID NO: 49 (AnTYH), having at least 60% identity thereto.

In one embodiment, the heterologous TYH is an Acleisanthes TYH. In one embodiment, the TYH is an Acleisanthes obtusa TYH, such as the TYH as set forth in SEQ ID NO: 83 (AoTYH). In some embodiments, the TYH is a functional variant of an Acleisanthes TYH, a functional variant of an Acleisanthes obtusa TYH or a functional variant of the TYH as set forth in SEQ ID NO: 83 (AoTYH), having at least 60% identity thereto.

In one embodiment, the heterologous TYH is a Basella TYH. In one embodiment, the TYH is a Basella alba TYH, such as the TYH as set forth in SEQ ID NO: 84 (BaTYH). In some embodiments, the TYH is a functional variant of a Basella TYH, a functional variant of a Basella alba TYH or a functional variant of the TYH as set forth in SEQ ID NO: 84 (BaTYH), having at least 60% identity thereto.

In one embodiment, the heterologous TYH is a Beta TYH. In one embodiment, the TYH is a Beta vulgaris TYH, such as the TYH as set forth in SEQ ID NO: 4 (BvCYP76AD1^{W13L}). In some embodiments, the TYH is a functional variant of a Beta TYH, a functional variant of a Beta vulgaris TYH or a functional variant of the TYH as set forth in SEQ ID NO: 4 (BvCYP76AD1^{W13L}), having at least 60% identity thereto.

In one embodiment, the heterologous TYH is a Cleretum TYH. In one embodiment, the TYH is a Cleretum bellidiforme TYH, such as the TYH as set forth in SEQ ID NO: 85 (CbTYH). In some embodiments, the TYH is a functional variant of a Cleretum TYH, a functional variant of a Cleretum bellidiforme TYH or a functional variant of the TYH as set forth in SEQ ID NO: 85 (CbTYH), having at least 60% identity thereto.

In one embodiment, the heterologous TYH is an Ercilla TYH. In one embodiment, the TYH is an Ercilla volubilis TYH, such as the TYH as set forth in SEQ ID NO: 6 (EvTYH). In some embodiments, the TYH is a functional variant of an Ercilla TYH, a functional variant of an Ercilla volubilis TYH or a functional variant of the TYH as set forth in SEQ ID NO: 6 (EvTYH), having at least 60% identity thereto.

In one embodiment, the heterologous TYH is a Mirabilis TYH. In one embodiment, the TYH is a Mirabilis multiflora TYH, such as the TYH as set forth in SEQ ID NO: 86 (MmTYH1) or the TYH as set forth in SEQ ID NO: 87 (MmTYH2). In some embodiments, the TYH is a functional variant of a Mirabilis multiflora TYH, a functional variant of a Mirabilis TYH or a functional variant of the TYH as set forth in SEQ ID NO: 86 (MmTYH1) or SEQ ID NO: 87 (MmTYH2), having at least 60% identity thereto.

In one embodiment, the heterologous TYH is an Opuntia TYH. In one embodiment, the TYH is an Opuntia ficus-indica TYH, such as the TYH as set forth in SEQ ID NO: 88 (OfTYH). In some embodiments, the TYH is a functional variant of an Opuntia TYH, a functional variant of an Opuntia ficus-indica TYH or a functional variant of the TYH as set forth in SEQ ID NO: 88 (OfTYH), having at least 60% identity thereto.

In one embodiment, the heterologous TYH is a Phytolacca TYH. In one embodiment, the TYH is a Phytolacca americana TYH, such as the TYH as set forth in SEQ ID NO: 89 (PaTYH), or a Phytolacca dioica TYH, such as the TYH set forth in SEQ ID NO: 90 (PdTYH). In some embodiments, the TYH is a functional variant of a Phytolacca TYH, a functional variant of a Phytolacca americana TYH, a functional variant of a Phytolacca dioica TYH, or a functional variant of the TYH as set forth in SEQ ID NO: 89 (PaTYH) or SEQ ID NO: 90 (PdTYH), having at least 60% identity thereto.

A functional variant of a TYH refers to a variant of a TYH, which retains at least some or all of the TYH activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

A functional variant of a TYH refers to a variant of TYH which retains at least some of the activity of the parent enzyme. Thus, a functional variant of a TYH can catalyze the same conversion as the TYH from which it is derived, although the efficiency of the reaction may be different, e.g. the efficiency may be decreased or increased compared to the parent enzyme. Testing whether or not an enzyme is a functional variant of a TYH can be tested using methods known in the art. For example, the TYH variant can be expressed in a cell, wherein the cell medium contains the substrate of TYH, i.e. L-tyrosine, or said substrate is produced in said cell. After incubating the cell for 24 hours, the amount of product, i.e. the amount of L-DOPA and/or L-dopaquinone, generated by the cell, i.e. by the TYH variant, can be measured. If the TYH variant generates the same product, i.e. L-DOPA and/or L-dopaquinone, as the TYH does, when said TYH is tested under the same conditions, i.e. expressed in a cell and incubated for 24 h, the TYH variant is a functional variant of said TYH.

### DOD

The term ‘4,5-DOPA extradiol dioxygenase’ and ‘DOD’ will be used herein interchangeably. The term ‘heterologous DOD’ refers to a DOD which is not naturally expressed by the yeast cell. A DOD as presented herein is an enzyme catalysing the following reaction:

L-DOPA→4,5-seco-DOPA

The EC number for the reaction is EC 1.13.11.29.

4,5-seco-DOPA subsequentially spontaneously cyclizes to form betalamic acid.

In some embodiments of the present invention, the DOD is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

In one embodiment, the DOD is a DOD selected from the group of DOD set forth in SEQ ID NO: 1, SEQ ID NO: 52, SEQ ID NO: 55 and SEQ ID NO: 91 to 99, or functional variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity a DOD selected from the group of DODs set forth in SEQ ID NO: 1, SEQ ID NO: 52, SEQ ID NO: 55 and SEQ ID NO: 91 to 99.

In one embodiment, the heterologous DOD is an Amaranthus DOD. In one embodiment, the DOD is an Amaranthus tricolour DOD, such as the DOD as set forth in SEQ ID NO: 91 (AtDOD). In one embodiment, the DOD is an Amaranthus hypochondriacus DOD, such as the DOD as set forth in SEQ ID NO: 92 (AhDOD). In some embodiments, the DOD is a functional variant of an Amaranthus DOD, a functional variant of an Amaranthus tricolour DOD, a functional variant of an Amaranthus hypochondriacus DOD, a functional variant of the DOD as set forth in SEQ ID NO: 91 (AtDOD), or a functional variant of the DOD as set forth in SEQ ID NO: 92 (AhDOD), having at least 60% identity thereto.

In one embodiment, the heterologous DOD is a Beta DOD. In one embodiment, the DOD is a Beta vulgaris DOD, such as the DOD as set forth in SEQ ID NO: 93 (BvDOD1), the DOD as set forth in SEQ ID NO: 94 (BvDOD2), or the DOD as set forth in SEQ ID NO: 95 (BvDOD3). In some embodiments, the DOD is a functional variant of a Beta DOD, a functional variant of a Beta vulgaris DOD, a functional variant of the DOD as set forth in SEQ ID NO: 93 (BvDOD1), a functional variant of the DOD as set forth in SEQ ID NO: 94 (BvDOD2) or a functional variant of the DOD as set forth in SEQ ID NO: 95 (BvDOD3), having at least 60% identity thereto.

In one embodiment, the heterologous DOD is a Bougainvillea DOD. In one embodiment, the DOD is a Bougainvillea glabra DOD, such as the DOD as set forth in SEQ ID NO: 96 (BgDOD1) or the DOD as set forth in SEQ ID NO: 52 (BgDOD2). In some embodiments, the DOD is a functional variant of a Bougainvillea DOD, a functional variant of a Bougainvillea glabra DOD, a functional variant of the DOD as set forth in SEQ ID NO: 96 (BgDOD1), or a functional variant of the DOD as set forth in SEQ ID NO: 52 (BgDOD2), having at least 60% identity thereto

In one embodiment, the heterologous DOD is a Mirabilis DOD. In one embodiment, the DOD is a Mirabilis jalapa DOD, such as the DOD as set forth in SEQ ID NO: 1 (MjDOD). In some embodiments, the DOD is a functional variant of a Mirabilis DOD, a functional variant of a Mirabilis jalapa DOD or a functional variant of the DOD as set forth in SEQ ID NO: 1 (MjDOD), having at least 60% identity thereto.

In one embodiment, the heterologous DOD is a Phytolacca DOD. In one embodiment, the DOD is a Phytolacca americana DOD, such as the DOD as set forth in SEQ ID NO: 97 (PaDOD). In some embodiments, the DOD is a functional variant of a Phytolacca DOD, a functional variant of a Phytolacca americana DOD or a functional variant of the DOD as set forth in SEQ ID NO: 97 (PaDOD), having at least 60% identity thereto.

In one embodiment, the heterologous DOD is a Portulaca DOD. In one embodiment, the DOD is a Portulaca grandiflora DOD, such as the DOD as set forth in SEQ ID NO: 55 (PgDOD). In some embodiments, the DOD is a functional variant of a Portulaca DOD, a functional variant of a Portulaca grandiflora DOD or a functional variant of the DOD as set forth in SEQ ID NO: 55 (PgDOD), having at least 60% identity thereto.

In one embodiment, the heterologous DOD is a Spinacia DOD. In one embodiment, the DOD is a Spinacia oleracea DOD, such as the DOD as set forth in SEQ ID NO: 98 (SoDOD). In some embodiments, the DOD is a functional variant of a Spinacia DOD, a functional variant of a Spinacia oleracea DOD or a functional variant of the DOD as set forth in SEQ ID NO: 98 (SoDOD), having at least 60% identity thereto.

In one embodiment, the heterologous DOD is a Suaeda DOD. In one embodiment, the DOD is a Suaeda salsa DOD, such as the DOD as set forth in SEQ ID NO: 99 (SsDOD). In some embodiments, the DOD is a v functional variant of a Suaeda DOD, a functional variant of a Suaeda salsa DOD or a functional variant of the DOD as set forth in SEQ ID NO: 99 (SsDOD), having at least 60% identity thereto.

A functional variant of a DOD refers to a variant of a DOD, which retains at least some or all of the DOD activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

A functional variant of a DOD refers to a variant of DOD that retains at least some of the activity of the parent enzyme. Thus, a functional variant of a DOD can catalyze the same conversion as the DOD from which it is derived, although the efficiency of the reaction may be different, e.g. the efficiency may be decreased or increased compared to the parent enzyme. Testing whether or not an enzyme is a functional variant of a DOD can be tested using methods known in the art. For example, the DOD variant can be expressed in a cell, wherein the cell medium contains the substrate of DOD, i.e. L-DOPA, or said substrate is produced in said cell. After incubating the cell for 24 hours, the amount of product, i.e. the amount of 4,5-seco-DOPA, generated by the cell, i.e. by the DOD variant, can be measured. If the DOD variant generates the same product, i.e. 4,5-seco-DOPA, as the DOD does, when said DOD is tested under the same conditions, i.e. expressed in a cell and incubated for 24 h, the DOD variant is a functional variant of said DOD.

### Enzyme Having Glycosyltransferase Activity

The term ‘enzyme having glycosyltransferase activity’, ‘scopoletin glucosyltransferase’ and ‘SGT’ will be used herein interchangeably. The term ‘heterologous enzyme having glycosyltransferase activity’ refers to an enzyme having glycosyltransferase activity, such as a glycosyltransferase, which is not naturally expressed by the organism, such as by the yeast cell. Glycosyltransferases (EC 2.4) are enzymes that establish natural glycosidic linkages. They catalyze the transfer of saccharide moieties from an activated nucleotide sugar (“the glycosyl donor”) to a glycosyl acceptor molecule:

UDP-sugar substrate (glycosyl donor)+glycosyltransferase substrate (glycosyl acceptor)→UDP+glycosylated glycosyltransferase substrate (EC. 2.4)

In some embodiments, the enzyme having glycosyltransferase activity is a scopoletin glucosyltransferase (SGT), which is an enzyme that catalyses the reaction:

UDP-glucose+scopoletin→UDP+scopolin (EC 2.4.1.128)

The enzyme belongs to the family of glycosyltransferases, specifically hexosyltransferases. The systematic name of this enzyme class is UDP-glucose:scopoletin O-beta-D-glucosyltransferase.

Preferably, the heterologous enzyme having glycosyltransferase activity presented herein has an activity selected from betanidin-5-O-glucosyltransferase (B5OGT) activity and cyclo-DOPA-5-O-glucosyltransferase (cDOPA5OGT) activity.

In one embodiment of the present invention, the enzyme having glycosyltransferase activity, such as the glycosyltransferase is native to a plant, such as of the genus Abronia, Beta, Bougainvillea, Chenopodium, Ercilla, or Portacula, such as Abronia nealleyi, Beta vulgaris, Bougainvillea glabra, Chenopodium quinoa, Ercilla volubilis, or Portacula grandiflora, or a functional variant thereof having at least 80% identity thereto.

In one embodiment, the heterologous enzyme having glycosyltransferase activity selected from the group set forth in SEQ ID NO: 8, SEQ ID NO: 11, SEQ ID NO: 57 and SEQ ID NO: 59, or functional variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity an enzyme having glycosyltransferase activity selected from the group of enzymes having glycosyltransferase activity set forth in SEQ ID NO: 8, SEQ ID NO: 11, SEQ ID NO: 57 and SEQ ID NO: 59.

In one embodiment, the heterologous enzyme having glycosyltransferase activity is a Beta glycosyltransferase. In one embodiment, the glycosyltransferase is a Beta vulgaris glycosyltransferase, such as the glycosyltransferase as set forth in SEQ ID NO: 8 (BvSGT2) or the glycosyltransferase as set forth in SEQ ID NO: 59 (BvSGT4). In some embodiments, the glycosyltransferase is a functional variant of a Beta glycosyltransferase, a functional variant of a Beta vulgaris glycosyltransferase or a functional variant of the glycosyltransferase as set forth in SEQ ID NO: 8 (BvSGT2) or the glycosyltransferase as set forth in SEQ ID NO: 59 (BvSGT4), having at least 60% identity thereto.

In one embodiment, the heterologous enzyme having glycosyltransferase activity is a Chenopodium glycosyltransferase. In one embodiment, the glycosyltransferase is a Chenopodium quinoa glycosyltransferase, such as the glycosyltransferase as set forth in SEQ ID NO: 57 (CqSGT2). In some embodiments, the glycosyltransferase is a functional variant of a Chenopodium glycosyltransferase, a functional variant of a Chenopodium quinoa glycosyltransferase or a functional variant of the glycosyltransferase as set forth in SEQ ID NO: 57 (CqSGT2), having at least 60% identity thereto.

In one embodiment, the heterologous enzyme having glycosyltransferase activity is a Bougainvillea glycosyltransferase. In one embodiment, the glycosyltransferase is a Bougainvillea glabra glycosyltransferase, such as the glycosyltransferase as set forth in SEQ ID NO: 11 (BgGT2). In some embodiments, the glycosyltransferase is a functional variant of a Bougainvillea glycosyltransferase, a functional variant of a Bougainvillea glabra glycosyltransferase or a functional variant of the glycosyltransferase as set forth in SEQ ID NO: 11 (BgGT2), having at least 60% identity thereto.

A functional variant of a glycosyltransferase refers to a variant of a glycosyltransferase, which retains at least some or all of the glycosyltransferase activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

A functional variant of a glycosyltransferase refers to a variant of glycosyltransferase that retains at least some of the activity of the parent enzyme. Thus, a functional variant of a glycosyltransferase can catalyze the same conversion as the glycosyltransferase from which it is derived, although the efficiency of the reaction may be different, e.g. the efficiency may be decreased or increased compared to the parent enzyme. Testing whether or not an enzyme is a functional variant of a glycosyltransferase can be tested using methods known in the art. For example, the glycosyltransferase variant can be expressed in a cell, wherein the cell medium contains the substrate of glycosyltransferase, i.e. cyclo-DOPA and/or betanidin, or said substrate is produced in said cell. After incubating the cell for 24 hours, the amount of product, i.e. the amount of cyclo-DOPA-5-O-glucoside and/or betanin and/or isobetanin, generated by the cell, i.e. by the glycosyltransferase variant, can be measured. If the glycosyltransferase variant generates the same product, i.e. cyclo-DOPA-5-O-glucoside and/or betanin and/or isobetanin, as the glycosyltransferase does, if said glycosyltransferase is tested under the same conditions, i.e. expressed in a cell and incubated for 24 h, the glycosyltransferase variant is a functional variant of said glycosyltransferase.

Any of the above enzymes having glycosyltransferase activity can be expressed in the cell together with any combination of TYHs and DODs described herein.

In one embodiment:
a. the TYH is selected from:
   i. Abronia nealleyi TYH, such as AnTYH as set forth in SEQ ID NO: 49 or a functional variant thereof having at least 70% sequence identity thereto;
   ii. Ercilla volubilis TYH, such as EvTYH as set forth in SEQ ID NO: 6 or a functional variant thereof having at least 70% sequence identity thereto;
   iii. Beta vulgaris TYH, such as BvCYP76AD1^{W13L} as set forth in SEQ ID NO: 4 or a functional variant thereof having at least 70% sequence identity thereto;
      and/or
b. the DOD is selected from:
   i. Mirabilis jalapa DOD, such as MjDOD as set forth in SEQ ID NO: 1 or a functional variant thereof having at least 70% sequence identity thereto;
   ii. Portulaca grandiflora DOD, such as PgDOD as set forth in SEQ ID NO: 55 or a functional variant thereof having at least 70% sequence identity thereto;
   iii. Bougainvillea glabra DOD, such as BgDOD2 as set forth in SEQ ID NO: 52 or a functional variant thereof having at least 70% sequence identity thereto;
      and/or
c. the enzyme having glycosyltransferase activity is selected from:
   i. Chenopodium quinoa glycosyltransferase CqSGT2 set forth in SEQ ID NO: 57, or a functional variant thereof having at least 70% sequence identity thereto;
   ii. Beta vulgaris glycosyltransferase BvSGT2 set forth in SEQ ID NO: 8, or a functional variant thereof having at least 70% sequence identity thereto;
   iii. Bougainvillea glabra glycosyltransferase BgGT2 set forth in SEQ ID NO: 11, or a functional variant thereof having at least 70% sequence identity thereto; and
   iv. Beta vulgaris glycosyltransferase BvSGT4 set forth in SEQ ID NO: 59, or a functional variant thereof having at least 70% sequence identity thereto.

### Useful Yeast Cells

In particular, any of the above TYH, DOD and enzyme having glycosyltransferase activity can be expressed in the yeast cell together with any combination of BAHD acyltransferase and/or glycosyltransferase described herein in the section “BAHD acyltransferase” and “Glycosyltransferase”, respectively.

Thus, in one embodiment of the invention, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD);
a. a third heterologous enzyme having glycosyltransferase activity; and
b. an enzyme selected from:
   i. a heterologous BAHD acyltransferase capable of acylating a betalain and/or glycosylated cyclo-DOPA; and
   ii. a heterologous glycosyltransferase capable of glycosylating a betalain and/or glycosylated cyclo-DOPA;
      whereby said yeast cell is capable of producing a modified betalains, wherein said modified betalain is selected from the group consisting of an acylated betalain and a di-glycosylated betalain.

In one embodiment, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. a heterologous BAHD acyltransferase capable of acylating betanin, isobetanin and/or glycosylated cyclo-DOPA, preferably wherein said BAHD acyltransferase is Hylocereus polyrhizus BAHD acyltransferase HpBAHD3 as set forth in SEQ ID NO: 29 or a functional variant thereof having at least 70% sequence identity thereto;
   whereby said yeast cell is capable of producing an acylated betalain, such as phyllocactin, isophyllocactin, phyllocactin II and/or isophyllocactin II.

In one embodiment, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. a heterologous glycosyltransferase capable of glycosylating betanin, isobetanin and/or glycosylated cyclo-DOPA, preferably wherein said glycosyltransferase is selected from the group consisting of the glycosyltransferase AhAmaSy1 as set forth in SEQ ID NO: 38, the glycosyltransferase CqAmaSy1 as set forth in SEQ ID NO: 41 and the glycosyltransferase CcAmaSy1 as set forth in SEQ ID NO: 44, or a functional variant thereof having at least 70% sequence identity thereto;
   whereby said yeast cell is capable of producing di-glycosylated betalain, such as bougainvillein-r-I, isobougainvillein-r-I, amaranthin and/or isoamaranthin.

In one embodiment, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. a heterologous glycosyltransferase capable of glycosylating betanin, isobetanin and/or glycosylated cyclo-DOPA, preferably wherein said glycosyltransferase is selected from the group consisting of the glycosyltransferase AhAmaSy1 as set forth in SEQ ID NO: 38 and the glycosyltransferase CcAmaSy1 as set forth in SEQ ID NO: 44, or a functional variant thereof having at least 70% sequence identity thereto;
   whereby said yeast cell is capable of producing bougainvillein-r-I and/or isobougainvillein-r-I.

In one embodiment, the yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity;
d. a native or a heterologous UDP glucose dehydrogenase; and
e. a heterologous glycosyltransferase capable of glycosylating betanin, isobetanin and/or glycosylated cyclo-DOPA, preferably wherein said glycosyltransferase is selected from the group consisting of the glycosyltransferase AhAmaSy1 as set forth in SEQ ID NO: 38, the glycosyltransferase CqAmaSy1 as set forth in SEQ ID NO: 41 and the glycosyltransferase CcAmaSy1 as set forth in SEQ ID NO: 44, or a functional variant thereof having at least 70% sequence identity thereto;
   whereby said yeast cell is capable of producing amaranthin and/or isoamaranthin, such as wherein said yeast cell produces amaranthin and/or isoamaranthin.

In one embodiment, the yeast cell is capable of producing an acylated betalain, such as wherein said yeast cell produces an acylated betalains, said yeast cell expressing:
a. a heterologous BAHD acyltransferase, such as a BAHD acyltransferase native to a plant, such as a plant of the genus Hylocereus, such as a plant of the genus Hylocereus polyrhizus, optionally BAHD acyltransferase HpBAHD3 as set forth in SEQ ID NO: 29 or a functional variant thereof having at least 70% sequence identity thereto;
b. a TYH selected from:
   i. Abronia nealleyi TYH, such as AnTYH as set forth in SEQ ID NO: 49 or a functional variant thereof having at least 70% sequence identity thereto;
   ii. Ercilla volubilis TYH, such as EvTYH as set forth in SEQ ID NO: 6 or a functional variant thereof having at least 70% sequence identity thereto;
   iii. Beta vulgaris TYH, such as BvCYP76AD1^{W13L} as set forth in SEQ ID NO: 4 or a functional variant thereof having at least 70% sequence identity thereto;
      and/or
c. a DOD selected from:
   i. Mirabilis jalapa DOD, such as MjDOD as set forth in SEQ ID NO: 1 or a functional variant thereof having at least 70% sequence identity thereto;
   ii. Portulaca grandiflora DOD, such as PgDOD as set forth in SEQ ID NO: 55 or a functional variant thereof having at least 70% sequence identity thereto;
   iii. Bougainvillea glabra DOD, such as BgDOD2 as set forth in SEQ ID NO: 52 or a functional variant thereof having at least 70% sequence identity thereto; and
d. an enzyme having glycosyltransferase activity selected from:
   i. Chenopodium quinoa glycosyltransferase CqSGT2 set forth in SEQ ID NO: 57, or a functional variant thereof having at least 70% sequence identity thereto;
   ii. Beta vulgaris glycosyltransferase BvSGT2 set forth in SEQ ID NO: 8, or a functional variant thereof having at least 70% sequence identity thereto;
   iii. Bougainvillea glabra glycosyltransferase BgGT2 set forth in SEQ ID NO: 11, or a functional variant thereof having at least 70% sequence identity thereto; and
   iv. Beta vulgaris glycosyltransferase BvSGT4 set forth in SEQ ID NO: 59, or a functional variant thereof having at least 70% sequence identity thereto.

In one embodiment, the yeast cell is capable of producing an acylated betalain, such as wherein said yeast cell produces an acylated betalain, said yeast cell expressing:
a. the Hylocereus polyrhizus BAHD acyltransferase HpBAHD3 as set forth in SEQ ID NO: 29 or a functional variant thereof having at least 70% sequence identity thereto;
b. the Beta vulgaris TYH BvCYP76AD1^{W13L} as set forth in SEQ ID NO: 4 or a functional variant thereof having at least 70% sequence identity thereto;
c. the Mirabilis jalapa DOD MjDOD as set forth in SEQ ID NO: 1 or a functional variant thereof having at least 70% sequence identity thereto; and
d. an enzyme having glycosyltransferase activity selected from:
   i. the Beta vulgaris glycosyltransferase BvSGT2 set forth in SEQ ID NO: 8, or a functional variant thereof having at least 70% sequence identity thereto; and
   ii. the Bougainvillea glabra glycosyltransferase BgGT2 set forth in SEQ ID NO: 11, or a functional variant thereof having at least 70% sequence identity thereto.

In one embodiment, the yeast cell is capable of producing a di-glycosylated betalain, such as wherein said yeast cell produces a di-glycosylated betalain, said yeast cell expressing:
a. a heterologous glycosyltransferase, such as a glycosyltransferase native to a plant, such as a plant of the genus Amaranthus, Chenopodium or Celosia, such as Amaranthus hypochondriacus, Chenopodium quinoa or Celosia cristata, optionally wherein the glycosyltransferase is selected from the group consisting of:
   i. Amaranthus hypochondriacus glycosyltransferase AhAmaSy1 as set forth in SEQ ID NO: 38 or a functional variant thereof having at least 70% sequence identity thereto;
   ii. Chenopodium quinoa glycosyltransferase CqAmaSy1 as set forth in SEQ ID NO: 41 or a functional variant thereof having at least 70% sequence identity thereto;
   iii. Celosia cristata glycosyltransferase CcAmaSy1 as set forth in SEQ ID NO: 44 or a functional variant thereof having at least 70% sequence identity thereto;
b. a TYH selected from:
   i. Abronia nealleyi TYH, such as AnTYH as set forth in SEQ ID NO: 49 or a functional variant thereof having at least 70% sequence identity thereto;
   ii. Ercilla volubilis TYH, such as EvTYH as set forth in SEQ ID NO: 6 or a functional variant thereof having at least 70% sequence identity thereto;
   iii. Beta vulgaris TYH, such as BvCYP76AD1^{W13L} as set forth in SEQ ID NO: 4 or a functional variant thereof having at least 70% sequence identity thereto;
      and/or
c. a DOD selected from:
   i. Mirabilis jalapa DOD, such as MjDOD as set forth in SEQ ID NO: 1 or a functional variant thereof having at least 70% sequence identity thereto;
   ii. Portulaca grandiflora DOD, such as PgDOD as set forth in SEQ ID NO: 55 or a functional variant thereof having at least 70% sequence identity thereto;
   iii. Bougainvillea glabra DOD, such as BgDOD2 as set forth in SEQ ID NO: 52 or a functional variant thereof having at least 70% sequence identity thereto; and
d. an enzyme having glycosyltransferase activity selected from:
   i. Chenopodium quinoa glycosyltransferase CqSGT2 set forth in SEQ ID NO: 57, or a functional variant thereof having at least 70% sequence identity thereto;
   ii. Beta vulgaris glycosyltransferase BvSGT2 set forth in SEQ ID NO: 8, or a functional variant thereof having at least 70% sequence identity thereto;
   iii. Bougainvillea glabra glycosyltransferase BgGT2 set forth in SEQ ID NO: 11, or a functional variant thereof having at least 70% sequence identity thereto; and
   iv. Beta vulgaris glycosyltransferase BvSGT4 set forth in SEQ ID NO: 59, or a functional variant thereof having at least 70% sequence identity thereto.

In one embodiment, the yeast cell is capable of producing a di-glycosylated betalain, such as wherein said yeast cell produces a di-glycosylated betalain, said yeast cell expressing:
a. a heterologous glycosyltransferase selected from the group consisting of:
   i. Amaranthus hypochondriacus glycosyltransferase AhAmaSy1 as set forth in SEQ ID NO: 38 or a functional variant thereof having at least 70% sequence identity thereto;
   ii. Chenopodium quinoa glycosyltransferase CqAmaSy1 as set forth in SEQ ID NO: 41 or a functional variant thereof having at least 70% sequence identity thereto;
   iii. Celosia cristata glycosyltransferase CcAmaSy1 as set forth in SEQ ID NO: 44 or a functional variant thereof having at least 70% sequence identity thereto;
b. the Beta vulgaris TYH BvCYP76AD1^{W13L} as set forth in SEQ ID NO: 4 or a functional variant thereof having at least 70% sequence identity thereto;
c. the Mirabilis jalapa DOD MjDOD as set forth in SEQ ID NO: 1 or a functional variant thereof having at least 70% sequence identity thereto; and
d. an enzyme having glycosyltransferase activity selected from:
   i. the Beta vulgaris glycosyltransferase BvSGT2 set forth in SEQ ID NO: 8, or a functional variant thereof having at least 70% sequence identity thereto; and
   ii. the Bougainvillea glabra glycosyltransferase BgGT2 set forth in SEQ ID NO: 11, or a functional variant thereof having at least 70% sequence identity thereto.

In one embodiment, the method comprises expressing in a yeast cell a BAHD acyltransferase from Hylocereus polyrhizus (HpBAHD3) as set forth in SEQ ID NO: 29, or a functional variant thereof having at least 70% sequence identity thereto, and:
a. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Chenopodium quinoa as set forth in SEQ ID NO: 57 (CqSGT2); or
b. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT2) as set forth in SEQ ID NO: 8; or
c. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Bougainvillea glabra as set forth in SEQ ID NO: 11, (BgGT2); or
d. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT4) as set forth in SEQ ID NO: 59; or
e. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Chenopodium quinoa as set forth in SEQ ID NO: 57, (CqSGT2); or
f. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT2) as set forth in SEQ ID NO: 8; or
g. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Bougainvillea glabra as set forth in SEQ ID NO: 11, (BgGT2); or
h. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT4) as set forth in SEQ ID NO: 59; or
i. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Chenopodium quinoa as set forth in SEQ ID NO: 57, (CqSGT2); or
j. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT2) as set forth in SEQ ID NO: 8; or
k. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Bougainvillea glabra as set forth in SEQ ID NO: 11, (BgGT2); or
l. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT4) as set forth in SEQ ID NO: 59; or
m. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Chenopodium quinoa as set forth in SEQ ID NO: 57 (CqSGT2); or
n. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT2) as set forth in SEQ ID NO: 8; or
o. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Bougainvillea glabra as set forth in SEQ ID NO: 11, (BgGT2); or
p. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT4) as set forth in SEQ ID NO: 59; or
q. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Chenopodium quinoa as set forth in SEQ ID NO: 57 (CqSGT2); or
r. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT2) as set forth in SEQ ID NO: 8; or
s. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Bougainvillea glabra as set forth in SEQ ID NO: 11, (BgGT2); or
t. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT4) as set forth in SEQ ID NO: 59; or
u. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Chenopodium quinoa as set forth in SEQ ID NO: 57 (CqSGT2); or
v. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT2) as set forth in SEQ ID NO: 8; or
w. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Bougainvillea glabra as set forth in SEQ ID NO: 11, (BgGT2); or
x. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT4) as set forth in SEQ ID NO: 59;
y. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Chenopodium quinoa as set forth in SEQ ID NO: 57 (CqSGT2); or
z. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT2) as set forth in SEQ ID NO: 8; or
aa. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Bougainvillea glabra as set forth in SEQ ID NO: 11, (BgGT2); or
bb. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT4) as set forth in SEQ ID NO: 59; or
cc. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Chenopodium quinoa as set forth in SEQ ID NO: 57 (CqSGT2); or
dd. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT2) as set forth in SEQ ID NO: 8; or
ee. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Bougainvillea glabra as set forth in SEQ ID NO: 11, (BgGT2); or
ff. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT4) as set forth in SEQ ID NO: 59; or
gg. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Chenopodium quinoa as set forth in SEQ ID NO: 57 (CqSGT2); or
hh. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT2) as set forth in SEQ ID NO: 8; or
ii. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Bougainvillea glabra as set forth in SEQ ID NO: 11, (BgGT2); or
jj. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT4) as set forth in SEQ ID NO: 59;
   or functional variants thereof having at least 80% identity thereto, whereby said yeast cell is capable of producing an acylated betalain, such as phyllocactin, isophyllocactin, phyllocactin II and/or isophyllocactin II, such as wherein said yeast cell produces an acylated betalain, such as phyllocactin, isophyllocactin, phyllocactin II and/or isophyllocactin II.

In one embodiment, the method comprises expressing in a yeast cell a glycosyltransferase from Amaranthus hypochondriacus (AhAmaSy1) as set forth in SEQ ID NO: 38, or a functional variant thereof having at least 70% sequence identity thereto, and:
a. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Chenopodium quinoa as set forth in SEQ ID NO: 57 (CqSGT2); or
b. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT2) as set forth in SEQ ID NO: 8; or
c. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Bougainvillea glabra as set forth in SEQ ID NO: 11, (BgGT2); or
d. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT4) as set forth in SEQ ID NO: 59; or
e. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Chenopodium quinoa as set forth in SEQ ID NO: 57, (CqSGT2); or
f. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT2) as set forth in SEQ ID NO: 8; or
g. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Bougainvillea glabra as set forth in SEQ ID NO: 11, (BgGT2); or
h. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT4) as set forth in SEQ ID NO: 59; or
i. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Chenopodium quinoa as set forth in SEQ ID NO: 57, (CqSGT2); or
j. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT2) as set forth in SEQ ID NO: 8; or
k. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Bougainvillea glabra as set forth in SEQ ID NO: 11, (BgGT2); or
l. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT4) as set forth in SEQ ID NO: 59; or
m. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Chenopodium quinoa as set forth in SEQ ID NO: 57 (CqSGT2); or
n. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT2) as set forth in SEQ ID NO: 8; or
o. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Bougainvillea glabra as set forth in SEQ ID NO: 11, (BgGT2); or
p. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT4) as set forth in SEQ ID NO: 59; or
q. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Chenopodium quinoa as set forth in SEQ ID NO: 57 (CqSGT2); or
r. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT2) as set forth in SEQ ID NO: 8; or
s. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Bougainvillea glabra as set forth in SEQ ID NO: 11, (BgGT2); or
t. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT4) as set forth in SEQ ID NO: 59; or
u. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Chenopodium quinoa as set forth in SEQ ID NO: 57 (CqSGT2); or
v. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT2) as set forth in SEQ ID NO: 8; or
w. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Bougainvillea glabra as set forth in SEQ ID NO: 11, (BgGT2); or
x. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT4) as set forth in SEQ ID NO: 59;
y. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Chenopodium quinoa as set forth in SEQ ID NO: 57 (CqSGT2); or
z. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT2) as set forth in SEQ ID NO: 8; or
aa. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Bougainvillea glabra as set forth in SEQ ID NO: 11, (BgGT2); or
bb. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT4) as set forth in SEQ ID NO: 59; or
cc. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Chenopodium quinoa as set forth in SEQ ID NO: 57 (CqSGT2); or
dd. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT2) as set forth in SEQ ID NO: 8; or
ee. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Bougainvillea glabra as set forth in SEQ ID NO: 11, (BgGT2); or
ff. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT4) as set forth in SEQ ID NO: 59; or
gg. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Chenopodium quinoa as set forth in SEQ ID NO: 57 (CqSGT2); or
hh. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT2) as set forth in SEQ ID NO: 8; or
ii. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Bougainvillea glabra as set forth in SEQ ID NO: 11, (BgGT2); or
jj. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT4) as set forth in SEQ ID NO: 59;
   or functional variants thereof having at least 80% identity thereto, whereby said yeast cell is capable of producing a di-glycosylated betalain, such as bougainvillein-r-I, isobougainvillein-r-I, amaranthin and/or isoamaranthin, such as wherein said yeast cell produces a di-glycosylated betalain, such as bougainvillein-r-I, isobougainvillein-r-I, amaranthin and/or isoamaranthin.

In one embodiment, the method comprises expressing in a yeast cell a glycosyltransferase from Chenopodium quinoa (CqAmaSy1) as set forth in SEQ ID NO: 41, or a functional variant thereof having at least 70% sequence identity thereto, and:
a. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Chenopodium quinoa as set forth in SEQ ID NO: 57 (CqSGT2); or
b. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT2) as set forth in SEQ ID NO: 8; or
c. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Bougainvillea glabra as set forth in SEQ ID NO: 11, (BgGT2); or
d. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT4) as set forth in SEQ ID NO: 59; or
e. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Chenopodium quinoa as set forth in SEQ ID NO: 57, (CqSGT2); or
f. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT2) as set forth in SEQ ID NO: 8; or
g. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Bougainvillea glabra as set forth in SEQ ID NO: 11, (BgGT2); or
h. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT4) as set forth in SEQ ID NO: 59; or
i. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Chenopodium quinoa as set forth in SEQ ID NO: 57, (CqSGT2); or
j. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT2) as set forth in SEQ ID NO: 8; or
k. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Bougainvillea glabra as set forth in SEQ ID NO: 11, (BgGT2); or
l. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT4) as set forth in SEQ ID NO: 59; or
m. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Chenopodium quinoa as set forth in SEQ ID NO: 57 (CqSGT2); or
n. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT2) as set forth in SEQ ID NO: 8; or
o. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Bougainvillea glabra as set forth in SEQ ID NO: 11, (BgGT2); or
p. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT4) as set forth in SEQ ID NO: 59; or
q. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Chenopodium quinoa as set forth in SEQ ID NO: 57 (CqSGT2); or
r. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT2) as set forth in SEQ ID NO: 8; or
s. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Bougainvillea glabra as set forth in SEQ ID NO: 11, (BgGT2); or
t. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT4) as set forth in SEQ ID NO: 59; or
u. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Chenopodium quinoa as set forth in SEQ ID NO: 57 (CqSGT2); or
v. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT2) as set forth in SEQ ID NO: 8; or
w. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Bougainvillea glabra as set forth in SEQ ID NO: 11, (BgGT2); or
x. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT4) as set forth in SEQ ID NO: 59; y. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Chenopodium quinoa as set forth in SEQ ID NO: 57 (CqSGT2); or
z. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT2) as set forth in SEQ ID NO: 8; or
aa. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Bougainvillea glabra as set forth in SEQ ID NO: 11, (BgGT2); or
bb. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT4) as set forth in SEQ ID NO: 59; or
cc. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Chenopodium quinoa as set forth in SEQ ID NO: 57 (CqSGT2); or
dd. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT2) as set forth in SEQ ID NO: 8; or
ee. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Bougainvillea glabra as set forth in SEQ ID NO: 11, (BgGT2); or
ff. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT4) as set forth in SEQ ID NO: 59; or
gg. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Chenopodium quinoa as set forth in SEQ ID NO: 57 (CqSGT2); or
hh. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT2) as set forth in SEQ ID NO: 8; or
ii. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Bougainvillea glabra as set forth in SEQ ID NO: 11, (BgGT2); or
jj. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT4) as set forth in SEQ ID NO: 59;
   or functional variants thereof having at least 80% identity thereto, whereby said yeast cell is capable of producing a di-glycosylated betalain, such as bougainvillein-r-I, isobougainvillein-r-I, amaranthin and/or isoamaranthin, such as wherein said yeast cell produces a di-glycosylated betalain, such as bougainvillein-r-I, isobougainvillein-r-I, amaranthin and/or isoamaranthin.

In one embodiment, the method comprises expressing in a yeast cell a glycosyltransferase from Celosia cristata (CcAmaSy1) as set forth in SEQ ID NO: 44, or a functional variant thereof having at least 70% sequence identity thereto, and:
a. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Chenopodium quinoa as set forth in SEQ ID NO: 57 (CqSGT2); or
b. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT2) as set forth in SEQ ID NO: 8; or
c. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Bougainvillea glabra as set forth in SEQ ID NO: 11, (BgGT2); or
d. a TYH from Abronia nealleyi (AnTYH) as set forth in SEQ ID NO: 49; a DOD from Bougainvillea glabra (BgDOD2) as set forth in SEQ ID NO: 52; and a glycosyltransferase from Beta vulgaris (BvSGT4) as set forth in SEQ ID NO: 59; or
e. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Chenopodium quinoa as set forth in SEQ ID NO: 57, (CqSGT2); or
f. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT2) as set forth in SEQ ID NO: 8; or
g. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Bougainvillea glabra as set forth in SEQ ID NO: 11, (BgGT2); or
h. a TYH from Abronia nealleyi (AnTYH) as set forth in SEQ ID NO: 49; a DOD from Mirabilis jalapa (MjDOD) as set forth in SEQ ID NO: 1; and a glycosyltransferase from Beta vulgaris (BvSGT4) as set forth in SEQ ID NO: 59; or
i. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Chenopodium quinoa as set forth in SEQ ID NO: 57, (CqSGT2); or
j. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT2) as set forth in SEQ ID NO: 8; or
k. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Bougainvillea glabra as set forth in SEQ ID NO: 11, (BgGT2); or
l. a TYH from Abronia nealleyi (AnTYH) as set forth in SEQ ID NO: 49; a DOD from Portulaca grandiflora (PgDOD) as set forth in SEQ ID NO: 55; and a glycosyltransferase from Beta vulgaris (BvSGT4) as set forth in SEQ ID NO: 59; or
m. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Chenopodium quinoa as set forth in SEQ ID NO: 57 (CqSGT2); or
n. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT2) as set forth in SEQ ID NO: 8; or
o. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Bougainvillea glabra as set forth in SEQ ID NO: 11, (BgGT2); or
p. a TYH from Ercilla volubilisi (EvTYH) as set forth in SEQ ID NO: 6; a DOD from Bougainvillea glabra (BgDOD2) as set forth in SEQ ID NO: 52; and a glycosyltransferase from Beta vulgaris (BvSGT4) as set forth in SEQ ID NO: 59; or
q. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Chenopodium quinoa as set forth in SEQ ID NO: 57 (CqSGT2); or
r. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT2) as set forth in SEQ ID NO: 8; or
s. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Bougainvillea glabra as set forth in SEQ ID NO: 11, (BgGT2); or
t. a TYH from Ercilla volubilisi (EvTYH) as set forth in SEQ ID NO: 6; a DOD from Mirabilis jalapa (MjDOD) as set forth in SEQ ID NO: 1; and a glycosyltransferase from Beta vulgaris (BvSGT4) as set forth in SEQ ID NO: 59; or
u. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Chenopodium quinoa as set forth in SEQ ID NO: 57 (CqSGT2); or
v. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT2) as set forth in SEQ ID NO: 8; or
w. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Bougainvillea glabra as set forth in SEQ ID NO: 11, (BgGT2); or
x. a TYH from Ercilla volubilisi (EvTYH) as set forth in SEQ ID NO: 6; a DOD from Portulaca grandiflora (PgDOD) as set forth in SEQ ID NO: 55; and a glycosyltransferase from Beta vulgaris (BvSGT4) as set forth in SEQ ID NO: 59;
y. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Chenopodium quinoa as set forth in SEQ ID NO: 57 (CqSGT2); or
z. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT2) as set forth in SEQ ID NO: 8; or
aa. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Bougainvillea glabra as set forth in SEQ ID NO: 11, (BgGT2); or
bb. a TYH from Beta vulgaris as set forth in SEQ ID NO: 4 (BvCYP76AD1^{W13L}); a DOD from Bougainvillea glabra (BgDOD2) as set forth in SEQ ID NO: 52; and a glycosyltransferase from Beta vulgaris (BvSGT4) as set forth in SEQ ID NO: 59; or
cc. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Chenopodium quinoa as set forth in SEQ ID NO: 57 (CqSGT2); or
dd. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT2) as set forth in SEQ ID NO: 8; or
ee. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Bougainvillea glabra as set forth in SEQ ID NO: 11, (BgGT2); or
ff. a TYH from Beta vulgaris as set forth in SEQ ID NO: 4 (BvCYP76AD1^{W13L}); a DOD from Mirabilis jalapa (MjDOD) as set forth in SEQ ID NO: 1; and a glycosyltransferase from Beta vulgaris (BvSGT4) as set forth in SEQ ID NO: 59; or
gg. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Chenopodium quinoa as set forth in SEQ ID NO: 57 (CqSGT2); or
hh. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT2) as set forth in SEQ ID NO: 8; or
ii. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Bougainvillea glabra as set forth in SEQ ID NO: 11, (BgGT2); or
jj. a TYH from Beta vulgaris as set forth in SEQ ID NO: 4 (BvCYP76AD1^{W13L}); a DOD from Portulaca grandiflora (PgDOD) as set forth in SEQ ID NO: 55; and a glycosyltransferase from Beta vulgaris (BvSGT4) as set forth in SEQ ID NO: 59;
   or functional variants thereof having at least 80% identity thereto, whereby said yeast cell is capable of producing a di-glycosylated betalain, such as bougainvillein-r-I, isobougainvillein-r-I, amaranthin and/or isoamaranthin, such as wherein said yeast cell produces a di-glycosylated betalain, such as bougainvillein-r-I, isobougainvillein-r-I, amaranthin and/or isoamaranthin.

Any of the yeast cells harbouring the various combinations of enzymes listed herein above in this section can be used in any of the methods disclosed in the section “Method of production of a modified betalain”.

In one embodiment, the yeast cell is as defined herein in the section “Yeast cell”. Preferably, the yeast cell is Saccharomyces cerevisiae or Yarrowia lipolytica.

### Method of Production of Modified Betalains

Provided herein is a method of producing a modified betalain in the presence of betanin, isobetanin and/or glycosylated cyclo-DOPA in a yeast cell, wherein the modified betalain is selected from the group consisting of acylated betalains and di-glycosylated betalains, said method comprising the steps of:
a. incubating a yeast cell in a medium;
b. expressing in said yeast cell:
   i. a heterologous BAHD acyltransferase capable of acylating a betalain and/or glycosylated cyclo-DOPA, thereby obtaining an acylated betalain; and/or
   ii. a heterologous glycosyltransferase capable of glycosylating a betalain and/or glycosylated cyclo-DOPA, thereby obtaining a di-glycosylated betalain.

Further provided is a method of increasing the titer and/or purity of a modified betalain in a yeast cell, wherein the modified betalain is selected from the group consisting of acylated betalains and di-glycosylated betalains, said method comprising the steps of:
a. incubating a yeast cell in a medium in the presence of betanin, isobetanin and/or glycosylated cyclo-DOPA, said yeast cell comprising a mutation resulting in reduced activity of one or more native β-glucosidases;
b. expressing in said yeast cell:
   i. a heterologous BAHD acyltransferase capable of acylating a betalain and/or glycosylated cyclo-DOPA, thereby obtaining an acylated betalain; and/or
   ii. a heterologous glycosyltransferase capable of glycosylating a betalain and/or glycosylated cyclo-DOPA, thereby producing a di-glycosylated betalain;
      thereby obtaining a modified betalain with an improved purity and/or titer as compared to the purity and/or titer of a modified betalain produced by a yeast cell expressing said heterologous BAHD acyltransferase and/or said heterologous glycosyltransferase, but not comprising a mutation resulting in reduced activity of one or more native β-glucosidases, and cultivated under the same conditions.

In one embodiment, the method further comprises a step of recovering the modified betalain.

In one embodiment, the method yields a modified betalain, such as an acylated and/or a di-glycosylated betalain, such as for example phyllocactin, phyllocactin II, amaranthin and/or bougainvillein-r-I, wherein the titer of said modified betalain is at least 0.05 mg/L, such as at least 0.1 mg/mL, such as at least 0.25 mg/mL, such as at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In another embodiment, the method increases the yield a modified betalain, such as an acylated and/or a di-glycosylated betalain, such as for example phyllocactin, phyllocactin II, amaranthin and/or bougainvillein-r-I, by at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 2.5-fold, such as at least 3-fold, such as at least 3.5-fold, such as at least 4-fold, such as at least 4.5-fold, such as at least 5-fold, such as at least 6-fold, such as at least 7-fold, such as at least 8-fold, such as at least 9-fold, such as at least 10-fold, such as at least 20-fold, such as at least 30-fold, such as at least 40-fold, such as at least 50-fold.

Further provided herein is a method for producing at least 0.05 mg/L of a modified betalain, such as an acylated and/or a di-glycosylated betalain, such as for example phyllocactin, phyllocactin II, amaranthin and/or bougainvillein-r-I, such as at least 0.1 mg/mL, such as at least 0.25 mg/mL, such as at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

Also provided herein is a method is for producing at least 0.05 mg/L of phyllocactin, phyllocactin II, amaranthin and/or bougainvillein-r-I, such as at least 0.1 mg/mL, such as at least 0.25 mg/mL, such as at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In one embodiment, the yeast cell is as described in the section “Yeast cell”. Preferably, the yeast cell is a Saccharomyces cerevisiae cell or a Yarrowia lipolytica cell.

In some embodiments, the yeast cell is engineered to express one or more enzymes in addition to the BAHD acyltransferase and/or the glycosyltransferase, such as described in the section “Yeast cell”.

In some embodiments, one or more genes encoding native enzymes having a negative impact on the titer of modified betalains have been deleted or mutated in the yeast cell, leading to total or partial loss of activity of the native enzyme. In particular, the yeast cell may comprise a mutation leading to reduced activity of 4-HPPD and/or one or more native β-glucosidases, as described in the section “Yeast cell”.

Thus, in one embodiment, one or more genes encoding native β-glucosidases, such as those descrived in the section “Yeast cell”, are deleted or mutated. In one embodiment, deletion of one or more β-glucosidases increases the titer of the one or more modified betalains, such as by preventing degradation of the one or more modified betalains. In one embodiment, the titer of the one or more modified betalains is increased by recovering the one or more modified betalains from the medium prior to the one or more modified betalains being degraded i.e. prior to that the titer of the one or more modified betalains are decreased. Thus, in one embodiment, the titer of the one or more modified betalains may be increased by deletion of one or more β-glucosidases and/or by recovering the one or more modified betalains prior to them being degraded, i.e. prior to that the titer of the one or more modified betalains starts to decrease.

In one embodiment, the modified betalain is an acylated betalain as described in the section “Betalains”. Preferably, the acylated betalain is phyllocactin, phyllocactin II, isophyllocactin and/or isophyllocactin II.

In one embodiment, the modified betalain is a di-glycosylated betalain as described in the section “Betalains”. Preferably, the di-glycosylated betalain is amaranthin, bougainvillein-r-I, isoamaranthin and/or isobougainvillein-r-I.

In one embodiment, the BAHD acyltransferase is as described herein in the section “BAHD acyltransferase”. In particular, the BAHD acyltransferase may be the Hylocereus polyrhizus BAHD acyltransferase as set forth in SEQ ID NO: 29 (HpBAHD3) or a functional variant thereof having at least 70% sequence identity thereto.

In one embodiment, the glycosyltransferase is as described herein in the section “Glycosyltransferase”. In particular, the glycosyltransferase may be the Amaranthus hypochondriacus glycosyltransferase as set forth in SEQ ID NO: 38 (AhAmaSy1), the Chenopodium quinoa glycosyltransferase as set forth in SEQ ID NO: 41 (CqAmaSy1) or the Celosia cristata glycosyltransferase as set forth in SEQ ID NO: 44 (CcAmaSy1) or a functional variant thereof having at least 70% sequence identity thereto.

In one embodiment, the medium is supplemented with one or more compounds selected from the group consisting of L-tyrosine, UDP-glucuronic acid, betanin, isobetanin and glycosylated cyclo-DOPA.

Thus, in one embodiment growth medium is supplemented with at least 100 mg/L, such as at least 200 mg/L, such as at least 400 mg/L, such as at least 600 mg/L, such as at least 800 mg/, such as at least 1.2 g/L, such as at least 1.4, such as at least 1.6 g/, such as at least 1.8 g/L, such as at least 2 g/L, such as at least 3 g/L, such as at least 4 g/L, such as at least 6 g/L, such as at least 8 g/of one or more compounds selected from the group consisting of L-tyrosine, UDP-glucuronic acid, betanin isobetanin and glycosylated cyclo-DOPA.

In one embodiment, the yeast cell expresses a UDP-glucose dehydrogenase. For example, the yeast cell may natively express the UDP-glucose dehydrogenase or it may be engineered to express a heterologous UDP-glucose dehydrogenase. In some embodiments, the UDP-glucose dehydrogenase is as defined the section “Yeast cell”.

In one embodiment, the yeast cell further expresses:
c. a first heterologous enzyme (TYH) capable of hydroxylating L-tyrosine and oxidizing L-DOPA;
d. a second heterologous enzyme which is a 4,5-DOPA extradiol dioxygenase (DOD); and
e. a third heterologous enzyme having glycosyltransferase activity.

In one embodiment, the TYH is as described in the section “TYH”. In particular, the TYH may be selected from the group consisting of the TYHs as set forth in SEQ ID NO: 4, SEQ ID NO: 6 and SEQ ID NO: 49 or functional variants thereof having at least 70% identity thereto.

In one embodiment, the DOD is as described in the section “DOD”. In particular, the DOD may be selected from the group consisting of the DODs as set forth in SEQ ID NO: 1, SEQ ID NO: 55 and SEQ ID NO: 52 or functional variants thereof having at least 70% identity thereto.

In one embodiment, the enzyme having glycosyltransferase activity, such as the glycosyltransferase is as described in the section “Enzyme having glycosyltransferase activity”. In particular, the enzyme having glycosyltransferase activity may be selected from the group consisting of the enzymes having glycosyltransferase activity as set forth in SEQ ID NO: 8, SEQ ID NO: 11, SEQ ID NO: 57 and SEQ ID NO: 59 or functional variants thereof having at least 70% identity thereto.

Thus, in one embodiment, the method comprises expressing in a yeast cell:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. an enzyme selected from:
   i. a heterologous BAHD acyltransferase capable of acylating a betalain and/or glycosylated cyclo-DOPA; and
   ii. a heterologous glycosyltransferase capable of glycosylating a betalain and/or glycosylated cyclo-DOPA;
      thereby obtaining a modified betalain, wherein said modified betalain is selected from the group consisting of an acylated betalain and a di-glycosylated betalain.

In one embodiment, the method comprises expressing in a yeast cell:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. a heterologous BAHD acyltransferase capable of acylating betanin, isobetanin and/or glycosylated cyclo-DOPA, preferably wherein said BAHD acyltransferase is Hylocereus polyrhizus BAHD acyltransferase HpBAHD3 as set forth in SEQ ID NO: 29 or a functional variant thereof having at least 70% sequence identity thereto;
   thereby obtaining an acylated betalain, such as phyllocactin, isophyllocactin, phyllocactin II and/or isophyllocactin II.

In one embodiment, the method comprises expressing in a yeast cell:
a. a first heterologous enzyme (TYH) capable of hydroxylating L-tyrosine and oxidizing L-DOPA;
b. a second heterologous enzyme which is a 4,5-DOPA extradiol dioxygenase (DOD);
c. a third heterologous enzyme having glycosyltransferase activity; and
d. a heterologous glycosyltransferase capable of glycosylating betanin, isobetanin and/or glycosylated cyclo-DOPA, preferably wherein said glycosyltransferase is selected from the group consisting of the glycosyltransferase AhAmaSy1 as set forth in SEQ ID NO: 38, the glycosyltransferase CqAmaSy1 as set forth in SEQ ID NO: 41 and the glycosyltransferase CcAmaSy1 as set forth in SEQ ID NO: 44, or a functional variant thereof having at least 70% sequence identity thereto;
   thereby obtaining a di-glycosylated betalain, such as bougainvillein-r-I, isobougainvillein-r-I, amaranthin and/or isoamaranthin.

In one embodiment, the method comprises expressing in a yeast cell:
a. a heterologous BAHD acyltransferase, such as a BAHD acyltransferase native to a plant, such as a plant of the genus Hylocereus, such as a plant of the genus Hylocereus polyrhizus, optionally BAHD acyltransferase HpBAHD3 as set forth in SEQ ID NO: 29 or a functional variant thereof having at least 70% sequence identity thereto;
b. a TYH selected from:
   i. Abronia nealleyi TYH, such as AnTYH as set forth in SEQ ID NO: 49 or a functional variant thereof having at least 70% sequence identity thereto;
   ii. Ercilla volubilis TYH, such as EvTYH as set forth in SEQ ID NO: 6 or a functional variant thereof having at least 70% sequence identity thereto;
   iii. Beta vulgaris TYH, such as BvCYP76AD1^{W13L} as set forth in SEQ ID NO: 4 or a functional variant thereof having at least 70% sequence identity thereto;
      and/or
c. a DOD selected from:
   i. Mirabilis jalapa DOD, such as MjDOD as set forth in SEQ ID NO: 1 or a functional variant thereof having at least 70% sequence identity thereto;
   ii. Portulaca grandiflora DOD, such as PgDOD as set forth in SEQ ID NO: 55 or a functional variant thereof having at least 70% sequence identity thereto;
   iii. Bougainvillea glabra DOD, such as BgDOD2 as set forth in SEQ ID NO: 52 or a functional variant thereof having at least 70% sequence identity thereto; and
d. an enzyme having glycosyltransferase activity selected from:
   i. Chenopodium quinoa glycosyltransferase CqSGT2 set forth in SEQ ID NO: 57, or a functional variant thereof having at least 70% sequence identity thereto;
   ii. Beta vulgaris glycosyltransferase BvSGT2 set forth in SEQ ID NO: 8, or a functional variant thereof having at least 70% sequence identity thereto;
   iii. Bougainvillea glabra glycosyltransferase BgGT2 set forth in SEQ ID NO: 11, or a functional variant thereof having at least 70% sequence identity thereto; and
   iv. Beta vulgaris glycosyltransferase BvSGT4 set forth in SEQ ID NO: 59, or a functional variant thereof having at least 70% sequence identity thereto;
      thereby obtaining an acylated betalain, such as phyllocactin, phyllocactin II, isophyllocactin and/or isophyllocactin II.

In one embodiment, the method comprises expressing in a yeast cell:
a. the Hylocereus polyrhizus BAHD acyltransferase HpBAHD3 as set forth in SEQ ID NO: 29 or a functional variant thereof having at least 70% sequence identity thereto;
b. the Beta vulgaris TYH BvCYP76AD1^{W13L} as set forth in SEQ ID NO: 4 or a functional variant thereof having at least 70% sequence identity thereto;
c. the Mirabilis jalapa DOD MjDOD as set forth in SEQ ID NO: 1 or a functional variant thereof having at least 70% sequence identity thereto; and
d. an enzyme having glycosyltransferase activity selected from:
   i. the Beta vulgaris glycosyltransferase BvSGT2 set forth in SEQ ID NO: 8, or a functional variant thereof having at least 70% sequence identity thereto; and
   ii. the Bougainvillea glabra glycosyltransferase BgGT2 set forth in SEQ ID NO: 11, or a functional variant thereof having at least 70% sequence identity thereto;
      thereby obtaining an acylated betalain, such as such as phyllocactin, phyllocactin II, isophyllocactin and/or isophyllocactin II.

In one embodiment, the method comprises expressing in a yeast cell:
a. a heterologous glycosyltransferase, such as a glycosyltransferase native to a plant, such as a plant of the genus Amaranthus, Chenopodium or Celosia, such as Amaranthus hypochondriacus, Chenopodium quinoa or Celosia cristata, optionally wherein the glycosyltransferase is selected from the group consisting of:
   i. Amaranthus hypochondriacus glycosyltransferase AhAmaSy1 as set forth in SEQ ID NO: 38 or a functional variant thereof having at least 70% sequence identity thereto;
   ii. Chenopodium quinoa glycosyltransferase CqAmaSy1 as set forth in SEQ ID NO: 41 or a functional variant thereof having at least 70% sequence identity thereto;
   iii. Celosia cristata glycosyltransferase CcAmaSy1 as set forth in SEQ ID NO: 44 or a functional variant thereof having at least 70% sequence identity thereto;
b. a TYH selected from:
   i. Abronia nealleyi TYH, such as AnTYH as set forth in SEQ ID NO: 49 or a functional variant thereof having at least 70% sequence identity thereto;
   ii. Ercilla volubilis TYH, such as EvTYH as set forth in SEQ ID NO: 6 or a functional variant thereof having at least 70% sequence identity thereto;
   iii. Beta vulgaris TYH, such as BvCYP76AD1^{W13L} as set forth in SEQ ID NO: 4 or a functional variant thereof having at least 70% sequence identity thereto;
      and/or
c. a DOD selected from:
   i. Mirabilis jalapa DOD, such as MjDOD as set forth in SEQ ID NO: 1 or a functional variant thereof having at least 70% sequence identity thereto;
   ii. Portulaca grandiflora DOD, such as PgDOD as set forth in SEQ ID NO: 55 or a functional variant thereof having at least 70% sequence identity thereto;
   iii. Bougainvillea glabra DOD, such as BgDOD2 as set forth in SEQ ID NO: 52 or a functional variant thereof having at least 70% sequence identity thereto; and
d. an enzyme having glycosyltransferase activity selected from:
   i. Chenopodium quinoa glycosyltransferase CqSGT2 set forth in SEQ ID NO: 57, or a functional variant thereof having at least 70% sequence identity thereto;
   ii. Beta vulgaris glycosyltransferase BvSGT2 set forth in SEQ ID NO: 8, or a functional variant thereof having at least 70% sequence identity thereto;
   iii. Bougainvillea glabra glycosyltransferase BgGT2 set forth in SEQ ID NO: 11, or a functional variant thereof having at least 70% sequence identity thereto; and
   iv. Beta vulgaris glycosyltransferase BvSGT4 set forth in SEQ ID NO: 59, or a functional variant thereof having at least 70% sequence identity thereto;
      thereby obtaining a di-glycosylated betalain, such as bougainvillein-r-I, isobougainvillein-r-I, amaranthin and/or isoamaranthin.

In one embodiment, the method comprises expressing in a yeast cell:
a. a heterologous glycosyltransferase selected from the group consisting of:
   i. Amaranthus hypochondriacus glycosyltransferase AhAmaSy1 as set forth in SEQ ID NO: 38 or a functional variant thereof having at least 70% sequence identity thereto;
   ii. Chenopodium quinoa glycosyltransferase CqAmaSy1 as set forth in SEQ ID NO: 41 or a functional variant thereof having at least 70% sequence identity thereto;
   iii. Celosia cristata glycosyltransferase CcAmaSy1 as set forth in SEQ ID NO: 44 or a functional variant thereof having at least 70% sequence identity thereto;
b. the Beta vulgaris TYH BvCYP76AD1^{W13L} as set forth in SEQ ID NO: 4 or a functional variant thereof having at least 70% sequence identity thereto;
c. the Mirabilis jalapa DOD MjDOD as set forth in SEQ ID NO: 1 or a functional variant thereof having at least 70% sequence identity thereto; and
d. an enzyme having glycosyltransferase activity selected from:
   i. the Beta vulgaris glycosyltransferase BvSGT2 set forth in SEQ ID NO: 8, or a functional variant thereof having at least 70% sequence identity thereto; and
   ii. the Bougainvillea glabra glycosyltransferase BgGT2 set forth in SEQ ID NO: 11, or a functional variant thereof having at least 70% sequence identity thereto;
      thereby obtaining a di-glycosylated betalain, such as bougainvillein-r-I, isobougainvillein-r-I, amaranthin and/or isoamaranthin.

In one embodiment, the method comprises expressing in a yeast cell any of the combinations of BAHD acyltransferase, TYH, DOD and enzyme having glycosyltransferase as disclosed herein in the section “Useful yeast cell”.

In one embodiment, the method comprises expressing in a yeast cell any of the combinations of glycosyltransferase, TYH, DOD and enzyme having glycosyltransferase as disclosed herein in the section “Useful yeast cell”.

In one embodiment, the method yields phyllocactin and/or isophyllocactin, wherein the titer of phyllocactin and/or isophyllocactin is at least 1.5 g/L, preferably at least 2 g/L. In one embodiment, the method yields phyllocactin and/or isophyllocactin, wherein the titer of phyllocactin and/or isophyllocactin is between 1 g/L and 4 g/L.

In other words, in one embodiment, the yeast cell as described herein is capable of producing, such as the yeast cell as described herein produces, at least 1.5 g/L of phyllocactin and/or isophyllocactin, such as at least 2 g/L of phyllocactin and/or isophyllocactin. In one embodiment, the yeast cell as described herein is capable of producing, such as the yeast cell as described herein produces, between 1 g/L and 4 g/L of phyllocactin and/or isophyllocactin.

Preferably, said yeast cell comprises a mutation leading to reduced activity of 4-HPPD as described in the section “Yeast cell”, and has further been modified to produce high amounts of L-tyrosine by mutation of one or more of the genes involved in L-tyrosine biosynthesis as described in the section “Yeast cell”. Even more preferably, said yeast cell is a Yarrowia lipolytica or Saccharomyces cerevisiae yeast cell that comprises: a mutation leading to partial or total loss of activity of 4-HPPD;
a point mutation in Aro4, such as wherein amino acid no. 221 of Yarrowia lipolytica Aro4 (SEQ ID NO: 71) is substituted with leucine or such as wherein amino acid no. 229 of Saccharomyces cerevisiae Aro4 (SEQ ID NO: 75) is substituted with leucine; and
a point mutation in Aro7, such as wherein amino acid no. 139 of Yarrowia lipolytica Aro7 (SEQ ID NO: 73) is substituted with serine or such as wherein amino acid no. 141 of Saccharomyces cerevisiae Aro7 (SEQ ID NO: 77) is substituted with serine.

In one embodiment, the method yields amaranthin and/or isoamaranthin, wherein the titer of amaranthin and/or isoamaranthin is at least 2.5 g/L, preferably at least 3 g/L. In one embodiment, the method yields amaranthin and/or isoamaranthin, wherein the titer of amaranthin and/or isoamaranthin is between 2 g/L and 5 g/L.

In other words, in one embodiment, the yeast cell as described herein is capable of producing, such as the yeast cell as described herein produces, at least 2.5 g/L of amaranthin and/or isoamaranthin, such as at least 3 g/L of amaranthin and/or isoamaranthin. In one embodiment, the yeast cell as described herein is capable of producing, such as the yeast cell as described herein produces, between 2 g/L and 5 g/L of amaranthin and/or isoamaranthin.

Preferably, said yeast cell comprises a mutation leading to reduced activity of 4-HPPD as described in the section “Yeast cell”, and has further been modified to produce high amounts of L-tyrosine by mutation of one or more of the genes involved in L-tyrosine biosynthesis as described in the section “Yeast cell”. Even more preferably, said yeast cell is a Yarrowia lipolytica or Saccharomyces cerevisiae yeast cell that comprises: a mutation leading to partial or total loss of activity of 4-HPPD;
a point mutation in Aro4, such as wherein amino acid no. 221 of Yarrowia lipolytica Aro4 (SEQ ID NO: 71) is substituted with leucine or such as wherein amino acid no. 229 of Saccharomyces cerevisiae Aro4 (SEQ ID NO: 75) is substituted with leucine; and
a point mutation in Aro7, such as wherein amino acid no. 139 of Yarrowia lipolytica Aro7 (SEQ ID NO: 73) is substituted with serine or such as wherein amino acid no. 141 of Saccharomyces cerevisiae Aro7 (SEQ ID NO: 77) is substituted with serine.

### Nucleic Acid

Provided herein is a system of nucleic acids encoding:
a. a BAHD acyltransferase capable of acylating glycosylated cyclo-DOPA and/or a betalain, such as a betanin and/or isobetanin; and/or
b. a glycosyltransferase capable of glycosylating glycosylated cyclo-DOPA and/or a betalain, such as a betanin and/or isobetanin.

In some embodiments, the system of nucleic acids further comprise nucleic acids encoding a TYH, a DOD and/or an enzyme having glycosyltransferase activity.

Thus, further provided herein is a system of nucleic acids encoding:
a. a BAHD acyltransferase capable of acylating glycosylated cyclo-DOPA and/or a betalain, such as a betanin and/or isobetanin;
b. a glycosyltransferase capable of glycosylating glycosylated cyclo-DOPA and/or a betalain, such as a betanin and/or isobetanin;
c. a TYH, preferably as described herein above, such as CYP76ADα capable of:
   i. hydroxylating L-tyrosine; and/or
   ii. oxidizing L-DOPA; and
d. a DOD, preferably as described herein above, capable of oxygenating L-DOPA; and/or
e. an enzyme having glycosyltransferase activity, preferably as described herein above, capable of:
   i. glycosylating cyclo-DOPA; and/or
   ii. glycosylating betanidin;

In one embodiment, the system is comprised in a vector, such as a plasmid, or in the genome of the yeast cell.

In one embodiment, the BAHD acyltransferase is as described in the section “BAHD acyltransferase”. In particular, the BAHD acyltransferase may be selected from the BAHD acyltransferase as set forth SEQ ID NO: 30 (HpBAHD3) or SEQ ID NO: 31 (HpBAHD3) or a polynucleotide having at least 60% sequence identity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 30 or SEQ ID NO: 31.

In one embodiment, the glycosyltransferase is as described in the section “Glycosyltransferase”. In particular, the glycosyltransferase may be selected from the glycosyltransferase as set forth SEQ ID NO: 39 (AhAmaSy1), SEQ ID NO: 40 (AhAmaSy1), SEQ ID NO: 42 (CqAmaSy1), SEQ ID NO: 43 (CqAmaSy1), SEQ ID NO: 45 (CcAmaSy1), SEQ ID NO: 46 (CcAmaSy1), or a polynucleotide having at least 60% sequence identity thereto, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to SEQ ID NO: 39, SEQ ID NO: 40, SEQ ID NO: 42, SEQ ID NO: 43, SEQ ID NO: 45 or SEQ ID NO: 46.

In one embodiment, the TYH is as described in the section “TYH”. In particular, the TYH may be selected from the group consisting of the TYHs as set forth in SEQ ID NO: 5, SEQ ID NO: 7, SEQ ID NO: 50 and SEQ ID NO: 51 or functional variants thereof having at least 70% identity thereto.

In one embodiment, the DOD is as described in the section “DOD”. In particular, the DOD may be selected from the group consisting of the DODs as set forth in SEQ ID NO: 2, SEQ ID NO: 3, SEQ ID NO: 53, SEQ ID NO: 54 and SEQ ID NO: 56 or functional variants thereof having at least 70% identity thereto.

In one embodiment, the enzyme having glycosyltransferase activity, such as the glycosyltransferase is as described in the section “Enzyme having glycosyltransferase activity”. In particular, the enzyme having glycosyltransferase activity may be selected from the group consisting of the enzymes having glycosyltransferase activity as set forth in SEQ ID NO: 9, SEQ ID NO: 10, SEQ ID NO: 12, SEQ ID NO: 58 and SEQ ID NO: 60 or functional variants thereof having at least 70% identity thereto.

Also procided herein is the use of a polynucleotide as set forth in SEQ ID NO: 30 or SEQ ID NO: 31, or a polynucleotide having at least 70% sequence identity thereto, for obtaining a protein capable of acylating glycosylated cyclo-DOPA and/or a glycosylated betalain, such as a betanin and/or isobetanin.

Also procided herein is the use of a polynucleotide as set forth in SEQ ID NO: 39, SEQ ID NO: 40, SEQ ID NO: 42, SEQ ID NO: 43, SEQ ID NO: 45 or SEQ ID NO: 46, or a polynucleotide having at least 70% sequence identity thereto, for obtaining a protein capable of glycosylating glycosylated cyclo-DOPA and/or a glycosylated betalain, such as a betanin and/or isobetanin.

### Uses of Modified Betalains

The compounds produced by the present yeast cells or by the present methods have a wide range of applications. For instance, the modified betalains, such as phyllocactin, phyllocactin II, amaranthin and/or bougainvillein-r-I and their respective isoforms, can be used as food colorants, i.e. as natural food dyes and/or as a colorants in the cosmetics and pharmaceutical industry.

Betalains have several advantages compared anthocyanins, another commonly used group of natural food dyes, including higher water solubility, higher tinctorial strength, and stability at a pH between 3 and 7.

Currently, the only existing commercial technology for production of betalains is via extraction from beetroot. This process is inefficient and provides an extract with undesirable flavour and aroma due to the presence of extract byproducts. Production of betalains in the cells and/or according to the methods disclosed herein provides higher product yield and titer, without undesired byproducts.

Production of Betalains in Yeast Cells with Reduced β-Glucosidase Activity

The inventors have surprisingly discovered that the titer and/or the purity of glycosylated betalains, such as for example betanin, isobetanin, phyllocactin, phyllocactin II, amaranthin and/or bougainvillein-r-I as well as their respective isoforms, produced in yeast cells can be improved by reducing the activity of one or more β-glucosidases natively expressed by said yeast cell, such as by mutating one or more β-glucosidases and/or by adding a β-glucosidase inhibitor to the growth medium.

In particular, the inventors have discovered that native β-glucosidases degrade the glycosylated betalains produced by the yeast cells described herein. Thus, yeast cells harbouring one or more mutations in one of more such native β-glucosidases do to a less extent, or not at all, degrade glycosylated betalains. In other words, said yeast cells have decreased, or no, degradation of glycosylated betalains.

Thus, in one embodiment, the yeast cell described herein has one or more mutations in one or more genes encoding for one or more β-glucosidase(s), wherein such mutations lead to partial or total loss of activity of said protein(s).

The non-functionality or reduced activity of one or more β-glucosidase(s) may for example be confirmed by the increased titter and/or purity of glycosylated betalains in a yeast cell engineered to produce those.

Thus, in one embodiment, the present invention provides a yeast cell capable of producing a glycosylated betalain, said yeast cell expressing:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
   whereby said yeast cell is capable of producing a glycosylated betalain, wherein said yeast cell comprises a mutation resulting in reduced activity of one or more native β-glucosidases.

Further provided herein is a method for producing a glycosylated betalain, said method comprising the steps of:
a. providing a yeast cell;
b. incubating said yeast cell in a medium;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      thereby obtaining a glycosylated betalain.

Further provided herein is a method for improving the titer and/or purity of one or more glycosylated betalains produced by a yeast cell, said method comprising the steps of:
a. providing a yeast cell comprising a mutation resulting in reduced activity of one or more native β-glucosidases, said yeast cell producing a glycosylated betalain;
b. incubating said yeast cell in a medium;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      thereby obtaining a glycosylated betalain with an improved purity and/or titer as compared to the purity and/or titer of a yeast cell expressing said TYH, DOD and enzyme having glycosyltransferase activity but not comprising a mutation resulting in reduced activity of one or more native β-glucosidases, wherein said yeast cells are cultivated under the same conditions.

In one embodiment, the yeast cell is a Yarrowia lipolytica yeast cell, such as a Yarrowia lipolytica W29 yeast cell, and the native β-glucosidase is encoded by a gene selected from the group consisting of:
YALI1_B23300g (SEQ ID NO: 61);
YALI1_F21504g (SEQ ID NO: 62);
YALI1_B05024g (SEQ ID NO: 63);
YALI1_B18845g (SEQ ID NO: 64);
YALI1_B18887g (SEQ ID NO: 65);
YALI1_D22997g (SEQ ID NO: 66);
YALI1_E40502g (SEQ ID NO: 67);
YALI1_F02592g (SEQ ID NO: 68);
YALI1_F08075g (SEQ ID NO: 69);
YALI1_F17788g (SEQ ID NO: 70);
YALI1_E23994g (SEQ ID NO: 79);
YALI1_E39796g (SEQ ID NO: 80);
YALI1_F03075g (SEQ ID NO: 81); and
YALI1_E25163g (SEQ ID NO: 82).

In one embodiment, the yeast cell is a Yarrowia lipolytica yeast cell, such as a Yarrowia lipolytica W29 yeast cell or a Yarrowia lipolytica CLIB122 yeast cell, and the native β-glucosidase is encoded by a gene selected from the group consisting of:
YALI1_B23300g (SEQ ID NO: 61);
YALI1_F21504g (SEQ ID NO: 62);
YALI1_B05024g (SEQ ID NO: 63);
YALI1_B18845g (SEQ ID NO: 64);
YALI1_B18887g (SEQ ID NO: 65);
YALI1_D22997g (SEQ ID NO: 66);
YALI1_E40502g (SEQ ID NO: 67);
YALI1_F02592g (SEQ ID NO: 68);
YALI1_F08075g (SEQ ID NO: 69);
YALI1_F17788g (SEQ ID NO: 70);
YALI1_E23994g (SEQ ID NO: 79);
YALI1_E39796g (SEQ ID NO: 80);
YALI1_F03075g (SEQ ID NO: 81);
YALI1_E25163g (SEQ ID NO: 82);
YALI0F16027g (SEQ ID NO: 217);
YALI0B14289g (SEQ ID NO: 218); and
YALI0F05390g (SEQ ID NO: 219).

In a preferred embodiment, the native β-glucosidase is encoded by a gene selected from the group consisting of YALI1_D22997g (SEQ ID NO: 66), YALI1_B23300g (SEQ ID NO: 61), YALI1_F21504g (SEQ ID NO: 62), and YALI1_E23994g (SEQ ID NO: 82).

In an equally preferred embodiment, the native β-glucosidase is encoded by a gene selected from the group consisting of YALI1_D22997g (SEQ ID NO: 66), YALI1_B23300g (SEQ ID NO: 61), YALI1_F21504g (SEQ ID NO: 62), YALI1_E23994g (SEQ ID NO: 82), YALI0F16027g (SEQ ID NO: 217), YALI0B14289g (SEQ ID NO: 218) and YALI0F05390g (SEQ ID NO: 219).

In an equally preferred embodiment, the native β-glucosidase is encoded by a gene selected from the group consisting of YALI1_F08075g (SEQ ID NO: 69), YALI1_B18845g (SEQ ID NO: 64), and/or YALI1_F21504g (SEQ ID NO: 62).

In an equally preferred embodiment, the native β-glucosidase is encoded by a gene selected from the group consisting of YALI0F16027g (SEQ ID NO: 217), YALI0B14289g (SEQ ID NO: 218) and/or YALI0F05390g (SEQ ID NO: 219).

In one embodiment, the activity of the one or more native β-glucosidases are reduced by supplementing to the growth medium a compound capable of inhibiting the activity of β-glucosidases, i.e. a β-glucosidase inhibitor. In one embodiment, the β-glucosidase inhibitor is cellobiose.

Thus, also provided herein is a method for producing a glycosylated betalain, said method comprising the steps of:
a. providing a yeast cell;
b. incubating said yeast cell in a medium comprising a β-glucosidase inhibitor, such as cellobiose;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      thereby obtaining a glycosylated betalain.

Further provided herein is a method for improving the titer and/or purity of one or more betalains produced by a yeast cell, said method comprising the steps of:
a. providing a yeast cell, said yeast cell producing a glycosylated betalain;
b. incubating said yeast cell in a medium comprising a β-glucosidase inhibitor, such as cellobiose;
c. expressing in said yeast cell:
   i. a first heterologous enzyme (TYH) capable of hydroxylating L-tyrosine and oxidizing L-DOPA;
   ii. a second heterologous enzyme which is a 4,5-DOPA extradiol dioxygenase (DOD); and
   iii. a third heterologous enzyme having glycosyltransferase activity;
      therby obtaining a betalain with an improved purity and/or titer as compared to the purity and/or titer of the yeast cell incubated in the absence of the β-glucosidase inhibitor, such as cellobiose, but otherwise cultivated under the same conditions.

In one embodiment, the method further comprises recovering the glycosylated betalain.

In one embodiment, the TYH is as described in the section “TYH”. In particular, the TYH may be selected from the group consisting of the TYHs as set forth in SEQ ID NO: 4, SEQ ID NO: 6 and SEQ ID NO: 49 or functional variants thereof having at least 70% identity thereto.

In one embodiment, the DOD is as described in the section “DOD”. In particular, the DOD may be selected from the group consisting of the DODs as set forth in SEQ ID NO: 1, SEQ ID NO: 55 and SEQ ID NO: 52 or functional variants thereof having at least 70% identity thereto.

In one embodiment, the enzyme having glycosyltransferase activity, such as the glycosyltransferase is as described in the section “Enzyme having glycosyltransferase activity”. In particular, the enzyme having glycosyltransferase activity may be selected from the group consisting of the enzymes having glycosyltransferase activity as set forth in SEQ ID NO: 8, SEQ ID NO: 11, SEQ ID NO: 57 and SEQ ID NO: 59 or functional variants thereof having at least 70% identity thereto.

In one embodiment, the method yields a glycosylated betalain, such as betanin and/or isobetanin, wherein the titer of said glycosylated betalain is at least 0.05 mg/L, such as at least 0.1 mg/mL, such as at least 0.25 mg/mL, such as at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In another embodiment, the method increases the yield a glycosylated betalain, such as betanin and/or isobetanin, by at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 2.5-fold, such as at least 3-fold, such as at least 3.5-fold, such as at least 4-fold, such as at least 4.5-fold, such as at least 5-fold, such as at least 6-fold, such as at least 7-fold, such as at least 8-fold, such as at least 9-fold, such as at least 10-fold, such as at least 20-fold, such as at least 30-fold, such as at least 40-fold, such as at least 50-fold.

In one embodiment, the yeast cell is as described herein in the section “Yeast cell”. Preferably, the yeast cell is a Saccharomyces cerevisiae or a Yarrowia lipolytica yeast cell.

In one embodiment, the growth medium is as defined herein in the section “Method of production of modified betalains”.

In one embodiment, the glycosylated betalain is betanin and/or isobetanin.

In one embodiment, the yeast cell is further engineered to produce a modified betalain, such as an acylated and/or di-glycosylated betalain.

Thus, in one embodiment said yeast cell further expresses:
a. a BAHD acyltransferase, whereby said yeast cell is capable of producing an acylated betalain; and/or
b. a glycosyltransferase, whereby said yeast cell is capable of producing a di-glycosylated betalain.

In one embodiment, the BAHD acyltransferase is as described herein in the section “BAHD acyltransferase”. In particular, the BAHD acyltransferase may be the Hylocereus polyrhizus BAHD acyltransferase as set forth in SEQ ID NO: 29 (HpBAHD3) or a functional variant thereof having at least 70% sequence identity thereto.

In one embodiment, the glycosyltransferase is as described herein in the section “Glycosyltransferase”. In particular, the glycosyltransferase may be the Amaranthus hypochondriacus glycosyltransferase as set forth in SEQ ID NO: 38 (AhAmaSy1), the Chenopodium quinoa glycosyltransferase as set forth in SEQ ID NO: 41 (CqAmaSy1) or the Celosia cristata glycosyltransferase as set forth in SEQ ID NO: 44 (CcAmaSy1) or a functional variant thereof having at least 70% sequence identity thereto.

In one embodiment, the modified betalain is an acylated betalain as described in the section “Betalains”. Preferably, the acylated betalain is phyllocactin or phyllocactin II.

In one embodiment, the modified betalain is a di-glycosylated betalain as described in the section “Betalains”. Preferably, the di-glycosylated betalain is bougainvillein-r-I or amaranthin.

In one embodiment, UDP-glucuronic acid is supplemented to the growth media. In one embodiment, the yeast cell is capable of producing UDP-glucuronic acid. The yeast cell may be naturally capable of producing UDP-glucuronic acid, such as wherein the yeast cell is a Yarrowia lipolytica yeast cell. Alternatively, the yeast cell may be engineered to produce UDP-glucuronic acid. In some embodiments, the yeast cell may be a natural UDP-glucuronic acid producer engineered for increased production of UDP-glucuronic acid. Thus, in some embodiments, the yeast cell as defined herein is capable of producing amaranthin.

### Items

1. A yeast cell capable of producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
   a. a heterologous BAHD acyltransferase capable of acylating a betalain and/or glycosylated cyclo-DOPA, thereby producing an acylated betalain; and/or
   b. a heterologous glycosyltransferase capable of glycosylating a betalain and/or glycosylated cyclo-DOPA, thereby producing a di-glycosylated betalain.
2. A yeast cell producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
   a. a heterologous BAHD acyltransferase capable of acylating a betalain and/or glycosylated cyclo-DOPA, thereby producing an acylated betalain; and/or
   b. a heterologous glycosyltransferase capable of glycosylating a betalain and/or glycosylated cyclo-DOPA, thereby producing a di-glycosylated betalain.
3. The yeast cell according to any one of the preceding items, wherein:
   a. the acylated betalain is an acylated betacyanin, such as an acylated betanin and/or isobetanin, such as a betanin and/or isobetanin acylated at position 4 of the glycosyl moiety of betanin and/or isobetanin, such as phyllocactin II and/or isophyllocactin II; and/or
   b. the acylated betalain is an acylated betacyanin, such as an acylated betanin and/or isobetanin, such as a betanin and/or isobetanin acylated at position 6 of the glycosyl moiety of betanin and/or isobetanin, such as phyllocactin and/or isophyllocactin; and/or
   c. the di-glycosylated betalain is a di-glycosylated betacyanin, such as a glycosylated betanin and/or isobetanin, such as a betanin and/or isobetanin glycosylated at position 2 of the glycosyl moiety of betanin and/or isobetanin, such as bougainvillein-r-I and/or isobougainvillein-r-I.
4. The yeast cell according to any one of the preceding items, wherein:
   a. the BAHD acyltransferase is native to a plant, such as a plant of the genus Hylocereus; and/or
   b. the glycosyltransferase is native to a plant, such as a plant of the genus Amaranthus, Chenopodium or Celosia.
5. The yeast cell according to any one of the preceding items, wherein:
   a. the BAHD acyltransferase is native to Hylocereus polyrhizus, optionally the BAHD acyltransferase is HpBAHD3 as set forth in SEQ ID NO: 29 or a functional variant thereof having at least 70% sequence identity thereto; and/or
   b. the glycosyltransferase is native to Amaranthus hypochondriacus, Chenopodium quinoa or Celosia cristata, optionally the glycosyltransferase is AhAmaSy1 as set forth in SEQ ID NO: 38, CqAmaSy1 as set forth in SEQ ID NO: 41 and/or CcAmaSy1 as set forth in SEQ ID NO: 44 or a functional variant thereof having at least 70% sequence identity to SEQ ID NO: 38, SEQ ID NO: 41 or SEQ ID NO 44.
6. The yeast cell according to any one of the preceding items, wherein the genus of said yeast cell is selected from the group consisting of Saccharomyces, Pichia, Yarrowia, Kluyveromyces, Candida, Rhodotorula, Rhodosporidium, Cryptococcus, Trichosporon or Lipomyces.
7. The yeast cell according to any one of the preceding items, wherein the yeast is of a species selected from the group consisting of Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan or Yarrowia lipolytica.
8. The yeast cell according to any one of the preceding items, wherein UDP-glucuronic acid is supplemented to the yeast cell.
9. The yeast cell according to any one of the preceding items, wherein the yeast cell is capable of producing UDP-glucuronic acid.
10. The yeast cell according to any one of the preceding items, wherein the yeast cell further expresses a UDP-glucose dehydrogenase, whereby the yeast cell is capable of producing UDP-glucuronic acid.
11. The yeast cell according to item 10, wherein the UDP-glucose dehydrogenase is native to a plant, such as a plant of the genus Arabidopsis, for example Arabidopsis thaliana.
12. The yeast cell according to any one of items 10 and 11, wherein the UDP-glucose dehydrogenase is AtUGD1 as set forth in SEQ ID NO: 47 or a functional variant thereof having at least 70% sequence identity thereto.
13. The yeast cell according to any one of items 8 to 12, wherein the di-glycosylated betalain is a di-glycosylated betacyanin, such as a glycosylated betanin and/or isobetanin, such as a betanin and/or isobetanin glucoronidated at position 2 of the glycosyl moiety of betanin and/or isobetanin, such as amaranthin and/or isoamaranthin.
14. The yeast cell according to any one of the preceding items, wherein glycosylated cyclo-DOPA, betanin and/or isobetanin is supplemented to the yeast cell.
15. The yeast cell according to any one of the preceding items, wherein said yeast cell is capable of producing glycosylated cyclo-DOPA, betanin and/or isobetanin.
16. The yeast cell according to any one of the preceding items, wherein said yeast cell further expresses:
   a. a first heterologous enzyme (TYH) capable of hydroxylating L-tyrosine and oxidizing L-DOPA;
   b. a second heterologous enzyme which is a 4,5-DOPA extradiol dioxygenase (DOD); and
   c. a third heterologous enzyme having glycosyltransferase activity; whereby the yeast cell is capable of producing betanin and/or isobetanin.
17. The yeast cell according to any one of the preceding items, wherein said yeast cell further expresses:
   a. a first heterologous enzyme which is a tyrosine hydroxylase (TYH) capable of hydroxylating L-tyrosine and oxidizing L-DOPA;
   b. a second heterologous enzyme which is a 4,5-DOPA extradiol dioxygenase (DOD); and
   c. a third heterologous enzyme having glycosyltransferase activity;
   whereby the yeast cell produces betanin and/or isobetanin.
18. The yeast cell according to any one of items 16 to 17, wherein:
   a. the TYH is native to a plant, such as of the genus Abronia, Acleisanthes, Basella, Beta, Cleretum, Ercilla, Mirabilis, Optunia or Phytolacca, such as Abronia nealleyi, Acleisanthes obtusa, Basella alba, Beta vulgaris, Cleretum bellidiforme, Ercilla volubilis, Mirabilis multiflora, Optunia ficus-indic, or Phytolacca dioica, or a functional variant thereof having at least 80% identity thereto; and/or
   b. the DOD is native to a plant, such as of the genus Amaranthus, Beta, Bougainvillea, Mirabilis Phytolacca, Portulaca, Spinacia or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea or Suaeda salsa, or a functional variant thereof having at least 80% identity thereto; and/or
   c. the enzyme having glycosyltransferase activity is native to a plant, such as of the genus Abronia, Beta, Bougainvillea, Ercilla or Portacula, such as Abronia nealleyi, Beta vulgaris, Bougainvillea glabra, Ercilla volubilis or Portacula grandiflora, or a functional variant thereof having at least 80% identity thereto.
19. The yeast cell according to any one of items 16 to 18, wherein the third heterologous enzyme having glycosyltransferase activity has an activity selected from betanidin-5-O-glucosyltransferase (B5OGT) activity and cyclo-DOPA-5-O-glucosyltransferase (cDOPA5OGT) activity.
20. The yeast cell according to any one of items 16 to 19 wherein:
   a. the TYH is selected from:
      i. Abronia nealleyi TYH, such as AnTYH as set forth in SEQ ID NO: 49 or a functional variant thereof having at least 70% sequence identity thereto;
      ii. Ercilla volubilis TYH, such as EvTYH as set forth in SEQ ID NO: 6 or a functional variant thereof having at least 70% sequence identity thereto;
      iii. Beta vulgaris TYH, such as BvCYP76AD1^{W13L} as set forth in SEQ ID NO: 4 or a functional variant thereof having at least 70% sequence identity thereto;
         and/or
   b. the DOD is selected from:
      i. Mirabilis jalapa DOD, such as MjDOD as set forth in SEQ ID NO: 1 or a functional variant thereof having at least 70% sequence identity thereto;
      ii. Portulaca grandiflora DOD, such as PgDOD as set forth in SEQ ID NO: 55 or a functional variant thereof having at least 70% sequence identity thereto;
      iii. Bougainvillea glabra DOD, such as BgDOD2 as set forth in SEQ ID NO: 52 or a functional variant thereof having at least 70% sequence identity thereto; and
   c. the enzyme having glycosyltransferase activity is selected from:
      i. Chenopodium quinoa glycosyltransferase CqSGT2 set forth in SEQ ID NO: 57, or a functional variant thereof having at least 70% sequence identity thereto;
      ii. Beta vulgaris glycosyltransferase BvSGT2 set forth in SEQ ID NO: 8, or a functional variant thereof having at least 70% sequence identity thereto;
      iii. Bougainvillea glabra glycosyltransferase BgGT2 set forth in SEQ ID NO: 11, or a functional variant thereof having at least 70% sequence identity thereto; and
      iv. Beta vulgaris glycosyltransferase BvSGT4 set forth in SEQ ID NO: 59, or a functional variant thereof having at least 70% sequence identity thereto.
21. The yeast cell according to any one of the preceding items, wherein at least one of the nucleic acids encoding the BAHD acyltransferase, the glycosyltransferase, the TYH, the DOD or the enzyme having glycosyltransferase activity is present in high copy number, such as wherein at least one of the genes encoding the BAHD acyltransferase, the glycosyltransferase, the TYH, the DOD or the enzyme having glycosyltransferase activity is present in at least two copies, such as at least three copies, such as at least four copies, such as at least five copies.
22. The yeast cell according to any one of the preceding items, wherein at least one of the nucleic acids encoding the BAHD acyltransferase, the glycosyltransferase, the TYH, the DOD or the enzyme having glycosyltransferase activity is under the control of an inducible promoter.
23. The yeast cell according to any one of the preceding items, wherein at least one of the nucleic acids encoding the BAHD acyltransferase, the glycosyltransferase, the TYH, the DOD or the enzyme having glycosyltransferase activity is codon-optimized for said yeast cell.
24. The yeast cell according to any one of the preceding items, wherein the nucleic acids encoding the BAHD acyltransferase, the glycosyltransferase, the TYH, the DOD or the enzyme having glycosyltransferase activity are each independently comprised within the genome of the yeast cell or whithin a vector comprised within the yeast cell.
25. The yeast cell according to any one of the preceding items, wherein said yeast cell comprises a mutation resulting in reduced activity in one or more genes encoding one or more native β-glucosidases, optionally wherein said one or more genes are selected from the group consisting of:
   i) YALI1_B23300g (SEQ ID NO: 61);
   ii) YALI1_F21504g (SEQ ID NO: 62);
   iii) YALI1_B05024g (SEQ ID NO: 63);
   iv) YALI1_B18845g (SEQ ID NO: 64);
   v) YALI1_B18887g (SEQ ID NO: 65);
   vi) YALI1_D22997g (SEQ ID NO: 66);
   vii) YALI1_E40502g (SEQ ID NO: 67);
   viii) YALI1_F02592g (SEQ ID NO: 68);
   ix) YALI1_F08075g (SEQ ID NO: 69);
   x) YALI1_F17788g (SEQ ID NO: 70);
   xi) YALI1_E23994g (SEQ ID NO: 79);
   xii) YALI1_E39796g (SEQ ID NO: 80);
   xiii) YALI1_F03075g (SEQ ID NO: 81);
   xiv) YALI1_E25163g (SEQ ID NO: 82);
   xv) YALI0F16027g (SEQ ID NO: 217);
   xvi) YALI0B14289g (SEQ ID NO: 218); and
   xvii) YALI0F05390g (SEQ ID NO: 219);
   preferably wherein said one or more genes are selected from the group consisting of YALI1_D22997g (SEQ ID NO: 66), YALI1_B23300g (SEQ ID NO: 61), YALI1_F21504g (SEQ ID NO: 62), YALI1_F08075g (SEQ ID NO: 69), YALI1_B18845g (SEQ ID NO: 64), YALI1_E23994g (SEQ ID NO: 82), YALI0F16027g (SEQ ID NO: 217), YALI0B14289g (SEQ ID NO: 218) and YALI0F05390g (SEQ ID NO: 219).
26. The yeast cell according to item 25, wherein the mutation is a mutation in one or more genes encoding one or more native β-glucosidases, such as a partial or total deletion of the one or more genes encoding the one or more native β-glucosidases.
27. The yeast cell according to any one of items 25 to 26, wherein the activity is reduced as compared the activity in a yeast cell in which the one or more native β-glucosidases have not been mutated, but otherwise identical, when cultivated in the same conditions.
28. The yeast cell according to any one of the preceding items, wherein said yeast cell comprises a mutation resulting in reduced activity of 4-hydroxyphenylpyruvate dioxygenase (4-HPPD).
29. The yeast cell according to item 28, wherein the mutation is a mutation in the gene encoding 4-HPPD, such as a partial or total deletion of the gene encoding 4-HPPD.
30. The yeast cell according to any one of items 28 to 29, wherein the activity is reduced as compared the activity in a yeast cell in which 4-HPPD has not been mutated, but otherwise identical, when cultivated in the same conditions.
31. The yeast cell according to any one of the preceding items, wherein said yeast cell comprises a mutation resulting in increased production of L-tyrosine, optionally wherein the production is increased as compared the production in a yeast cell not carrying said mutation resulting in increased production of L-tyrosine, but otherwise identical, when cultivated in the same conditions.
32. The yeast cell according to any one of the preceding items, wherein said yeast cell comprises a mutation resulting in less sensitivity to feedback inhibition by aromatic amino acids, optionally wherein the sensitivity is reduced as compared the sensitivity in a yeast cell not carrying said mutation resulting in less sensitivity to feedback inhibition by aromatic amino acids, but otherwise identical, when cultivated in the same conditions.
33. The yeast cell according to item 32, wherein said yeast cell comprises a mutation in:
   a. 3-deoxy-7-phosphoheptulonate synthase, such as in Yarrowia lipolytica Aro4 or Saccharomyces cerevisiae Aro4, such as wherein amino acid no. 221 of Yarrowia lipolytica Aro4 is substituted with a leucine or wherein amino acid no. 229 of Saccharomyces cerevisiae Aro4 is substituted with leucine; optionally the mutation is a point mutation; and/or
   b. chorismate mutase, such as in Yarrowia lipolytica Aro7 or Saccharomyces cerevisiae Aro7, such as wherein amino acid no. 139 of Yarrowia lipolytica Aro7 is substituted with a serine or wherein amino acid no. 141 of Saccharomyces cerevisiae Aro7 is substituted with a serine; optionally the mutation is a point mutation.
34. A method of producing a modified betalain in the presence of betanin, isobetanin and/or glycosylated cyclo-DOPA in a yeast cell, wherein the modified betalain is selected from the group consisting of acylated betalains and di-glycosylated betalains, said method comprising the steps of:
   a. incubating a yeast cell in a medium;
   b. expressing in said yeast cell:
      i. a heterologous BAHD acyltransferase capable of acylating a betalain and/or glycosylated cyclo-DOPA, thereby obtaining an acylated betalain; and/or
      ii. a heterologous glycosyltransferase capable of glycosylating a betalain and/or glycosylated cyclo-DOPA, thereby obtaining a di-glycosylated betalain.
35. A method of increasing the titer and/or purity of a modified betalain produced by a yeast cell, wherein the modified betalain is selected from the group consisting of acylated betalains and di-glycosylated betalains, said method comprising the steps of:
   a. incubating a yeast cell in a medium in the presence of betanin, isobetanin and/or glycosylated cyclo-DOPA, said yeast cell comprising a mutation resulting in reduced activity of one or more native β-glucosidases;
   b. expressing in said yeast cell:
      i. a heterologous BAHD acyltransferase capable of acylating a betalain and/or glycosylated cyclo-DOPA, thereby obtaining an acylated betalain; and/or
      ii. a heterologous glycosyltransferase capable of glycosylating a betalain and/or glycosylated cyclo-DOPA, thereby producing a di-glycosylated betalain;
   thereby obtaining a modified betalain with an improved purity and/or titer as compared to the purity and/or titer of a modified betalain produced by a yeast cell expressing said heterologous BAHD acyltransferase and/or said heterologous glycosyltransferase, but not comprising a mutation resulting in reduced activity of one or more native β-glucosidases, and cultivated under the same conditions.
36. The method according to any one of items 34 to 35, further comprising the step of recovering the modified betalain.
37. The method according to any one of items 34 to 36, wherein:
   a. the yeast cell is as defined in any one of items 1 to 33;
   b. the acylated betalain is as defined in any one of the preceding items;
   c. the di-glycosylated betalain is as defined in any one of the preceding items;
   d. the BAHD acyltransferase is as defined in any one of the preceding items;
   e. the glycosyltransferase is as defined in any one of the preceding items.
38. The method according to any one of items 34 to 37, wherein the titer of the modified betalain is at least 0.1 mg/L, such as at least 0.25 mg/L, such as at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.
39. The method according to any one of items 34 to 38, wherein the medium is supplemented with:
   a. L-tyrosine; and/or
   b. UDP-glucuronic acid; and/or
   c. betanin; and/or
   d. isobetanin; and/or
   e. glycosylated cyclo-DOPA.
40. The method according to any one of items 34 to 39, wherein the yeast cell further expresses a UDP-glucose dehydrogenase, wherein said UDP-glucose dehydrogenase is as defined in any one of the preceding items.
41. The method according to any one of items 39 to 40, wherein the di-glycosylated betalain is a di-glycosylated betacyanin, such as a glycosylated betanin and/or isobetanin, such as a betanin and/or isobetanin glucoronidated at position 2 of the glycosyl moiety of betanin and/or isobetanin, such as amaranthin and/or isoamaranthin.
42. The method according to any one of items 34 to 41, wherein the yeast cell further expresses:
   a. a first heterologous enzyme (TYH) capable of hydroxylating L-tyrosine and oxidizing L-DOPA;
   b. a second heterologous enzyme which is a 4,5-DOPA extradiol dioxygenase (DOD); and
   c. a third heterologous enzyme having glycosyltransferase activity; wherein said TYH, the DOD, and/or the enzyme having glycosyltransferase activity is as defined in any one of the preceding items.
43. The method according to any one of items 34 to 41, wherein the yeast cell further expresses:
   a. a first heterologous enzyme which is a tyrosine hydroxylase (TYH) capable of hydroxylating L-tyrosine and oxidizing L-DOPA;
   b. a second heterologous enzyme which is a 4,5-DOPA extradiol dioxygenase (DOD); and
   c. a third heterologous enzyme having glycosyltransferase activity;
   whereby the yeast cell produces a glycosylated betalains, such as betanin and/or isobetanin.
44. Use of a BAHD acyltransferase as defined in any one of the preceding items in a method of producing an acylated betalain, such as an acylated betanin and/or isobetanin.
45. Use of a glycosyltransferase as defined in any one of the preceding items in a method of producing a di-glycosylated betalain, such as a glycosylated betanin and/or isobetanin.
46. Use of a BAHD acyltransferase to catalyse the acylation of glycosylated cyclo-DOPA and/or a betalain, such as betanin and/or isobetanin, thereby obtaining an acylated betalain.
47. Use of a glycosyltransferase to catalyse the glycosylation of glycosylated cyclo-DOPA and/or a betalain, such as betanin and/or isobetanin, thereby obtaining a di-glycosylated betalain.
48. The use according to any one of items 43 to 47, wherein:
   a. the BAHD acyltransferase is as defined in any one of the preceding items;
   b. the glycosyltransferase is as defined in any one of the preceding items;
   c. the acylated betalain is as defined in any one of the preceding items;
   d. the di-glycosylated betalain is as defined in any one of the preceding items;
   e. the use is performed in a yeast cell.
49. Use of a BAHD acyltransferase as defined in any one of the preceding items for acylation of position 4 of the glycosyl moiety of betanin and/or isobetanin.
50. Use of a BAHD acyltransferase as defined in any one of the preceding items for acylation of position 6 of the glycosyl moiety of betanin and/or isobetanin.
51. Use of a glycosyltransferase as defined in any one of the preceding items for glucosylation of position 2 of the glycosyl moiety of betanin and/or isobetanin.
52. Use of a glycosyltransferase as defined in any one of the preceding items for glucuronidation of position 2 of the glycosyl moiety of betanin and/or isobetanin.
53. The use according to any one of items 49 to 52, wherein the use is performed in a yeast cell.
54. A system of nucleic acids encoding:
   a. a BAHD acyltransferase capable of acylating glycosylated cyclo-DOPA and/or a betalain, such as a betanin and/or isobetanin; and/or
   b. a glycosyltransferase capable of glycosylating glycosylated cyclo-DOPA and/or a betalain, such as a betanin and/or isobetanin.
55. The system according to item 53, wherein:
   a. the BAHD is encoded by the polynucleotide set forth in SEQ ID NO: 30 or SEQ ID NO: 31 or a polynucleotide having at least 70% sequence identity thereto; and/or
   b. the glycosyltransferase is encoded by the polynucleotide as set forth in SEQ ID NO: 39, SEQ ID NO: 40, SEQ ID NO: 42, SEQ ID NO: 43, SEQ ID NO: 45 or SEQ ID NO: 46 or a polynucleotide having at least 70% sequence identity thereto.
56. Use of a polynucleotide as set forth in SEQ ID NO: 30 or SEQ ID NO: 31, or a polynucleotide having at least 70% sequence identity thereto, for obtaining a protein capable of acylating glycosylated cyclo-DOPA and/or a betalain, such as a betanin and/or isobetanin.
57. Use of a polynucleotide as set forth in SEQ ID NO: 39, SEQ ID NO: 40, SEQ ID NO: 42, SEQ ID NO: 43, SEQ ID NO: 45 or SEQ ID NO: 46, or a polynucleotide having at least 70% sequence identity thereto, for obtaining a protein capable of glycosylating glycosylated cyclo-DOPA and/or a betalain, such as a betanin and/or isobetanin.
58. A yeast cell capable of producing a glycosylated betalain, said yeast cell expressing:
   a. a first heterologous enzyme (TYH) capable of hydroxylating L-tyrosine and oxidizing L-DOPA;
   b. a second heterologous enzyme which is a 4,5-DOPA extradiol dioxygenase (DOD); and
   c. a third heterologous enzyme having glycosyltransferase activity;
   whereby said yeast cell is capable of producing a glycosylated betalain, wherein said yeast cell comprises a mutation resulting in reduced activity of one or more native β-glucosidases.
59. The yeast cell according to item 58, wherein:
   a. the glycosylated betalain is a glycosylated betacyanin, betanin and/or
   isobetanin; and/or
   b. the TYH is as defined in any one of the preceding items; and/or
   c. the DOD is as defined in any one of the preceding items; and/or
   d. the enzyme having glycosyltransferase activity is as defined in any one of the preceding items.
60. The yeast cell according to any one of items 58 to 59 wherein said yeast cell comprises a mutation in one or more genes encoding for one or more native β-glucosidases, wherein said one or more genes are selected from the group consisting of:
   i) YALI1_B23300g (SEQ ID NO: 61);
   ii) YALI1_F21504g (SEQ ID NO: 62);
   iii) YALI1_B05024g (SEQ ID NO: 63);
   iv) YALI1_B18845g (SEQ ID NO: 64);
   v) YALI1_B18887g (SEQ ID NO: 65);
   vi) YALI1_D22997g (SEQ ID NO: 66);
   vii) YALI1_E40502g (SEQ ID NO: 67);
   viii) YALI1_F02592g (SEQ ID NO: 68);
   ix) YALI1_F08075g (SEQ ID NO: 69);
   x) YALI1_F17788g (SEQ ID NO: 70);
   xi) YALI1_E23994g (SEQ ID NO: 79);
   xii) YALI1_E39796g (SEQ ID NO: 80);
   xiii) YALI1_F03075g (SEQ ID NO: 81);
   xiv) YALI1_E25163g (SEQ ID NO: 82);
   xv) YALI0F16027g (SEQ ID NO: 217);
   xvi) YALI0B14289g (SEQ ID NO: 218); and
   xvii) YALI0F05390g (SEQ ID NO: 219);
   preferably wherein said one or more genes are selected from the group consisting of YALI1_D22997g (SEQ ID NO: 66), YALI1_B23300g (SEQ ID NO: 61), YALI1_F21504g (SEQ ID NO: 62), YALI1_F08075g (SEQ ID NO: 69), YALI1_B18845g (SEQ ID NO: 64), YALI1_E23994g (SEQ ID NO: 82), YALI0F16027g (SEQ ID NO: 217), YALI0B14289g (SEQ ID NO: 218) and YALI0F05390g (SEQ ID NO: 219).
61. The yeast cell according to any one of items 58 to 60, wherein said yeast cell further expresses:
   a. a BAHD acyltransferase, whereby said yeast cell is capable of producing an acylated betalain, optionally wherein said BAHD acyltransferase and/or said acylated betalain is as defined in any one of the preceding items; and/or
   b. a glycosyltransferase, whereby said yeast cell is capable of producing a di-glycosylated betalain, optionally wherein said glycosyltransferase and/or said di-glycosylated betalain is as defined in any one of the preceding items; and/or
   c. a UDP-glucose dehydrogenase, whereby the yeast cell is capable of producing UDP-glucuronic acid, optionally wherein the UDP-glucose dehydrogenase is as defined in any one of the preceding items.
62. A method for producing a glycosylated betalain, said method comprising the steps of:
   a. providing the yeast cell according to any one of items 58 to 61, said yeast cell producing a glycosylated betalain;
   b. incubating said yeast cell in a medium;
   c. expressing in said yeast cell:
      i. a first heterologous enzyme (TYH) capable of hydroxylating L-tyrosine and oxidizing L-DOPA;
      ii. a second heterologous enzyme which is a 4,5-DOPA extradiol dioxygenase (DOD); and
      iii. a third heterologous enzyme having glycosyltransferase activity;
   thereby obtaining a glycosylated betalain.
63. A method for improving the titer and/or purity of one or more glycosylated betalains produced by a yeast cell, said method comprising the steps of:
   a. providing the yeast cell according to any one of items 58 to 61:
   b. incubating said yeast cell in a medium;
   c. expressing in said yeast cell:
      i. a first heterologous enzyme (TYH) capable of hydroxylating L-tyrosine and oxidizing L-DOPA;
      ii. a second heterologous enzyme which is a 4,5-DOPA extradiol dioxygenase (DOD); and
      iii. a third heterologous enzyme having glycosyltransferase activity;
   thereby obtaining a glycosylated betalain with an improved purity and/or titer as compared to the purity and/or titer of a yeast cell expressing said TYH, DOD and enzyme having glycosyltransferase activity but not comprising a mutation resulting in reduced activity of one or more native β-glucosidases, wherein said yeast cells are cultivated under the same conditions.
64. A method for producing a glycosylated betalain, said method comprising the steps of:
   a. providing the yeast cell according to any one of items 58 to 61;
   b. incubating said yeast cell in a medium comprising a β-glucosidase inhibitor, such as cellobiose;
   c. expressing in said yeast cell:
      i. a first heterologous enzyme (TYH) capable of hydroxylating L-tyrosine and oxidizing L-DOPA;
      ii. a second heterologous enzyme which is a 4,5-DOPA extradiol dioxygenase (DOD); and
      iii. a third heterologous enzyme having glycosyltransferase activity;
   thereby obtaining a glycosylated betalain.
65. A method for improving the titer and/or purity of one or more betalains produced by a yeast cell, said method comprising the steps of:
   a. providing the yeast cell according to any one of items 58 to 61;
   b. incubating said yeast cell in a medium comprising a β-glucosidase inhibitor, such as cellobiose;
   c. expressing in said yeast cell:
      i. a first heterologous enzyme (TYH) capable of hydroxylating L-tyrosine and oxidizing L-DOPA;
      ii. a second heterologous enzyme which is a 4,5-DOPA extradiol dioxygenase (DOD); and
      iii. a third heterologous enzyme having glycosyltransferase activity;
   thereby obtaining a betalain with an improved purity and/or titer as compared to the purity and/or titer of the yeast cell incubated in the absence of the β-glucosidase inhibitor, such as cellobiose, but otherwise cultivated under the same conditions.
66. The method according to any one of items 62 to 65, further comprising the step of recovering the betalain.
67. The method according to any one of items 62 to 66, wherein:
   a. the glycosylated betalain is betanin and/or isobetanin; and/or
   b. the TYH is as defined in any one of the preceding items; and/or
   c. the DOD is as defined in any one of the preceding items; and/or
   d. the enzyme having glycosyltransferase activity is as defined in any one of the preceding items; and/or
   e. the medium is as defined in any one of the preceding items.

### EXAMPLES

### Example 1—Materials and Methods

### Strains and Media

To clone and store plasmids, E. coli strain DH5a was used. The cultivations were carried out at 37° C. in Lysogeny Broth (LB) broth or on agar-plates supplemented with 100 mg/L ampicillin as selection marker. The prototrophic yeast strain CEN.PK113-7D (MATa MAL2-8c SUC2 URA3 HIS3 LEU2 TRP1) harboring episomal vector for Cas9 protein expression (Ptef1-Cas9-Tcyc1_kanMX) was used as the parent strain (ST7574) in this study (Milne et al. 2020). To keep the selection for Cas9, the cultivations for all the yeast strains were supplemented with 200 mg/L G418 (Sigma-Aldrich). The construction of yeast strains was carried out by EasyClone MarkerFree method (Jessop-Fabre et al. 2016).

For betalain production, the MM (pABA⁻) medium, i.e. modified mineral medium without p-aminobenzoic acid, was used to culture the cells. This medium consisted of 20 g/L glucose, 7.5 g/L (NH₄)₂SO₄, 14.4 g/L KH₂PO₄, 0.5 g/L MgSO₄·7H₂O, 2 mL/L trace metal solution (3.0 g/L FeSO₄·7H₂O, 4.5 g/L ZnSO₄·7H₂O, 4.5 g/L CaCl₂·2H₂O, 0.84 g/L MnCl₂·2H₂O, 0.3 g/L CoCl₂·6H₂O, 0.3 g/L CuSO₄·5H₂O, 0.4 g/L Na₂MoO₄·2H₂O, 1.0 g/L H₃BO₃, 0.1 g/L KI, and 19.0 g/L Na₂EDTA·2H₂O), and 1 mL/L vitamin solution (0.05 g/L D-biotin, 1.0 g/L D-pantothenic acid hemicalcium salt, 1.0 g/L thiamin-HCl, 1.0 g/L pyridoxin-HCl, 1.0 g/L nicotinic acid, and 25.0 g/L myo-inositol). The constructed yeast cells were inoculated to this medium using the overnight culture that was grown in YPD media (10 g/L yeast extract, 20 g/L peptone (10 g/L) and 20 g/L glucose) at 30° C. and 250 rpm. The betalain production was carried out for 48 h at 30° C. and 250 rpm. For cultivation of S. cerevisiae, the cultivation supernatant was used for analytical liquid chromatography to detect the compound of interest. For cultivation of Y. lipolytica, the total betalain content (intracellular and extracellular) was asessed by lysing the cultivation broth including the cells with glass beads and using a Precellys 24 homogenizer (Bertin Technologies, FR). After cell disruption, the debris was spun down and the betalain content in the supernatant measured by HPLC and LC-MS.

For qualitative assessment of β-glucosidase activity in yeast strains, esculin glycerol agar (EGA) medium was used (Pérez et al. 2011). This medium consisted of 1 g/L esculin, 0.3 g/L ferric chloride, 1 g/L casein hydrolysate, 25 g/L yeast extract, 8 mL/L glycerol, and 20 g/L agar. The final pH of the medium was 6.0. The medium was autoclaved at 121° C. for 15 min and poured into petri dishes (20 mL per plate). After solidifying, 10 μL of different yeast strains with OD₆₀₀=1 were inoculated from 24 h cultures in YPD. Each plate was inoculated with four cultures, incubated at 30° C., and examined after 5 days.

### Synthetic Genes and DNA Materials

The heterologous genes (Table 1) were synthesized by Twist Bioscience and GeneArt in codon-optimized versions for S. cerevisiae. All DNA parts were PCR amplified using Phusion U DNA polymerase (ThermoFisher) according to the manufacturer's instructions. The DNA fragments (BioBricks) are listed in Table 2. The DNA fragments obtained by PCR were separated in 1%-agarose containing RedSafe™ (iNtRON Biotechnology), and purified using the Nucleospin Gel and PCR Clean-up kit (Macherey-Nagel). Intergrative vectors were constructed as descibed in EasyClone MarkerFree method (Jessop-Fabre et al. 2016).

### Query for New BAHD Acyltransferase Enzymes

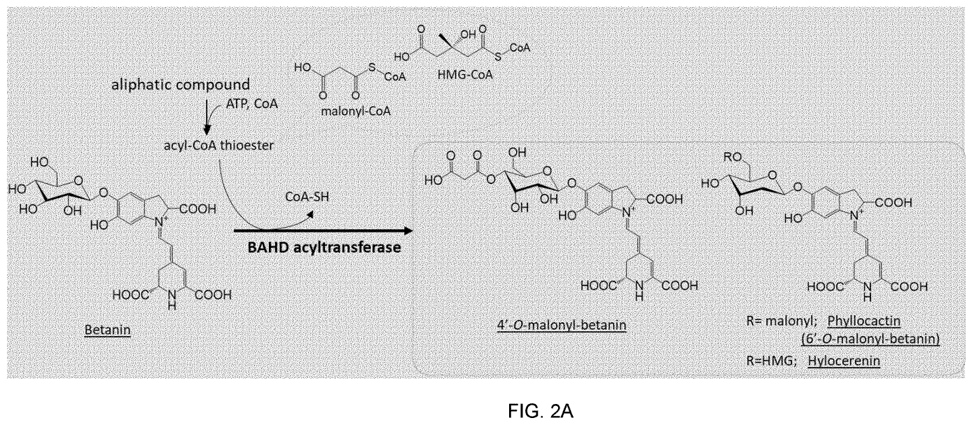

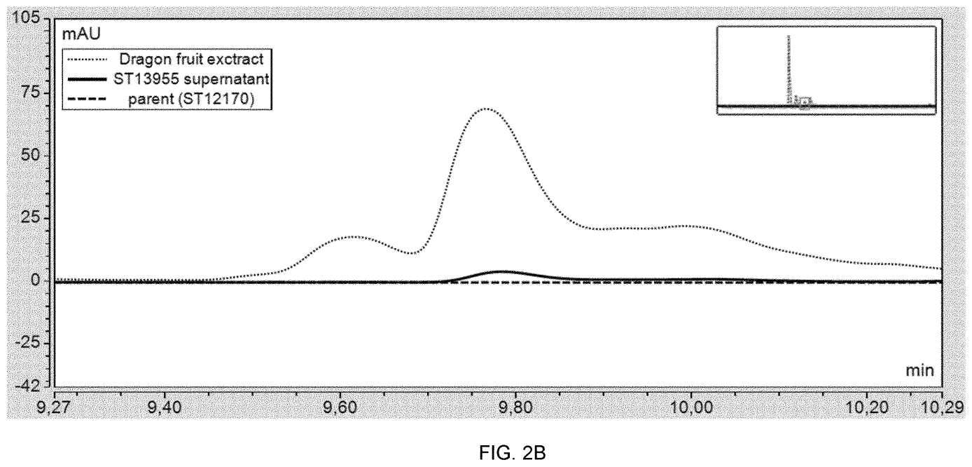

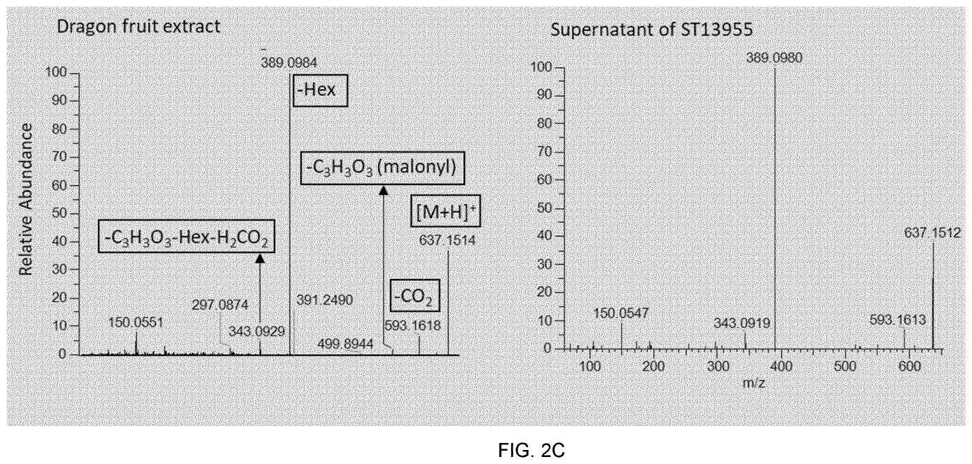

To find novel BAHD acyltransferases for production of acylated betalains in yeast (FIG. 2a), we BLASTed the protein sequences of malonyl-CoA:isoflavone 7-O-glucoside-6″-O-malonyltransferase from Glycine max (Soybean) (UniProtID: A7BIC9_SOYBN) and malonyl-CoA:anthocyanidin 5-O-glucoside-6″-O-malonyltransferase from Arabidopsis thaliana (Mouse-ear cress) (UniProtID: 5MAT_ARATH) into the assembled Celosia cristata flower transcriptome (NCBI: SRR9095475) and the differentially expressed assembled transcriptomes of Hylocereus polyrhizus (red pulp stage dragon fruit sample) (NCBI: SRR3203780) and Hylocereus polyrhizus (white pulp stage dragon fruit sample)(NCBI: SRR2924904). In addition, sequences belonging to PFAM PF02458 and annotated as BAHD are extracted from the Celosia cristata transcriptome and the differentially expresssed Hylocereus polyrhizus transcriptome. The resulting sequence hits were filtered for presence of conserved HXXXD motif, and those starting with a start codon were extracted and identical sequences removed. Of the remaining sequences from Celosia cristata, the 3 highest expressed genes were selected and named CcBAHD1-CcBAHD3. Of the remaining sequences from Hylocereus polyrhizus, 6 were selected based on their expression level in red dragon fruit or because of significantly (2-fold) higher expression in red pulp stage dragonfruit than in white pulp stage dragon fruit and named HpBAHD1-HpBAHD6. These 9 genes were ordered codon-optimized for S. cerevisiae as gene strings.

### Query for New Glucuronosyl-Transferases Enzymes

To find novel glucuronosyl-transferases for production of glucuronosylated betalains we BLASTed cyanidin-3-O-glucoside 2-O-glucuronosyltransferase from Bellis perennis (Daisy) (UniProtID: UGAT_BELPE) into the assembled Celosia cristata transcriptome (NCBI: SRR9095475). The resulting sequence hits were filtered for a length of at least 300 amino acids, presence of the Prosite pattern PS00375. Those starting with a start codon were extracted, identical sequences removed and the 10 highest expressed sequences were selected. 3 sequences of experimentally validated glucuronosyl-transferases from literature (Imamura et al. 2019) AhUGT79B30-like4, hereinafter referred to as AhAmaSy1, CqAmaSy1 and Bv3GGT-like1, hereinafter referred to as BvAmaSy1, were BLASTed into the transcriptome of C. cristata and the best hit to those sequences in the subset of 10 sequences was selected and named CcAmaSy1. AhAmaSy1, CqAmaSy1 and CcAmaSy1 were ordered codon-optimized for S. cerevisiae as gene strings.

### Analytics

To identify and quantify betalain pigments, the analytical liquid chromatography was performed by Dionex Ultimate 3000 HPLC system (Thermo Fisher Scientific, US) and Liquid chromatography-mass spectrometry (LC-MS). For HPLC analysis, 10 μL sample was injected into a Zorbax Eclipse Plus C18 reverse-phased column (particle size 3.5 μm, pore size 95 Å, 4.6×100 mm). The column oven temperature was set to 30° C., and the flow rate was 1.0 mL/min. Solvent A was water+0.1% formic acid, and solvent B was 100% acetonitrile. The solvent composition was initially set to A=98.0% and B=2.0%, and kept steady for 3 min. Hereafter, the solvent composition was adjusted following a linear gradient, until reaching A=75.0% and B=25.0% at 19 min. The column was then flushed by setting A=2.0% and B=98% at 21 min. These conditions were kept steady until 22.5 min and were then returned to the initial conditions of A=98.0% and B=2.0% at 23 min, at which point the solvent composition remained unchanged until the end of the run at 24.5 min. All betalains were detected with a UV-VIS detector at a wavelength of 540 nm. The UV-Vis detector captured data at 390 nm, 410 nm, 480 nm, and 540 nm. The Chromeleon 7 software (Thermo Fisher Scientific, US) was used to analyze HPLC results and generate standard curves. For quantification of betanin and isobetanin, peaks corresponding to betanin and isobetanin were identified by comparison to red beet extract diluted with dextrin which is the only commercially available standard for betanin (product ID:901266-5G). This commercial red beet extract contains an equimolar ratio of betanin and isobetanin as was determined by HPLC. By using the Beer-Lambert equation assuming a molar extinction coefficient of E=6.5×10⁴ M⁻¹cm⁻¹ for betanin and isobetanin, it was calculated that 1 g/L of this extract contains 0.837 mg/L of betanin and isobetanin respectively. The LC-MS analysis was performed using a UHPLC Ultimate 3000 binary system (Thermo Fisher Scientific, USA) coupled to a DAD-(ESI)Fusion Orbitrap Mass Spectrometer (Thermo Fisher Scientific, USA). The chromatographic separation was achieved using a Waters ACQUITY BEH C18 (10 cm×2.1 mm, 1.7 μm) equipped with an ACQUITY BEH C18 guard column kept at 30 C and mobile phase consisting of MilliQ water+0.1% Formic acid (A) and Acetonitrile+0.1% Formic acid (B) using a flow rate of 0.35 mL/min. The mobile phase gradient was as follows: 0.2% B was held for 3 min, followed by a linear increase till 25% B in 20 min and kept for 1 min. After that the concentration of B increased to 100% in 4 min and stayed at 100% for 2 min before going back to initial conditions. Re-equilibration time was 2 min. The sample injection was 1 μL. The DAD settings were the following: data collection rate was 10 Hz and the wavelength range 190-600 nm with a bandwidth of 2 nm. The MS acquisition was set in positive-heated electrospray ionization (+HESI) mode with a voltage of 3500 V acquiring in full MS/MS spectra (Data dependent Acquisition-driven MS/MS) in the mass range of 70-1000 Da. The DDA acquisition settings were the following: automatic gain control (AGC) target value set at 4e5 for the full MS and 5e4 for the MS/MS spectral acquisition, the mass resolution was set to 120,000 for full scan MS and 30,000 for MS/MS events. Precursor ions were fragmented by stepped High-energy collision dissociation (HCD) using collision energies of 20, 40, and 60.

To obtain pure fractions of the betalain variants, preparative HPLC was performed. 100 μL of sample were injected into a Zorbax Eclipse Plus C18 reverse-phased column (particle size 3.5 μm, pore size 95 Å, 4.6×100 mm). The column oven temperature was set to 30° C., and the flow rate was 0.8 mL/min. Solvent A was water+0.1% formic acid, and solvent B was 100% acetonitrile. The solvent composition was initially set to A=98.0% and B=2.0%, and kept steady for 2 min. Hereafter, the solvent composition was adjusted following a linear gradient, until reaching A=90.0% and B=10.0% at 12 min. Then, the solvent composition was adjusted following a second linear gradient, until reaching A=80.0% and B=20.0% at 17 min. The column was then flushed by setting A=2.0% and B=98% at 18 min. These conditions were kept steady until 18.5 min and were then returned to the initial conditions of A=98.0% and B=2.0% at 19 min, at which point the solvent composition remained unchanged until the end of the run at 20.5 min. All betalains were detected with a UV-VIS detector at a wavelength of 540 nm. The UV-Vis detector captured data at 280 nm, 410 nm, 480 nm, and 540 nm. To obtain the desired compound fraction, the respective time span in which that compound elutes was selected

### Example 2—Plant Extraction and Results of Analytics

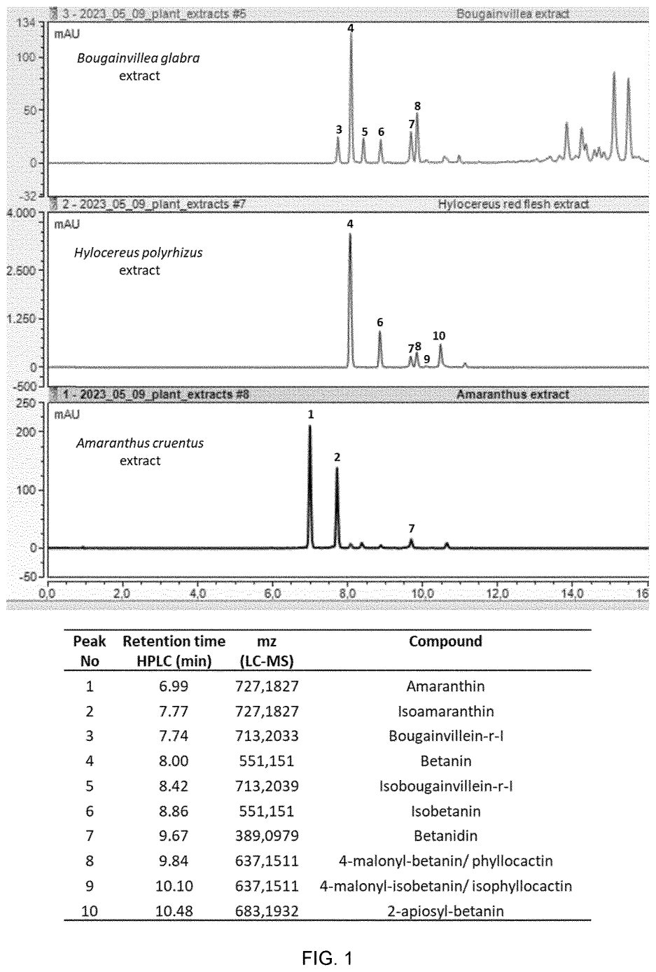

The coloured petals of dried pink Bougainvillea glabra plants and dried red Amaranthus cruentus were grinded with 10 mM ascorbic acid with mortar and pestle until the liquid was deeply coloured. The solid plant residues were removed by centrifugation (4° C., 11.000 g, 5 min) and the supernatant was first filtered through a 0.45 μm syringe filter, then through a 0.2 μm syringe filter. For extraction of pigments from the red flesh dragon fruit Hylocereus polyrhizus, a fresh fruit was peeled, the fruit flesh cut into pieces and crushed with mortar and pestle. The crushed fruit flesh was mixed with 10 mM ascorbic acid in water and stirred for 1 h at 4° C. on a magnetic stirrer. The solid plant residues were removed by centrifuging the extract twice (4° C., 11.000 g, 5 min), followed by filtration through a 0.45 μm syringe filter, then a 0.2 μm syringe filter. The extracts were stored at −20° C. until analysis by HPLC and LC-MS. HPLC and LC-MS analysis of the extracts was performed to identify the betalains in the extracts (FIG. 1).

### Example 3—Screening of BAHD Acyltransferases and Production of Malonyl-Betanin

To obtain the yeast strains with acylated betanin, first the parent strain with integrated betanin synthesis pathway was constructed. To do this, the parent strain ST7574 (CEN.PK113-7D harboring episomal plasmid for Cas9 expression under the KanMX antibiotic-resistance selection marker) was engineered for chromosomal integration of betanin pathway, consisting of BvCYP76AD1^{W13L} (SEQ ID NO:4-5), MjDOD (SEQ ID NO:1-2), and two different types of UGTs: BvSGT2 (SEQ ID NO:8-9), betanidin-5-O-GT and BgGT2 (SEQ ID NO:11-12), cycloDOPA-5-O-GT to get strains ST12160 and ST12170, respectively. These two strains were then used as the parent strains for chromosomal integration of BAHD acyltransferases (SEQ ID NO: 19 to 37), and the strains ST13941 to ST13958 (Table 4) were obtained. These strains were then cultivated 24-deep-well plate containing 2 mL MM (pABA⁻) (Example 1) with air-penetrable metal lid (EnzyScreen, The Netherlands) at 30° C. and 250 rpm for 48 h. The culture supernatant was then analyzed by liquid chromatography along with plant extract of the red dragon fruit Hylocereus polyrhizus. The HPLC data showed that from all the constructed strains, there was a new betalain with elution time of 9.78 min in the strains expressing HpBAHD3 (SEQ ID NO:29-30), which was present in the chromatogram of the red dragon fruit extract as well (FIG. 2b). Further characterization of this compound by mass spectrophotometry (2c) showed an MS peak of [M+H]⁺ at 637.1514 and further MS/MS fractionation at 619; 593; 551 and 389, which corresponds to phyllocactin (6′-O-malonyl-betanin) and/or 4′-O-malonyl-betanin (Wybraniec et al. 2007; Piattelli and Minale 1964; Khan and Giridhar 2015).

### Example 4—Screening of Glucuronosyl-Transferases and Production of Bougainvillein-r-I in S. cerevisiae

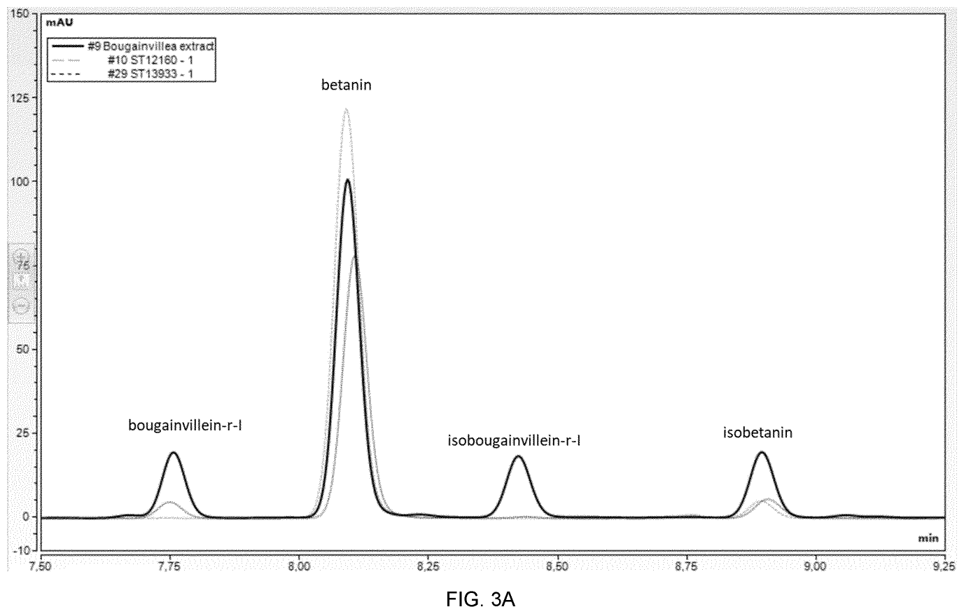

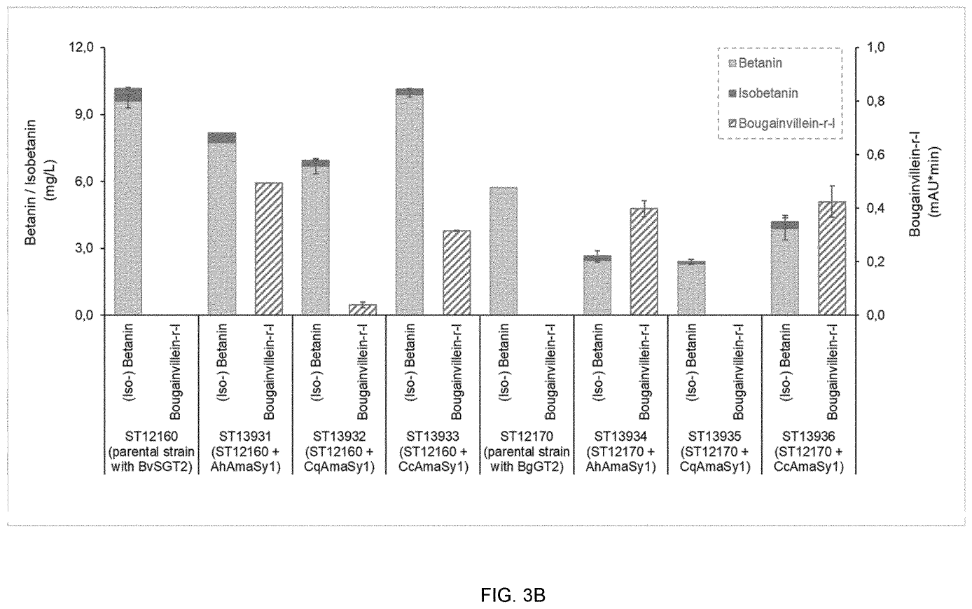

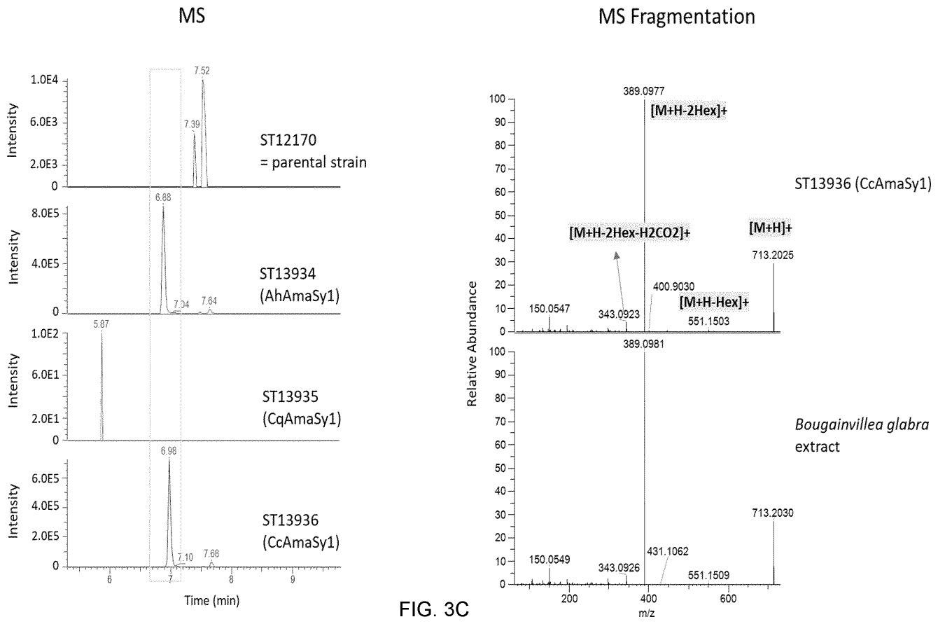

The three glucuronosyltransferases AhAmaSy1 (SEQ ID NO: 38-39), CqAmaSy1 (SEQ ID NO:41-42) and CcAmaSy1 (SEQ ID NO: 44-45) were codon-optimized for S. cerevisiae and integrated into the betanin-producing strains ST12160 and ST12170 (Example 3). The strains were cultivated in 2 mL minimal media in 24-well plates and the supernatant was analysed by HPLC and LC-MS. The two enzymes AhAmaSy1 and CqAmaSy1 have been reported to produce amaranthin when recombinantly expressed in Nicotiana benthamiana leaves that co-express the betanin pathway genes from C. quinoa CqCYP76AD1-1, CqCDOPA5GT, CqDODA-1. (Imamura et al. 2019). CcAmaSy1 has not been characterised before. Surprisingly, strains that expressed AhAmaSy1 or CcAmaSy1 produced bougainvillein-r-I in addition to betanin and isobetanin, but not amaranthin (FIG. 3a, FIG. 3b). ST13932 (ST12160+CqAmaSy1) produced very low amounts of bougainvillein-r-I while ST13935 (ST12170+CqAmaSy1) produced exclusively betanin and isobetanin and no bougainvillein-r-I at all. Characterization of the cultivation broth by mass spectrophotometry showed an MS peak of [M+H]⁺ at 713.20 and further MS/MS fragmentation at 551, 389, 343 which corresponds to bougainvillein-r-I (Sutor and Wybraniec 2020), confirming the production of the compound by the yeast strains (FIG. 3c).

### Example 5—Production of Amaranthin with Glucuronosyl-Transferases in S. cerevisiae

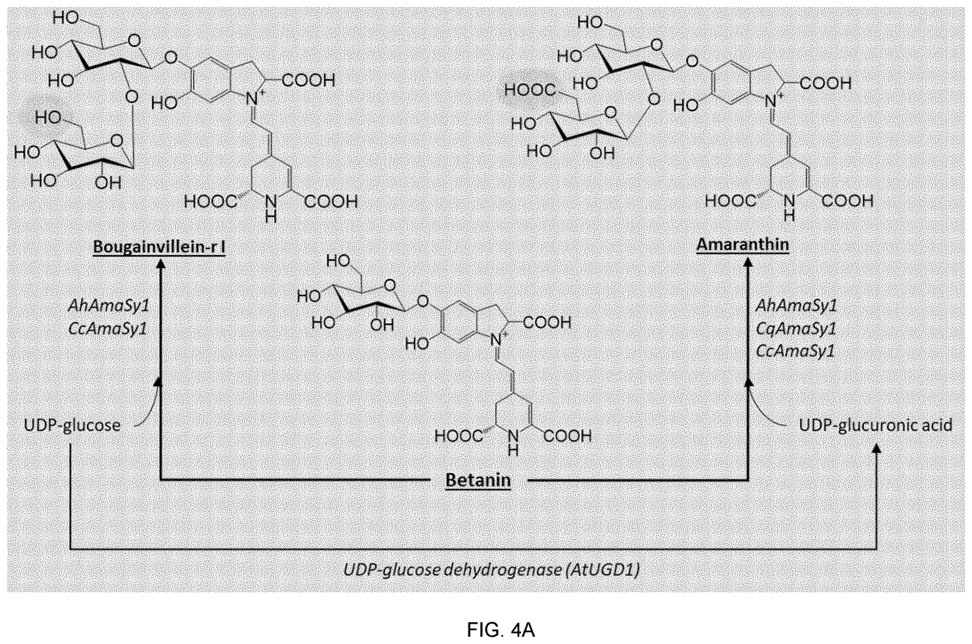

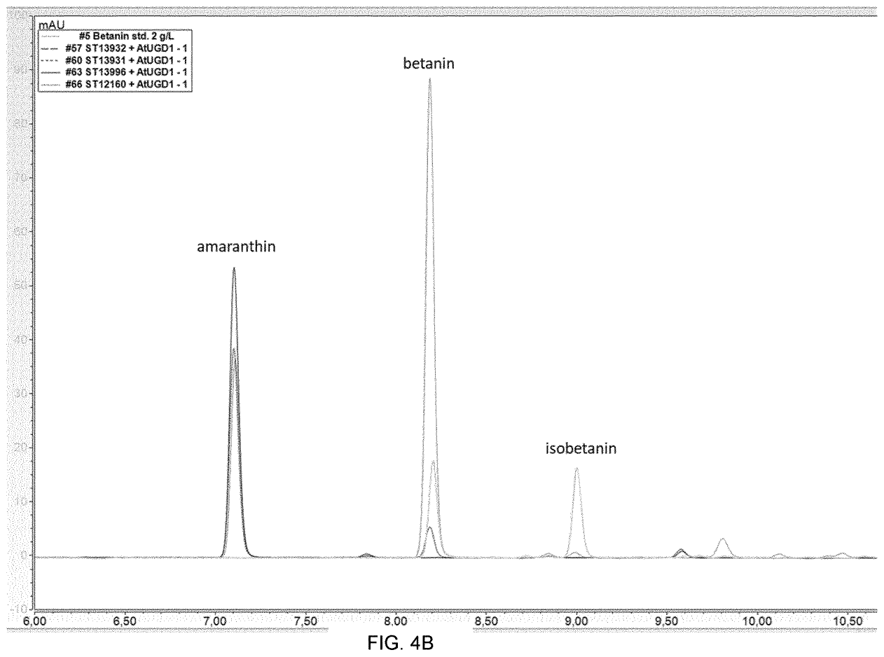

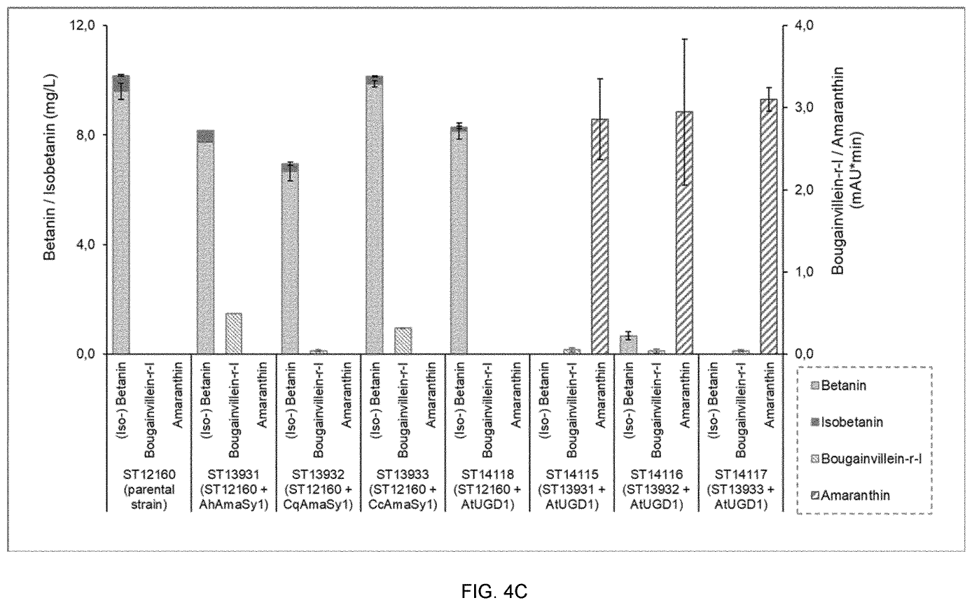

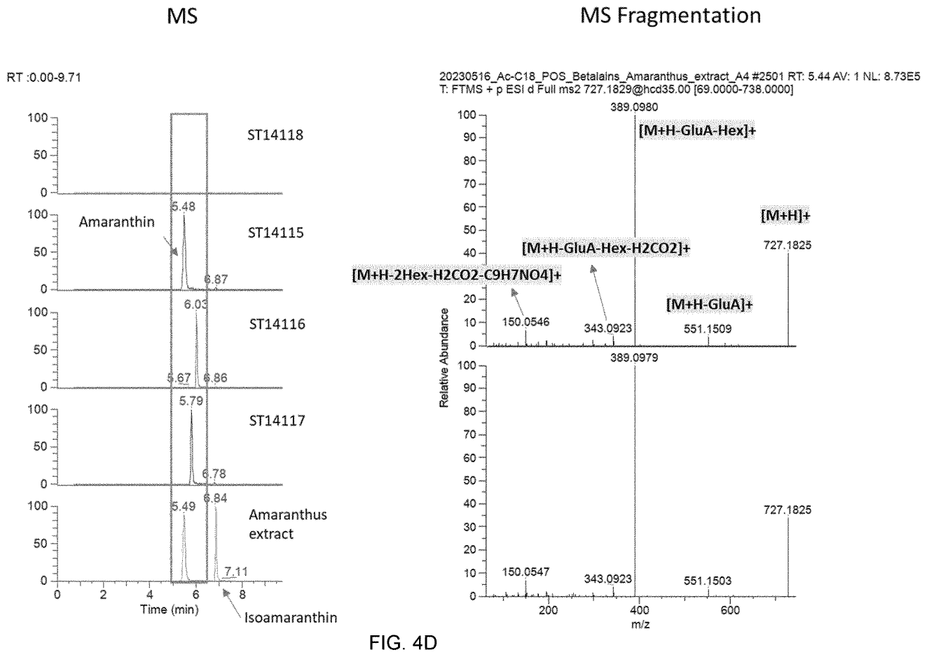

Amaranthin is formed by addition of glucuronic acid to the 2′-hydroxyl group of the glucose moiety of the betanin molecule. This reaction requires the presence of UDP-glucuronic acid, which is not endogenously present in S. cerevisiae. It has been shown that the recombinant expression of the UDP-glucose dehydrogenase from Arabidopsis thaliana (AtUGD1) in S. cerevisiae leads to the formation of UDP-glucuronic acid from UDP-glucose (Oka and Jigami 2006). To enable the production of amaranthin instead of bougainvillein-r-I by S. cerevisiae strains, codon-optimized AtUGD1 (SEQ ID NO:47-48) was expressed together with one of the glucuronosyltransferases AhAmaSy1 (SEQ ID NO:38-39), CqAmaSy1 (SEQ ID NO:41-42) and CcAmaSy1 (SEQ ID NO:44-45) in the betanin-producing strain ST12160 (FIG. 4a). The resulting strains were cultivated in small-scale in minimal media for 48 h and the supernatant was analysed by HPLC and LC-MS. ST12160 only expressing AtUGD1 and no glucuronosyltransferase (ST14118) produces only betanin, small amounts of isobetanin and betanidin. In all three strains expressing a glucuronsyltransferase on top of AtUGD1 (ST14115, ST14116, ST14117), the main betalain produced was amaranthin instead of betanin, whereby AhAmaSy1- and CcAmaSy1-expressing strains exclusively produced amaranthin, indicating that all betanin has been glycosylated to amaranthin. Only small amounts of bougainvillein-r-I were produced by the strains (FIG. 4b, FIG. 4c). Characterization of the cultivation broth by mass spectrophotometry showed an MS peak of [M+H]⁺ at 727.18 and further MS/MS fragmentation at 551, 389, 343, 150 which corresponds to amaranthin, confirming the production of the compound by the yeast strains (FIG. 4d).

### Example 6—Production of Amaranthin, Bougainvillein-r-I and Phyllocactin in Yarrowia lipolytica

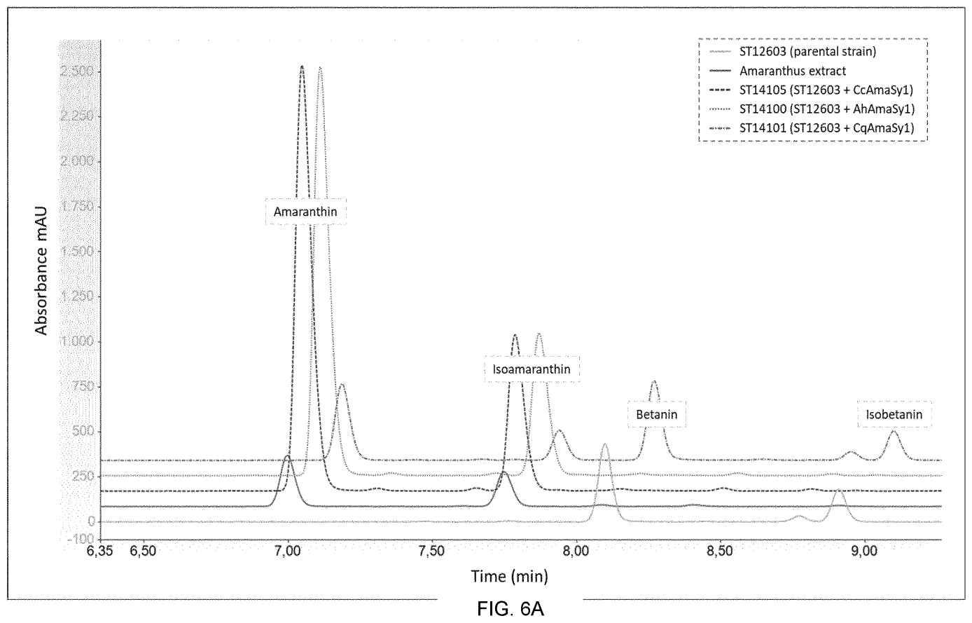

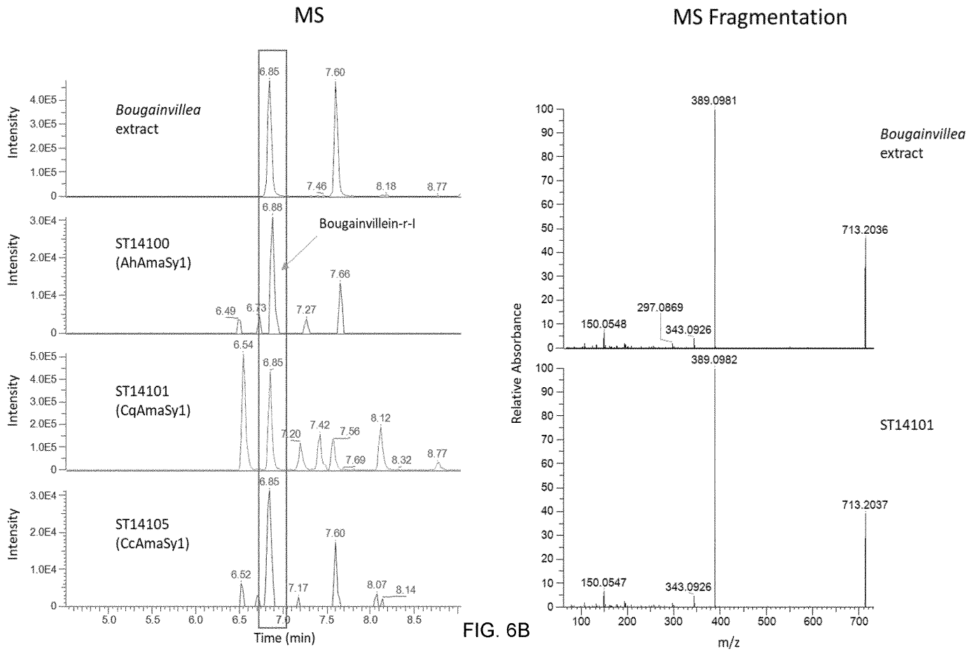

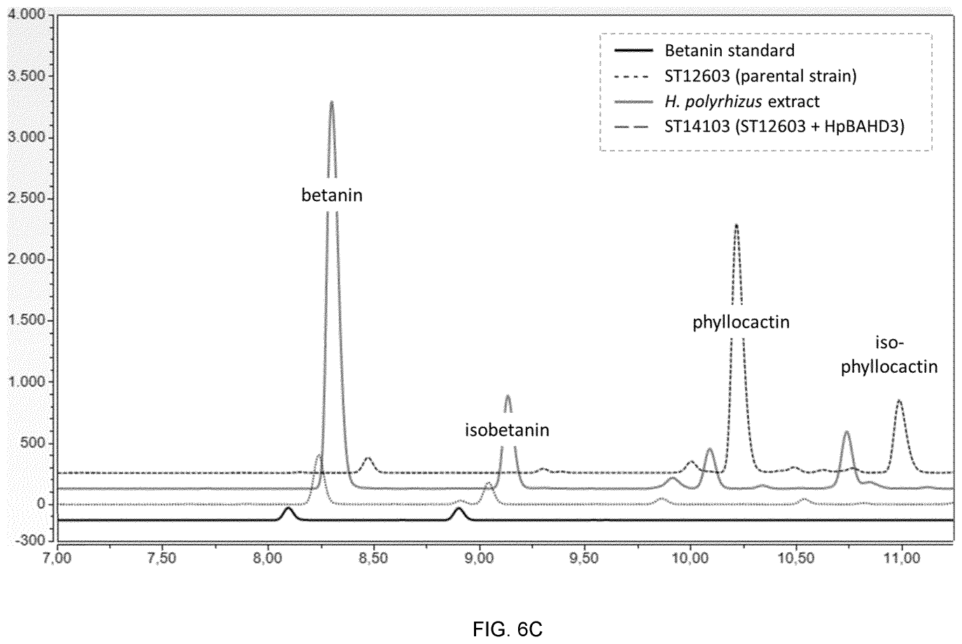

After successful production of amaranthin, bougainvillein-r-I and phyllocactin in S. cerevisiae, we wanted to examine the production of these betalain variants in another organism such as the oleaginous yeast Yarrowia lipolytica. We've previously seen that this yeast can be an efficient cell factory for the heterologous production of betanin (WO2022253815A1). Therefore, one of the glucuronosyltransferases AhAmaSy1_YI (SEQ ID NO: 38, 40), CqAmaSy1_YI (SEQ ID NO: 41, 43) or CcAmaSy1_YI (SEQ ID NO: 44, 46), codon-optimized for Y. lipolytica, was integrated into strain ST11193, which only expresses one copy of the betanin pathway genes without any other modifications, and into the highly engineered strain ST12603 that has the following modifications: three copies of the biosynthetic pathway (MjDOD-EvTYH-BvSGT2), feedback-inhibition-resistant Aro4 (YIARO4^{K221L}) and Aro7 (YIARO7^{G139S}) and deletion of the 4-hydroxyphenylpyruvate acid dioxygenase (A4HPPD). The codon-optimized BAHD acyltransferase HpBAHD3 (SEQ ID NO: 29, 31) was also integrated into the strains ST11193 and ST12603. The resulting strains were cultivated together with the parental strain in a 24-deep-well plate containing 2 mL MM (pABA⁻) for 60 hours. The total betalain content (intra- and extracellular) of the cultivation was analysed by HPLC and LC-MS. All three strains expressing one of the glucuronosyltransferases produced large amounts of amaranthin and isoamaranthin, whereby in the strains expressing AhAmaSy1 (ST14100) and CcAmaSy1 (ST14105) in ST12603 all the betanin has been converted to amaranthin and bougainvillein-r-I (FIG. 6a). LC-MS analysis of ST14100, ST14101 and ST14105 also confirmed the production of bougainvillein-r-I and isobougainvillein-r-I by all three strains (FIG. 6b). The strain ST14103 (ST12603+HpBAHD3) produced large amounts of phyllocactin and isophyllocactin and only small amounts of betanin and isobetanin, whereas the respective parental strain only produced betanin and isobetanin (FIG. 6c).

### Example 7—Betalains Degradation Through Deglycosylation in Yeast Strains

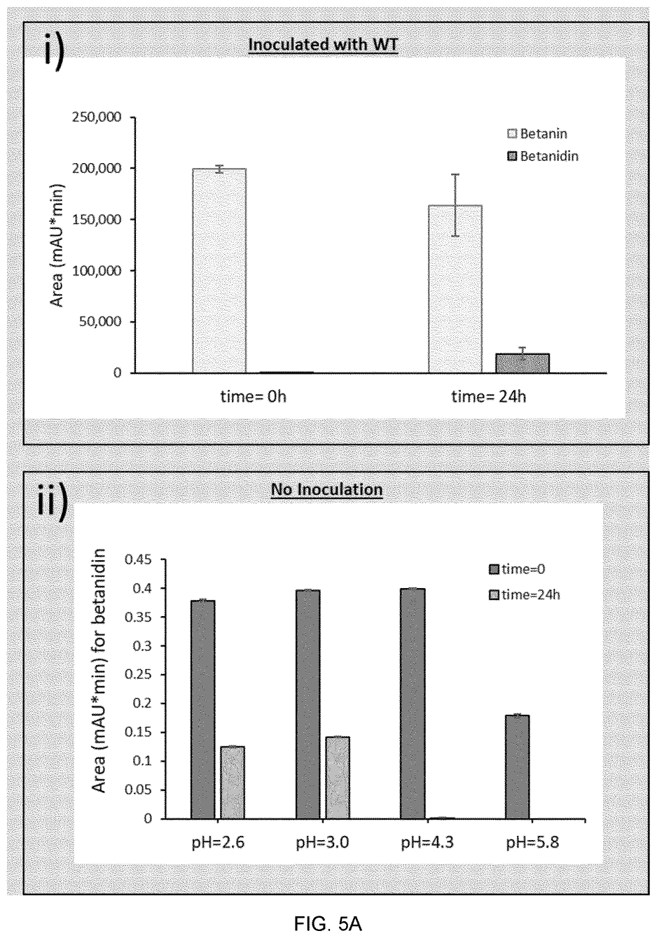

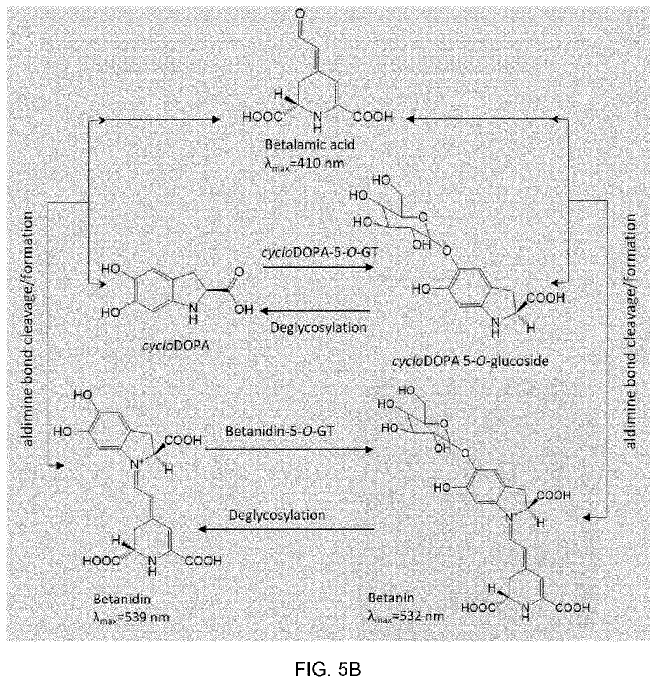

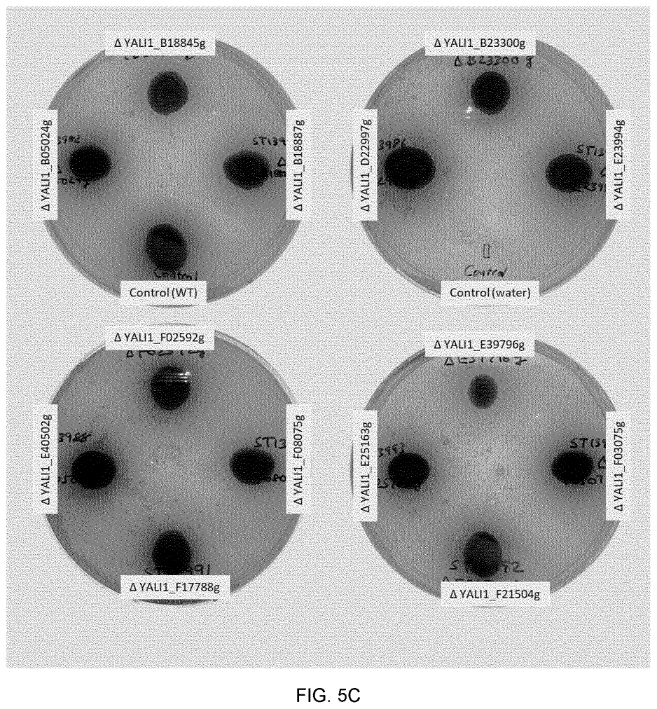

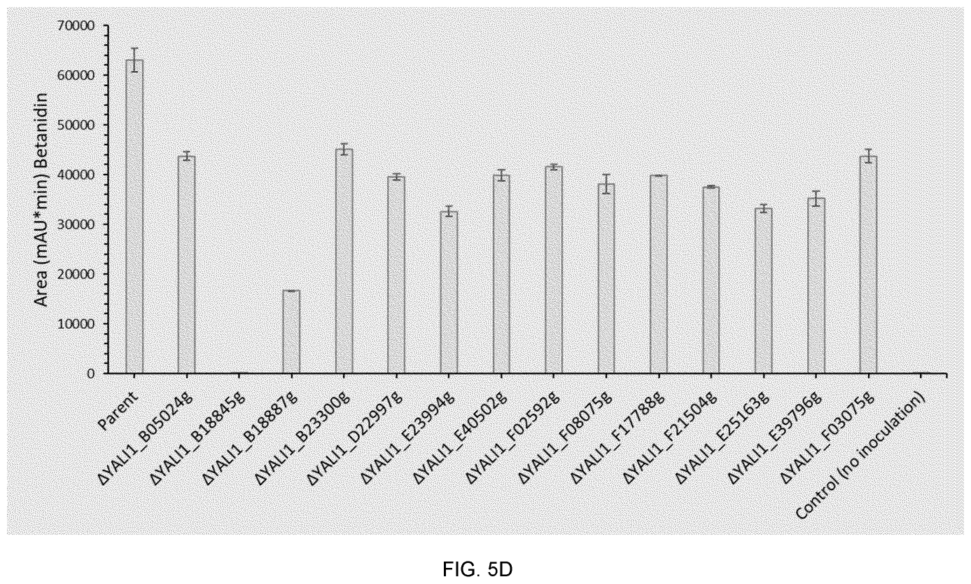

We observed that Y. lipolytica cells can hydrolyze betanin into betanidin (FIG. 5a), which leads to undesirable break down of the product and appearance of degradation by-products. Betanidin is unstable and can spontaneously hydrolyze into betalamic acid and cyclo-DOPA that could undergo further various reactions and create colorful products (e.g., brown, yellow, black, and other) that will cause undesired discoloration of the broth. Other betalains would likely also be a subject to analogous degradation. We hypothesized that betanin degradation in yeast cultures is caused by glucosidase(s) produced by the yeast cells FIG. 5b). From the genome sequencing data, we identified at least 14 β-glucosidase-encoding genes in Y. lipolytica W29 strain (SEQ ID NO: 61-70 and 79-82):
1) YALI1_B23300g
2) YALI1_F21504g
3) YALI1_B05024g
4) YALI1_B18845g
5) YALI1_B18887g
6) YALI1_D22997g
7) YALI1_E40502g
8) YALI1_F02592g
9) YALI1_F08075g
10) YALI1_F17788g
11) YALI1_E23994g
12) YALI1_E39796g
13) YALI1_F03075g
14) YALI1_E25163g

In other Y. lipolytica isolates, the numbering of the genes will be different, but corresponding homologues exist. Next, we individually knocked out each β-glucosidase-encoding gene in strain W29 expressing Cas9 protein for convenient genome-editing (ST6512). The knock-out was accomplished by replacing the open reading frame of the target gene with hygromycin-resistance cassette, and the transformants were selected on YPD supplemented with hygromycin. The constructed knock-out cells were then plated on esculin glycerol agar (EGA) media (FIG. 5c). The yeast cells with β-glucosidase activity are supposed to hydrolyze the substrate and develop a dark brown colour in the agar. Considering that the diameter of the brown halo is directly correlated to β-glucosidase activity, the most promising targets for reducing β-glucosidase activity are YALI1_B18845g, YALI1_B18887g, YALI1_F08075g and YALI1_E39796g. Further investigation of β-glucosidase-KO strains for their ability to hydrolyze betanin into aglycone form (betanidin) showed that deletion of every single β-glucosidase-encoding gene would decrease the betanidin formation compared to wild type (FIG. 5d). More importantly, deletion of β-glucosidases YALI1_B18845g and/or YALI1_B18887g results in significant suppression of betanidin formation from betanin by yeast cells. Deletion of one or more of these β-glucosidase-encoding genes in betalain-producing yeast strains will lead to improvement of betalains production and decrease the formation of by-products and, consequently, reduce the discolouration.

### Example 8—Chemical Inhibition of β-Glucosidases to Improve Betanin Stability

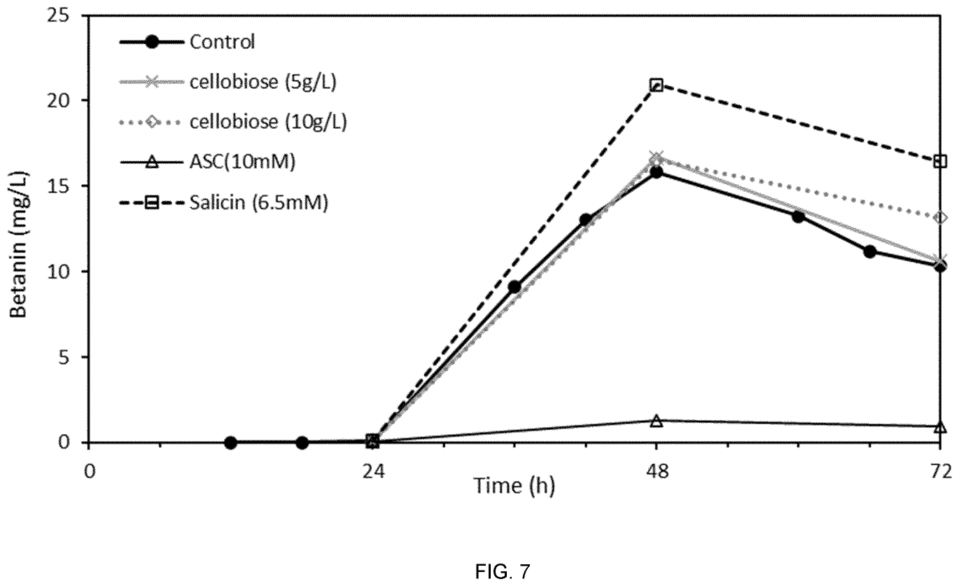

We tested whether inhibition of β-glucosidase activity by some chemicals may improve the titer and purity of betalains. For this, Y. lipolytica strain with integrated betanin production pathway (ST12603) was cultivated in 250-mL shake flasks containing 50 mL mineral medium with 40 g/L glucose (the control in FIG. 7). We further supplemented the medium with different chemicals that may inhibit glucosidase activity, such as cellobiose (final concentrations of 5 g/L or 10 g/L), ascorbic acid (ASC, final concentration of 10 mM), and salicin (final concentration of 6.5 mM). The cultivations were done in biological duplicates, with samples taken out regularly for betanin content measurement in cultures supernatants. Of the tested compounds, supplementation of salicin at 6.5 mM resulted in the highest boost in betanin production titer, with 20.92 mg/L betanin produced compared to 15.80 mg/L in control condition without supplementation. Addition of cellobiose at 10 g/L also resulted in improved betanin titer of 16.53 mg/L. Importantly, the addition of cellobiose (10 g/L) or salicin (6.5 mM) had a profound effect on the fraction of betanin that retained in the fermentation medium after the 48 hours. In control cultivation (no compound added), about 35% of betanin produced at 48 h was degraded at 72 h. However this number was 20% for cultivation with 10 g/L cellobiose, and 21.4% for salicin. In other words, addition of cellobiose (10 g/L) and salicin (6.5 mM) helps to retain larger fraction of betanin that was produced by yeast cells after the production was ceased at 48 h. Thus, addition of glucosidase inhibitors improves the titer of betalains and prevents their breakdown and formation of by-products.

### Example 9—Improving Betalains Production by Preventing Deglycosylation

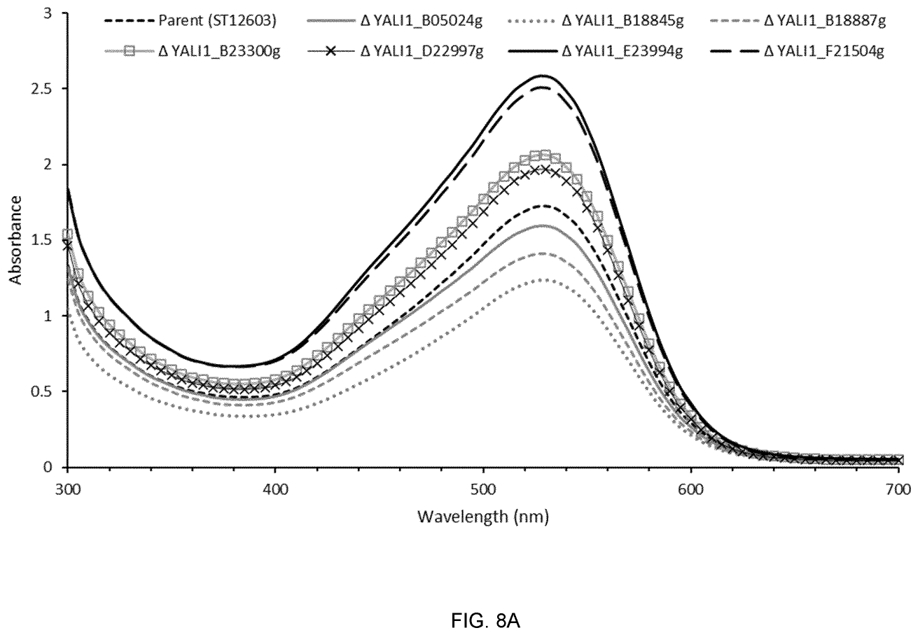

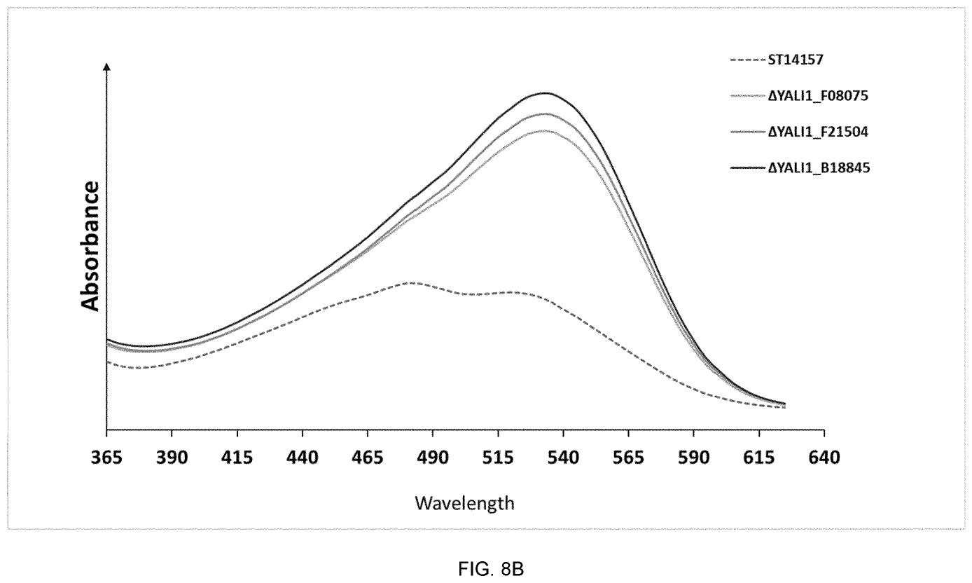

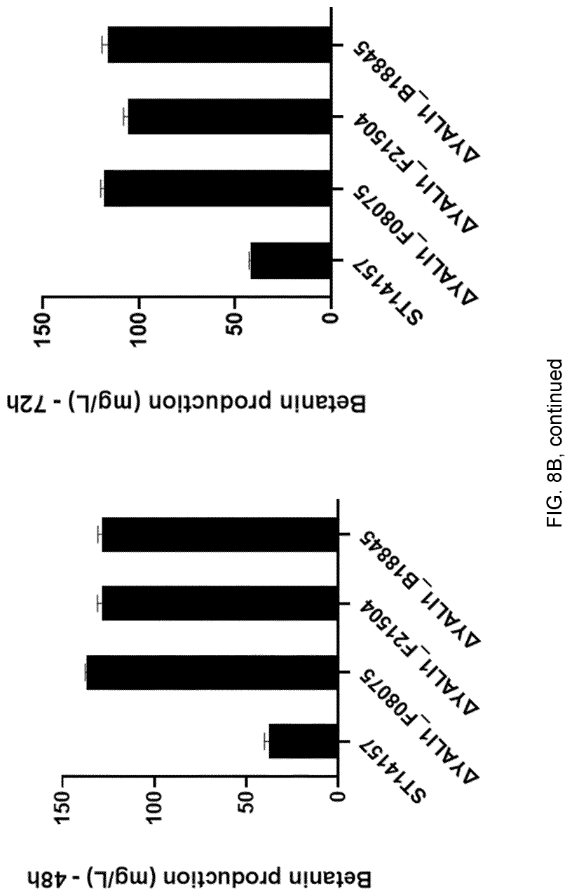

To see the effect of β-glucosidase knockout on betalain production, we deleted seven β-glucosidases YALI1_B05024g (SEQ ID NO: 63), YALI1_B18845g (SEQ ID NO: 64), YALI1_B18887g (SEQ ID NO: 65), YALI1_B23300g (SEQ ID NO: 61), YALI1_D22997g (SEQ ID NO: 66), YALI1_E23994g (SEQ ID NO: 79), and YALI1_F21504g (SEQ ID NO: 62) in betanin-producing platform strain ST12603, and the constructed yeast strains were tested for betalain production. The strains were cultivated in 250-mL shake flasks containing 50 mL mineral medium with 40 g/L glucose for 96 hours, and the UV-vis spectra of the culture supernatant was analyzed (FIG. 8a). For the strains with deleted YALI1_D22997g (SEQ ID NO: 66), YALI1_B23300g (SEQ ID NO: 61), YALI1_F21504g (SEQ ID NO: 62), and YALI1_E23994g (SEQ ID NO: 82) there was higher accumulation of betacyanins (with maximum absorbance level at 540 nm) as there was higher peak areas for these strains compared to their parent (ST12603).

Similar improvement was observed for another strain of Y. lipolytica (ST14157), which contains three copies of betanin biosynthesis pathway genes, some genetic modifications that improve betanin precursor supply and has the 4HPPD gene deletion. In this strain, we knocked out three β-glucosidases YALI1_F08075g (SEQ ID NO: 69), YALI1_B18845g (SEQ ID NO: 64), and YALI1_F21504g (SEQ ID NO: 62) and tested these strains for betalain production (FIG. 8b). Here, we also saw a dramatic increase in betacyanin accumulation in final product for β-glucosidase-deleted strain compared to the parent strain ST14517. We then knocked out three β-glucosidases YALI1_F08075g (SEQ ID NO: 69), YALI1_B18845g (SEQ ID NO: 64), and YALI1_F21504g (SEQ ID NO: 62) in ST14157 and tested these strains for betalain production (FIG. 8b). Here, we also saw a dramatic increase in betacyanin accumulation in final product for β-glucosidase deleted strain compared to parent (ST14157). Furthermore, deletion of β-glucosidases resulted in higher fraction of betacyanins (max. absorbance at 540 nm) compared to betaxanthins (max. absorbance at 475 nm), which is due to abolishing of betacyanin-degrading enzymes that would break down the red betacyanins to yellow betaxanthins and betalamic acid. Overall, deletion of β-glucosidases show a stabilizing effect on betacyanins production and increased betanin titers in yeast cell factories.

### Example 10—Gram-Scale Production of Phyllocactin and Amaranthin in Fed-Batch Fermentations

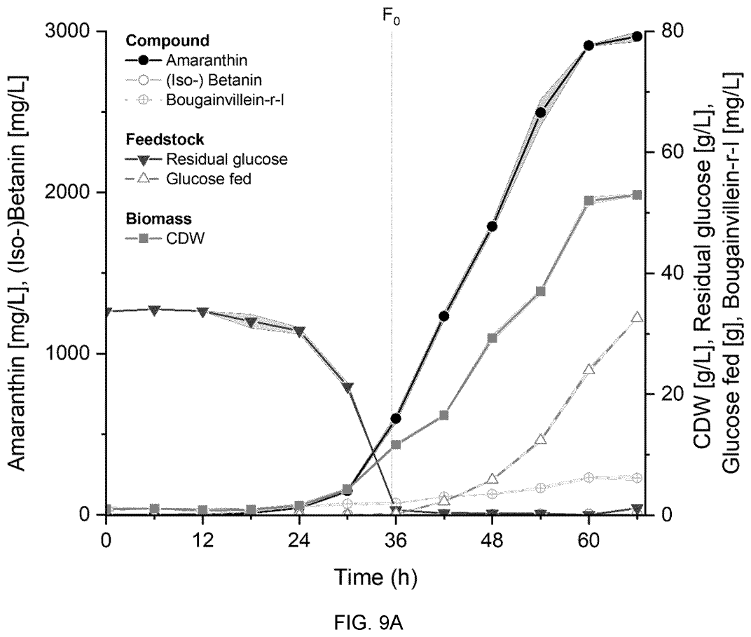

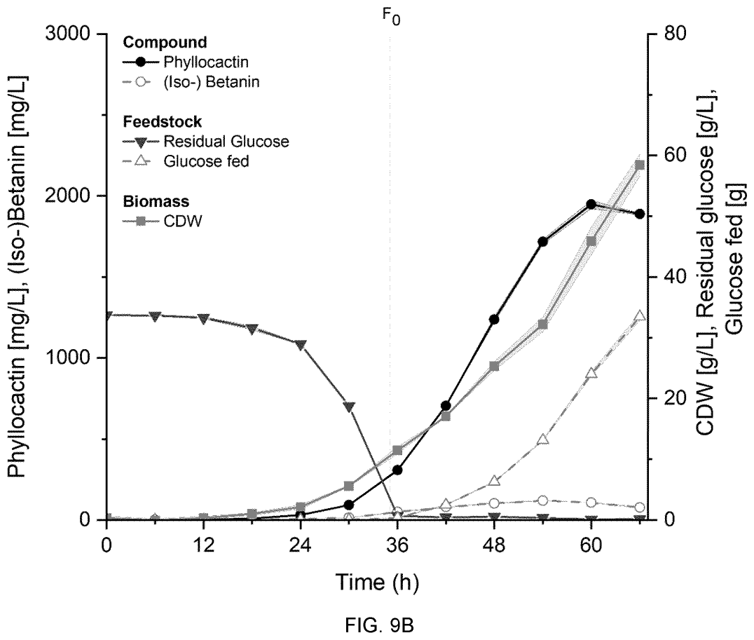

The previously constructed Y. lipolytica strains ST14103, expressing the BAHD acyltransferase HpBAHD3 (SEQ ID NO: 29, 31) and the Y. lipolytica strain ST14102, expressing the glucuronosyltransferase CcAmaSy1 (SEQ ID NO: 44, 46), were cultivated in a fed-batch fermentation in 250 mL bioreactors (AMBR250) in duplicates. The cultivations were performed at 30° C. in MM (−pABA) with 4% D-glucose in the batch media and a starting OD of 1. Samples were taken every 6 h, and the production of betalains (intra- and extracellular) and glucose concentration in the media was quantified by HPLC. Betanin and isobetanin were quantified as described before (Example 1) using a commercially available betanin standard. Bougainvillein-r-I, phyllocactin and amaranthin were quantified with a standard made from the respective variants isolated from plants (Example 2). The pure compounds were purified from the plant extracts by preparative HPLC (Example 1), up-concentrated, and by using the Beer-Lambert equation assuming a molar extinction coefficient of ε=6.5×10⁴ M⁻¹cm⁻¹ for all three betalain variants, calibration curves were made. The highest titer of amaranthin was achieved at the end of the fermentation (66 h) when 3 g/L amaranthin had been produced with ST14102 (FIG. 9a). With ST14103, a titer of 1.9 g/L phyllocactin was reached after 60 h of fermentation (FIG. 9b). Both strains produced mainly the compound of interest and only minimal amounts of (iso-)betanin or bougainvillein-r-I.

### Example 11—Comparison of the Color Characteristics of Pure Amaranthin and Phyllocactin to Betanin

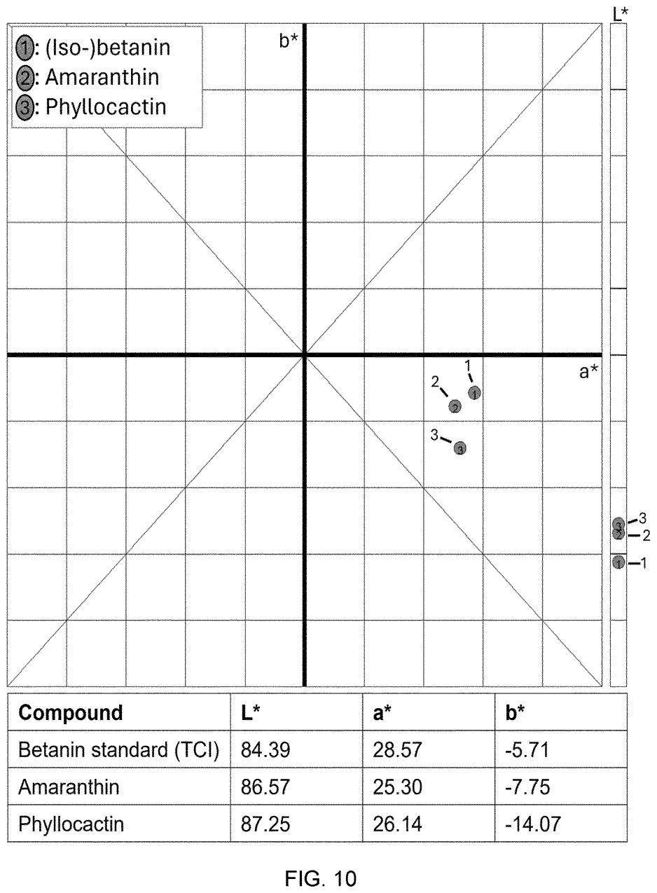

The main use of betalains is as food colourants. Characterising the colour of the newly produced variants is therefore of high interest. For that purpose, amaranthin and phyllocactin were purified from the fermentation broth of ST14102 and ST14103 cultivations. The fermentation broth was centrifuged, and the supernatant was sterile-filtered (0.2 μm filter). Afterwards, the supernatant was analysed by preparative HPLC (Example 1) and fractions corresponding to pure amaranthin and phyllocactin were taken. These samples, containing only the compound of interest and no betaxanthins or other betalains produced in the fermentation, were diluted to OD₅₃₅=0.345 in citrate-phosphate buffer (pH 4) made from 0.2 M Na₂HPO₄ and 0.1 M citric acid. A commercial betanin standard (TCI America), which contains only betanin and isobetanin, was also diluted to OD₅₃₅=0.345 in the same buffer. The thus prepared pure betalain samples were analysed with a HunterLab Vista spectrophotometer according to the CIELAB colour space (FIG. 10). The three betalains have different shades as distinguishable by eye and by spectrophotometer measurements. For example, phyllocactin has a higher violet/blue shade than betanin, which is also aligned with a lower b* value.

### Example 12—Deletions of Beta-Glucosidases—Effect on Betanin Production in Small-Scale and Bioreactors

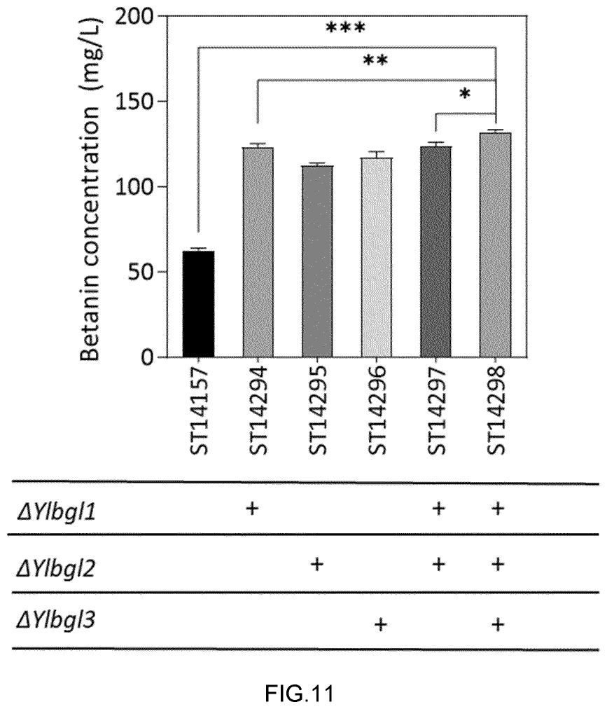

To improve the stability of betanin, several beta-glucosidases, which can potentially degrade beta-glucosidases, with high activities were deleted. Three main beta-glucosidases (YIBLGL1 (YALI1_F21504g (SEQ ID NO: 62)), YIBGL2 (YALI1_B18845g (SEQ ID NO: 64)) and YIBGL3 (YALI1_F08075g (SEQ ID NO: 69)) were tested. As FIG. 11 shows, after 48 hours of culture in 24 deep well plates with MM medium, compared with parental strain ST14157, deletion of any of YIBGL1, YIBGL2, and YIBGL3 improved betanin titer from 63 mg/L (parental strain) to 112-123 mg/L. However, the double knockout mutant (Δbgl1Δbgl2) did not differ from the single Δbgl1 or Δbgl2 or Δbgl3 strains. The triple knockout strain gave a higher titer of 131.76±0.74 mg/L.

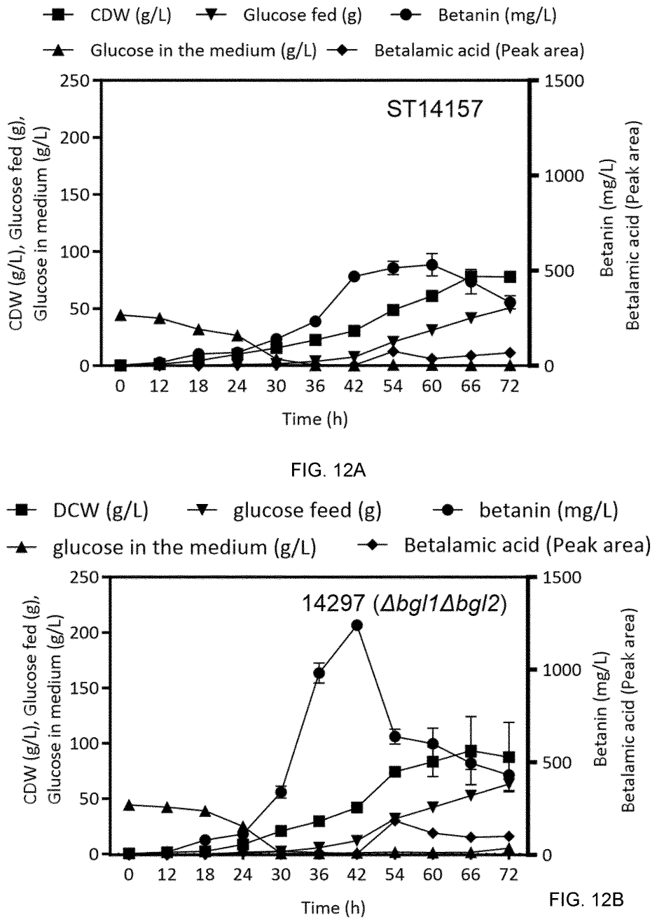

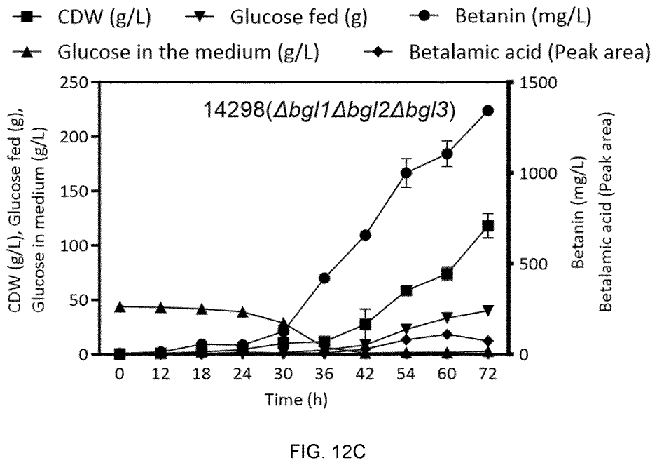

The performance of the parental strain ST14157, double beta-glucosidases knockout strain (Δbgl1Δbgl2) and triple beta-glucosidases knockout strain (Δbgl1Δbgl2Δbgl3) were validated in fed-batch bioreactors. The fermentations were carried in two biological replicates in AMBR® 250 fermenters with 250 mL working volume. After an 18-hour batch phase on 40 g/L glucose, the fed-batch was started with exponential feeding of glucose and salt solution. As FIG. 12a and FIG. 12b shows, the betanin titer of the double beta-glucosidases knockout strain significantly improved, which achieved 1.24 g/L, compared with the parental strain ST14157 (0.53 g/L). However, after reaching the peak value, betanin titer decreased both in the control strain ST14157 and in the double beta-glucosidases knockout strain (Δbgl1Δbgl2). But, the betanin degradation did not happen on the triple glucosidase knockout strain (Δbgl1Δbgl2Δbgl3), which reached 1.34 g/L at 72 hours (FIG. 12c).

TABLE 1
Sequence overview

| SEQ | |
|---|---|
| ID NO | Description |
| 1 | MjDOD protein from Mirabilis jalapa (garden four-o'clock), |
| | NCBI reference sequence: B6F0W8.1 |
| 2 | Gene for MjDOD from Mirabilis jalapa codon-optimized for |
| | S. cerevisiae |
| 3 | Gene for MjDOD from Mirabilis jalapa codon-optimized for |
| | Y. lipolytica (MjDOD_YI) |
| 4 | BvCYP76AD1^{W13L} protein from Beta vulgaris, |
| | GenBank: AET43289.1 |
| 5 | Gene for BvCYP76AD1^{W13L} from Beta vulgaris, |
| | codon-optimized for S. cerevisiae |
| 6 | EvTYH protein from Ercilla volubilisi, GenBank: AKI33945.1 |
| 7 | Gene for EvTYH from Ercilla volubilisi codon-optimized for |
| | Y. lipolytica (EvTYH_YI) |
| 8 | BvSGT2 protein from Beta vulgaris, |
| | NCBI reference sequence: XP_010691725.1 |
| 9 | Gene for BvSGT2 from Beta vulgaris |
| | codon-optimized for S. cerevisiae |
| 10 | Gene for BvSGT2 from Beta vulgaris |
| | codon-optimized for Y. lipolytica (BvSGT2_YI) |
| 11 | BgGT2 protein from Bougainvillea glabra |
| 12 | Gene for BgGT2 protein from Bougainvillea glabra |
| | codon-optimized for S. cerevisiae |
| 13 | ARO4^{K221L} protein in Y. lipolytica |
| 14 | Gene for ARO4^{K221L} |
| 15 | ARO7^{G139S} protein in Y. lipolytica |
| 16 | Gene for ARO7^{G139S} |
| 17 | 4-hydroxyphenylpyruvate dioxygenase (4-HPPD) protein |
| | sequence from Yarrowia lipolytica, UniPROT reference |
| | number: A0A1H6PW31 |
| 18 | Gene for 4-hydroxyphenylpyruvate dioxygenase from |
| | Yarrowia lipolytica, UniPROT reference number: |
| | YALI1_B28454g |
| 19 | CcBAHD1 protein from Celosia cristata |
| 20 | Gene for CcBAHD1 from Celosia cristata codon-optimized |
| | for S. cerevisiae |
| 21 | CcBAHD2 protein from Celosia cristata |
| 22 | Gene for CcBAHD2 from Celosia cristata codon-optimized |
| | for S. cerevisiae |
| 23 | CcBAHD3 protein from Celosia cristata |
| 24 | Gene for CcBAHD3 from Celosia cristata codon-optimized |
| | for S. cerevisiae |
| 25 | HpBAHD1 protein from Hylocereus polyrhizus |
| 26 | Gene for HpBAHD1 from Hylocereus polyrhizus |
| | codon-optimized for S. cerevisiae |
| 27 | HpBAHD2 protein from Hylocereus polyrhizus |
| 28 | Gene for HpBAHD2 from Hylocereus polyrhizus |
| | codon-optimized for S. cerevisiae |
| 29 | HpBAHD3 protein from Hylocereus polyrhizus |
| 30 | Gene for HpBAHD3 from Hylocereus polyrhizus |
| | codon-optimized for S. cerevisiae |
| 31 | Gene for HpBAHD3 from Hylocereus polyrhizus |
| | codon-optimized for Y. lipolytica (HpBAHD3_YI) |
| 32 | HpBAHD4 protein from Hylocereus polyrhizus |
| 33 | Gene for HpBAHD4 from Hylocereus polyrhizus |
| | codon-optimized for S. cerevisiae |
| 34 | HpBAHD5 protein from Hylocereus polyrhizus |
| 35 | Gene for HpBAHD5 from Hylocereus polyrhizus |
| | codon-optimized for S. cerevisiae |
| 36 | HpBAHD6 protein from Hylocereus polyrhizus |
| 37 | Gene for HpBAHD6 from Hylocereus polyrhizus |
| | codon-optimized for S. cerevisiae |
| 38 | AhAmaSy1 protein from Amaranthus hypochondriacus |
| 39 | Gene for AhAmaSy1 from Amaranthus hypochondriacus |
| | codon-optimized for S. cerevisiae |
| 40 | Gene for AhAmaSy1 from Amaranthus hypochondriacus |
| | codon-optimized for Y. lipolytica |
| 41 | CqAmaSy1 protein from Chenopodium quinoa |
| 42 | Gene for CqAmaSy1 from Chenopodium quinoa |
| | codon-optimized for S. cerevisiae |
| 43 | Gene for CqAmaSy1 from Chenopodium quinoa |
| | codon-optimized for Y. lipolytica |
| 44 | CcAmaSy1 protein from Celosia argentea var. cristata |
| 45 | Gene for CcAmaSy1 from Celosia argentea var. cristata |
| | codon-optimized for S. cerevisiae |
| 46 | Gene for CcAmaSy1 from Celosia argentea var. cristata |
| | codon-optimized for Y. lipolytica |
| 47 | AtUGD1 protein from Arabidopsis thaliana |
| 48 | Gene for AtUGD1 from Arabidopsis thaliana |
| 49 | AnTYH protein from Abronia nealleyi, |
| | GenBank: AKI33952.1 |
| 50 | Gene for AnTYH from Abronia nealleyi, codon-optimized |
| | for S. cerevisiae |
| 51 | Gene for AnTyH from Abronia nealleyi, codon-optimized |
| | for Y. lipolytica |
| 52 | BgDOD2 protein from Bougainvillea glabra, |
| | GenBank: BAG80687.1 |
| 53 | Gene for BgDOD2 from Bougainvillea glabra, |
| | codon-optimized for S. cerevisiae |
| 54 | Gene for BgDOD2 from Bougainvillea glabra, |
| | codon-optimized for Y. lipolytica |
| 55 | PgDOD protein from Portulaca grandiflora, |
| | NCBI reference sequence: CAE45178.1 |
| 56 | Gene for PgDOD from Portulaca grandiflora, |
| | codon-optimized for S. cerevisiae |
| 57 | CqSGT2 protein sequence from Chenopodium quinoa, NCBI |
| | reference sequence: XP_021753346.1 |
| 58 | Gene for CqSGT2 from Chenopodium quinoa, |
| | codon-optimized for S. cerevisiae |
| 59 | BvSGT4 protein from Beta vulgaris, NCBI reference |
| | sequence: XP_010691730.1 |
| 60 | Gene for BvSGT4 from Beta vulgaris, codon-optimized |
| | for S. cerevisiae |
| 61 | YALI1_B23300g |
| 62 | YALI1_F21504g |
| 63 | YALI1_B05024g |
| 64 | YALI1_B18845g |
| 65 | YALI1_B18887g |
| 66 | YALI1_D22997g |
| 67 | YALI1_E40502g |
| 68 | YALI1_F02592g |
| 69 | YALI1_F08075g |
| 70 | YALI1_F17788g |
| 71 | 3-deoxy-7-phosphoheptulonate synthase (Aro4) protein |
| | sequence from Yarrowia lipolytica, UniPROT/NCBI |
| | reference number: A0A1D8N9Z6; AOW02456.1 |
| 72 | Gene for Aro4 from Yarrowia lipolytica, UniPROT reference |
| | number: YALI1_C09308g |
| 73 | Chorismate mutase (Aro7) protein sequence from |
| | Yarrowia lipolytica, UniPROT/NCBI reference number: |
| | A0A1D8NIT5; AOW05549.1 |
| 74 | Gene for Aro7 from Yarrowia lipolytica, UniPROT reference |
| | number: YALI1_E20751g |
| 75 | 3-deoxy-7-phosphoheptulonate synthase (Aro4) protein |
| | sequence from Saccharomyces cerevisiae, UniPROT |
| | reference number: QHB07012.1 |
| 76 | Gene for Aro4 from Saccharomyces cerevisiae, |
| | UniPROT/NCBI reference number: SCEN0B03630; YBR249C |
| 77 | Chorismate mutase (Aro7) protein sequence from |
| | Saccharomyces cerevisiae, UniPROT reference |
| | number: QHB12310.1 |
| 78 | Gene for Aro7 from Saccharomyces cerevisiae, |
| | UniPROT/NCBI reference number: SCEN0P03550; YPR060C |
| 79 | YALI1_E23994g |
| 80 | YALI1_E39796g |
| 81 | YALI1_F03075g |
| 82 | YALI1_E25163g |
| 83 | AoTYH protein from Acleisanthes obtusa, |
| | GenBank: AKI33950.1 |
| 84 | BaTYH protein from Basella alba, GenBank: AJD87470.1 |
| 85 | CbTYH protein from Cleretum bellidiforme, |
| | GenBank: AJD87468.1 |
| 86 | MmTYH1 protein from Mirabilis multiflora, |
| | GenBank: AKI33948.1 |
| 87 | MmTYH2 protein from Mirabilis multiflora, |
| | GenBank: AKI33937.1 |
| 88 | OfTYH protein from Opuntia ficus-indica, |
| | GenBank: AJD87464.1 |
| 89 | PaTYH protein from Phytolacca americana, |
| | GenBank: AJD87467.1 |
| 90 | PdTYH protein from Phytolacca dioica, |
| | GenBank: AKI33942.1 |
| 91 | AtDOD protein from Amaranthus tricolour, |
| | GenBank: AJW81119.1 |
| 92 | AhDOD protein from Amaranthus hypochondriacus |
| | (grain amaranth), GenBank: ADZ48644.1 |
| 93 | BvDOD1 protein from Beta vulgaris, NCBI reference |
| | sequence: Q70FG7.1 |
| 94 | BvDOD2 protein from Beta vulgaris, GenBank: AET43293.1 |
| 95 | BvDOD3 protein from Beta vulgaris, |
| | GenBank: AET43287.1 |
| 96 | BgDOD1 protein from Bougainvillea glabra, |
| | GenBank: ASW22755.1 |
| 97 | PaDOD protein from Phytolacca americana (American |
| | pokeweed), NCBI reference sequence: BAH66635.1 |
| 98 | SoDOD protein from Spinacia oleracea (spinach), NCBI |
| | reference sequence: XP_021836119.1 |
| 99 | SsDOD protein from Suaeda salsa, |
| | GenBank: ACO59903.1 |
| 217 | YALI0F16027g |
| 218 | YALI0B14289g |
| 219 | YALI0F05390g |

TABLE 2
BioBricks

| BioBrick name | Fw-primer | Rv-primer | Template DNA |
|---|---|---|---|
| BB0008 (PTEF1<-) | PR-1750 | PR-6 | CEN.PK 113-7D |
| | | | genomic DNA |
| BB0010 (<-PTEF1- | PR-1750 | PR-8 | Synthetic DNA |
| PPGK1->) | | | |
| BB3908 | PR-23995 | PR-23967 | Synthetic DNA |
| (PrTEFin-> _single) | | | |
| BB4151 (DOD_U1) | PR-25066 | PR-25067 | Synthetic DNA |
| BB4440 (TyrH | PR-25064 | PR-25065 | Synthetic DNA |
| (W13L)_U2) | | | |
| BB5049 | PR-27980 | PR-27981 | Synthetic DNA |
| (MjDOD_U1_Gene 1) | | | |
| BB5001 | PR-27830 | PR-27831 | Synthetic DNA |
| (YI_EvTYH_Tefint_U2) | | | |
| BB5052 | PR-27948 | PR-27949 | Synthetic DNA |
| (BvSGT2_Gene1) | | | |
| BB5218 | PR-28834 | PR-28835 | Synthetic DNA |
| (YI_BvSGT2_G2) | | | |
| BB5715 (BgGT2_Gene1) | PR-25056 | PR-27831 | Synthetic DNA |
| BB5894 (BvSGT2_YI_W/ | PR-29863 | PR-29970 | Synthetic DNA |
| O_ATG-U1) | | | |
| BB5898 | PR-23967 | PR-25055 | Synthetic DNA |
| (PrTEFin_single-U1) | | | |
| BB5893 (TPex20<- | PR-25056 | PR-27831 | Synthetic DNA |
| MjDOD_YI<- | | | |
| PrGPD::PrTEFin-> | | | |
| EvTYH_YI-> TLip2) | | | |
| BB5998 (HphSyn-Loxp- | PR-31421 | PR-31422 | Synthetic DNA |
| cassette) | (HphSyn-U1 Fwd) | (HphSyn-U2 Rev) | |
| BB6208 | PR-31983 | PR-31984 | W29 genomic DNA |
| (YALI1_B05024g-UP) | (YALI1_B05024g- | (YALI1_B05024g- | |
| | UP Fwd) | UP Rev) | |
| BB6209 | PR-31985 | PR-31986 | W29 genomic DNA |
| (YALI1_B05024g-DW) | (YALI1_B05024g- | (YALI1_B05024g- | |
| | DW Fwd) | DW Rev) | |
| BB6210 | PR-31987 | PR-31988 | W29 genomic DNA |
| (YALI1_B18845g-UP) | (YALI1_B18845g- | (YALI1_B18845g- | |
| | UP Fwd) | UP Rev) | |
| BB6211 | PR-31989 | PR-31990 | W29 genomic DNA |
| (YALI1_B18845g-DW) | (YALI1_B18845g- | (YALI1_B18845g- | |
| | DW Fwd) | DW Rev) | |
| BB6212 | PR-31991 | PR-31992 | W29 genomic DNA |
| (YALI1_B18887g-UP) | (YALI1_B18887g- | (YALI1_B18887g- | |
| | UP Fwd) | UP Rev) | |
| BB6213 | PR-31993 | PR-31994 | W29 genomic DNA |
| (YALI1_B18887g-DW) | (YALI1_B18887g- | (YALI1_B18887g- | |
| | DW Fwd) | DW Rev) | |
| BB6214 | PR-31995 | PR-31996 | W29 genomic DNA |
| (YALI1_B23300g-UP) | (YALI1_B23300g- | (YALI1_B23300g- | |
| | UP Fwd) | UP Rev) | |
| BB6215 | PR-31997 | PR-31998 | W29 genomic DNA |
| (YALI1_B23300g-DW) | (YALI1_B23300g- | (YALI1_B23300g- | |
| | DW Fwd) | DW Rev) | |
| BB6216 | PR-31999 | PR-32000 | W29 genomic DNA |
| (YALI1_D22997g-UP) | (YALI1_D22997g- | (YALI1_D22997g- | |
| | UP Fwd) | UP Rev) | |
| BB6217 | PR-32001 | PR-32002 | W29 genomic DNA |
| (YALI1_D22997g-DW) | (YALI1_D22997g- | (YALI1_D22997g- | |
| | DW Fwd) | DW Rev) | |
| BB6218 | PR-32003 | PR-32004 | W29 genomic DNA |
| (YALI1_E23994g-UP) | (YALI1_E23994g- | (YALI1_E23994g- | |
| | UP Fwd) | UP Rev) | |
| BB6219 | PR-32005 | PR-32006 | W29 genomic DNA |
| (YALI1_E23994g-DW) | (YALI1_E23994g- | (YALI1_E23994g- | |
| | DW Fwd) | DW Rev) | |
| BB6220 | PR-32007 | PR-32008 | W29 genomic DNA |
| (YALI1_E40502g-UP) | (YALI1_E40502g- | (YALI1_E40502g- | |
| | UP Fwd) | UP Rev) | |
| BB6221 | PR-32009 | PR-32010 | W29 genomic DNA |
| (YALI1_E40502g-DW) | (YALI1_E40502g- | (YALI1_E40502g- | |
| | DW Fwd) | DW Rev) | |
| BB6222 | PR-32011 | PR-32012 | W29 genomic DNA |
| (YALI1_F02592g-UP) | (YALI1_F02592g- | (YALI1_F02592g- | |
| | UP Fwd) | UP Rev) | |
| BB6223 | PR-32013 | PR-32014 | W29 genomic DNA |
| (YALI1_F02592g-DW) | (YALI1_F02592g- | (YALI1_F02592g- | |
| | DW Fwd) | DW Rev) | |
| BB6224 | PR-32015 | PR-32016 | W29 genomic DNA |
| (YALI1_F08075g-UP) | (YALI1_F08075g- | (YALI1_F08075g- | |
| | UP Fwd) | UP Rev) | |
| BB6225 | PR-32017 | PR-32018 | W29 genomic DNA |
| (YALI1_F08075g-DW) | (YALI1_F08075g- | (YALI1_F08075g- | |
| | DW Fwd) | DW Rev) | |
| BB6226 | PR-32019 | PR-32020 | W29 genomic DNA |
| (YALI1_F17788g-UP) | (YALI1_F17788g- | (YALI1_F17788g- | |
| | UP Fwd) | UP Rev) | |
| BB6227 | PR-32021 | PR-32022 | W29 genomic DNA |
| (YALI1_F17788g-DW) | (YALI1_F17788g- | (YALI1_F17788g- | |
| | DW Fwd) | DW Rev) | |
| BB6228 | PR-32023 | PR-32024 | W29 genomic DNA |
| (YALI1_F21504g-UP) | (YALI1_F21504g- | (YALI1_F21504g- | |
| | UP Fwd) | UP Rev) | |
| BB6229 | PR-32025 | PR-32026 | W29 genomic DNA |
| (YALI1_F21504g-DW) | (YALI1_F21504g- | (YALI1_F21504g- | |
| | DW Fwd) | DW Rev) | |
| BB6230 (YALI1_B05024g- | PR-31983 | PR-31986 | Ligation product of |
| HphSyn-LoxP) | (YALI1_B05024g- | (YALI1_B05024g- | BB5998, BB6208, |
| | UP Fwd) | DW Rev) | BB6209 |
| BB6231 (YALI1_B18845g- | PR-31987 | PR-31990 | Ligation product of |
| HphSyn-LoxP) | (YALI1_B18845g- | (YALI1_B18845g- | BB5998, BB6210, |
| | UP Fwd) | DW Rev) | BB6211 |
| BB6232 (YALI1_B18887g- | PR-31991 | PR-31994 | Ligation product of |
| HphSyn-LoxP) | (YALI1_B18887g- | (YALI1_B18887g- | BB5998, BB6212, |
| | UP Fwd) | DW Rev) | BB6213 |
| BB6233 (YALI1_B23300g- | PR-31995 | PR-31998 | Ligation product of |
| HphSyn-LoxP) | (YALI1_B23300g- | (YALI1_B23300g- | BB5998, BB6214, |
| | UP Fwd) | DW Rev) | BB6215 |
| BB6234 (YALI1_D22997g- | PR-31999 | PR-32002 | Ligation product of |
| HphSyn-LoxP) | (YALI1_D22997g- | (YALI1_D22997g- | BB5998, BB6216, |
| | UP Fwd) | DW Rev) | BB6217 |
| BB6235 (YALI1_E23994g- | PR-32003 | PR-32006 | Ligation product of |
| HphSyn-LoxP) | (YALI1_E23994g- | (YALI1_E23994g- | BB5998, BB6218, |
| | UP Fwd) | DW Rev) | BB6219 |
| BB6236 (YALI1_E40502g- | PR-32007 | PR-32010 | Ligation product of |
| HphSyn-LoxP) | (YALI1_E40502g- | (YALI1_E40502g- | BB5998, BB6220, |
| | UP Fwd) | DW Rev) | BB6221 |
| BB6237 (YALI1_F02592g- | PR-32011 | PR-32014 | Ligation product of |
| HphSyn-LoxP) | (YALI1_F02592g- | (YALI1_F02592g- | BB5998, BB6222, |
| | UP Fwd) | DW Rev) | BB6223 |
| BB6238 (YALI1_F08075g- | PR-32015 | PR-32018 | Ligation product of |
| HphSyn-LoxP) | (YALI1_F08075g- | (YALI1_F08075g- | BB5998, BB6224, |
| | UP Fwd) | DW Rev) | BB6225 |
| BB6239 (YALI1_F17788g- | PR-32019 | PR-32022 | Ligation product of |
| HphSyn-LoxP) | (YALI1_F17788g- | (YALI1_F17788g- | BB5998, BB6226, |
| | UP Fwd) | DW Rev) | BB6227 |
| BB6240 (YALI1_F21504g- | PR-32023 | PR-32026 | Ligation product of |
| HphSyn-LoxP) | (YALI1_F21504g- | (YALI1_F21504g- | BB5998, BB6228, |
| | UP Fwd) | DW Rev) | BB6229 |
| BB6365 | PR-32308 | PR-32309 | W29 genomic DNA |
| (YALI1_E25163g-UP) | (YALI1_E25163g- | (YALI1_E25163g- | |
| | UP Fwd) | UP Rev) | |
| BB6366 | PR-32310 | PR-32311 | W29 genomic DNA |
| (YALI1_E25163g-DW) | (YALI1_E25163g- | (YALI1_E25163g- | |
| | DW Fwd) | DW Rev) | |
| BB6367 | PR-32312 | PR-32313 | W29 genomic DNA |
| (YALI1_E39796g-UP) | (YALI1_E39796g- | (YALI1_E39796g- | |
| | UP Fwd) | UP Rev) | |
| BB6368 | PR-32314 | PR-32315 | W29 genomic DNA |
| (YALI1_E39796g-DW) | (YALI1_E39796g- | (YALI1_E39796g- | |
| | DW Fwd) | DW Rev) | |
| BB6369 | PR-32316 | PR-32317 | W29 genomic DNA |
| (YALI1_F03075g-UP) | (YALI1_F03075g- | (YALI1_F03075g- | |
| | UP Fwd) | UP Rev) | |
| BB6370 | PR-32318 | PR-32319 | W29 genomic DNA |
| (YALI1_F03075g-DW) | (YALI1_F03075g- | (YALI1_F03075g- | |
| | DW Fwd) | DW Rev) | |
| BB6371 (YALI1_E25163g- | PR-32308 | PR-32311 | Ligation product of |
| HphSyn-LoxP) | (YALI1_E25163g- | (YALI1_E25163g- | BB5998, BB6365, |
| | UP Fwd) | DW Rev) | BB6366 |
| BB6372 (YALI1_E39796g- | PR-32312 | PR-32315 | Ligation product of |
| HphSyn-LoxP) | (YALI1_E39796g- | (YALI1_E39796g- | BB5998, BB6367, |
| | UP Fwd) | DW Rev) | BB6368 |
| BB6373 (YALI1_F03075g- | PR-32316 | PR-32319 | Ligation product of |
| HphSyn-LoxP) | (YALI1_F03075g- | (YALI1_F03075g- | BB5998, BB6369, |
| | UP Fwd) | DW Rev) | BB6370 |
| BB6559 (CcBAHD1_U1) | PR-32543 | PR-32544 | Synthetic DNA |
| BB6560 (CcBAHD2_U1) | PR-32545 | PR-32546 | Synthetic DNA |
| BB6561 (CcBAHD3_U1) | PR-32547 | PR-32548 | Synthetic DNA |
| BB6562 (HpBAHD1_U1) | PR-32549 | PR-32550 | Synthetic DNA |
| BB6563 (HpBAHD2_U1) | PR-32551 | PR-32552 | Synthetic DNA |
| BB6564 (HpBAHD3_U1) | PR-32553 | PR-32554 | Synthetic DNA |
| BB6565 (HpBAHD4_U1) | PR-32555 | PR-32556 | Synthetic DNA |
| BB6566 (HpBAHD5_U1) | PR-32557 | PR-32558 | Synthetic DNA |
| BB6567 (HpBAHD6_U1) | PR-32559 | PR-32560 | Synthetic DNA |
| BB6571 (AhAmaSy1_U1) | PR-32630 | PR-32631 | Synthetic DNA |
| BB6572 (CqAmaSy1_U1) | PR-32632 | PR-32633 | Synthetic DNA |
| BB6573 (CcAmaSy1_U1) | PR-32634 | PR-32635 | Synthetic DNA |
| BB6700 (AtUGD1_U1) | PR-33125 | PR-33126 | Synthetic DNA |
| BB6732 | PR-33146 | PR-33147 | Synthetic DNA |
| BB6733 | PR-33150 | PR-33151 | Synthetic DNA |
| BB6734 | PR-33154 | PR-33155 | Synthetic DNA |
| BB6735 | PR-33144 | PR-33145 | Synthetic DNA |
| BB6738 | PR-33237 | PR-23967 | W29 genomic DNA |
| BB6739 | PR-33237 | PR-33238 | W29 genomic DNA |
| BB6750 | PR-33156 | PR-33157 | Synthetic DNA |

TABLE 3
Integrative, episomal and helper (gRNA) vectors

| | | DNA elements to |
|---|---|---|
| Name | Type of vector | construct/Reference |
| pCfB2312 (TEF1p-Cas9- | episomal vector for | (Jessop-Fabre et al. 2016) |
| CYC1t_kanMX) | Cas9p expression | |
| pCfB2899 (X-2-MarkerFree) | Plasmid backbone | (Jessop-Fabre et al. 2016) |
| | for integrative | |
| | vectors | |
| pCfB2903 (XI-2- | Plasmid backbone | (Jessop-Fabre et al. 2016) |
| MarkerFree) | for integrative | |
| | vectors | |
| pCfB2909 (XII-5- | Plasmid backbone | (Jessop-Fabre et al. 2016) |
| MarkerFree) | for integrative | |
| | vectors | |
| pCfB3020 (p-gRNA-X-2) | gRNA vector | (Jessop-Fabre et al. 2016) |
| pCfB3034 (X-3-MarkerFree) | Plasmid backbone | (Jessop-Fabre et al. 2016) |
| | for integrative | |
| | vectors | |
| pCfB3037 | Plasmid backbone | (Jessop-Fabre et al. 2016) |
| (XI-5-MarkerFree) | for integrative | |
| | vectors | |
| pCfB3041 (p-gRNA-X-3) | gRNA vector | (Jessop-Fabre et al. 2016) |
| pCfB3046 (p-gRNA-XI-5) | gRNA vector | (Jessop-Fabre et al. 2016) |
| pCfB3050 (p-gRNA-XII-5) | gRNA vector | (Jessop-Fabre et al. 2016) |
| pCfB5250 (pIntC_2- | Plasmid backbone | (Holkenbrink et al. 2018) |
| TPex20-TLip2) | for integrative | |
| | vectors | |
| pCfB6370 | Plasmid backbone | (Holkenbrink et al. 2018) |
| (pIntF_3-TPex20-TLip2) | for integrative | |
| | vectors | |
| pCfB6371 | Plasmid backbone | (Holkenbrink et al. 2018) |
| (pIntC_3-TPex20-TLip2) | for integrative | |
| | vectors | |
| pCfB6630 (pNat- | gRNA vector | (Holkenbrink et al. 2018) |
| YLgRNA3_IntC_3) | | |
| pCfB6631 (pNat- | gRNA vector | (Holkenbrink et al. 2018) |
| YLgRNA3_IntD_1) | | |
| pCfB6637 (pNat- | gRNA vector | (Holkenbrink et al. 2018) |
| YLgRNA3_IntE_3) | | |
| pCfB6679 (pIntE_4- | Plasmid backbone | (Holkenbrink et al. 2018) |
| TPex20-TLip2) | for integrative | |
| | vectors | |
| pCfB6681 (pIntE_3- | Plasmid backbone | (Holkenbrink et al. 2018) |
| TPex20-TLip2) | for integrative | |
| | vectors | |
| pCfB6682 (pIntC_2- | Plasmid backbone | (Holkenbrink et al. 2018) |
| TPex20-TLip2) | for integrative | |
| | vectors | |
| pCfB6684 (pIntD_1- | Plasmid backbone | (Holkenbrink et al. 2018) |
| TPex20-TLip2) | for integrative | |
| | vectors | |
| pCfB6364 (pku70-TPex20- | Integrative vector | (Holkenbrink et al. 2018) |
| PrTef-_Cas9-Ttef-Lip2- | for Cas9p | |
| dsdSyn) | expression | |
| pCfB8977 (Int E_1- | Integrative vector | (Sáez-Sáez et al. 2020) |
| tPex20<-YIARO7_G139S<- | | |
| PrGPD::PrTEFin-> | | |
| YIARO4_K221L-> tLip2) | | |
| pCfB9703 (pX-2<-DOD- | Integrative vector | pCfB2899 (X-2-MarkerFree), |
| TyrH(W13L)->) | | BB10, BB4440, BB4151 |
| pCfB10607 (IntD_BvSGT2) | Integrative vector | pCfB6684; BB3908 |
| | | (PrTEFin-> _single), BB5218 |
| | | (BvSGT2_YI_W/O_ATG-U2) |
| pCfB10626 | Integrative vector | pCfB5250 (pIntC_2-TPex20- |
| (IntC2_MjDOD_EvTYH) | | TLip2), BB5049, BB5001 |
| pCfB11394 (X-3_Ptef<- | Integrative vector | pCfB3034 (X-3-MarkerFree), |
| BgGT2<-Tadh1) | | BB8, BB5715 |
| pCfB11421 (X-3_Ptef<- | Integrative vector | pCfB3034 (X-3-MarkerFree), |
| BvSGT2<-Tadh1) | | BB8, BB5052 |
| pCfB11497 (IntE_3- | Integrative vector | pCfB6681; BB5894 |
| TPex20<-BvSGT2_YI<- | | (BvSGT2_YI_W/O_ATG-U1), |
| PrTEFin, <-TPex20<- | | BB5898 (PrTEFin_single-U1), |
| MjDOD_YI- | | BB5893 (TPex20<-MjDOD_YI<- |
| PrGPD::PrTEFin-> | | PrGPD::PrTEFin-> |
| EvTYH_YI-> TLip2) | | EvTYH_YI-> TLip2) |
| pCfB11611 (IntE_4- | Integrative vector | pCfB6679; BB5894 |
| TPex20<-BvSGT2_YI<- | | (BvSGT2_YI_W/O_ATG-U1, |
| PrTEFin, <-TPex20<- | | BB5898 (PrTEFin_single-U1), |
| MjDOD_YI- | | BB5893 (TPex20<-MjDOD_YI<- |
| PrGPD::PrTEFin-> | | PrGPD::PrTEFin-> |
| EvTYH_YI-> TLip2) | | EvTYH_YI-> TLip2) |
| pCfB11113 (IntF_3- | Integrative vector | pCfB6370; BB5894 |
| TPex20<-BvSGT2_YI<- | | (BvSGT2_YI_W/O_ATG-U1), |
| PrTEFin, <-TPex20<- | | BB5898 (PrTEFin_single-U1), |
| MjDOD_YI- | | BB5893 (TPex20<-MjDOD_YI<- |
| PrGPD::PrTEFin-> | | PrGPD::PrTEFin-> |
| EvTYH_YI-> TLip2) | | EvTYH_YI-> TLip2) |
| pCfB12298 (XI- | Integrative vector | pCfB3037 (XI-5-MarkerFree), |
| 5_CcBAHD1) | | BB8, BB6559 |
| pCfB12299 (XI- | Integrative vector | pCfB3037 (XI-5-MarkerFree), |
| 5_CcBAHD2) | | BB8, BB6560 |
| pCfB12300 (XI- | Integrative vector | pCfB3037 (XI-5-MarkerFree), |
| 5_CcBAHD3) | | BB8, BB6561 |
| pCfB12301 (XI- | Integrative vector | pCfB3037 (XI-5-MarkerFree), |
| 5_HpBAHD1) | | BB8, BB6562 |
| pCfB12302 (XI- | Integrative vector | pCfB3037 (XI-5-MarkerFree), |
| 5_HpBAHD2) | | BB8, BB6563 |
| pCfB12303 (XI- | Integrative vector | pCfB3037 (XI-5-MarkerFree), |
| 5_HpBAHD3) | | BB8, BB6564 |
| pCfB12304 (XI- | Integrative vector | pCfB3037 (XI-5-MarkerFree), |
| 5_HpBAHD4) | | BB8, BB6565 |
| pCfB12305 (XI- | Integrative vector | pCfB3037 (XI-5-MarkerFree), |
| 5_HpBAHD5) | | BB8, BB6566 |
| pCfB12306 (XI- | Integrative vector | pCfB3037 (XI-5-MarkerFree), |
| 5_HpBAHD6) | | BB8, BB6567 |
| pCfB12307 (XII- | Integrative vector | pCfB2909 (XII-5-MarkerFree), |
| 5_Ptef_AhAmaSy1_Tadh1) | | BB8, BB6571 |
| pCfB12308 (XII- | Integrative vector | pCfB2909 (XII-5-MarkerFree), |
| 5_Ptef_CqAmaSy1_Tadh1 | | BB8, BB6572 |
| pCfB12309 (XII- | Integrative vector | pCfB2909 (XII-5-MarkerFree), |
| 5_Ptef_CcAmaSy1_Tadh1 | | BB8, BB6573 |
| pCfB12413 (pXI-2-Tadh1<- | Integrative vector | pCfB2903 (XI-2-MarkerFree), |
| AtUGD1<-pTEF1) | | BB8, BB6700 |
| pCfB12418 (IntC_3- | Integrative vector | pCfB6371 (pIntC_3-TPex20- |
| TPex20<-AhAmaSy1_YI<- | | TLip2), BB6739, BB6732 |
| PrGPD) | | |
| pCfB12420 (IntC_3- | Integrative vector | pCfB6371 (pIntC_3-TPex20- |
| TPex20<-CqAmaSy1_YI<- | | TLip2), BB6739, BB6733 |
| PrGPD) | | |
| pCfB12422 (IntC_3- | Integrative vector | pCfB6371 (pIntC_3-TPex20- |
| TPex20<-CcAmaSy1_YI<- | | TLip2), BB6739, BB6734 |
| PrGPD) | | |
| pCfB12441 | Integrative vector | pCfB6681 (pIntE_3-TPex20- |
| (IntE_3-PrTEFin-> | | TLip2), BB6738, BB6735 |
| HpBAHD3_YI-> Tlip2) | | |
| pCfB12430 | Integrative vector | pCfB6684 (pIntD_1-TPex20- |
| (IntD_1-PrTEFin-> | | TLip2), BB6738, BB6735 |
| HpBAHD3_YI-> _Tlip2) | | |
| pCfB12438 | Integrative vector | pCfB6371 (pIntC_3-TPex20- |
| (IntC_3-PrTEFin-> | | TLip2), BB6738, BB6750 |
| CcAmaSy1_YI-> Tlip2) | | |

TABLE 4
Yeast Strains

| Strain | | Parent | Integrated |
|---|---|---|---|
| name | Characteristics | strain | DNA element |
| ST6512 | W29 MATA ku70Δ::PrTEF1-Cas9- | W29 | pCfB6364 (pku70-TPex20- |
| | TTef12::PrGPD-DsdA-TLip2 | | PrTef-_Cas9-Ttef- |
| | | | Lip2-dsdSyn) |
| ST7574 | CEN.PK 113-7D ↑Cas9 | CEN.PK | pCfB2312 (TEF1p-Cas9- |
| | | 113-7D | CYC1t_kanMX) |
| ST9942 | CEN.PK 113-7D ↑Cas9, ↑MjDOD, | ST7574 | pCfB9703 (pX-2<-DOD- |
| | ↑BvCYP76AD1^{W13L} | | TyrH(W13L)->) |
| ST12160 | CEN.PK 113-7D ↑Cas9, ↑MjDOD, | ST9942 | pCfB11421 (X-3_Ptef<- |
| | ↑BvCYP76AD1^{W13L}, ↑BvSGT2 | | BvSGT2<-Tadh1), |
| ST12170 | CEN.PK 113-7D ↑Cas9, ↑MjDOD, | ST9942 | pCfB11394 (X-3_Ptef<- |
| | ↑BvCYP76AD1^{W13L}, ↑BgGT2 | | BgGT2<-Tadh1) |
| ST12603 | MATA ku70Δ::PrTEF1-Cas9- | ST6512 | pCfB8977 (IntE_1- |
| | TTef12::PrGPD-DsdA-TLip2 | | tPex20<-YIARO7_G139S<- |
| | ↑↑↑MjDOD, ↑↑↑EvTYH, | | PrGPD::PrTEFin-> |
| | ↑↑↑BvSGT2, ↑Aro4*, | | YIARO4_K221L-> tLip2), |
| | ↑Aro7*, Δ4hppd | | pCfB11497 (IntE_3-TPex20<- |
| | | | BvSGT2_YI<-PrTEFin, <- |
| | | | TPex20<-MjDOD_YI- |
| | | | PrGPD::PrTEFin-> |
| | | | EvTYH_YI-> TLip2), |
| | | | pCfB11611 (IntE_4- |
| | | | TPex20<-BvSGT2_YI<- |
| | | | PrTEFin, <-TPex20<- |
| | | | MjDOD_YI-PrGPD::PrTEFin-> |
| | | | EvTYH_YI-> TLip2), |
| | | | pCfB11113 (IntF_3- |
| | | | TPex20<-BvSGT2_YI<- |
| | | | PrTEFin, <-TPex20<- |
| | | | MjDOD_YI-PrGPD::PrTEFin-> |
| | | | EvTYH_YI-> TLip2) |
| ST12661 | MATA ku70Δ::PrTEF1-Cas9- | ST12603 | BB6230 |
| | TTef12::PrGPD-DsdA-TLip2 | | (YALI1_B05024g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_B05024g | | |
| ST12662 | MATA ku70Δ::PrTEF1-Cas9- | ST12603 | BB6231 |
| | TTef12::PrGPD-DsdA-TLip2 | | (YALI1_B18845g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_B18845g | | |
| ST12663 | MATA ku70Δ::PrTEF1-Cas9- | ST12603 | BB6232 |
| | TTef12::PrGPD-DsdA-TLip2 | | (YALI1_B18887g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_B18887g | | |
| ST12664 | MATA ku70Δ::PrTEF1-Cas9- | ST12603 | BB6233 |
| | TTef12::PrGPD-DsdA-TLip2 | | (YALI1_B23300g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_B23300g | | |
| ST12665 | MATA ku70Δ::PrTEF1-Cas9- | ST12603 | BB6234 |
| | TTef12::PrGPD-DsdA-TLip2 | | (YALI1_D22997g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_D22997g | | |
| ST12666 | MATA ku70Δ::PrTEF1-Cas9- | ST12603 | BB6235 |
| | TTef12::PrGPD-DsdA-TLip2 | | (YALI1_E23994g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_E23994g | | |
| ST12667 | MATA ku70Δ::PrTEF1-Cas9- | ST12603 | BB6240 |
| | TTef12::PrGPD-DsdA-TLip2 | | (YALI1_F21504g- |
| | ↑↑↑MjDOD, ↑↑↑EvTYH, | | HphSyn-LoxP) |
| | ↑↑↑BvSGT2, ↑Aro4*, ↑Aro7*, | | |
| | Δ4hppd, ΔYALI1_F21504g | | |
| ST13941 | CEN.PK 113-7D ↑Cas9, ↑MjDOD, | ST12160 | pCfB12298 (XI- |
| | ↑BvCYP76AD1^{W13L}, ↑BvSGT2, | | 5_CcBAHD1) |
| | ↑CcBAHD1 | | |
| ST13942 | CEN.PK 113-7D ↑Cas9, ↑MjDOD, | ST12160 | pCfB12299 (XI- |
| | ↑BvCYP76AD1^{W13L}, ↑BvSGT2, | | 5_CcBAHD2) |
| | ↑CcBAHD2 | | |
| ST13943 | CEN.PK 113-7D ↑Cas9, ↑MjDOD, | ST12160 | pCfB12300 (XI- |
| | ↑BvCYP76AD1^{W13L}, ↑BvSGT2, | | 5_CcBAHD3) |
| | ↑CcBAHD3 | | |
| ST13944 | CEN.PK 113-7D ↑Cas9, ↑MjDOD, | ST12160 | pCfB12301 (XI- |
| | ↑BvCYP76AD1^{W13L}, ↑BvSGT2, | | 5_HpBAHD1) |
| | ↑HpBAHD1 | | |
| ST13945 | CEN.PK 113-7D ↑Cas9, ↑MjDOD, | ST12160 | pCfB12302 (XI- |
| | ↑BvCYP76AD1^{W13L}, ↑BvSGT2, | | 5_HpBAHD2) |
| | ↑HpBAHD2 | | |
| ST13946 | CEN.PK 113-7D ↑Cas9, ↑MjDOD, | ST12160 | pCfB12303 (XI- |
| | ↑BvCYP76AD1^{W13L}, ↑BvSGT2, | | 5_HpBAHD3) |
| | ↑HpBAHD3 | | |
| ST13947 | CEN.PK 113-7D ↑Cas9, ↑MjDOD, | ST12160 | pCfB12304 (XI- |
| | ↑BvCYP76AD1^{W13L}, ↑BvSGT2, | | 5_HpBAHD4) |
| | ↑HpBAHD4 | | |
| ST13948 | CEN.PK 113-7D ↑Cas9, ↑MjDOD, | ST12160 | pCfB12305 (XI- |
| | ↑BvCYP76AD1^{W13L}, ↑BvSGT2, | | 5_HpBAHD5) |
| | ↑HpBAHD5 | | |
| ST13949 | CEN.PK 113-7D ↑Cas9, ↑MjDOD, | ST12160 | pCfB12306 (XI- |
| | ↑BvCYP76AD1^{W13L}, ↑BvSGT2, | | 5_HpBAHD6) |
| | ↑HpBAHD6 | | |
| ST13950 | CEN.PK 113-7D ↑Cas9, ↑MjDOD, | ST12170 | pCfB12298 (XI- |
| | ↑BvCYP76AD1^{W13L}, ↑BgGT2, | | 5_CcBAHD1) |
| | ↑CcBAHD1 | | |
| ST13951 | CEN.PK 113-7D ↑Cas9, ↑MjDOD, | ST12170 | pCfB12299 (XI- |
| | ↑BvCYP76AD1^{W13L}, ↑BgGT2, | | 5_CcBAHD2) |
| | ↑CcBAHD2 | | |
| ST13952 | CEN.PK 113-7D ↑Cas9, ↑MjDOD, | ST12170 | pCfB12300 (XI- |
| | ↑BvCYP76AD1^{W13L}, ↑BgGT2, | | 5_CcBAHD3) |
| | ↑CcBAHD3 | | |
| ST13953 | CEN.PK 113-7D ↑Cas9, ↑MjDOD, | ST12170 | pCfB12301 (XI- |
| | ↑BvCYP76AD1^{W13L}, ↑BgGT2, | | 5_HpBAHD1) |
| | ↑HpBAHD1 | | |
| ST13954 | CEN.PK 113-7D ↑Cas9, ↑MjDOD, | ST12170 | pCfB12302 (XI- |
| | ↑BvCYP76AD1^{W13L}, ↑BgGT2, | | 5_HpBAHD2) |
| | ↑HpBAHD2 | | |
| ST13955 | CEN.PK 113-7D ↑Cas9, ↑MjDOD, | ST12170 | pCfB12303 (XI- |
| | ↑BvCYP76AD1^{W13L}, ↑BgGT2, | | 5_HpBAHD3) |
| | ↑HpBAHD3 | | |
| ST13956 | CEN.PK 113-7D ↑Cas9, ↑MjDOD, | ST12170 | pCfB12304 (XI- |
| | ↑BvCYP76AD1^{W13L}, ↑BgGT2, | | 5_HpBAHD4) |
| | ↑HpBAHD4 | | |
| ST13957 | CEN.PK 113-7D ↑Cas9, ↑MjDOD, | ST12170 | pCfB12305 (XI- |
| | ↑BvCYP76AD1^{W13L}, ↑BgGT2, | | 5_HpBAHD5) |
| | ↑HpBAHD5 | | |
| ST13958 | CEN.PK 113-7D ↑Cas9, ↑MjDOD, | ST12170 | pCfB12306 (XI- |
| | ↑BvCYP76AD1^{W13L}, ↑BgGT2, | | 5_HpBAHD6) |
| | ↑HpBAHD6 | | |
| ST13931 | CEN.PK 113-7D ↑Cas9, ↑MjDOD, | ST12160 | pCfB12307 (XII- |
| | ↑BvCYP76AD1^{W13L}, ↑BvSGT2, | | 5_Ptef_AhAmaSy1_Tadh1) |
| | ↑AhAmaSy1 | | |
| ST13932 | CEN.PK 113-7D ↑Cas9, ↑MjDOD, | ST12160 | pCfB12308 (XII- |
| | ↑BvCYP76AD1^{W13L}, ↑BvSGT2, | | 5_Ptef_CqAmaSy1_Tadh1 |
| | ↑CqAmaSy1 | | |
| ST13933 | CEN.PK 113-7D ↑Cas9, ↑MjDOD, | ST12160 | pCfB12309 (XII- |
| | ↑BvCYP76AD1^{W13L}, ↑BvSGT2, | | 5_Ptef_CcAmaSy1_Tadh1 |
| | ↑CcAmaSy1 | | |
| ST13934 | CEN.PK 113-7D ↑Cas9, ↑MjDOD, | ST12170 | pCfB12307 (XII- |
| | ↑BvCYP76AD1^{W13L}, ↑BgGT2, | | 5_Ptef_AhAmaSy1_Tadh1) |
| | ↑AhAmaSy1 | | |
| ST13935 | CEN.PK 113-7D ↑Cas9, ↑MjDOD, | ST12170 | pCfB12308 (XII- |
| | ↑BvCYP76AD1^{W13L}, ↑BgGT2, | | 5_Ptef_CqAmaSy1_Tadh1 |
| | ↑CqAmaSy1 | | |
| ST13936 | CEN.PK 113-7D ↑Cas9, ↑MjDOD, | ST12170 | pCfB12309 (XII- |
| | ↑BvCYP76AD1^{W13L}, ↑BgGT2, | | 5_Ptef_CcAmaSy1_Tadh1 |
| | ↑CcAmaSy1 | | |
| ST13982 | W29 ku70Δ:: Cas9-DsdA, | ST6512 | BB6230 |
| | ΔYALI1_B05024g | | (YALI1_B05024g- |
| | | | HphSyn-LoxP) |
| ST13983 | W29 ku70Δ:: Cas9-DsdA, | ST6512 | BB6231 |
| | ΔYALI1_B18845g | | (YALI1_B18845g- |
| | | | HphSyn-LoxP) |
| ST13984 | W29 ku70Δ:: Cas9-DsdA, | ST6512 | BB6232 |
| | ΔYALI1_B18887g | | (YALI1_B18887g- |
| | | | HphSyn-LoxP) |
| ST13985 | W29 ku70Δ:: Cas9-DsdA, | ST6512 | BB6233 |
| | ΔYALI1_B23300g | | (YALI1_B23300g- |
| | | | HphSyn-LoxP) |
| ST13986 | W29 ku70Δ:: Cas9-DsdA, | ST6512 | BB6234 |
| | ΔYALI1_D22997g | | (YALI1_D22997g- |
| | | | HphSyn-LoxP) |
| ST13987 | W29 ku70Δ:: Cas9-DsdA, | ST6512 | BB6235 |
| | ΔYALI1_E23994g | | (YALI1_E23994g- |
| | | | HphSyn-LoxP) |
| ST13988 | W29 ku70Δ:: Cas9-DsdA, | ST6512 | BB6236 |
| | ΔYALI1_E40502g | | (YALI1_E40502g- |
| | | | HphSyn-LoxP) |
| ST13989 | W29 ku70Δ:: Cas9-DsdA, | ST6512 | BB6237 |
| | ΔYALI1_F02592g | | (YALI1_F02592g- |
| | | | HphSyn-LoxP) |
| ST13990 | W29 ku70Δ:: Cas9-DsdA, | ST6512 | BB6238 |
| | ΔYALI1_F08075g | | (YALI1_F08075g- |
| | | | HphSyn-LoxP) |
| ST13991 | W29 ku70Δ:: Cas9-DsdA, | ST6512 | BB6239 |
| | ΔYALI1_F17788g | | (YALI1_F17788g- |
| | | | HphSyn-LoxP) |
| ST13992 | W29 ku70Δ:: Cas9-DsdA, | ST6512 | BB6240 |
| | ΔYALI1_F21504g | | (YALI1_F21504g- |
| | | | HphSyn-LoxP) |
| ST13993 | W29 ku70Δ:: Cas9-DsdA, | ST6512 | BB6371 |
| | ΔYALI1_E25163g | | (YALI1_E25163g- |
| | | | HphSyn-LoxP) |
| ST13994 | W29 ku70Δ:: Cas9-DsdA, | ST6512 | BB6372 |
| | ΔYALI1_E39796g | | (YALI1_E39796g- |
| | | | HphSyn-LoxP) |
| ST13995 | W29 ku70Δ:: Cas9-DsdA, | ST6512 | BB6373 |
| | ΔYALI1_F03075g | | (YALI1_F03075g- |
| | | | HphSyn-LoxP) |
| ST14115 | CEN.PK 113-7D ↑Cas9, ↑MjDOD, | ST13931 | pCfB12413 (pXI-2- |
| | ↑BvCYP76AD1^{W13L}, ↑BvSGT2, | | Tadh1<-AtUGD1<-pTEF1) |
| | ↑AhAmaSy1, ↑AtUGD1 | | |
| ST14116 | CEN.PK 113-7D ↑Cas9, ↑MjDOD, | ST13932 | pCfB12413 (pXI-2- |
| | ↑BvCYP76AD1^{W13L}, ↑BvSGT2, | | Tadh1<-AtUGD1<-pTEF1) |
| | ↑CqAmaSy1, ↑AtUGD1 | | |
| ST14117 | CEN.PK 113-7D ↑Cas9, ↑MjDOD, | ST13933 | pCfB12413 (pXI-2- |
| | ↑BvCYP76AD1^{W13L}, ↑BvSGT2, | | Tadh1<-AtUGD1<-pTEF1) |
| | ↑CcAmaSy1, ↑AtUGD1 | | |
| ST14118 | CEN.PK 113-7D ↑Cas9, ↑MjDOD, | ST12160 | pCfB12413 (pXI-2- |
| | ↑BvCYP76AD1^{W13L}, ↑BvSGT2, | | Tadh1<-AtUGD1<-pTEF1) |
| | ↑AtUGD1 | | |
| ST11193 | MATA ku70Δ::PrTEF1-Cas9- | ST6512 | pCfB10607 (IntD_BvSGT2), |
| | TTef12::PrGPD-DsdA-TLip2 ↑MjDOD | | pCfB10626 |
| | ↑EvTYH | | (IntC2_MjDOD_EvTYH) |
| | ↑BvSGT2 | | |
| ST14096 | MATA ku70Δ::PrTEF1-Cas9- | ST11193 | pCfB12418 |
| | TTef12::PrGPD-DsdA-TLip2 ↑MjDOD | | (IntC_3-TPex20<- |
| | ↑EvTYH | | AhAmaSy1_YI<-PrGPD) |
| | ↑BvSGT2 ↑AhAmaSy1 | | |
| ST14097 | MATA ku70Δ::PrTEF1-Cas9- | ST11193 | pCfB12420 |
| | TTef12::PrGPD-DsdA-TLip2 ↑MjDOD | | (IntC_3-TPex20<- |
| | ↑EvTYH | | CqAmaSy1_YI<-PrGPD) |
| | ↑BvSGT2 ↑CqAmaSy1 | | |
| ST14098 | MATA ku70Δ::PrTEF1-Cas9- | ST11193 | pCfB12422 |
| | TTef12::PrGPD-DsdA-TLip2 ↑MjDOD | | (IntC_3-TPex20<- |
| | ↑EvTYH | | CcAmaSy1_YI<-PrGPD) |
| | ↑BvSGT2 ↑CcAmaSy1 | | |
| ST14099 | MATA ku70Δ::PrTEF1-Cas9- | ST11193 | pCfB12441 |
| | TTef12::PrGPD-DsdA-TLip2 ↑MjDOD | | (IntE_3-PrTEFin-> |
| | ↑EvTYH | | HpBAHD3_YI-> Tlip2) |
| | ↑BvSGT2 ↑HpBAHD3 | | |
| ST14100 | MATA ku70Δ::PrTEF1-Cas9- | ST12603 | pCfB12418 |
| | TTef12::PrGPD-DsdA-TLip2 | | (IntC_3-TPex20<- |
| | ↑↑↑MjDOD, ↑↑↑EvTYH, | | AhAmaSy1_YI<-PrGPD) |
| | ↑↑↑BvSGT2, ↑Aro4*, ↑Aro7*, | | |
| | Δ4hppd, ↑AhAmaSy1 | | |
| ST14101 | MATA ku70Δ::PrTEF1-Cas9- | ST12603 | pCfB12420 |
| | TTef12::PrGPD-DsdA-TLip2 | | (IntC_3-TPex20<- |
| | ↑↑↑MjDOD, ↑↑↑EvTYH, | | CqAmaSy1_YI<-PrGPD) |
| | ↑↑↑BvSGT2, ↑Aro4*, ↑Aro7*, | | |
| | Δ4hppd, ↑CqAmaSy1 | | |
| ST14102 | MATA ku70Δ::PrTEF1-Cas9- | ST12603 | pCfB12422 |
| | TTef12::PrGPD-DsdA-TLip2 | | (IntC_3-TPex20<- |
| | ↑↑↑MjDOD, ↑↑↑EvTYH, | | |
| | ↑↑↑BvSGT2, ↑Aro4*, ↑Aro7*, | | CcAmaSy1_YI<-PrGPD) |
| | Δ4hppd, ↑CcAmaSy1 | | |
| ST14103 | MATA ku70Δ::PrTEF1-Cas9- | ST12603 | pCfB12430 |
| | TTef12::PrGPD-DsdA-TLip2 | | (IntD_1-PrTEFin-> |
| | ↑↑↑MjDOD, ↑↑↑EvTYH, | | HpBAHD3_YI-> _Tlip2) |
| | ↑↑↑BvSGT2, ↑Aro4*, ↑Aro7*, | | |
| | Δ4hppd, ↑HpBAHD3 | | |
| ST14105 | MATA ku70Δ::PrTEF1-Cas9- | ST12603 | pCfB12438 |
| | TTef12::PrGPD-DsdA-TLip2 | | (IntC_3-PrTEFin-> |
| | ↑↑↑MjDOD, ↑↑↑EvTYH, | | CcAmaSy1_YI-> Tlip2) |
| | ↑↑↑BvSGT2, ↑Aro4*, ↑Aro7*, | | |
| | Δ4hppd, ↑CcAmaSy1 | | |
| ST14157 | MATA ku70Δ::PrTEF1-Cas9- | | |
| | TTef12::PrGPD-DsdA-TLip2, | | |
| | ↑Aro4*, ↑Aro7*, ↑TYR, ↑PGM, | | |
| | ↑AtTT12, ↑Bbx pfk, ↑UDP, | | |
| | ↑MdGSTF6, ↑EvTYH, ↑↑HmoDODA, | | |
| | ↑BgGT2, ↑CqGT1, | | |
| | 4hppdΔ::Pr8UASTEF-AnTYH-tLip2- | | |
| | Pr8UASTEF-MjDOD-tLip2- | | |
| | Pr8UASTEF-BgGT2-tLip2 | | |

TABLE 5
Primer sequences

| Primer name | Sequence |
|---|---|
| PR-6 | CACGCGAUGCACACACCATAGCTTC |
| PR-8 | ATGACAGAUTTGTTTTATATTTGTTG |
| PR-1750 | ACCTGCACUTTGTAATTAAAACTTAGATTAGAT |
| | TG |
| PR-23967 | AGTACTGCAAAAAGUGCTGGTCGGA |
| PR-23995 | ACGTGCGAUAGAGACCGGGTTGGCGGC |
| PR-25055 | AGCATAAGUAGAGACCGGGTTGGCGGC |
| PR-25056 | ACTTATGCUACGCAACTAACATGAATGAATAC |
| | GATATACATC |
| PR-25064 | ATCTGTCAUAAAACAATG |
| | GATCACGCAACATTAGCTAT |
| PR-25065 | CACGCGAUTTAGTATCTTGGAATTGGGATCAA |
| PR-25066 | AGTGCAGGUAAAACAATGAAGGGAACCTACT |
| | ACATCAA |
| PR-25067 | CGTGCGAUTTACGTCGGTCTTTTGGGTGG |
| PR-27830 | ACTTTTTGCAGTACUAACCGCAGGACCACAC |
| | CACTCTGGCCAT |
| PR-27831 | CACGCGAUTTAGTACTTCAGCACGGGGA |
| PR-27948 | AGTGCAGGUAAAACAATGGACGACAAGTCCC |
| | AACA |
| PR-27949 | CGTGCGAUTTAGGTAGACAAACCTCTCA |
| PR-27980 | ATCTGTCAUGCCACAATGAAGGGCACCTACT |
| | ACAT |
| PR-27981 | CGTGCGAUTTACACGTCGGTCTTTTGAG |
| PR-28834 | ACTTTTTGCAGTACUAACCGCAGGACGACAA |
| | GTCTCAGCAGCT |
| PR-28835 | CACGCGAUTTAGGTAGACAGGCCTCGCA |
| PR-29863 | CGTGCGAUTTAGGTAGACAGGCCTCGCAG |
| PR-29970 | ACTTTTTGCAGTACUAACCGCAGGACGACAA |
| | GTCTCAGCAGCTGC |
| PR-31421 (HphSyn-U1 Fwd) | CGTGCGAUTCAGCTGAAGCTTCGTAC |
| PR-31422 (HphSyn-U2 Rev) | CCTGCACUCACTAGTGGATCTGATATCA |
| PR-31983 (YALI1_B05024g- | TACTTCAAGACTTGTGCTGAGG |
| UP Fwd) | |
| PR-31984 (YALI1_B05024g- | ATCGCACGUAATAAATAACGAGTGTGGTGGT |
| UP Rev) | G |
| PR-31985 (YALI1_B05024g- | AGTGCAGGUGTGTCATTTGAATAGATACCATA |
| DW Fwd) | C |
| PR-31986 (YALI1_B05024g- | CTTGAAGGAGCACCTTCAG |
| DW Rev) | |
| PR-31987 (YALI1_B18845g- | CATGGCATGGCGTACAACTG |
| UP Fwd) | |
| PR-31988 (YALI1_B18845g- | ATCGCACGUTTTGAAAGTTATTCAGATGACTG |
| UP Rev) | |
| PR-31989 (YALI1_B18845g- | AGTGCAGGUGATTCCACTTGAGAGTGAATCA |
| DW Fwd) | C |
| PR-31990 (YALI1_B18845g- | CGTCGGGACTGTGTTAGTAATC |
| DW Rev) | |
| PR-31991 (YALI1_B18887g- | ACATTACTGGAAACCCCATCAC |
| UP Fwd) | |
| PR-31992 (YALI1_B18887g- | ATCGCACGUGTTTTTATTTCTGGTGTGTAGGA |
| UP Rev) | G |
| PR-31993 (YALI1_B18887g- | AGTGCAGGUGTGAGAGAGTTCAGTGCTGAG |
| DW Fwd) | |
| PR-31994 (YALI1_B18887g- | CTGGAACGTCTCTGAGAACC |
| DW Rev) | |
| PR-31995 (YALI1_B23300g- | TGACACAGCGACGATGTAATC |
| UP Fwd) | |
| PR-31996 (YALI1_B23300g- | ATCGCACGUTGTGATAGGACTGTTGTTGAC |
| UP Rev) | |
| PR-31997 (YALI1_B23300g- | AGTGCAGGUTCGGACCCGCAGAGCAAA |
| DW Fwd) | |
| PR-31998 (YALI1_B23300g- | AGCAACTGTGTCTAACAAACAC |
| DW Rev) | |
| PR-31999 (YALI1_D22997g- | GCTCATCGCCTTAGCCTAATC |
| UP Fwd) | |
| PR-32000 (YALI1_D22997g- | ATCGCACGUGGAGGGTGGTTGGATCTAG |
| UP Rev) | |
| PR-32001 (YALI1_D22997g- | AGTGCAGGUGAGGATGAACTAATTACTTTAGA |
| DW Fwd) | TTC |
| PR-32002 (YALI1_D22997g- | CGAGGAGGTTCTCCAGATCAC |
| DW Rev) | |
| PR-32003 (YALI1_E23994g- | GAGGTTTCGAGAGCCTTGTTAG |
| UP Fwd) | |
| PR-32004 (YALI1_E23994g- | ATCGCACGUGTTGTGATGGGGTGGGAATAG |
| UP Rev) | |
| PR-32005 (YALI1_E23994g- | AGTGCAGGUCCCGCATCTATTGTATGTAT |
| DW Fwd) | |
| PR-32006 (YALI1_E23994g- | AGTGGAGATTATCACCCAATGG |
| DW Rev) | |
| PR-32007 (YALI1_E40502g- | TCCAGCCCATTGTTCGTGTG |
| UP Fwd) | |
| PR-32008 (YALI1_E40502g- | ATCGCACGUCGCAAATGAAAGTGAATAGCG |
| UP Rev) | |
| PR-32009 (YALI1_E40502g- | AGTGCAGGUGCAGATCGCTAGGGCCACTTCA |
| DW Fwd) | AAA |
| PR-32010 (YALI1_E40502g- | GGAGGATCTCTGCTGTCTTCTACT |
| DW Rev) | |
| PR-32011 (YALI1_F02592g- | CGAAATCTGGCTGTTTCCGTA |
| UP Fwd) | |
| PR-32012 (YALI1_F02592g- | ATCGCACGUAAATACTATCACCCAAAAGAAGA |
| UP Rev) | TCTAAC |
| PR-32013 (YALI1_F02592g- | AGTGCAGGUTCTCATTATGTAACGTCATA |
| DW Fwd) | |
| PR-32014 (YALI1_F02592g- | TGTCCACCTTCGTTGAAGTC |
| DW Rev) | |
| PR-32015 (YALI1_F08075g- | TATGATTGCCAAGGCCAACAGG |
| UP Fwd) | |
| PR-32016 (YALI1_F08075g- | ATCGCACGUGTTGGAGAGACCAAGAGACCGT |
| UP Rev) | |
| PR-32017 (YALI1_F08075g- | AGTGCAGGUTACCCCATAGATATTTATTCAGA |
| DW Fwd) | TTC |
| PR-32018 (YALI1_F08075g- | AGTCTGTCAAGATGGAAGTGGAC |
| DW Rev) | |
| PR-32019 (YALI1_F17788g- | GTACAAGTAGCTTGGAGATGGAC |
| UP Fwd) | |
| PR-32020 (YALI1_F17788g- | ATCGCACGUGGGTAGCAATGGGCAGTGTA |
| UP Rev) | |
| PR-32021 (YALI1_F17788g- | AGTGCAGGUTGTCGTTTCCACGAGCAAAAT |
| DW Fwd) | |
| PR-32022 (YALI1_F17788g- | CAGGTCTGGAGCTTTATTAACCA |
| DW Rev) | |
| PR-32023 (YALI1_F21504g- | CGGGTTACTTGGTTTGTAATCTG |
| UP Fwd) | |
| PR-32024 (YALI1_F21504g- | ATCGCACGUTCTTGAGGAACATCGGATGAG |
| UP Rev) | |
| PR-32025 (YALI1_F21504g- | AGTGCAGGUTAGTCAGTCACCCGTATTAAC |
| DW Fwd) | |
| PR-32026 (YALI1_F21504g- | ACTTTGTGTGTTCTGACGGAG |
| DW Rev) | |
| PR-32308 (YALI1_E25163g- | GACCCTGCTCGTATTCTGC |
| UP Fwd) | |
| PR-32309 (YALI1_E25163g- | ATCGCACGUGGGAAGTGGGCTGGGGAG |
| UP Rev) | |
| PR-32310 (YALI1_E25163g- | AGTGCAGGUGTGCCTTTGACCACCTAC |
| DW Fwd) | |
| PR-32311 (YALI1_E25163g- | ACAGGACCAAAGTGTCGATTG |
| DW Rev) | |
| PR-32312 (YALI1_E39796g- | CACTGGGTTGAGCGATCATC |
| UP Fwd) | |
| PR-32313 (YALI1_E39796g- | ATCGCACGUGGCTGAGGTGTTAGGTGG |
| UP Rev) | |
| PR-32314 (YALI1_E39796g- | AGTGCAGGUAAGGTCTCAGGGTATTTATG |
| DW Fwd) | |
| PR-32315 (YALI1_E39796g- | CAGTAAATTGTCCGCCTCAACT |
| DW Rev) | |
| PR-32316 (YALI1_F03075g- | CCTAGTTTGACGTCTAATACCCAA |
| UP Fwd) | |
| PR-32317 (YALI1_F03075g- | ATCGCACGUGAAGTGGGATATATGAAAGGTG |
| UP Rev) | |
| PR-32318 (YALI1_F03075g- | AGTGCAGGUACGACAAAACAGTAACTAAC |
| DW Fwd) | |
| PR-32319 (YALI1_F03075g- | TCAGTCACTGGGCCAGATG |
| DW Rev) | |
| PR-32543 | AGTGCAGGUAAAACAATGGATAATGGCATCG |
| | AGAA |
| PR-32544 | CGTGCGAUTTACACGACTTCACCATCCA |
| PR-32545 | AGTGCAGGUAAAACAATGACATGCTCTGAGTT |
| | GCT |
| PR-32546 | CGTGCGAUTTACTAGCAACTACTAACGAACT |
| PR-32547 | AGTGCAGGUAAAACAATGGTAGTTTCGAGTG |
| | TGAA |
| PR-32548 | CGTGCGAUTTACTAGGCAATTAGGCACCATT |
| PR-32549 | AGTGCAGGUAAAACAATGAGGGAAAAGATGC |
| | CATG |
| PR-32550 | CGTGCGAUTTACTAGAACCTGCTTACGTACT |
| PR-32551 | AGTGCAGGUAAAACAATGGCTAACGAAATTG |
| | AGAA |
| PR-32552 | CGTGCGAUTTACTAAGCAACTTCCAGGTTCA |
| PR-32553 | AGTGCAGGUAAAACAATGGTGAATCAAGAGG |
| | GGAT |
| PR-32554 | CGTGCGAUTTAGCCCATAGAGACGAAGT |
| PR-32555 | AGTGCAGGUAAAACA ATG |
| | GAGATAAGTTCTTTCAA |
| PR-32556 | CGTGCGAUTTATACCTGCATGATCTCTT |
| PR-32557 | AGTGCAGGUAAAACAATGGTGAACCAAGAAG |
| | GGAC |
| PR-32558 | CGTGCGAUTCAGACAACAGTTACGAAGC |
| PR-32559 | AGTGCAGGUAAAACAATGCAGAATTACGAGA |
| | AAAG |
| PR-32560 | CGTGCGAUTTACTAGAACAATGATGAAGAGA |
| PR-32630 | CGTGCGAUTCACACGATCAATAATTGCTG |
| PR-32631 | AGTGCAGGUAAAACAATGAGCCACAATAAAG |
| PR-32632 | CGTGCGAUCTAAGATCCGATCAGCTG |
| PR-32633 | AGTGCAGGUAAAACAATGTCACAGAACAAGG |
| PR-32634 | CGTGCGAUTCATCCAAGTAATTGC |
| PR-32635 | AGTGCAGGUAAAACAATGAGTTCTAATCAGG |
| PR-33125 | CGTGCGAUTCAAGCCACTGCAGGC |
| PR-33126 | AGTGCAGGUAAAACAATGGTAAAGATTTGCTG |
| PR-33144 | ACTTTTTGCAGTACUAACCGCAGGTGAACCA |
| | GGAAGGAATC |
| PR-33145 | ACACGCGAUTTAGCCCATGGAGACAAAATTC |
| PR-33146 | ACGTGCGAUTCAGACGATGAGGAGTTGC |
| PR-33147 | AGTGCAGGUGCCACAATGTCCCACAACAAGG |
| | AGTCG |
| PR-33150 | ACGTGCGAUTTAGCTTCCAATCAGC |
| PR-33151 | AGTGCAGGUGCCACAATGTCCCAAAATAAAG |
| | AC |
| PR-33154 | ACGTGCGAUCTAGCCAAGCAGCTGTCTG |
| PR-33155 | AGTGCAGGUGCCACAATGTCGTCCAACCAGG |
| | AC |
| PR-33156 | ACTTTTTGCAGTACUAACCGCAGTCGTCCAAC |
| | CAGGAC |
| PR-33237 | ACGTGCGAUAGAGACCGGGTTGGCGGCGC |
| PR-33237 | ACACGCGAUGACGCAGTAGGATGTCC |
| PR-33238 | ACCTGCACUTTGTAATTAAAACTTAGATTAGAT |
| | TG |

### REFERENCES

Holkenbrink, Carina, Marie I. Dam, Kanchana R. Kildegaard, Johannes Beder, Jonathan Dahlin, David Doménech Belda, and Irina Borodina. 2018. “EasyCloneYALI: CRISPR/Cas9-Based Synthetic Toolbox for Engineering of the Yeast Yarrowia Lipolytica.” Biotechnology Journal 13 (9): 1700543. https://doi.org/10.1002/biot.201700543.
Imamura, Tomohiro, Noriyoshi Isozumi, Yasuki Higashimura, Akio Miyazato, Hiroharu Mizukoshi, Shinya Ohki, and Masashi Mori. 2019. “Isolation of Amaranthin Synthetase from Chenopodium Quinoa and Construction of an Amaranthin Production System Using Suspension-Cultured Tobacco BY-2 Cells.” Plant Biotechnology Journal 17 (5): 969-81. https://doi.org/10.1111/pbi.13032.
Jessop-Fabre, Mathew M., Tadas Jakoc̆iūnas, Vratislav Stovicek, Zongjie Dai, Michael K. Jensen, Jay D. Keasling, and Irina Borodina. 2016. “EasyClone-MarkerFree: A Vector Toolkit for Marker-Less Integration of Genes into Saccharomyces Cerevisiae via CRISPR-Cas9.” Biotechnology Journal. https://doi.org/10.1002/biot.201600147. Khan, Mohammad Imtiyaj, and P. Giridhar. 2015. “Plant Betalains: Chemistry and Biochemistry.” Phytochemistry 117 (September): 267-95. https://doi.org/10.1016/j.phytochem.2015.06.008.
Lubuta, Patrice, Mhairi Workman, Eduard J. Kerkhoven, and Christopher T. Workman. 2019. “Investigating the Influence of Glycerol on the Utilization of Glucose in Yarrowia Lipolytica Using RNA-Seq-Based Transcriptomis.” G3 (Bethesda, Md.) 9 (12): 4059-71. https://doi.org/10.1534/g3.119.400469.
Milne, N., P. Thomsen, N. Mølgaard Knudsen, P. Rubaszka, M. Kristensen, and I. Borodina. 2020. “Metabolic Engineering of Saccharomyces Cerevisiae for the de Novo Production of Psilocybin and Related Tryptamine Derivatives.” Metabolic Engineering 60 (July): 25-36. https://doi.org/10.1016/j.ymben.2019.12.007.
Moyo, Batanai, and Stanley Mukanganyama. 2015. “The Anticandidal and Toxicity Properties of Lampranthus francisci.” Journal of Mycology 2015 (October): e898202. https://doi.org/10.1155/2015/898202.
Oka, Takuji, and Yoshifumi Jigami. 2006. “Reconstruction of de Novo Pathway for Synthesis of UDP-Glucuronic Acid and UDP-Xylose from Intrinsic UDP-Glucose in Saccharomyces cerevisiae.” The FEBS Journal 273 (12): 2645-57. https://doi.org/10.1111/j.1742-4658.2006.05281.x.
Pérez, Gabriel, Laura Fariña, Marianne Barquet, Eduardo Boido, Carina Gaggero, Eduardo Dellacassa, and Francisco Carrau. 2011. “A Quick Screening Method to Identify β-Glucosidase Activity in Native Wine Yeast Strains: Application of Esculin Glycerol Agar (EGA) Medium.” World Journal of Microbiology and Biotechnology 27 (1): 47-55. https://doi.org/10.1007/s11274-010-0425-4.
Piattelli, Mario, and Luigi Minale. 1964. “Pigments of Centrospermae-I.: Betacyanins from Phyllocactus hybridus Hort. And Opuntia ficus-indica Mill.” Phytochemistry 3 (2): 307-11. https://doi.org/10.1016/S0031-9422(00)88056-5.
Polturak, Guy, Uwe Heinig, Noam Grossman, Maor Battat, Dena Leshkowitz, Sergey Malitsky, Ilana Rogachev, and Asaph Aharoni. 2018. “Transcriptome and Metabolic Profiling Provides Insights into Betalain Biosynthesis and Evolution in Mirabilis jalapa.” Molecular Plant 11 (1): 189-204. https://doi.org/10.1016/j.molp.2017.12.002.
Sáez-Sáez, Javier, Guokun Wang, Eko Roy Marella, Suresh Sudarsan, Marc Cernuda Pastor, and Irina Borodina. 2020. “Engineering the Oleaginous Yeast Yarrowia lipolytica for High-Level Resveratrol Production.” Metabolic Engineering 62 (November): 51-61. https://doi.org/10.1016/j.ymben.2020.08.009.
Schliemann, Willibald, and Dieter Strack. 1998. “Intramolecular Stabilization of Acylated Betacyanins.” Phytochemistry 49 (2): 585-88. https://doi.org/10.1016/S0031-9422(98)00047-8.
Sutor, Katarzyna, and Slawomir Wybraniec. 2020. “Identification and Determination of Betacyanins in Fruit Extracts of Melocactus Species.” Journal of Agricultural and Food Chemistry 68 (41): 11459-67. https://doi.org/10.1021/acs.jafc.0c04746.
Wybraniec, Slawomir, Barbara Nowak-Wydra, Katarzyna Mitka, Piotr Kowalski, and Yosef Mizrahi. 2007. “Minor Betalains in Fruits of Hylocereus Species.” Phytochemistry 68 (2): 251-59. https://doi.org/10.1016/j.phytochem.2006.10.002.
WO2022253815A1, Borodina et al., METHODS FOR PRODUCING BETALAINS IN YEAST (2022)

1. A yeast cell producing a modified betalain in the presence of betanin, isobetanin and/or glycosylated cyclo-DOPA, wherein the modified betalain is selected from the group consisting of acylated betalains and di-glycosylated betalains, and wherein the yeast cell expresses:
a. a heterologous BAHD acyltransferase capable of acylating a betalain and/or glycosylated cyclo-DOPA, thereby producing an acylated betalain; and/or
b. a heterologous glycosyltransferase capable of glycosylating a betalain and/or glycosylated cyclo-DOPA, thereby producing a di-glycosylated betalain.

2. The yeast cell according to any one of the preceding claims, wherein:
a. the acylated betalain is an acylated betacyanin, such as an acylated betanin and/or isobetanin, such as a betanin and/or isobetanin acylated at position 4 of the glycosyl moiety of betanin and/or isobetanin, such as phyllocactin II and/or isophyllocactin II; and/or
b. the acylated betalain is an acylated betacyanin, such as an acylated betanin and/or isobetanin, such as a betanin and/or isobetanin acylated at position 6 of the glycosyl moiety of betanin and/or isobetanin, such as phyllocactin and/or isophyllocactin; and/or
c. the di-glycosylated betalain is a di-glycosylated betacyanin, such as a glycosylated betanin and/or isobetanin, such as a betanin and/or isobetanin glycosylated at position 2 of the glycosyl moiety of betanin and/or isobetanin, such as a betanin and/or isobetanin glucosylated at position 2 of the glycosyl moiety of betanin and/or isobetanin, such as bougainvillein-r-I and/or isobougainvilleinen-r-I; and/or
d. the yeast cell is supplemented with and/or capable of producing UDP-glucuronic acid and the di-glycosylated betalains is a di-glycosylated betacyanin, such as a glycosylated betanin and/or isobetanin, such as a betanin and/or isobetanin glycosylated at position 2 of the glycosyl moiety of betanin and/or isobetanin, such as a betanin and/or isobetanin glucoronidated at position 2 of the glycosyl moiety of betanin and/or isobetanin, such as amaranthin and/or isoamaranthin.

3. The yeast cell according to any one of the preceding claims, wherein:
a. the BAHD acyltransferase is native to a plant, such as a plant of the genus Hylocereus, such as Hylocereus polyrhizus, optionally wherein the BAHD acyltransferase is HpBAHD3 as set forth in SEQ ID NO: 29 or a functional variant thereof having at least 70% sequence identity thereto; and/or
b. the glycosyltransferase is native to a plant, such as a plant of the genus Amaranthus, Chenopodium and/or Celosia, such as Amaranthus hypochondriacus, Chenopodium quinoa or Celosia cristata, optionally the wherein the glycosyltransferase is AhAmaSy1 as set forth in SEQ ID NO: 38, CqAmaSy1 as set forth in SEQ ID NO: 41 and/or CcAmaSy1 as set forth in SEQ ID NO: 44 or a functional variant thereof having at least 70% sequence identity to SEQ ID NO: 38, SEQ ID NO: 41 or SEQ ID NO 44.

4. The yeast cell according to any one of the preceding claims, wherein the genus of said yeast cell is selected from the group consisting of Saccharomyces, Pichia, Yarrowia, Kluyveromyces, Candida, Rhodotorula, Rhodosporidium, Cryptococcus, Trichosporon or Lipomyces, such as wherein the yeast is of a species selected from the group consisting of Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan or Yarrowia lipolytica, preferably wherein the yeast is Saccharomyces cerevisiae or Yarrowia lipolytica.

5. The yeast cell according to any one of the preceding claims, wherein said yeast cell further expresses:
a. a first heterologous enzyme which is a tyrosine hydroxylase (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
whereby the yeast cell produces betanin and/or isobetanin;
optionally wherein:
a. the TYH is native to a plant, such as of the genus Abronia, Acleisanthes, Basella, Beta, Cleretum, Ercilla, Mirabilis, Optunia or Phytolacca, such as Abronia nealleyi, Acleisanthes obtusa, Basella alba, Beta vulgaris, Cleretum bellidiforme, Ercilla volubilis, Mirabilis multiflora, Optunia ficus-indic, or Phytolacca dioica, or a functional variant thereof having at least 80% identity thereto; and/or
b. the DOD is native to a plant, such as of the genus Amaranthus, Beta, Bougainvillea, Mirabilis Phytolacca, Portulaca, Spinacia or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea or Suaeda salsa, or a functional variant thereof having at least 80% identity thereto; and/or
c. the enzyme having glycosyltransferase activity is native to a plant, such as of the genus Abronia, Beta, Bougainvillea, Ercilla or Portacula, such as Abronia nealleyi, Beta vulgaris, Bougainvillea glabra, Ercilla volubilis or Portacula grandiflora, or a functional variant thereof having at least 80% identity thereto;
optionally wherein:
a. the TYH is selected from:
i. Abronia nealleyi TYH, such as AnTYH as set forth in SEQ ID NO: 49 or a functional variant thereof having at least 70% sequence identity thereto;
ii. Ercilla volubilis TYH, such as EvTYH as set forth in SEQ ID NO: 6 or a functional variant thereof having at least 70% sequence identity thereto;
iii. Beta vulgaris TYH, such as BvCYP76AD1^{W13L} as set forth in SEQ ID NO: 4 or a functional variant thereof having at least 70% sequence identity thereto;
and/or
b. the DOD is selected from:
i. Mirabilis jalapa DOD, such as MjDOD as set forth in SEQ ID NO: 1 or a functional variant thereof having at least 70% sequence identity thereto;
ii. Portulaca grandiflora DOD, such as PgDOD as set forth in SEQ ID NO: 55 or a functional variant thereof having at least 70% sequence identity thereto;
iii. Bougainvillea glabra DOD, such as BgDOD2 as set forth in SEQ ID NO: 52 or a functional variant thereof having at least 70% sequence identity thereto; and
c. the enzyme having glycosyltransferase activity is selected from:
i. Chenopodium quinoa glycosyltransferase CqSGT2 set forth in SEQ ID NO: 57, or a functional variant thereof having at least 70% sequence identity thereto;
ii. Beta vulgaris glycosyltransferase BvSGT2 set forth in SEQ ID NO: 8, or a functional variant thereof having at least 70% sequence identity thereto;
iii. Bougainvillea glabra glycosyltransferase BgGT2 set forth in SEQ ID NO: 11, or a functional variant thereof having at least 70% sequence identity thereto; and
iv. Beta vulgaris glycosyltransferase BvSGT4 set forth in SEQ ID NO: 59, or a functional variant thereof having at least 70% sequence identity thereto.

6. The yeast cell according to any one of the preceding claims, wherein said yeast cell comprises a mutation resulting in reduced activity in one or more genes encoding one or more native β-glucosidases,
wherein the activity is reduced as compared the activity in a yeast cell in which the one or more native β-glucosidases have not been mutated, but otherwise identical, when cultivated in the same conditions,
optionally wherein said one or more genes are selected from the group consisting of:
i) YALI1_B23300g (SEQ ID NO: 61);
ii) YALI1_F21504g (SEQ ID NO: 62);
iii) YALI1_B05024g (SEQ ID NO: 63);
iv) YALI1_B18845g (SEQ ID NO: 64);
v) YALI1_B18887g (SEQ ID NO: 65);
vi) YALI1_D22997g (SEQ ID NO: 66);
vii) YALI1_E40502g (SEQ ID NO: 67);
viii) YALI1_F02592g (SEQ ID NO: 68);
ix) YALI1_F08075g (SEQ ID NO: 69);
x) YALI1_F17788g (SEQ ID NO: 70);
xi) YALI1_E23994g (SEQ ID NO: 79);
xii) YALI1_E39796g (SEQ ID NO: 80);
xiii) YALI1_F03075g (SEQ ID NO: 81);
xiv) YALI1_E25163g (SEQ ID NO: 82);
xv) YALI0F16027g (SEQ ID NO: 217);
xvi) YALI0B14289g (SEQ ID NO: 218); and
xvii) YALI0F05390g (SEQ ID NO: 219);
preferably wherein said one or more genes are selected from the group consisting of YALI1_D22997g (SEQ ID NO: 66), YALI1_B23300g (SEQ ID NO: 61), YALI1_F21504g (SEQ ID NO: 62), YALI1_F08075g (SEQ ID NO: 69), YALI1_B18845g (SEQ ID NO: 64), YALI1_E23994g (SEQ ID NO: 82), YALI0F16027g (SEQ ID NO: 217), YALI0B14289g (SEQ ID NO: 218) and YALI0F05390g (SEQ ID NO: 219).

7. The yeast cell according to any one of the preceding claims, wherein said yeast cell comprises:
a. a mutation resulting in reduced activity of 4-hydroxyphenylpyruvate dioxygenase (4-HPPD), wherein the activity is reduced as compared the activity in a yeast cell in which 4-HPPD has not been mutated, but otherwise identical, when cultivated in the same conditions;
b. a mutation resulting in increased production of L-tyrosine, wherein the production is increased as compared the production in a yeast cell not carrying said mutation resulting in increased production of L-tyrosine, but otherwise identical, when cultivated in the same conditions; and/or
c. a mutation resulting in less sensitivity to feedback inhibition by aromatic amino acids, wherein the sensitivity is reduced as compared the sensitivity in a yeast cell not carrying said mutation resulting in less sensitivity to feedback inhibition by aromatic amino acids, but otherwise identical, when cultivated in the same conditions.

8. The yeast cell according to any one of the preceding claims, wherein said yeast cell comprises a mutation in:
a. 3-deoxy-7-phosphoheptulonate synthase, such as in Yarrowia lipolytica Aro4 or Saccharomyces cerevisiae Aro4, such as wherein amino acid no. 221 of Yarrowia lipolytica Aro4 is substituted with a leucine or wherein amino acid no. 229 of Saccharomyces cerevisiae Aro4 is substituted with leucine; and/or
b. chorismate mutase, such as in Yarrowia lipolytica Aro7 or Saccharomyces cerevisiae Aro7, such as wherein amino acid no. 139 of Yarrowia lipolytica Aro7 is substituted with a serine or wherein amino acid no. 141 of Saccharomyces cerevisiae Aro7 is substituted with a serine.

9. The yeast cell according to any one of the preceding claims, wherein said yeast cell comprises as a partial or total deletion of the gene encoding 4-HPPD.

10. A method of producing a modified betalain in the presence of betanin, isobetanin and/or glycosylated cyclo-DOPA in a yeast cell, wherein the modified betalain is selected from the group consisting of acylated betalains and di-glycosylated betalains, said method comprising the steps of:
a. incubating a yeast cell in a medium;
b. expressing in said yeast cell:
i. a heterologous BAHD acyltransferase capable of acylating a betalain and/or glycosylated cyclo-DOPA, thereby obtaining an acylated betalain; and/or
ii. a heterologous glycosyltransferase capable of glycosylating a betalain and/or glycosylated cyclo-DOPA, thereby obtaining a di-glycosylated betalain.

11. The method according to claim 10, wherein:
a. the method further comprises the step of recovering the modified betalain;
b. the yeast cell is as defined in any one of claims 1 to 9;
c. the acylated betalain is as defined in claim 2;
d. the di-glycosylated betalain is as defined in claim 2;
e. the BAHD acyltransferase is as defined in claim 3;
f. the glycosyltransferase is as defined claim 3; and/or
g. the medium is supplemented with:
i. L-tyrosine;
ii. betanin;
iii. isobetanin;
iv. UDP-glucuronic acid; and/or
v. glycosylated cyclo-DOPA.

12. The method according to any one of claims 10 to 11, wherein the yeast cell further expresses:
a. a first heterologous enzyme which is a tyrosine hydroxylase (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
whereby the yeast cell produces a glycosylated betalains, such as betanin and/or isobetanin.

13. The method according to claim 12, wherein the TYH, the DOD, and/or the enzyme having glycosyltransferase activity is as defined in any one of the preceding claims.

14. Use of a glycosyltransferase:
a. in a method of producing a di-glycosylated betalain, such as a glycosylated betanin and/or isobetanin;
b. to catalyse the glycosylation of glycosylated cyclo-DOPA and/or a betalain, such as betanin and/or isobetanin, thereby obtaining a di-glycosylated betalain; and/or
c. for glucuronidation of position 2 of the glycosyl moiety of betanin and/or isobetanin.

15. The use according to claim 14, wherein the method of producing a di-glycosylated betalain; the catalyzing of the glycosylation of glycosylated cyclo-DOPA and/or a betalain; and/or the glucuronidation of position 2 of the glycosyl moiety of betanin and/or isobetanin is performed in a yeast cell.

16. The use according to any one of claims 14 to 15, wherein:
a. the glycosyltransferase is as defined in claim 3;
b. the acylated betalain is as defined in claim 2;
c. the di-glycosylated betalain is as defined in claim 2.

17. A method of increasing the titer and/or purity of a betalain produced by a yeast cell, wherein the betalain is selected from the group consisting of glycosylated betalains, acylated betalains and di-glycosylated betalains, said method comprising the steps of:
a. providing a yeast cell comprising a mutation resulting in reduced activity of one or more native β-glucosidases, said yeast cell producing a glycosylated betalain;
b. incubating said yeast cell in a medium;
c. expressing in said yeast cell:
i. a first heterologous enzyme which is a tyrosine hydroxylase (TYH) capable of hydroxylating L-tyrosine and oxidizing L-DOPA;
ii. a second heterologous enzyme which is a 4,5-DOPA extradiol dioxygenase (DOD); and
iii. a third heterologous enzyme having glycosyltransferase activity;
d. optionally, further expressing in said yeast cell:
i. a heterologous BAHD acyltransferase capable of acylating a betalain and/or glycosylated cyclo-DOPA; and/or
ii. a heterologous glycosyltransferase capable of glycosylating a betalain and/or glycosylated cyclo-DOPA;
e. optionally recovering the modified betalain;
thereby obtaining a betalain with an improved purity and/or titer as compared to the purity and/or titer of a betalain produced by a yeast cell expressing said TYH, DOD, enzyme having glycosyltransferase activity and optionally said BAHD acyltransferase and/or glycosyltransferase, but not comprising a mutation resulting in reduced activity of one or more native β-glucosidases, and cultivated under the same conditions.
